(19) United States
(12) Patent Application Publication
(10) Pub. No.: US 2023/0127944 A1
(43) Pub. Date: Apr. 27, 2023

(54) BROAD-SPECTRUM CARBAPENEMS

(71) Applicant: Venatorx Pharmaceuticals, Inc., Malvern, PA (US)

(72) Inventors: Christopher J. BURNS, Malvern, PA (US); Guo-Hua CHU, Exton, PA (US); Jodie HAMRICK, New Holland, PA (US); Steven A. BOYD, Chester Springs, PA (US); Stephen M. CONDON, Glenmoore, PA (US)

(21) Appl. No.: 17/778,199

(22) PCT Filed: Sep. 17, 2020
(86) PCT No.: PCT/US2020/051163

§ 371 Date: May 19, 2022

Related U.S. Application Data
(60) Provisional application No. 62/938,837, filed on Nov. 21, 2019.

(51) Int. Cl. A61K 31/407; A61K 9/20; A61K 9/48; A61K 9/00; C07D 477/20; A61K 31/69; A61K 31/506; A61K 31/4439; A61K 31/454; A61P 31/04

(52) U.S. Cl. CPC A61K 31/407; A61K 9/2059; A61K 9/2054; A61K 9/2018; A61K 9/2013; A61K 9/4825; A61K 9/4858; A61K 9/0019; C07D 477/20; A61K 31/69; A61K 31/506; A61K 31/4439; A61K 31/454; A61P 31/04

(57) ABSTRACT

The present disclosure provides broad-spectrum carbapenem derivatives and pharmaceutical compositions useful in the treatment of bacterial infections and methods for treating such infections using such derivatives and/or compositions.

### CROSS-REFERENCE

This application claims the benefit of U.S. Provisional Application Ser. No. 62/938,837 filed Nov. 21, 2019, which is hereby incorporated by reference in its entirety.

### STATEMENT AS TO FEDERALLY SPONSORED RESEARCH

This invention was made with government support under SBIR Grant numbers R43AI120392 and R44AI120392 awarded by the National Institutes of Health (NIH). The government has certain rights in the invention.

### BACKGROUND OF THE INVENTION

Antibiotics are the most effective drugs for curing bacteria-related infectious diseases clinically. They are incredibly valuable therapeutic options that are currently losing efficacy due to the evolution and spread of drug resistance genes. A dramatic increase in the prevalence of infections caused by Multi Drug Resistant (MDR) Gram positive and Gram negative microorganisms is now occurring, both in the hospital and in the community. Carbapenem beta-lactams face two important issues with regard to their utility: (1) inactivity against MDR Gram positive bacteria (e.g. methicillin-resistant Staphylococcus aureus (MRSA), vancomycin-resistant Staphylococcus aureus (VRSA)), and (2) clinical failure due to spread of beta-lactamase-producing Gram negative Enterobacteriaceae and Pseudomonas aeruginosa. There is a growing consensus on the need for agents with activity against these MDR bacteria as first-line empiric treatment of many nosocomial infections, such as pneumonia, skin and soft tissue, intra-abdominal, and complicated urinary tract infections. These infections are caused by either Gram positive or negative pathogens, or a polymicrobial mix potentially including anaerobes. There is a significant need to develop a safe and bactericidal agent with the widest known spectrum of activity, including against MDR Gram-positives, Gram-negatives, and anaerobes, to treat empirically this wide range of infections.

### SUMMARY OF THE INVENTION

Described herein are carbapenem compounds that provide significant antibacterial activity in vitro against a range of Gram positive and Gram negative bacteria.

Also disclosed herein is a compound of Formula (III), or a pharmaceutically acceptable salt, solvate, or stereoisomer thereof:

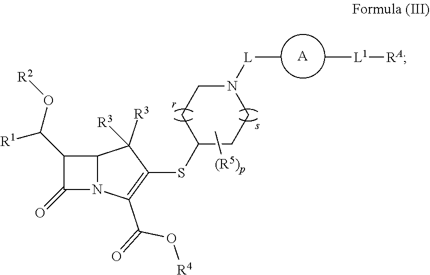

Ring A is cycloalkyl optionally substituted with one, two, or three oxo, halogen, C₁-C₆ alkyl, C₁-C₆ haloalkyl, —OR^{a}, —CN, —NO₂, —NR^{c}R^{d}, —S(═O)₂R^{b}, —S(═O)₂NR^{c}R^{d}, —C(═O)OR^{a}, —C(═O)R^{b}, —C(═O)NR^{c}R^{d}, —NR^{c}C(═NR^{c})NR^{c}R^{d}, or —NR^{c}C(═NR^{c})R^{b};
L¹ is a bond or C₁-C₆ alkylene optionally substituted with halogen, —OR^{a}, or —NR^{c}R^{d};
R^{A} is halogen, C₁-C₆ alkyl, C₂-C₆ alkenyl, C₂-C₆ alkynyl, C₁-C₆ haloalkyl, C₁-C₆ hydroxyalkyl, —OR¹⁰, —CN, —NO₂, —S(═O)₂R¹⁰, —S(═O)₂NR¹⁰R¹¹, —C(═O)OR¹⁰, —C(═O)NR¹⁰R¹¹, —NR¹⁰C(═NR¹¹)NR¹⁰R¹¹, —C(═NR¹⁰)R¹⁰, —NR¹⁰C(═NR¹¹)R¹⁰, —NR¹⁰(CR¹²R¹³)_{w}NR¹⁰R¹¹, —NR¹⁰C(═NR¹¹)NR¹⁰(CR¹²R¹³)_{w}NR¹⁰R¹¹, —C(═NR¹¹)NR¹⁰R¹¹, C₃-C₈ cycloalkyl, C₂-C₈ heterocycloalkyl, aryl, or heteroaryl; wherein each alkyl, alkenyl, alkynyl, cycloalkyl, heterocycloalkyl, aryl, and heteroaryl is independently optionally substituted with one, two, or three R^{A1};
each R^{A1} is independently halogen, C₁-C₆ alkyl, C₁-C₆ haloalkyl, —OR^{a}, —CN, —NO₂, —NR^{c}R^{d}, —S(═O)₂R^{b}, —S(═O)₂NR^{c}R^{d}, —C(═O)OR^{a}, —C(═O)R^{b}, —C(═O)NR^{c}R^{d}, —NR^{c}C(═NR)NR^{c}R^{d}, or —NR^{c}C(═NR^{c})R^{b};
L is a bond or C₁-C₆ alkylene optionally substituted with halogen, —OR^{a}, or —NR^{c}R^{d};
R¹ is C₁-C₆ alkyl, C₁-C₆ haloalkyl, C₂-C₆ alkenyl, or C₂-C₆ alkynyl; R² is hydrogen, C₁-C₆ alkyl, C₁-C₆ haloalkyl, C₂-C₆ alkenyl, C₂-C₆ alkynyl, or —Si(R^{b})₃; each R³ is independently hydrogen, C₁-C₆ alkyl, C₁-C₆ haloalkyl, C₂-C₆ alkenyl, C₂-C₆ alkynyl, or —OR^{a};
R⁴ is hydrogen, C₁-C₆ alkyl, C₁-C₆ haloalkyl, C₂-C₆ alkenyl, C₂-C₆ alkynyl, C₃-C₈ cycloalkyl, C₂-C₈ heterocycloalkyl, aryl, heteroaryl, —(C₁-C₆ alkyl)(C₃-C₈ cycloalkyl), —(C₁-C₆ alkyl)(C₂-C₈ heterocycloalkyl), —(C₁-C₆ alkyl)(aryl), or —(C₁-C₆ alkyl)(heteroaryl);
each R⁵ is independently halogen, C₁-C₆ alkyl, C₁-C₆ haloalkyl, C₂-C₆ alkenyl, C₂-C₆ alkynyl, —OR^{a}, —CN, —NO₂, —S(═O)₂R^{b}, —S(═O)₂NR^{c}R^{d}, —C(═O)OR^{a}, —C(═O)R^{b}, —C(═O)NR^{c}R^{d}, —NR^{c}R^{d}, C₃-C₈ cycloalkyl, or C₂-C₈ heterocycloalkyl;
or two R⁵ on the same carbon are taken together to form an oxo;
each R¹⁰ and R¹¹ is independently hydrogen, C₁-C₆ alkyl, C₁-C₆ haloalkyl, C₂-C₆ alkenyl, C₂-C₆ alkynyl, C₃-C₈ cycloalkyl, C₂-C₈ heterocycloalkyl, aryl, heteroaryl, —(C₁-C₆ alkyl)(C₃-C₈ cycloalkyl), —(C₁-C₆ alkyl)(C₂-C₈ heterocycloalkyl), —(C₁-C₆ alkyl)(aryl), or —(C₁-C₆ alkyl)(heteroaryl);
or R¹⁰ and R¹¹ are taken together with the nitrogen to which they are attached to form a C₂-C₈ heterocycloalkyl;
each R¹² and R¹³ is independently hydrogen, halogen, —CN, —OR^{a}, —NR^{c}R^{d}, C₁-C₆ alkyl, C₁-C₆ haloalkyl, C₂-C₆ alkenyl, C₂-C₆ alkynyl, C₃-C₈ cycloalkyl, C₂-C₈ heterocycloalkyl, aryl, or heteroaryl;
each R^{a} is independently hydrogen, C₁-C₆ alkyl, C₁-C₆ haloalkyl, C₂-C₆ alkenyl, C₂-C₆ alkynyl, C₃-C₈ cycloalkyl, or C₂-C₈ heterocycloalkyl;
each R^{b} is independently C₁-C₆ alkyl, C₁-C₆ haloalkyl, C₂-C₆ alkenyl, C₂-C₆ alkynyl, C₃-C₈ cycloalkyl, or C₂-C₈ heterocycloalkyl;
each R^{c} and R^{d} are independently hydrogen, C₁-C₆ alkyl, C₁-C₆ haloalkyl, C₂-C₆ alkenyl, C₂-C₆ alkynyl, C₃-C₈ cycloalkyl, or C₂-C₈ heterocycloalkyl;
or R^{c} and R^{d} are taken together with the nitrogen to which they are attached to form a C₂-C₈ heterocycloalkyl;
w is 2-4;
r is 1-2;
s is 0-2; and
p is 0-5.

Disclosed herein is a compound of Formula (I), or a pharmaceutically acceptable salt, solvate, or stereoisomer thereof:

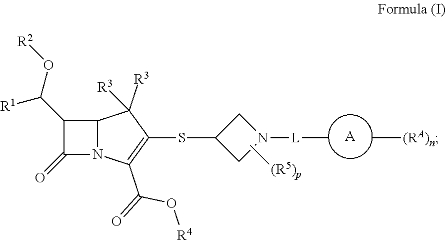

wherein
Ring A is cyclobutyl or cyclopentyl;
each R^{A} is independently halogen, C₁-C₆ alkyl, C₂-C₆ alkenyl, C₂-C₆ alkynyl, C₁-C₆ haloalkyl, C₁-C₆ hydroxyalkyl, —(CR¹²R¹³)ᵥOR¹⁰, —(CR¹²R¹³)ᵥNR¹⁰R¹¹, —CN, —NO₂, —(CR¹²R¹³)ᵥS(═O)₂R¹⁰, —(CR¹²R¹³)ᵥS(═O)₂NR¹⁰R¹¹, —(CR¹²R¹³)ᵥC(═O)OR¹⁰, —(CR¹²R¹³)ᵥC(═O)NR¹⁰R¹¹, —(CR¹²R¹³)ᵥNR¹⁰C(═NR¹¹)NR¹⁰R¹¹, —(CR¹²R¹³)ᵥC(═NR¹⁰)R¹⁰, —(CR¹²R¹³)ᵥNR¹⁰C(═NR¹¹)R¹⁰, —(CR¹²R¹³)ᵥNR¹⁰(CR¹²R¹³)_{w}NR¹⁰R¹¹, —(CR¹²R¹³)_{w}NR¹⁰C(═NR¹¹)NR¹⁰(CR¹²R¹³)_{w}NR¹⁰R¹¹, —(CR¹²R¹³)ᵥC(═NR¹¹)NR¹⁰R¹¹, C₃-C₈ cycloalkyl, C₂-C₈ heterocycloalkyl, aryl, or heteroaryl; wherein each alkyl, alkenyl, alkynyl, cycloalkyl, heterocycloalkyl, aryl, and heteroaryl is independently optionally substituted with one, two, or three R^{A1};
each R^{A1} is independently halogen, C₁-C₆ alkyl, C₁-C₆ haloalkyl, —OR^{a}, —CN, —NO₂, —NR^{c}R^{d}, —S(═O)₂R^{b}, —S(═O)₂NR^{c}R^{d}, —C(═O)OR^{a}, —C(═O)R^{b}, —C(═O)NR^{c}R^{d}, —NR^{c}C(═NR^{c})NR^{c}R^{d}, or —NR^{c}C(═NR^{c})R^{b};
L is a bond or C₁-C₆ alkylene optionally substituted with halogen, —OR^{a}, —CN, —NO₂, or —NR^{c}R^{d};
R¹ is C₁-C₆ alkyl, C₁-C₆ haloalkyl, C₂-C₆ alkenyl, or C₂-C₆ alkynyl;
R² is hydrogen, C₁-C₆ alkyl, C₁-C₆ haloalkyl, C₂-C₆ alkenyl, C₂-C₆ alkynyl, or —Si(R^{b})₃; each R³ is independently hydrogen, C₁-C₆ alkyl, C₁-C₆ haloalkyl, C₂-C₆ alkenyl, C₂-C₆ alkynyl, or —OR^{a};
R⁴ is hydrogen, C₁-C₆ alkyl, C₁-C₆ haloalkyl, C₂-C₆ alkenyl, C₂-C₆ alkynyl, C₃-C₈ cycloalkyl, C₂-C₈ heterocycloalkyl, aryl, heteroaryl, —(C₁-C₆ alkyl)(C₃-C₈ cycloalkyl), —(C₁-C₆ alkyl)(C₂-C₈ heterocycloalkyl), —(C₁-C₆ alkyl)(aryl), or —(C₁-C₆ alkyl)(heteroaryl);
each R⁵ is independently halogen, C₁-C₆ alkyl, C₁-C₆ haloalkyl, C₂-C₆ alkenyl, C₂-C₆ alkynyl, —OR^{a}, —CN, —NO₂, —S(═O)₂R^{b}, —S(═O)₂NR^{c}R^{d}, —C(═O)OR^{a}, —C(═O)R^{b}, —C(═O)NR^{c}R^{d}, —NR^{c}R^{d}, C₃-C₈ cycloalkyl, or C₂-C₈ heterocycloalkyl;
or two R⁵ on the same carbon are taken together to form an oxo;
each R¹⁰ and R¹¹ is independently hydrogen, C₁-C₆ alkyl, C₁-C₆ haloalkyl, C₂-C₆ alkenyl, C₂-C₆ alkynyl, C₃-C₈ cycloalkyl, C₂-C₈ heterocycloalkyl, aryl, heteroaryl, —(C₁-C₆ alkyl)(C₃-C₈ cycloalkyl), —(C₁-C₆ alkyl)(C₂-C₈ heterocycloalkyl), —(C₁-C₆ alkyl)(aryl), or —(C₁-C₆ alkyl)(heteroaryl);
or R¹⁰ and R¹¹ are taken together with the nitrogen to which they are attached to form a C₂-C₈ heterocycloalkyl;
each R¹² and R¹³ is independently hydrogen, halogen, —CN, —OR^{a}, —NR^{c}R^{d}, C₁-C₆ alkyl, C₁-C₆ haloalkyl, C₂-C₆ alkenyl, C₂-C₆ alkynyl, C₃-C₈ cycloalkyl, C₂-C₈ heterocycloalkyl, aryl, or heteroaryl;
each R^{a} is independently hydrogen, C₁-C₆ alkyl, C₁-C₆ haloalkyl, C₂-C₆ alkenyl, C₂-C₆ alkynyl, C₃-C₈ cycloalkyl, or C₂-C₈ heterocycloalkyl;
each R^{b} is independently C₁-C₆ alkyl, C₁-C₆ haloalkyl, C₂-C₆ alkenyl, C₂-C₆ alkynyl, C₃-C₈ cycloalkyl, or C₂-C₈ heterocycloalkyl;
each R^{c} and R^{d} are independently hydrogen, C₁-C₆ alkyl, C₁-C₆ haloalkyl, C₂-C₆ alkenyl, C₂-C₆ alkynyl, C₃-C₈ cycloalkyl, or C₂-C₈ heterocycloalkyl;
or R^{c} and R^{d} are taken together with the nitrogen to which they are attached to form a C₂-C₈ heterocycloalkyl;
v is 0-4;
w is 2-4;
n is 0-4; and
p is 0-5.

Also disclosed herein is a compound of Formula (Ia), or a pharmaceutically acceptable salt, solvate, or stereoisomer thereof:

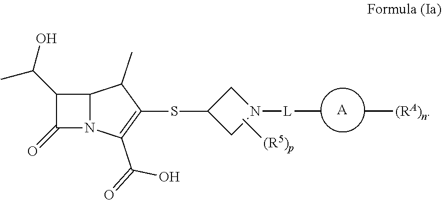

Also disclosed herein is a compound of Formula (Ib), or a pharmaceutically acceptable salt, solvate, or stereoisomer thereof:

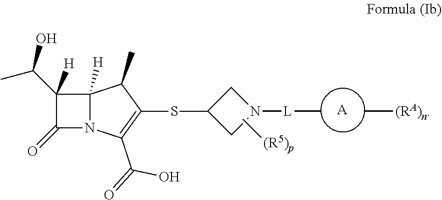

Also disclosed herein is a compound of Formula (II), or a pharmaceutically acceptable salt, solvate, or stereoisomer thereof:

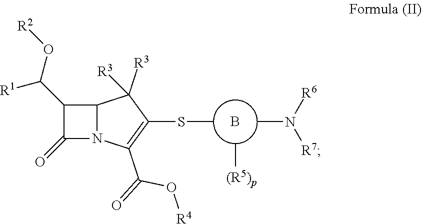

wherein
Ring B is C₃-C₈ cycloalkyl;
R¹ is C₁-C₆ alkyl, C₁-C₆ haloalkyl, C₂-C₆ alkenyl, or C₂-C₆ alkynyl;
R² is hydrogen, C₁-C₆ alkyl, C₁-C₆ haloalkyl, C₂-C₆ alkenyl, C₂-C₆ alkynyl, or —Si(R^{b})₃;
each R³ is independently hydrogen, C₁-C₆ alkyl, C₁-C₆ haloalkyl, C₂-C₆ alkenyl, C₂-C₆ alkynyl, or —OR^{a};
R⁴ is hydrogen, C₁-C₆ alkyl, C₁-C₆ haloalkyl, C₂-C₆ alkenyl, C₂-C₆ alkynyl, C₃-C₈ cycloalkyl, C₂-C₈ heterocycloalkyl, aryl, heteroaryl, —(C₁-C₆ alkyl)(C₃-C₈ cycloalkyl), —(C₁-C₆ alkyl)(C₂-C₈ heterocycloalkyl), —(C₁-C₆ alkyl)(aryl), or —(C₁-C₆ alkyl)(heteroaryl);
each R⁵ is independently halogen, C₁-C₆ alkyl, C₁-C₆ haloalkyl, C₂-C₆ alkenyl, C₂-C₆ alkynyl, —OR^{a}, —CN, —NO₂, —S(═O)₂R^{b}, —S(═O)₂NR^{c}R^{d}, —C(═O)OR^{a}, —C(═O)R^{b}, —C(═O)NR^{c}R^{d}, —NR^{c}R^{d}, C₁-C₆ alkyl, C₂-C₆ alkenyl, C₂-C₆ alkynyl, C₃-C₈ cycloalkyl, or C₂-C₈ heterocycloalkyl; or two R⁵ on the same carbon are taken together to form an oxo;
R⁶ is hydrogen, C₁-C₆ alkyl, C₁-C₆ haloalkyl, C₂-C₆ alkenyl, C₂-C₆ alkynyl, C₃-C₈ cycloalkyl, or C₂-C₈ heterocycloalkyl;
R⁷ is C₃-C₈ cycloalkyl, C₂-C₈ heterocycloalkyl, aryl, and heteroaryl; each independently optionally substituted with one, two, or three R^{7a};
or R⁶ and R⁷ are taken together with the nitrogen to which they are attached to form a C₂-C₈ heterocycloalkyl optionally substituted with one, two, or three R^{7a};
each R^{7a} is independently halogen, C₁-C₆ alkyl, C₂-C₆ alkenyl, C₂-C₆ alkynyl, C₁-C₆ haloalkyl, C₁-C₆ hydroxyalkyl, —(CR¹²R¹³)ᵥOR¹⁰, —(CR¹²R¹³)ᵥNR¹⁰R¹¹, —CN, —NO₂, —(CR¹²R¹³)ᵥS(═O)₂R¹⁰, —(CR¹²R¹³)ᵥS(═O)₂NR¹⁰R¹¹, —(CR¹²R¹³)ᵥC(═O)OR¹⁰, —(CR¹²R¹³)ᵥC(═O)NR¹⁰R¹¹, —(CR¹²R¹³)ᵥNR¹⁰C(═NR¹¹)NR¹⁰R¹¹, —(CR¹²R¹³)ᵥC(═NR¹⁰)R¹⁰, —(CR¹²R¹³)ᵥNR¹⁰C(═NR¹¹)R¹⁰, —(CR¹²R¹³)ᵥNR¹⁰(CR¹²R¹³)_{w}NR¹⁰R¹¹, —(CR¹²R¹³)ᵥNR¹⁰C(═NR¹¹)NR¹⁰(CR¹²R¹³)_{w}NR¹⁰R¹¹, —(CR¹²R¹³)ᵥC(═NR¹¹)NR¹⁰R¹¹, C₃-C₈ cycloalkyl, C₂-C₈ heterocycloalkyl, aryl, or heteroaryl; wherein each alkyl, alkenyl, alkynyl, cycloalkyl, heterocycloalkyl, aryl, and heteroaryl is independently optionally substituted with one, two, or three R^{7b};
each R^{7b} is independently halogen, C₁-C₆ alkyl, C₁-C₆ haloalkyl, —OR^{a}, —CN, —NO₂, —NR^{c}R^{d}, —S(═O)₂R^{b}, —S(═O)₂NR^{c}R^{d}, —C(═O)OR^{a}, —C(═O)R^{b}, —C(═O)NR^{c}R^{d}, —NR^{c}C(═NR)NR^{c}R^{d}, or —NR^{c}C(═NR^{c})R^{b};
each R¹⁰ and R¹¹ is independently hydrogen, C₁-C₆ alkyl, C₁-C₆ haloalkyl, C₂-C₆ alkenyl, C₂-C₆ alkynyl, C₃-C₈ cycloalkyl, C₂-C₈ heterocycloalkyl, aryl, heteroaryl, —(C₁-C₆ alkyl)(C₃-C₈ cycloalkyl), —(C₁-C₆ alkyl)(C₂-C₈ heterocycloalkyl), —(C₁-C₆ alkyl)(aryl), or —(C₁-C₆ alkyl)(heteroaryl);
or R¹⁰ and R¹¹ are taken together with the nitrogen to which they are attached to form a C₂-C₈ heterocycloalkyl;
each R¹² and R¹³ is independently hydrogen, halogen, —CN, —OR^{a}, —NR^{c}R^{d}, C₁-C₆ alkyl, C₁-C₆ haloalkyl, C₂-C₆ alkenyl, C₂-C₆ alkynyl, C₃-C₈ cycloalkyl, C₂-C₈ heterocycloalkyl, aryl, or heteroaryl;
each R^{a} is independently hydrogen, C₁-C₆ alkyl, C₁-C₆ haloalkyl, C₂-C₆ alkenyl, C₂-C₆ alkynyl, C₃-C₈ cycloalkyl, or C₂-C₈ heterocycloalkyl;
each R^{b} is independently C₁-C₆ alkyl, C₁-C₆ haloalkyl, C₂-C₆ alkenyl, C₂-C₆ alkynyl, C₃-C₈ cycloalkyl, or C₂-C₈ heterocycloalkyl;
each R^{c} and R^{d} are independently hydrogen, C₁-C₆ alkyl, C₁-C₆ haloalkyl, C₂-C₆ alkenyl, C₂-C₆ alkynyl, C₃-C₈ cycloalkyl, or C₂-C₈ heterocycloalkyl;
or R^{c} and R^{d} are taken together with the nitrogen to which they are attached to form a C₂-C₈ heterocycloalkyl;
v is 0-4;
w is 2-4; and
p is 0-5.

Also disclosed herein is a compound of Formula (IIa), or a pharmaceutically acceptable salt, solvate, or stereoisomer thereof:

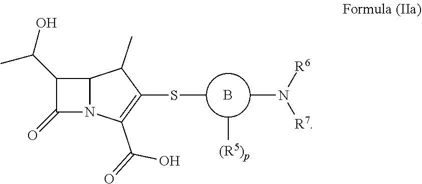

Also disclosed herein is a compound of Formula (IIb), or a pharmaceutically acceptable salt, solvate, or stereoisomer thereof:

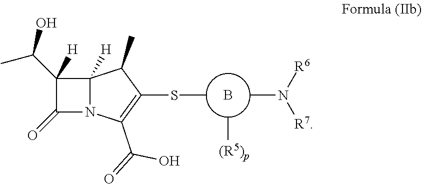

Also disclosed herein is a compound of Formula (IIc), or a pharmaceutically acceptable salt, solvate, or stereoisomer thereof:

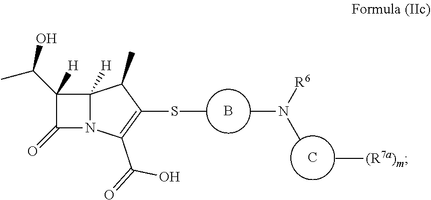

wherein
Ring C is C₃-C₈ cycloalkyl or C₂-C₈ heterocycloalkyl;
each R^{7a} is independently halogen, C₁-C₆ alkyl, C₂-C₆ alkenyl, C₂-C₆ alkynyl, C₁-C₆ haloalkyl, C₁-C₆ hydroxyalkyl, —(CR¹²R¹³)ᵥOR¹⁰, —(CR¹²R¹³)ᵥNR¹⁰R¹¹, —CN, —NO₂, —(CR¹²R¹³)ᵥS(═O)₂R¹⁰, —(CR¹²R¹³)ᵥS(═O)₂NR¹⁰R¹¹, —(CR¹²R¹³)ᵥC(═O)OR¹⁰, —(CR¹²R¹³)ᵥC(═O)NR¹⁰R¹¹, —(CR¹²R¹³)ᵥNR¹⁰C(═NR¹¹)NR¹⁰R¹¹, —(CR¹²R¹³)ᵥC(═NR¹⁰)R¹⁰, —(CR¹²R¹³)ᵥNR¹⁰C(═NR¹¹)R¹⁰, —(CR¹²R¹³)ᵥNR¹⁰(CR¹²R¹³)_{w}NR¹⁰R¹¹, —(CR¹²R¹³)ᵥ(NR¹⁰C(═NR¹¹)NR¹⁰(CR¹²R¹³)_{w}NR¹⁰R¹¹, —(CR¹²R¹³)ᵥC(═NR¹¹)NR¹⁰R¹¹, C₃-C₈ cycloalkyl, C₂-C₈ heterocycloalkyl, aryl, or heteroaryl; wherein each alkyl, alkenyl, alkynyl, cycloalkyl, heterocycloalkyl, aryl, and heteroaryl is independently optionally substituted with one, two, or three R^{7b};
each R^{7b} is independently halogen, C₁-C₆ alkyl, C₁-C₆ haloalkyl, —OR^{a}, —CN, —NO₂, —NR^{c}R^{d}, —S(═O)₂R^{b}, —S(═O)₂NR^{c}R^{d}, —C(═O)OR^{a}, —C(═O)R^{b}, —C(═O)NR^{c}R^{d}, —NR^{c}C(═NR^{c})NR^{c}R^{d}, or —NR^{c}C(═NR^{c})R^{b};
each R¹⁰ and R¹¹ is independently hydrogen, C₁-C₆ alkyl, C₁-C₆ haloalkyl, C₂-C₆ alkenyl, C₂-C₆ alkynyl, C₃-C₈ cycloalkyl, C₂-C₈ heterocycloalkyl, aryl, heteroaryl, —(C₁-C₆ alkyl)(C₃-C₈ cycloalkyl), —(C₁-C₆ alkyl)(C₂-C₈ heterocycloalkyl), —(C₁-C₆ alkyl)(aryl), or —(C₁-C₆ alkyl)(heteroaryl);
or R¹⁰ and R¹¹ are taken together with the nitrogen to which they are attached to form a C₂-C₈ heterocycloalkyl;
each R¹² and R¹³ is independently hydrogen, halogen, —CN, —OR^{a}, —NR^{c}R^{d}, C₁-C₆ alkyl, C₁-C₆ haloalkyl, C₂-C₆ alkenyl, C₂-C₆ alkynyl, C₃-C₈ cycloalkyl, C₂-C₈ heterocycloalkyl, aryl, or heteroaryl;
v is 0-4;
w is 2-4; and
m is 0-3.

Also disclosed herein is a compound of Formula (IId), or a pharmaceutically acceptable salt, solvate, or stereoisomer thereof:

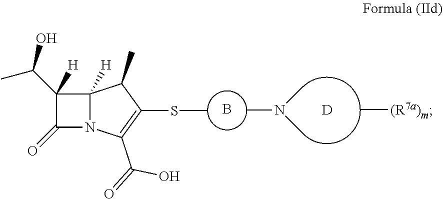

wherein
Ring D is C₂-C₆ heterocycloalkyl;
each R^{7a} is independently halogen, C₁-C₆ alkyl, C₂-C₆ alkenyl, C₂-C₆ alkynyl, C₁-C₆ haloalkyl, C₁-C₆ hydroxyalkyl, —(CR¹²R¹³)ᵥOR¹⁰, —(CR¹²R¹³)ᵥNR¹⁰R¹¹, —CN, —NO₂, —(CR¹²R¹³)ᵥS(═O)₂R¹⁰, —(CR¹²R¹³)ᵥS(═O)₂NR¹⁰R¹¹, —(CR¹²R¹³)ᵥC(═O)OR¹⁰, —(CR¹²R¹³)ᵥC(═O)NR⁰R¹, —(CR¹²R¹³)ᵥNR¹⁰C(═NR¹¹)NR¹⁰R¹¹, —(CR¹²R¹³)ᵥC(═NR¹⁰)R¹⁰, —(CR¹²R¹³)ᵥNR¹⁰C(═NR¹¹)R¹⁰, —(CR¹²R¹³)ᵥNR¹⁰(CR¹²R¹³)_{w}NR¹⁰R¹¹, —(CR¹²R¹³)ᵥ(NR¹⁰C(═NR¹¹)NR¹⁰(CR¹²R¹³)_{w}NR¹⁰R¹¹, —(CR¹²R¹³)ᵥC(═NR¹¹)NR¹⁰R¹¹, C₃-C₈ cycloalkyl, C₂-C₈ heterocycloalkyl, aryl, or heteroaryl; wherein each alkyl, alkenyl, alkynyl, cycloalkyl, heterocycloalkyl, aryl, and heteroaryl is independently optionally substituted with one, two, or three R^{7b};
each R^{7b} is independently halogen, C₁-C₆ alkyl, C₁-C₆ haloalkyl, —OR^{a}, —CN, —NO₂, —NR^{c}R^{d}, —S(═O)₂R^{b}, —S(═O)₂NR^{c}R^{d}, —C(═O)OR^{a}, —C(═O)R^{b}, —C(═O)NR^{c}R^{d}, —NR^{c}C(═NR)NR^{c}R^{d}, or —NR^{c}C(═NR^{c})R^{b};
each R¹⁰ and R¹¹ is independently hydrogen, C₁-C₆ alkyl, C₁-C₆ haloalkyl, C₂-C₆ alkenyl, C₂-C₆ alkynyl, C₃-C₈ cycloalkyl, C₂-C₈ heterocycloalkyl, aryl, heteroaryl, —(C₁-C₆ alkyl)(C₃-C₈ cycloalkyl), —(C₁-C₆ alkyl)(C₂-C₈ heterocycloalkyl), —(C₁-C₆ alkyl)(aryl), or —(C₁-C₆ alkyl)(heteroaryl);
or R¹⁰ and R¹¹ are taken together with the nitrogen to which they are attached to form a C₂-C₈ heterocycloalkyl;
each R¹² and R¹³ is independently hydrogen, halogen, —CN, —OR^{a}, —NR^{c}R^{d}, C₁-C₆ alkyl, C₁-C₆ haloalkyl, C₂-C₆ alkenyl, C₂-C₆ alkynyl, C₃-C₈ cycloalkyl, C₂-C₈ heterocycloalkyl, aryl, or heteroaryl;
v is 0-4;
w is 2-4; and
m is 0-3.

Disclosed herein are pharmaceutical compositions comprising a compound disclosed herein, or a pharmaceutically acceptable salt, solvate, or stereoisomer thereof and a pharmaceutically acceptable excipient.

Disclosed herein are pharmaceutical compositions comprising a compound disclosed herein, or a pharmaceutically acceptable salt, solvate, or stereoisomer thereof; a β-lactamase inhibitor; and a pharmaceutically acceptable excipient.

Disclosed herein are methods of treating a bacterial infection in a subject, comprising administering to the subject an effective amount of a compound disclosed herein, or a pharmaceutically acceptable salt, solvate, or stereoisomer thereof.

Disclosed herein are methods of treating a bacterial infection in a subject, comprising administering to the subject an effective amount of a compound disclosed herein, or a pharmaceutically acceptable salt, solvate, or stereoisomer thereof, in combination with a β-lactamase inhibitor.

Described herein are methods of treating a bacterial infection in a subject, comprising administering to the subject a pharmaceutical composition comprising a compound disclosed herein, or a pharmaceutically acceptable salt, solvate, or stereoisomer thereof, and a pharmaceutically acceptable excipient.

Described herein are methods of treating a bacterial infection in a subject, comprising administering to the subject an effective amount of a compound disclosed herein, or a pharmaceutically acceptable salt, solvate, or stereoisomer thereof in combination with a β-lactamase inhibitor.

In some embodiments, the bacterial infection is caused by gram-negative bacteria. In some embodiments, the bacterial infection is caused by gram-positive bacteria. In some embodiments, the bacterial infection is caused by multidrug-resistant (MDR) bacteria. In some embodiments, the bacterial infection is caused by carbapenem resistant Enterobacteriaceae (CRE).

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION OF THE INVENTION

Decades of clinical use of antibiotics have led to a dramatic increase in the prevalence of infections caused by Multi Drug Resistant (MDR) bacteria. For gram-positive pathogens, methicillin resistant Staphylococcus aureus (MRSA) and Penicillin-Resistant pneumococcus (PRP) are increasingly prevalent. For gram-negatives, the extensive use of β-lactam antibiotics has pressured bacteria to develop several mechanisms of resistance, the most widely diffused and efficient of which is the production of Extended Spectrum β-lactamase enzymes (ESBLs) and Ambler Class C cephalosporinases. Because MRSA and ESBL producing gram-negative bacteria frequently demonstrate cross-resistance to other antibiotic classes (e.g., aminoglycosides, quinolones) the choice for treatment of infections has diminished. In addition, the spread of Class A/D (serine) and B (metallo) carbapenemases is now eroding efficacy of carbapenems, which is regarded as the most potent sub class of β-lactams.

Novel carbapenems with activity against MRSA, ESBL and Class C producing Enterobacteriaceae, and Pseudomonas aeruginosa would represent a major advance in the treatment of many nosocomial infections. These novel carbapenems may also be paired with a proprietary pan-β-lactamase inhibitor to include even broader coverage of infections from carbapenem resistant Enterobacteriaceae (CRE). These ultra-broad spectrum carbapenems would provide coverage that usually requires co-administration of two or even three antibacterials. This would provide a single product first-line empiric therapy for either gram-positive or negative pathogens, a polymicrobial mix, and/or anaerobes. The present disclosure is directed to certain carbapenem compounds which are antibacterials active against the bacterial Penicillin Binding Proteins (PBPs). Some embodiments include compounds, compositions, pharmaceutical compositions, use and preparation thereof.

### Definitions

In the following description, certain specific details are set forth in order to provide a thorough understanding of various embodiments. However, one skilled in the art will understand that the invention may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed invention.

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments. Also, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

The term “antibiotic” refers to a compound or composition which decreases the viability of a microorganism, or which inhibits the growth or proliferation of a microorganism. The phrase “inhibits the growth or proliferation” means increasing the generation time (i.e., the time required for the bacterial cell to divide or for the population to double) by at least about 2-fold. Examples antibiotics are those which can increase the generation time by at least about 10-fold or more (e.g., at least about 100-fold or even indefinitely, as in total cell death). As used in this disclosure, an antibiotic is further intended to include an antimicrobial, bacteriostatic, or bactericidal agent. Examples of antibiotics suitable for use with respect to the present invention include penicillins, cephalosporins, and carbapenems.

The term “β-lactam antibiotic” refers to a compound with antibiotic properties that contains a β-lactam functionality. Non-limiting examples of 3-lactam antibiotics useful with respect to the invention include penicillins, cephalosporins, penems, carbapenems, and monobactams.

The term “β-lactamase” denotes a protein capable of inactivating a β-lactam antibiotic. The β-lactamase can be an enzyme which catalyzes the hydrolysis of the β-lactam ring of a β-lactam antibiotic. Of particular interest herein are microbial β-lactamases. The β-lactamase may be, for example, a serine β-lactamase or a metallo-β-lactamase.

The terms below, as used herein, have the following meanings, unless indicated otherwise:

“Alkyl” refers to a straight or branched chain hydrocarbon monoradical, which is fully saturated, having from one to about ten carbon atoms, or from one to six carbon atoms. Examples of alkyls include, but are not limited to, methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, t-butyl, n-pentyl, isopentyl, neopentyl, tert-amyl and hexyl, and longer alkyl groups, such as heptyl, octyl, and the like. Whenever it appears herein, a numerical range such as “C₁-C₆ alkyl” or “C₁₋₆ alkyl” means that the alkyl group consists of 1 carbon atom, 2 carbon atoms, 3 carbon atoms, 4 carbon atoms, 5 carbon atoms or 6 carbon atoms, although the present definition also covers the occurrence of the term “alkyl” where no numerical range is designated. In some embodiments, the alkyl is a C₁-C₁₀ alkyl, a C₁-C₉ alkyl, a C₁-C₈ alkyl, a C₁-C₇ alkyl, a C₁-C₆ alkyl, a C₁-C₅ alkyl, a C₁-C₄ alkyl, a C₁-C₃ alkyl, a C₁-C₂ alkyl, or a C₁ alkyl. Unless stated otherwise specifically in the specification, an alkyl group is optionally substituted as described below, for example, with oxo, halogen, amino, nitrile, nitro, hydroxyl, haloalkyl, alkoxy, aryl, cycloalkyl, heterocycloalkyl, heteroaryl, and the like. In some embodiments, the alkyl is optionally substituted with oxo, halogen, —CN, —CF₃, —OH, —OMe, —NH₂, or —NO₂. In some embodiments, the alkyl is optionally substituted with oxo, halogen, —CN, —CF₃, —OH, or —OMe. In some embodiments, the alkyl is optionally substituted with halogen.

“Alkenyl” refers to an optionally substituted straight-chain, or optionally substituted branched-chain hydrocarbon monoradical having one or more carbon-carbon double-bonds and having from two to about ten carbon atoms, more preferably two to about six carbon atoms. The group may be in either the cis or trans conformation about the double bond(s), and should be understood to include both isomers. Examples include, but are not limited to ethenyl (—CH═CH₂), 1-propenyl (—CH₂CH═CH₂), isopropenyl [—C(CH₃)═CH₂], butenyl, 1,3-butadienyl and the like. Whenever it appears herein, a numerical range such as “C₂-C₆ alkenyl” or “C₂₋₆ alkenyl”, means that the alkenyl group may consist of 2 carbon atoms, 3 carbon atoms, 4 carbon atoms, 5 carbon atoms or 6 carbon atoms, although the present definition also covers the occurrence of the term “alkenyl” where no numerical range is designated. Unless stated otherwise specifically in the specification, an alkenyl group may be optionally substituted as described below, for example, with oxo, halogen, amino, nitrile, nitro, hydroxyl, haloalkyl, alkoxy, aryl, cycloalkyl, heterocycloalkyl, heteroaryl, and the like. In some embodiments, an alkenyl is optionally substituted with oxo, halogen, —CN, —CF₃, —OH, —OMe, —NH₂, or —NO₂. In some embodiments, an alkenyl is optionally substituted with oxo, halogen, —CN, —CF₃, —OH, or —OMe. In some embodiments, the alkenyl is optionally substituted with halogen.

“Alkynyl” refers to an optionally substituted straight-chain or optionally substituted branched-chain hydrocarbon monoradical having one or more carbon-carbon triple-bonds and having from two to about ten carbon atoms, more preferably from two to about six carbon atoms. Examples include, but are not limited to ethynyl, 2-propynyl, 2-butynyl, 1,3-butadiynyl and the like. Whenever it appears herein, a numerical range such as “C₂-C₆ alkynyl” or “C₂₋₆ alkynyl”, means that the alkynyl group may consist of 2 carbon atoms, 3 carbon atoms, 4 carbon atoms, 5 carbon atoms or 6 carbon atoms, although the present definition also covers the occurrence of the term “alkynyl” where no numerical range is designated. Unless stated otherwise specifically in the specification, an alkynyl group may be optionally substituted as described below, for example, with oxo, halogen, amino, nitrile, nitro, hydroxyl, haloalkyl, alkoxy, aryl, cycloalkyl, heterocycloalkyl, heteroaryl, and the like. In some embodiments, an alkynyl is optionally substituted with oxo, halogen, —CN, —CF₃, —OH, —OMe, —NH₂, or —NO₂. In some embodiments, an alkynyl is optionally substituted with oxo, halogen, —CN, —CF₃, —OH, or —OMe. In some embodiments, the alkynyl is optionally substituted with halogen.

“Alkylene” refers to a straight or branched divalent hydrocarbon chain. Unless stated otherwise specifically in the specification, an alkylene group may be optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkylene is optionally substituted with oxo, halogen, —CN, —CF₃, —OH, —OMe, —NH₂, or —NO₂. In some embodiments, an alkylene is optionally substituted with oxo, halogen, —CN, —CF₃, —OH, or —OMe. In some embodiments, the alkylene is optionally substituted with halogen.

“Alkoxy” refers to a radical of the formula —Oalkyl where alkyl is as defined above. Unless stated otherwise specifically in the specification, an alkoxy group may be optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkoxy is optionally substituted with oxo, halogen, —CN, —CF₃, —OH, —OMe, —NH₂, or —NO₂. In some embodiments, an alkoxy is optionally substituted with oxo, halogen, —CN, —CF₃, —OH, or —OMe. In some embodiments, the alkoxy is optionally substituted with halogen.

“Aryl” refers to a radical derived from a hydrocarbon ring system comprising hydrogen, 6 to 30 carbon atoms and at least one aromatic ring. The aryl radical may be a monocyclic, bicyclic, tricyclic or tetracyclic ring system, which may include fused (when fused with a cycloalkyl or heterocycloalkyl ring, the aryl is bonded through an aromatic ring atom) or bridged ring systems. In some embodiments, the aryl is a 6- to 10-membered aryl. In some embodiments, the aryl is a 6-membered aryl. Aryl radicals include, but are not limited to, aryl radicals derived from the hydrocarbon ring systems of anthrylene, naphthylene, phenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. In some embodiments, the aryl is phenyl. Unless stated otherwise specifically in the specification, an aryl may be optionally substituted, for example, with halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, an aryl is optionally substituted with halogen, methyl, ethyl, —CN, —CF₃, —OH, —OMe, —NH₂, or —NO₂. In some embodiments, an aryl is optionally substituted with halogen, methyl, ethyl, —CN, —CF₃, —OH, or —OMe. In some embodiments, the aryl is optionally substituted with halogen.

“Cycloalkyl” refers to a partially or fully saturated, monocyclic or polycyclic carbocyclic ring, which may include fused (when fused with an aryl or a heteroaryl ring, the cycloalkyl is bonded through a non-aromatic ring atom) or bridged ring systems. Representative cycloalkyls include, but are not limited to, cycloalkyls having from three to fifteen carbon atoms (C₃-C₁₅ cycloalkyl), from three to ten carbon atoms (C₃-C₁₀ cycloalkyl), from three to eight carbon atoms (C₃-C₈ cycloalkyl), from three to six carbon atoms (C₃-C₆ cycloalkyl), from three to five carbon atoms (C₃-C₈ cycloalkyl), or three to four carbon atoms (C₃-C₄ cycloalkyl). In some embodiments, the cycloalkyl is a 3- to 6-membered cycloalkyl. In some embodiments, the cycloalkyl is a 5- to 6-membered cycloalkyl. Monocyclic cycloalkyls include, for example, cyclopropyl, cyclobutyl, cyclopentyl, cyclohexyl, cycloheptyl, and cyclooctyl. Polycyclic cycloalkyls or carbocycles include, for example, adamantyl, norbornyl, decalinyl, bicyclo[3.3.0]octane, bicyclo[4.3.0]nonane, cis-decalin, trans-decalin, bicyclo[2.1.1]hexane, bicyclo[2.2.1]heptane, bicyclo[2.2.2]octane, bicyclo[3.2.2]nonane, and bicyclo[3.3.2]decane, and 7,7-dimethyl-bicyclo[2.2.1]heptanyl. Partially saturated cycloalkyls include, for example cyclopentenyl, cyclohexenyl, cycloheptenyl, and cyclooctenyl. Unless stated otherwise specifically in the specification, a cycloalkyl is optionally substituted, for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a cycloalkyl is optionally substituted with oxo, halogen, methyl, ethyl, —CN, —CF₃, —OH, —OMe, —NH₂, or —NO₂. In some embodiments, a cycloalkyl is optionally substituted with oxo, halogen, methyl, ethyl, —CN, —CF₃, —OH, or —OMe. In some embodiments, the cycloalkyl is optionally substituted with halogen.

“Halo” or “halogen” refers to bromo, chloro, fluoro or iodo. In some embodiments, halogen is fluoro or chloro. In some embodiments, halogen is fluoro.

“Haloalkyl” refers to an alkyl radical, as defined above, that is substituted by one or more halo radicals, as defined above, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like.

“Heterocycloalkyl” refers to a 3- to 24-membered partially or fully saturated ring radical comprising 2 to 23 carbon atoms and from one to 8 heteroatoms selected from nitrogen, oxygen, phosphorous, and sulfur. Unless stated otherwise specifically in the specification, the heterocycloalkyl radical may be a monocyclic, bicyclic, tricyclic or tetracyclic ring system, which may include fused (when fused with an aryl or a heteroaryl ring, the heterocycloalkyl is bonded through a non-aromatic ring atom) or bridged ring systems; and the nitrogen, carbon or sulfur atoms in the heterocycloalkyl radical may be optionally oxidized; the nitrogen atom may be optionally quaternized. In some embodiments, the heterocycloalkyl is a 3- to 6-membered heterocycloalkyl. In some embodiments, the heterocycloalkyl is a 5- to 6-membered heterocycloalkyl. Examples of such heterocycloalkyl radicals include, but are not limited to, aziridinyl, azetidinyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, 1,3-dihydroisobenzofuran-1-yl, 3-oxo-1,3-dihydroisobenzofuran-1-yl, methyl-2-oxo-1,3-dioxol-4-yl, and 2-oxo-1,3-dioxol-4-yl. The term heterocycloalkyl also includes all ring forms of the carbohydrates, including but not limited to the monosaccharides, the disaccharides and the oligosaccharides. Unless otherwise noted, heterocycloalkyls have from 2 to 10 carbons in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). Unless stated otherwise specifically in the specification, a heterocycloalkyl is optionally substituted, for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heterocycloalkyl is optionally substituted with oxo, halogen, methyl, ethyl, —CN, —CF₃, —OH, —OMe, —NH₂, or —NO₂. In some embodiments, a heterocycloalkyl is optionally substituted with oxo, halogen, methyl, ethyl, —CN, —CF₃, —OH, or —OMe. In some embodiments, the heterocycloalkyl is optionally substituted with halogen.

“Heteroalkyl” refers to an alkyl group in which one or more skeletal atoms of the alkyl are selected from an atom other than carbon, e.g., oxygen, nitrogen (e.g. —NH—, —N(alkyl)-), sulfur, or combinations thereof. A heteroalkyl is attached to the rest of the molecule at a carbon atom of the heteroalkyl. In one aspect, a heteroalkyl is a C₁-C₆ heteroalkyl wherein the heteroalkyl is comprised of 1 to 6 carbon atoms and one or more atoms other than carbon, e.g., oxygen, nitrogen (e.g. —NH—, —N(alkyl)-), sulfur, or combinations thereof wherein the heteroalkyl is attached to the rest of the molecule at a carbon atom of the heteroalkyl. Unless stated otherwise specifically in the specification, a heteroalkyl is optionally substituted, for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroalkyl is optionally substituted with oxo, halogen, methyl, ethyl, —CN, —CF₃, —OH, —OMe, —NH₂, or —NO₂. In some embodiments, a heteroalkyl is optionally substituted with oxo, halogen, methyl, ethyl, —CN, —CF₃, —OH, or —OMe. In some embodiments, the heteroalkyl is optionally substituted with halogen.

“Heteroaryl” refers to a 5- to 14-membered ring system radical comprising hydrogen atoms, one to thirteen carbon atoms, one to six heteroatoms selected from nitrogen, oxygen, phosphorous, and sulfur, and at least one aromatic ring. The heteroaryl radical may be a monocyclic, bicyclic, tricyclic or tetracyclic ring system, which may include fused (when fused with a cycloalkyl or heterocycloalkyl ring, the heteroaryl is bonded through an aromatic ring atom) or bridged ring systems; and the nitrogen, carbon or sulfur atoms in the heteroaryl radical may be optionally oxidized; the nitrogen atom may be optionally quaternized. In some embodiments, the heteroaryl is a 5- to 10-membered heteroaryl. In some embodiments, the heteroaryl is a 5- to 6-membered heteroaryl. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl is optionally substituted, for example, with halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroaryl is optionally substituted with halogen, methyl, ethyl, —CN, —CF₃, —OH, —OMe, —NH₂, or —NO₂. In some embodiments, a heteroaryl is optionally substituted with halogen, methyl, ethyl, —CN, —CF₃, —OH, or —OMe. In some embodiments, the heteroaryl is optionally substituted with halogen.

The term “optional” or “optionally” means that the subsequently described event or circumstance may or may not occur, and that the description includes instances where said event or circumstance occurs and instances in which it does not. For example, “optionally substituted alkyl” means either “alkyl” or “substituted alkyl” as defined above. Further, an optionally substituted group may be un-substituted (e.g., —CH₂CH₃), fully substituted (e.g., —CF₂CF₃), mono-substituted (e.g., —CH₂CH₂F) or substituted at a level anywhere in-between fully substituted and mono-substituted (e.g., —CH₂CHF₂, —CH₂CF₃, —CF₂CH₃, —CFHCHF₂, etc.). It will be understood by those skilled in the art with respect to any group containing one or more substituents that such groups are not intended to introduce any substitution or substitution patterns (e.g., substituted alkyl includes optionally substituted cycloalkyl groups, which in turn are defined as including optionally substituted alkyl groups, potentially ad infinitum) that are sterically impractical and/or synthetically non-feasible. Thus, any substituents described should generally be understood as having a maximum molecular weight of about 1,000 daltons, and more typically, up to about 500 daltons.

An “effective amount” or “therapeutically effective amount” refers to an amount of a compound administered to a mammalian subject, either as a single dose or as part of a series of doses, which is effective to produce a desired therapeutic effect.

“Treatment” of an individual (e.g. a mammal, such as a human) or a cell is any type of intervention used in an attempt to alter the natural course of the individual or cell. In some embodiments, treatment includes administration of a pharmaceutical composition, subsequent to the initiation of a pathologic event or contact with an etiologic agent and includes stabilization of the condition (e.g., condition does not worsen) or alleviation of the condition. In some embodiments, treatment also includes prophylactic treatment (e.g., administration of a composition described herein when an individual is suspected to be suffering from a bacterial infection).

### Compounds

Described herein are compounds, or a pharmaceutically acceptable salt, solvate, or stereoisomer thereof, useful in the treatment of bacterial infections. In some embodiments, the bacterial infection is an upper or lower respiratory tract infection, a urinary tract infection, an intra-abdominal infection, a skin infection or septicemia.

Described herein are compounds of Formula (I), or a pharmaceutically acceptable salt, solvate, or stereoisomer thereof:

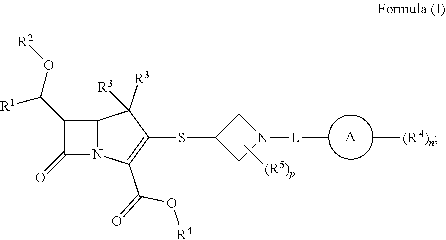

wherein
Ring A is cyclobutyl or cyclopentyl;
each R^{A} is independently halogen, C₁-C₆ alkyl, C₂-C₆ alkenyl, C₂-C₆ alkynyl, C₁-C₆ haloalkyl, C₁-C₆ hydroxyalkyl, —(CR¹²R¹³)ᵥOR¹⁰, —(CR¹²R¹³)ᵥNR¹⁰R¹¹, —CN, —NO₂, —(CR¹²R¹³)ᵥS(═O)₂R¹⁰, —(CR¹²R¹³)ᵥS(═O)₂NR¹⁰R¹¹, —(CR¹²R¹³)ᵥC(═O)OR¹⁰, —(CR¹²R¹³)ᵥC(═O)NR¹⁰R¹¹, —(CR¹²R¹³)ᵥNR¹⁰C(═NR¹¹)NR¹⁰R¹¹, —(CR¹²R¹³)ᵥC(═NR¹⁰)R¹⁰, —(CR¹²R¹³)ᵥNR¹⁰C(═NR¹¹)R¹⁰, —(CR¹²R¹³)ᵥNR¹⁰(CR¹²R¹³)_{w}NR¹⁰R¹¹, —(CR¹²R¹³)ᵥ(NR¹⁰C(═NR¹¹)NR¹⁰(CR¹²R¹³)_{w}NR¹⁰R¹¹, —(CR¹²R¹³)ᵥC(═NR¹¹)NR¹⁰R¹¹, C₃-C₈ cycloalkyl, C₂-C₈ heterocycloalkyl, aryl, or heteroaryl; wherein each alkyl, alkenyl, alkynyl, cycloalkyl, heterocycloalkyl, aryl, and heteroaryl is independently optionally substituted with one, two, or three R^{A1};
each R^{A1} is independently halogen, C₁-C₆ alkyl, C₁-C₆ haloalkyl, —OR^{a}, —CN, —NO₂, —NR^{c}R^{d}, —S(═O)₂R^{b}, —S(═O)₂NR^{c}R^{d}, —C(═O)OR^{a}, —C(═O)R^{b}, —C(═O)NR^{c}R^{d}, —NR^{c}C(═NR^{c})NR^{c}R^{d}, or —NR^{c}C(═NR^{c})R^{b};
L is a bond or C₁-C₆ alkylene optionally substituted with halogen, —OR^{a}, —CN, —NO₂, or —NR^{c}R^{d};
R¹ is C₁-C₆ alkyl, C₁-C₆ haloalkyl, C₂-C₆ alkenyl, or C₂-C₆ alkynyl;
R² is hydrogen, C₁-C₆ alkyl, C₁-C₆ haloalkyl, C₂-C₆ alkenyl, C₂-C₆ alkynyl, or —Si(R^{b})₃; each R³ is independently hydrogen, C₁-C₆ alkyl, C₁-C₆ haloalkyl, C₂-C₆ alkenyl, C₂-C₆ alkynyl, or —OR^{a};
R⁴ is hydrogen, C₁-C₆ alkyl, C₁-C₆ haloalkyl, C₂-C₆ alkenyl, C₂-C₆ alkynyl, C₃-C₈ cycloalkyl, C₂-C₈ heterocycloalkyl, aryl, heteroaryl, —(C₁-C₆ alkyl)(C₃-C₈ cycloalkyl), —(C₁-C₆ alkyl)(C₂-C₈ heterocycloalkyl), —(C₁-C₆ alkyl)(aryl), or —(C₁-C₆ alkyl)(heteroaryl);
each R⁵ is independently halogen, C₁-C₆ alkyl, C₁-C₆ haloalkyl, C₂-C₆ alkenyl, C₂-C₆ alkynyl, —OR^{a}, —CN, —NO₂, —S(═O)₂R^{b}, —S(═O)₂NR^{c}R^{d}, —C(═O)OR^{a}, —C(═O)R^{b}, —C(═O)NR^{c}R^{d}, —NR^{c}R^{d}, C₃-C₈ cycloalkyl, or C₂-C₈ heterocycloalkyl;
or two R⁵ on the same carbon are taken together to form an oxo;
each R¹⁰ and R¹¹ is independently hydrogen, C₁-C₆ alkyl, C₁-C₆ haloalkyl, C₂-C₆ alkenyl, C₂-C₆ alkynyl, C₃-C₈ cycloalkyl, C₂-C₈ heterocycloalkyl, aryl, heteroaryl, —(C₁-C₆ alkyl)(C₃-C₈ cycloalkyl), —(C₁-C₆ alkyl)(C₂-C₈ heterocycloalkyl), —(C₁-C₆ alkyl)(aryl), or —(C₁-C₆ alkyl)(heteroaryl);
or R¹⁰ and R¹¹ are taken together with the nitrogen to which they are attached to form a C₂-C₈ heterocycloalkyl;
each R¹² and R¹³ is independently hydrogen, halogen, —CN, —OR^{a}, —NR^{c}R^{d}, C₁-C₆ alkyl, C₁-C₆ haloalkyl, C₂-C₆ alkenyl, C₂-C₆ alkynyl, C₃-C₈ cycloalkyl, C₂-C₈ heterocycloalkyl, aryl, or heteroaryl;
each R^{a} is independently hydrogen, C₁-C₆ alkyl, C₁-C₆ haloalkyl, C₂-C₆ alkenyl, C₂-C₆ alkynyl, C₃-C₈ cycloalkyl, or C₂-C₈ heterocycloalkyl;
each R^{b} is independently C₁-C₆ alkyl, C₁-C₆ haloalkyl, C₂-C₆ alkenyl, C₂-C₆ alkynyl, C₃-C₈ cycloalkyl, or C₂-C₈ heterocycloalkyl;
each R^{c} and R^{d} are independently hydrogen, C₁-C₆ alkyl, C₁-C₆ haloalkyl, C₂-C₆ alkenyl, C₂-C₆ alkynyl, C₃-C₈ cycloalkyl, or C₂-C₈ heterocycloalkyl;
or R^{c} and R^{d} are taken together with the nitrogen to which they are attached to form a C₂-C₈ heterocycloalkyl;
v is 0-4;
w is 2-4;
n is 0-4; and
p is 0-5.

In some embodiments of a compound of Formula (I), R¹ is C₁-C₆ alkyl. In some embodiments of a compound of Formula (I), R¹ is methyl. In some embodiments of a compound of Formula (I), R¹ is C₂-C₆ alkenyl. In some embodiments of a compound of Formula (I), R¹ is C₂-C₆ alkynyl.

In some embodiments of a compound of Formula (I) R² is hydrogen. In some embodiments of a compound of Formula (I), R² is C₁-C₆ alkyl. In some embodiments of a compound of Formula (I), R² is methyl. In some embodiments of a compound of Formula (I), R² is C₂-C₆ alkenyl. In some embodiments of a compound of Formula (I), R² is C₂-C₆ alkynyl. In some embodiments of a compound of Formula (I), R² is —Si(R^{b})₃.

In some embodiments of a compound of Formula (I), each R³ is hydrogen. In some embodiments of a compound of Formula (I), each R³ is independently hydrogen or C₁-C₆ alkyl. In some embodiments of a compound of Formula (I), each R³ is independently hydrogen, C₁-C₆ alkyl, or OR^{a}. In some embodiments of a compound of Formula (I), each R³ is independently hydrogen, C₁-C₆ alkyl, or OH. In some embodiments of a compound of Formula (I), each R³ is independently C₁-C₆ alkyl. In some embodiments of a compound of Formula (I), one R³ is methyl and the other R³ is hydrogen. In some embodiments of a compound of Formula (I), each R³ is independently hydrogen or C₂-C₆ alkenyl. In some embodiments of a compound of Formula (I), each R³ is independently hydrogen or C₂-C₆ alkynyl.

In some embodiments of a compound of Formula (I), R⁴ is hydrogen. In some embodiments of a compound of Formula (I), R⁴ is C₁-C₆ alkyl. In some embodiments of a compound of Formula (I), R⁴ is C₂-C₆ alkenyl. In some embodiments of a compound of Formula (I), R⁴ is C₂-C₆ alkynyl. In some embodiments of a compound of Formula (I), R⁴ is —(C₁-C₆ alkyl)(aryl).

In some embodiments of a compound of Formula (I), the compound is of Formula (Ia):

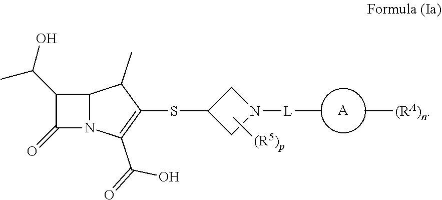

In some embodiments of a compound of Formula (I), the compound is of Formula (Ib):

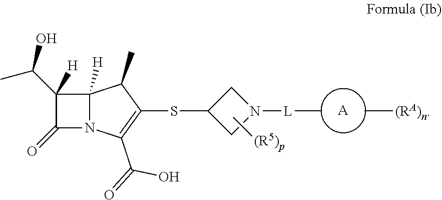

In some embodiments of a compound of Formula (I), (Ia), or (Ib), p is 0. In some embodiments of a compound of Formula (I), (Ia), or (Ib), p is 1. In some embodiments of a compound of Formula (I), (Ia), or (Ib), p is 2. In some embodiments of a compound of Formula (I), (Ia), or (Ib), p is 3. In some embodiments of a compound of Formula (I), (Ia), or (Ib), p is 4. In some embodiments of a compound of Formula (I), (Ia), or (Ib), p is 5. In some embodiments of a compound of Formula (I), (Ia), or (Ib), p is 0 or 1. In some embodiments of a compound of Formula (I), (Ia), or (Ib), p is 0, 1, or 2. In some embodiments of a compound of Formula (I), (Ia), or (Ib), p is 1 or 2. In some embodiments of a compound of Formula (I), (Ia), or (Ib), p is 2 or 3.

In some embodiments of a compound of Formula (I), (Ia), or (Ib), each R is independently halogen, C₁-C₆ alkyl, C₁-C₆ haloalkyl, —OR^{a}, —CN, —C(═O)R^{b}, —NR^{c}R^{d}, C₃-C₈ cycloalkyl, or C₂-C₈ heterocycloalkyl. In some embodiments of a compound of Formula (I), (Ia), or (Ib), each R⁵ is independently halogen, C₁-C₆ alkyl, C₁-C₆ haloalkyl, or —OR^{a}. In some embodiments of a compound of Formula (I), (Ia), or (Ib), each R is independently halogen or C₁-C₆ alkyl. In some embodiments of a compound of Formula (I), (Ia), or (Ib), two R on the same carbon are taken together to form an oxo.

In some embodiments of a compound of Formula (I), (Ia), or (Ib), L is a C₁-C₆ alkylene optionally substituted with halogen, —OW, —CN, —NO₂, or —NR^{c}R^{d}. In some embodiments of a compound of Formula (I), (Ia), or (Ib), L is a bond. In some embodiments of a compound of Formula (I), (Ia), or (Ib), L is C₁ alkylene. In some embodiments of a compound of Formula (I), (Ia), or (Ib), L is C₁-C₂ alkylene.

In some embodiments of a compound of Formula (I), (Ia), or (Ib), Ring A is cyclobutyl. In some embodiments of a compound of Formula (I), (Ia), or (Ib), Ring A is cyclopentyl.

In some embodiments of a compound of Formula (I), (Ia), or (Ib), n is 1 or 2. In some embodiments of a compound of Formula (I), (Ia), or (Ib), n is 1. In some embodiments of a compound of Formula (I), (Ia), or (Ib), n is 2. In some embodiments of a compound of Formula (I), (Ia), or (Ib), n is 3. In some embodiments of a compound of Formula (I), (Ia), or (Ib), n is 4. In some embodiments of a compound of Formula (I), (Ia), or (Ib), n is 0, 1, or 2. In some embodiments of a compound of Formula (I), (Ia), or (Ib), n is 2 or 3.

In some embodiments of a compound of Formula (I), (Ia), or (Ib), each R^{A} is independently —(CR¹²R¹³)ᵥNR¹⁰R¹¹, —(CR¹²R¹³)ᵥS(═O)₂NR¹⁰R¹¹, —(CR¹²R¹³)ᵥC(═O)NR¹⁰R¹¹, —(CR¹²R¹³)ᵥNR¹⁰C(═NR¹¹)NR¹⁰R¹¹, —(CR¹²R¹³)ᵥC(═NR¹⁰)R¹⁰, —(CR¹²R¹³)ᵥNR¹⁰C(═NR¹¹)R¹⁰, —(CR¹²R¹³)ᵥNR¹⁰(CR¹²R¹³)_{w}NR¹⁰R¹¹, —(CR¹²R¹³)ᵥ(NR¹⁰C(═NR¹¹)NR¹⁰(CR¹²R¹³)_{w}NR¹⁰R¹¹, or —(CR¹²R¹³)ᵥC(═NR¹¹)NR¹⁰R¹¹.

In some embodiments of a compound of Formula (I), (Ia), or (Ib), each R^{A} is independently —(CR¹²R¹³)ᵥNR¹⁰R¹¹, —(CR¹²R¹³)ᵥNR¹⁰C(═NR¹¹)NR¹⁰R¹¹, —(CR¹²R¹³)ᵥC(═NR¹⁰)R¹⁰, —(CR¹²R¹³)ᵥNR¹⁰C(═NR¹¹)R¹⁰, —(CR¹²R¹³)ᵥNR¹⁰(CR¹²R¹³)ᵥNR¹⁰R¹¹, —(CR¹²R¹³)ᵥ(NR¹⁰C(═NR¹¹)NR¹⁰(CR¹²R¹³)_{w}NR¹⁰R¹¹, or —(CR¹²R¹³)ᵥC(═NR¹¹)NR¹⁰R¹¹.

In some embodiments of a compound of Formula (I), (Ia), or (Ib), each R^{A} is independently —(CR¹²R¹³)ᵥNR¹⁰R¹¹, —(CR¹²R¹³)ᵥNR¹⁰C(═NR¹¹)NR¹⁰R¹¹, —(CR¹²R¹³)ᵥC(═NR¹⁰)R¹⁰, —(CR¹²R¹³)ᵥNR¹⁰C(═NR¹¹)R¹⁰.

In some embodiments of a compound of Formula (I), (Ia), or (Ib), each R^{A} is independently —(CR¹²R¹³)ᵥNR¹⁰R¹¹ or —(CR¹²R¹³)ᵥNR¹⁰C(═NR¹¹)NR¹⁰R¹¹.

In some embodiments of a compound of Formula (I), (Ia), or (Ib), each R^{A} is independently —(CR¹²R¹³)ᵥNR¹⁰R¹¹, —(CR¹²R¹³)ᵥNR¹⁰C(═NR¹¹)NR¹⁰R¹¹, —(CR¹²R¹³)ᵥC(═NR¹⁰)R¹⁰, —(CR¹²R¹³)ᵥNR¹⁰C(═NR¹¹)R¹⁰, or —(CR¹²R¹³)ᵥC(═NR¹¹)NR¹⁰R¹¹.

In some embodiments of a compound of Formula (I), (Ia), or (Ib), each R^{A} is independently —(CR¹²R¹³)ᵥNR¹⁰R¹¹, —(CR¹²R¹³)ᵥNR¹⁰C(═NR¹¹)NR¹⁰R¹¹, or —(CR¹²R¹³)ᵥC(═NR¹¹)NR¹⁰R¹¹.

In some embodiments of a compound of Formula (I), (Ia), or (Ib), v is 0 or 1. In some embodiments of a compound of Formula (I), (Ia), or (Ib), v is 0. In some embodiments of a compound of Formula (I), (Ia), or (Ib), v is 1. In some embodiments of a compound of Formula (I), (Ia), or (Ib), v is 2. In some embodiments of a compound of Formula (I), (Ia), or (Ib), v is 3. In some embodiments of a compound of Formula (I), (Ia), or (Ib), v is 4. In some embodiments of a compound of Formula (I), (Ia), or (Ib), v is 0, 1, or 2. In some embodiments of a compound of Formula (I), (Ia), or (Ib), v is 1 or 2.

In some embodiments of a compound of Formula (I), (Ia), or (Ib), w is 2 or 3. In some embodiments of a compound of Formula (I), (Ia), or (Ib), w is 2. In some embodiments of a compound of Formula (I), (Ia), or (Ib), w is 3. In some embodiments of a compound of Formula (I), (Ia), or (Ib), w is 4.

In some embodiments of a compound of Formula (I), (Ia), or (Ib), each R¹⁰ and R¹¹ is independently hydrogen, C₁-C₆ alkyl, C₁-C₆ haloalkyl, C₃-C₈ cycloalkyl, C₂-C₈ heterocycloalkyl, aryl, heteroaryl, —(C₁-C₆ alkyl)(C₃-C₈ cycloalkyl), —(C₁-C₆ alkyl)(C₂-C₈ heterocycloalkyl), —(C₁-C₆ alkyl)(aryl), or —(C₁-C₆ alkyl)(heteroaryl). In some embodiments of a compound of Formula (I), (Ia), or (Ib), each R¹⁰ and R¹¹ is independently hydrogen, C₁-C₆ alkyl, C₁-C₆ haloalkyl, C₃-C₈ cycloalkyl, C₂-C₈ heterocycloalkyl, —(C₁-C₆ alkyl)(C₃-C₈ cycloalkyl), —(C₁-C₆ alkyl)(C₂-C₈ heterocycloalkyl), —(C₁-C₆ alkyl)(aryl), or —(C₁-C₆ alkyl)(heteroaryl). In some embodiments of a compound of Formula (I), (Ia), or (Ib), each R¹⁰ and R¹¹ is independently hydrogen, C₁-C₆ alkyl, C₁-C₆ haloalkyl, C₃-C₈ cycloalkyl, C₂-C₈ heterocycloalkyl, aryl, or heteroaryl. In some embodiments of a compound of Formula (I), (Ia), or (Ib), each R¹⁰ and R¹¹ is independently hydrogen, C₁-C₆ alkyl, or C₁-C₆ haloalkyl. In some embodiments of a compound of Formula (I), (Ia), or (Ib), each R¹⁰ and R¹¹ is independently hydrogen or C₁-C₆ alkyl. In some embodiments of a compound of Formula (I), (Ia), or (Ib), each R¹⁰ and R¹¹ is C₁-C₆ alkyl. In some embodiments of a compound of Formula (I), (Ia), or (Ib), each R¹⁰ and R¹¹ is hydrogen. In some embodiments of a compound of Formula (I), (Ia), or (Ib), R¹⁰ and R¹¹ are taken together with the nitrogen to which they are attached to form a C₂-C₈ heterocycloalkyl. In some embodiments of a compound of Formula (I), (Ia), or (Ib), each R¹² and R¹³ is independently hydrogen, halogen, —CN, —OR^{a}, —NR^{c}R^{d}, C₁-C₆ alkyl, C₁-C₆ haloalkyl, C₃-C₈ cycloalkyl, C₂-C₈ heterocycloalkyl, aryl, or heteroaryl. In some embodiments of a compound of Formula (I), (Ia), or (Ib), each R¹² and R¹³ is independently hydrogen, halogen, —CN, —OR^{a}, —NR^{c}R^{d}, C₁-C₆ alkyl, C₁-C₆ haloalkyl, C₃-C₈ cycloalkyl, or C₂-C₈ heterocycloalkyl. In some embodiments of a compound of Formula (I), (Ia), or (Ib), each R¹² and R¹³ is independently hydrogen, halogen, —CN, —OR^{a}, —NR^{c}R^{d}, C₁-C₆ alkyl, or C₁-C₆ haloalkyl. In some embodiments of a compound of Formula (I), (Ia), or (Ib), each R¹² and R¹³ is independently hydrogen, halogen, C₁-C₆ alkyl, or C₁-C₆ haloalkyl. In some embodiments of a compound of Formula (I), (Ia), or (Ib), each R¹² and R¹³ is independently hydrogen, halogen, or C₁-C₆ alkyl. In some embodiments of a compound of Formula (I), (Ia), or (Ib), each R¹² and R¹³ is hydrogen.

In some embodiments of a compound of Formula (I), (Ia), or (Ib), each R^{a} is independently hydrogen, C₁-C₆ alkyl, C₁-C₆ haloalkyl, C₃-C₈ cycloalkyl, or C₂-C₈ heterocycloalkyl. In some embodiments of a compound of Formula (I), (Ia), or (Ib), each R^{a} is independently hydrogen, C₁-C₆ alkyl, or C₁-C₆ haloalkyl. In some embodiments of a compound of Formula (I), (Ia), or (Ib), each R^{a} is independently hydrogen or C₁-C₆ alkyl. In some embodiments of a compound of Formula (I), (Ia), or (Ib), each R^{a} is independently C₁-C₆ alkyl. In some embodiments of a compound of Formula (I), (Ia), or (Ib), each R^{a} is hydrogen.

In some embodiments of a compound of Formula (I), (Ia), or (Ib), each R^{b} is independently C₁-C₆ alkyl, C₁-C₆ haloalkyl, C₃-C₈ cycloalkyl, or C₂-C₈ heterocycloalkyl. In some embodiments of a compound of Formula (I), (Ia), or (Ib), each R^{b} is independently C₁-C₆ alkyl, or C₁-C₆ haloalkyl. In some embodiments of a compound of Formula (I), (Ia), or (Ib), each R^{b} is independently C₁-C₆ alkyl.

In some embodiments of a compound of Formula (I), (Ia), or (Ib), each R^{c} and R^{d} are independently hydrogen, C₁-C₆ alkyl, C₁-C₆ haloalkyl, C₃-C₈ cycloalkyl, or C₂-C₈ heterocycloalkyl. In some embodiments of a compound of Formula (I), (Ia), or (Ib), each R and R^{a} are independently hydrogen, C₁-C₆ alkyl, or C₁-C₆ haloalkyl. In some embodiments of a compound of Formula (I), (Ia), or (Ib), each R and R^{a} are independently hydrogen or C₁-C₆ alkyl. In some embodiments of a compound of Formula (I), (Ia), or (Ib), each R^{c} and R^{d} are independently C₁-C₆ alkyl. In some embodiments of a compound of Formula (I), (Ia), or (Ib), each R^{c} and R^{d} are hydrogen.

In some embodiments of a compound of Formula (I), (Ia), or (Ib), R^{c} and R^{d} are taken together with the nitrogen to which they are attached to form a C₂-C₈ heterocycloalkyl. In some embodiments of a compound of Formula (I), (Ia), or (Ib), R^{c} and R^{d} are taken together with the nitrogen to which they are attached to form a pyrrolidine, piperidine, morpholine, or piperazine.

Also disclosed herein is a compound of Formula (II), or a pharmaceutically acceptable salt, solvate, or stereoisomer thereof:

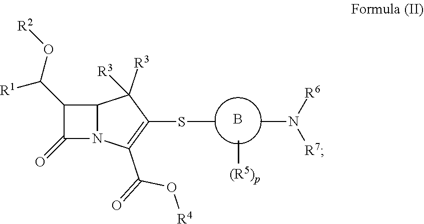

wherein
Ring B is C₃-C₈ cycloalkyl;
R¹ is C₁-C₆ alkyl, C₁-C₆ haloalkyl, C₂-C₆ alkenyl, or C₂-C₆ alkynyl;
R² is hydrogen, C₁-C₆ alkyl, C₁-C₆ haloalkyl, C₂-C₆ alkenyl, C₂-C₆ alkynyl, or —Si(R^{b})₃; each R³ is independently hydrogen, C₁-C₆ alkyl, C₁-C₆ haloalkyl, C₂-C₆ alkenyl, C₂-C₆ alkynyl, or —OR^{a};
R⁴ is hydrogen, C₁-C₆ alkyl, C₁-C₆ haloalkyl, C₂-C₆ alkenyl, C₂-C₆ alkynyl, C₃-C₈ cycloalkyl, C₂-C₈ heterocycloalkyl, aryl, heteroaryl, —(C₁-C₆ alkyl)(C₃-C₈ cycloalkyl), —(C₁-C₆ alkyl)(C₂-C₈ heterocycloalkyl), —(C₁-C₆ alkyl)(aryl), or —(C₁-C₆ alkyl)(heteroaryl);
each R⁵ is independently halogen, C₁-C₆ alkyl, C₁-C₆ haloalkyl, C₂-C₆ alkenyl, C₂-C₆ alkynyl, —OR^{a}, —CN, —NO₂, —S(═O)₂R^{b}, —S(═O)₂NR^{c}R^{d}, —C(═O)OR^{a}, —C(═O)R^{b}, —C(═O)NR^{c}R^{d}, —NR^{c}R^{d}, C₁-C₆ alkyl, C₂-C₆ alkenyl, C₂-C₆ alkynyl, C₃-C₈ cycloalkyl, or C₂-C₈ heterocycloalkyl; or two R⁵ on the same carbon are taken together to form an oxo;
R⁶ is hydrogen, C₁-C₆ alkyl, C₁-C₆ haloalkyl, C₂-C₆ alkenyl, C₂-C₆ alkynyl, C₃-C₈ cycloalkyl, or C₂-C₈ heterocycloalkyl;
R⁷ is C₃-C₈ cycloalkyl, C₂-C₈ heterocycloalkyl, aryl, and heteroaryl; each independently optionally substituted with one, two, or three R^{7a};
or R⁶ and R⁷ are taken together with the nitrogen to which they are attached to form a C₂-C₈ heterocycloalkyl optionally substituted with one, two, or three R^{7a};
each R^{7a} is independently halogen, C₁-C₆ alkyl, C₂-C₆ alkenyl, C₂-C₆ alkynyl, C₁-C₆ haloalkyl, C₁-C₆ hydroxyalkyl, —(CR¹²R¹³)ᵥOR¹⁰, —(CR¹²R¹³)ᵥNR¹⁰R¹¹, —CN, —NO₂, —(CR¹²R¹³)ᵥS(═O)₂R¹⁰, —(CR¹²R¹³)ᵥS(═O)₂NR¹⁰R¹¹, —(CR¹²R¹³)ᵥC(═O)OR¹⁰, —(CR¹²R¹³)ᵥC(═O)NR¹⁰R¹¹, —(CR¹²R¹³)ᵥNR¹⁰C(═NR¹¹)NR¹⁰R¹¹, —(CR¹²R¹³)ᵥC(═NR¹⁰)R¹⁰, —(CR¹²R¹³)ᵥNR¹⁰C(═NR¹¹)R¹⁰, —(CR¹²R¹³)ᵥNR¹⁰(CR¹²R¹³)_{w}NR¹⁰R¹¹, —(CR¹²R¹³)ᵥ(NR¹⁰C(═NR¹¹)NR¹⁰(CR¹²R¹³)_{w}NR¹⁰R¹¹, —(CR¹²R¹³)ᵥC(═NR¹¹)NR¹⁰R¹¹, C₃-C₈ cycloalkyl, C₂-C₈ heterocycloalkyl, aryl, or heteroaryl; wherein each alkyl, alkenyl, alkynyl, cycloalkyl, heterocycloalkyl, aryl, and heteroaryl is independently optionally substituted with one, two, or three R^{7b};
each R^{7b} is independently halogen, C₁-C₆ alkyl, C₁-C₆ haloalkyl, —OR^{a}, —CN, —NO₂, —NR^{c}R^{d}, —S(═O)₂R^{b}, —S(═O)₂NR^{c}R^{d}, —C(═O)OR^{a}, —C(═O)R^{b}, —C(═O)NR^{c}R^{d}, —NR^{c}C(═NR)NR^{c}R^{d}, or —NR^{c}C(═NR^{c})R^{b};
each R¹⁰ and R¹¹ is independently hydrogen, C₁-C₆ alkyl, C₁-C₆ haloalkyl, C₂-C₆ alkenyl, C₂-C₆ alkynyl, C₃-C₈ cycloalkyl, C₂-C₈ heterocycloalkyl, aryl, heteroaryl, —(C₁-C₆ alkyl)(C₃-C₈ cycloalkyl), —(C₁-C₆ alkyl)(C₂-C₈ heterocycloalkyl), —(C₁-C₆ alkyl)(aryl), or —(C₁-C₆ alkyl)(heteroaryl);
or R¹⁰ and R¹¹ are taken together with the nitrogen to which they are attached to form a C₂-C₈ heterocycloalkyl;
each R¹² and R¹³ is independently hydrogen, halogen, —CN, —OR^{a}, —NR^{c}R^{d}, C₁-C₆ alkyl, C₁-C₆ haloalkyl, C₂-C₆ alkenyl, C₂-C₆ alkynyl, C₃-C₈ cycloalkyl, C₂-C₈ heterocycloalkyl, aryl, or heteroaryl;
each R^{a} is independently hydrogen, C₁-C₆ alkyl, C₁-C₆ haloalkyl, C₂-C₆ alkenyl, C₂-C₆ alkynyl, C₃-C₈ cycloalkyl, or C₂-C₈ heterocycloalkyl;
each R^{b} is independently C₁-C₆ alkyl, C₁-C₆ haloalkyl, C₂-C₆ alkenyl, C₂-C₆ alkynyl, C₃-C₈ cycloalkyl, or C₂-C₈ heterocycloalkyl;
each R^{c} and R^{d} are independently hydrogen, C₁-C₆ alkyl, C₁-C₆ haloalkyl, C₂-C₆ alkenyl, C₂-C₆ alkynyl, C₃-C₈ cycloalkyl, or C₂-C₈ heterocycloalkyl;
or R^{c} and R^{d} are taken together with the nitrogen to which they are attached to form a C₂-C₈ heterocycloalkyl;
v is 0-4;
w is 2-4; and
p is 0-5.

In some embodiments of a compound of Formula (II), R¹ is C₁-C₆ alkyl. In some embodiments of a compound of Formula (II), R¹ is methyl. In some embodiments of a compound of Formula (II), R¹ is C₂-C₆ alkenyl. In some embodiments of a compound of Formula (II), R¹ is C₂-C₆ alkynyl.

In some embodiments of a compound of Formula (II) R² is hydrogen. In some embodiments of a compound of Formula (II), R² is C₁-C₆ alkyl. In some embodiments of a compound of Formula (II), R² is methyl. In some embodiments of a compound of Formula (II), R² is C₂-C₆ alkenyl. In some embodiments of a compound of Formula (II), R² is C₂-C₆ alkynyl. In some embodiments of a compound of Formula (II), R² is —Si(R^{b})₃.

In some embodiments of a compound of Formula (II), each R³ is hydrogen. In some embodiments of a compound of Formula (II), each R³ is independently hydrogen or C₁-C₆ alkyl. In some embodiments of a compound of Formula (II), each R³ is independently hydrogen, C₁-C₆ alkyl, or OR^{a}. In some embodiments of a compound of Formula (II), each R³ is independently hydrogen, C₁-C₆ alkyl, or OH. In some embodiments of a compound of Formula (II), each R³ is independently C₁-C₆ alkyl. In some embodiments of a compound of Formula (II), one R³ is methyl and the other R³ is hydrogen. In some embodiments of a compound of Formula (II), each R³ is independently hydrogen or C₂-C₆ alkenyl. In some embodiments of a compound of Formula (II), each R³ is independently hydrogen or C₂-C₆ alkynyl.

In some embodiments of a compound of Formula (II), R⁴ is hydrogen. In some embodiments of a compound of Formula (II), R⁴ is C₁-C₆ alkyl. In some embodiments of a compound of Formula (II), R⁴ is C₂-C₆ alkenyl. In some embodiments of a compound of Formula (II), R⁴ is C₂-C₆ alkynyl. In some embodiments of a compound of Formula (II), R⁴ is —(C₁-C₆ alkyl)(aryl).

In some embodiments of a compound of Formula (II), the compound is of Formula (IIa):

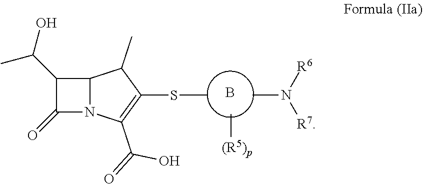

In some embodiments of a compound of Formula (II), the compound is of Formula (IIb):

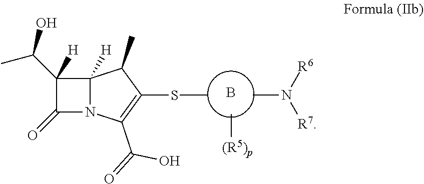

In some embodiments of a compound of Formula (II), (IIa), or (IIb), p is 0. In some embodiments of a compound of Formula (II), (IIa), or (IIb), p is 1. In some embodiments of a compound of Formula (II), (IIa), or (IIb), p is 2. In some embodiments of a compound of Formula (II), (IIa), or (IIb), p is 3. In some embodiments of a compound of Formula (II), (IIa), or (IIb), p is 4. In some embodiments of a compound of Formula (II), (IIa), or (IIb), p is 5. In some embodiments of a compound of Formula (II), (IIa), or (IIb), p is 0 or 1. In some embodiments of a compound of Formula (II), (IIa), or (IIb), p is 0, 1, or 2. In some embodiments of a compound of Formula (II), (IIa), or (IIb), p is 1 or 2. In some embodiments of a compound of Formula (II), (IIa), or (IIb), p is 2 or 3.

In some embodiments of a compound of Formula (II), (IIa), or (IIb), each R⁵ is independently halogen, C₁-C₆ alkyl, C₁-C₆ haloalkyl, —OR^{a}, —CN, —C(═O)R^{b}, —NR^{c}R^{d}, C₃-C₈ cycloalkyl, or C₂-C₈ heterocycloalkyl. In some embodiments of a compound of Formula (II), (IIa), or (IIb), each R⁵ is independently halogen, C₁-C₆ alkyl, C₁-C₆ haloalkyl, or —OR^{a}. In some embodiments of a compound of Formula (II), (IIa), or (IIb), each R⁵ is independently halogen or C₁-C₆ alkyl. In some embodiments of a compound of Formula (II), (IIa), or (IIb), two R⁵ on the same carbon are taken together to form an oxo.

In some embodiments of a compound of Formula (II), (IIa), or (IIb), R⁷ is C₃-C₈ cycloalkyl or C₂-C₈ heterocycloalkyl; each independently optionally substituted with one, two, or three R^{a}.

In some embodiments of a compound of Formula (II), (IIa), or (IIb), R⁷ is C₂-C₈ heterocycloalkyl optionally substituted with one, two, or three R^{7a}. In some embodiments of a compound of Formula (II), (IIa), or (IIb), R⁷ is C₂-C₈ heterocycloalkyl optionally substituted with one, two, or three R^{7a}. In some embodiments of a compound of Formula (II), (IIa), or (IIb), R⁷ is C₂-C₄ heterocycloalkyl optionally substituted with one, two, or three R^{7a}. In some embodiments of a compound of Formula (II), (IIa), or (IIb), R⁷ is pyrrolidine or piperidine, each optionally substituted with one, two, or three R^{7a}. In some embodiments of a compound of Formula (II), (IIa), or (IIb), R⁷ is piperidine optionally substituted with one, two, or three R^{7a}. In some embodiments of a compound of Formula (II), (IIa), or (IIb), R⁷ is piperazine optionally substituted with one, two, or three R^{7a}. In some embodiments of a compound of Formula (II), (IIa), or (IIb), R⁷ is morpholine optionally substituted with one, two, or three R^{7a}.

In some embodiments of a compound of Formula (II), (IIa), or (IIb), R⁷ is C₃-C₈ cycloalkyl optionally substituted with one, two, or three R^{7a}. In some embodiments of a compound of Formula (II), (IIa), or (IIb), R⁷ is C₃-C₆ cycloalkyl optionally substituted with one, two, or three R^{7a}. In some embodiments of a compound of Formula (II), (IIa), or (IIb), R⁷ is C₃-C₈ cycloalkyl optionally substituted with one, two, or three R^{7a}. In some embodiments of a compound of Formula (II), (IIa), or (IIb), R⁷ is cyclohexyl optionally substituted with one, two, or three R^{7a}. In some embodiments of a compound of Formula (II), (IIa), or (IIb), R⁷ is cyclopentyl optionally substituted with one, two, or three R^{7a}. In some embodiments of a compound of Formula (II), (IIa), or (IIb), R⁷ is cyclobutyl optionally substituted with one, two, or three R^{7a}.

In some embodiments of a compound of Formula (II), (IIa), or (IIb), R⁷ is aryl or heteroaryl; each independently optionally substituted with one, two, or three R^{7a}.

In some embodiments of a compound of Formula (II), (IIa), or (IIb), R⁷ is phenyl or pyridyl; each independently optionally substituted with one, two, or three R^{7a}.

In some embodiments of a compound of Formula (II), (IIa), or (IIb), the compound of Formula (II) is of Formula (IIc):

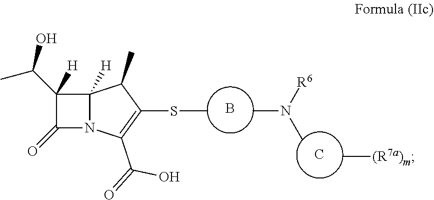

wherein
Ring C is C₃-C₈ cycloalkyl or C₂-C₈ heterocycloalkyl;
each R^{7a} is independently halogen, C₁-C₆ alkyl, C₂-C₆ alkenyl, C₂-C₆ alkynyl, C₁-C₆ haloalkyl, C₁-C₆ hydroxyalkyl, —(CR¹²R¹³)ᵥOR¹⁰, —(CR¹²R¹³)ᵥNR¹⁰R¹¹, —CN, —NO₂, —(CR¹²R¹³)ᵥS(═O)₂R¹⁰, —(CR¹²R¹³)ᵥS(═O)₂NR¹⁰R¹¹, —(CR¹²R¹³)ᵥC(═O)OR¹⁰, —(CR¹²R¹³)ᵥC(═O)NR⁰R¹, —(CR¹²R¹³)ᵥNR¹⁰C(═NR¹¹)NR¹⁰R¹¹, —(CR¹²R¹³)ᵥC(═NR¹⁰)R¹⁰, —(CR¹²R¹³)ᵥNR¹⁰C(═NR¹¹)R¹⁰, —(CR¹²R¹³)ᵥNR¹⁰(CR¹²R¹³)_{w}NR¹⁰R¹¹, —(CR¹²R¹³)ᵥ(NR¹⁰C(═NR¹¹)NR¹⁰(CR¹²R¹³)_{w}NR¹⁰R¹¹, —(CR¹²R¹³)ᵥC(═NR¹¹)NR¹⁰R¹¹, C₃-C₈ cycloalkyl, C₂-C₈ heterocycloalkyl, aryl, or heteroaryl; wherein each alkyl, alkenyl, alkynyl, cycloalkyl, heterocycloalkyl, aryl, and heteroaryl is independently optionally substituted with one, two, or three R^{7b};
each R^{7b} is independently halogen, C₁-C₆ alkyl, C₁-C₆ haloalkyl, —OW, —CN, —NO₂, —NR^{c}R^{d}, —S(═O)₂R^{b}, —S(═O)₂NR^{c}R^{d}, —C(═O)OR^{a}, —C(═O)R^{b}, —C(═O)NR^{c}R^{d}, —NR^{c}C(═NR^{c})NR^{c}R^{d}, or —NR^{c}C(═NR^{c})R^{b};
each R¹⁰ and R¹¹ is independently hydrogen, C₁-C₆ alkyl, C₁-C₆ haloalkyl, C₂-C₆ alkenyl, C₂-C₆ alkynyl, C₃-C₈ cycloalkyl, C₂-C₈ heterocycloalkyl, aryl, heteroaryl, —(C₁-C₆ alkyl)(C₃-C₈ cycloalkyl), —(C₁-C₆ alkyl)(C₂-C₈ heterocycloalkyl), —(C₁-C₆ alkyl)(aryl), or —(C₁-C₆ alkyl)(heteroaryl);
or R¹⁰ and R¹¹ are taken together with the nitrogen to which they are attached to form a C₂-C₈ heterocycloalkyl;
each R¹² and R¹³ is independently hydrogen, halogen, —CN, —OR^{a}, —NR^{c}R^{d}, C₁-C₆ alkyl, C₁-C₆ haloalkyl, C₂-C₆ alkenyl, C₂-C₆ alkynyl, C₃-C₈ cycloalkyl, C₂-C₈ heterocycloalkyl, aryl, or heteroaryl;
v is 0-4;
w is 2-4; and
m is 0-3.

In some embodiments of a compound of Formula (II), (IIa), (IIb), or (IIc), R⁶ is hydrogen, C₁-C₆ alkyl, C₁-C₆ haloalkyl, C₃-C₈ cycloalkyl, or C₂-C₈ heterocycloalkyl. In some embodiments of a compound of Formula (II), (IIa), (IIb), or (IIc), R⁶ is hydrogen, C₁-C₆ alkyl, or C₁-C₆ haloalkyl. In some embodiments of a compound of Formula (II), (IIa), (IIb), or (IIc), R⁶ is hydrogen or C₁-C₆ alkyl. In some embodiments of a compound of Formula (II), (IIa), (IIb), or (IIc), R⁶ is hydrogen. In some embodiments of a compound of Formula (II), (IIa), (IIb), or (IIc), R⁶ is C₁-C₆ alkyl.

In some embodiments of a compound of Formula (II), (IIa), (IIb), or (IIc), Ring C is C₂-C₈ heterocycloalkyl optionally substituted with one, two, or three R^{7a}. In some embodiments of a compound of Formula (II), (IIa), (IIb), or (IIc), Ring C is C₂-C₈ heterocycloalkyl optionally substituted with one, two, or three R^{7a}. In some embodiments of a compound of Formula (II), (IIa), (IIb), or (IIc), Ring C is C₂-C₄ heterocycloalkyl optionally substituted with one, two, or three R^{7a}. In some embodiments of a compound of Formula (II), (IIa), (IIb), or (IIc), Ring C is pyrrolidine or piperidine, each optionally substituted with one, two, or three R^{7a}. In some embodiments of a compound of Formula (II), (IIa), (IIb), or (IIc), Ring C is piperidine optionally substituted with one, two, or three R^{7a}. In some embodiments of a compound of Formula (II), (IIa), (IIb), or (IIc), Ring C is piperazine optionally substituted with one, two, or three R^{7a}. In some embodiments of a compound of Formula (II), (IIa), (IIb), or (IIc), Ring C is morpholine optionally substituted with one, two, or three R^{7a}.

In some embodiments of a compound of Formula (II), (IIa), (IIb), or (IIc), Ring C is C₃-C₈ cycloalkyl optionally substituted with one, two, or three R^{7a}. In some embodiments of a compound of Formula (II), (IIa), (IIb), or (IIc), Ring C is C₃-C₆ cycloalkyl optionally substituted with one, two, or three R^{7a}. In some embodiments of a compound of Formula (II), (IIa), (IIb), or (IIc), Ring C is C₃-C₈ cycloalkyl optionally substituted with one, two, or three R^{7a}. In some embodiments of a compound of Formula (II), (IIa), (IIb), or (IIc), Ring C is cyclohexyl optionally substituted with one, two, or three R^{7a}. In some embodiments of a compound of Formula (II), (IIa), (IIb), or (IIc), Ring C is cyclopentyl optionally substituted with one, two, or three R^{7a}. In some embodiments of a compound of Formula (II), (IIa), (IIb), or (IIc), Ring C is cyclobutyl optionally substituted with one, two, or three R^{7a}.

In some embodiments of a compound of Formula (II), (IIa), or (IIb), R⁶ and R⁷ are taken together with the nitrogen to which they are attached to form a C₂-C₈ heterocycloalkyl optionally substituted with one, two, or three R^{7a}. In some embodiments of a compound of Formula (II), (IIa), or (IIb), R⁶ and R⁷ are taken together with the nitrogen to which they are attached to form a C₂-C₈ heterocycloalkyl optionally substituted with one, two, or three R^{7a}. In some embodiments of a compound of Formula (II), (IIa), or (IIb), R⁶ and R⁷ are taken together with the nitrogen to which they are attached to form a C₂-C₄ heterocycloalkyl optionally substituted with one, two, or three R^{7a}. In some embodiments of a compound of Formula (II), (IIa), or (IIb), R⁶ and R⁷ are taken together with the nitrogen to which they are attached to form a pyrrolidine or piperidine, each optionally substituted with one, two, or three R^{7a}. In some embodiments of a compound of Formula (II), (IIa), or (IIb), R⁶ and R⁷ are taken together with the nitrogen to which they are attached to form a piperidine optionally substituted with one, two, or three R^{7a}. In some embodiments of a compound of Formula (II), (IIa), or (IIb), R⁶ and R⁷ are taken together with the nitrogen to which they are attached to form a piperazine optionally substituted with one, two, or three R^{7a}. In some embodiments of a compound of Formula (II), (IIa), or (IIb), R⁶ and R⁷ are taken together with the nitrogen to which they are attached to form a morpholine optionally substituted with one, two, or three R^{7a}.

In some embodiments of a compound of Formula (II), (IIa), or (IIb), the compound of Formula (II) is of Formula (IId):

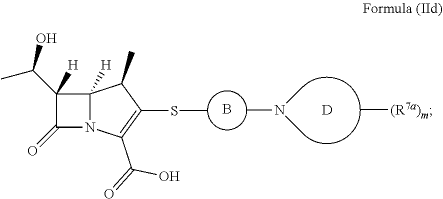

wherein
Ring D is C₂-C₆ heterocycloalkyl;
each R^{7a} is independently halogen, C₁-C₆ alkyl, C₂-C₆ alkenyl, C₂-C₆ alkynyl, C₁-C₆ haloalkyl, C₁-C₆ hydroxyalkyl, —(CR¹²R¹³)ᵥOR¹⁰, —(CR¹²R¹³)ᵥNR¹⁰R¹¹, —CN, —NO₂, —(CR¹²R¹³)ᵥS(═O)₂R¹⁰, —(CR¹²R¹³)ᵥS(═O)₂NR¹⁰R¹¹, —(CR¹²R¹³)ᵥC(═O)OR¹⁰, —(CR¹²R¹³)ᵥC(═O)NR⁰R¹, —(CR¹²R¹³)ᵥNR¹⁰C(═NR¹¹)NR¹⁰R¹¹, —(CR¹²R¹³)ᵥC(═NR¹⁰)R¹⁰, —(CR¹²R¹³)ᵥNR¹⁰C(═NR¹¹)R¹⁰, —(CR¹²R¹³)ᵥNR¹⁰(CR¹²R¹³)_{w}NR¹⁰R¹¹, —(CR¹²R¹³)ᵥ(NR¹⁰C(═NR¹¹)NR¹⁰(CR¹²R¹³)_{w}NR¹⁰R¹¹, —(CR¹²R¹³)ᵥC(═NR¹¹)NR¹⁰R¹¹, C₃-C₈ cycloalkyl, C₂-C₈ heterocycloalkyl, aryl, or heteroaryl; wherein each alkyl, alkenyl, alkynyl, cycloalkyl, heterocycloalkyl, aryl, and heteroaryl is independently optionally substituted with one, two, or three R^{7b};
each R^{7b} is independently halogen, C₁-C₆ alkyl, C₁-C₆ haloalkyl, —OR^{a}, —CN, —NO₂, —NR^{c}R^{d}, —S(═O)₂R^{b}, —S(═O)₂NR^{c}R^{d}, —C(═O)OR^{a}, —C(═O)R^{b}, —C(═O)NR^{c}R^{d}, —NR^{c}C(═NR^{c})NR^{c}R^{d}, or —NR^{c}C(═NR^{c})R^{b};
each R¹⁰ and R¹¹ is independently hydrogen, C₁-C₆ alkyl, C₁-C₆ haloalkyl, C₂-C₆ alkenyl, C₂-C₆ alkynyl, C₃-C₈ cycloalkyl, C₂-C₈ heterocycloalkyl, aryl, heteroaryl, —(C₁-C₆ alkyl)(C₃-C₈ cycloalkyl), —(C₁-C₆ alkyl)(C₂-C₈ heterocycloalkyl), —(C₁-C₆ alkyl)(aryl), or —(C₁-C₆ alkyl)(heteroaryl);
or R¹⁰ and R¹¹ are taken together with the nitrogen to which they are attached to form a C₂-C₈ heterocycloalkyl;
each R¹² and R¹³ is independently hydrogen, halogen, —CN, —OR^{a}, —NR^{c}R^{d}, C₁-C₆ alkyl, C₁-C₆ haloalkyl, C₂-C₆ alkenyl, C₂-C₆ alkynyl, C₃-C₈ cycloalkyl, C₂-C₈ heterocycloalkyl, aryl, or heteroaryl;
v is 0-4;
w is 2-4; and
m is 0-3.

In some embodiments of a compound of Formula (II), (IIa), (IIb), or (IId), Ring D is C₂-C₈ heterocycloalkyl. In some embodiments of a compound of Formula (II), (IIa), (IIb), or (IId), Ring D is C₂-C₄ heterocycloalkyl. In some embodiments of a compound of Formula (II), (IIa), (IIb), or (IId), Ring D is pyrrolidine or piperidine. In some embodiments of a compound of Formula (II), Ring D is piperidine. In some embodiments of a compound of Formula (II), Ring D is piperazine. In some embodiments of a compound of Formula (II), Ring D is morpholine.

In some embodiments of a compound of Formula (II) or (IIa)-(IId), m is 0-2. In some embodiments of a compound of Formula (II) or (IIa)-(IId), m is 0 or 1. In some embodiments of a compound of Formula (II) or (IIa)-(IId), m is 0. In some embodiments of a compound of Formula (II) or (IIa)-(IId), m is 1. In some embodiments of a compound of Formula (II) or (IIa)-(IId), m is 2. In some embodiments of a compound of Formula (II) or (IIa)-(IId), m is 3. In some embodiments of a compound of Formula (II) or (IIa)-(IId), m is 1 or 2.

In some embodiments of a compound of Formula (II) or (IIa)-(IId), Ring B is cyclobutyl, cyclopentyl, or cyclohexyl. In some embodiments of a compound of Formula (II), Ring B is cyclobutyl. In some embodiments of a compound of Formula (II), Ring B is cyclopentyl. In some embodiments of a compound of Formula (II), Ring B is cyclohexyl.

In some embodiments of a compound of Formula (II) or (IIa)-(IId), each R^{7a} is independently —(CR¹²R¹³)ᵥNR¹⁰R¹¹, —(CR¹²R¹³)ᵥS(═O)₂NR¹⁰R¹¹, —(CR¹²R¹³)ᵥC(═O)NR¹⁰R¹¹, —(CR¹²R¹³)ᵥNR¹⁰C(═NR¹¹)NR¹⁰R¹¹, —(CR¹²R¹³)ᵥC(═NR¹⁰)R¹⁰, —(CR¹²R¹³)ᵥNR¹⁰C(═NR¹¹)R¹⁰, —(CR¹²R¹³)ᵥNR¹⁰(CR¹²R¹³)_{w}NR¹⁰R¹¹, —(CR¹²R¹³)ᵥ(NR¹⁰C(═NR¹¹)NR¹⁰(CR¹²R¹³)_{w}NR¹⁰R¹¹, or —(CR¹²R¹³)ᵥC(═NR¹¹)NR¹⁰R¹¹.

In some embodiments of a compound of Formula (II) or (IIa)-(IId), each R^{7a} is independently —(CR¹²R¹³)ᵥNR¹⁰R¹¹, —(CR¹²R¹³)ᵥNR¹⁰C(═NR¹¹)NR¹⁰R¹¹, —(CR¹²R¹³)C(═NR¹⁰)R¹⁰, —(CR¹²R¹³)ᵥNR¹⁰C(═NR¹¹)R¹⁰, —(CR¹²R¹³)ᵥNR¹⁰(CR¹²R¹³)_{w}NR¹⁰R¹¹, —(CR¹²R¹³)ᵥ(NR¹⁰C(═NR¹¹)NR¹⁰(CR¹²R¹³)_{w}NR¹⁰R¹¹, or —(CR¹²R¹³)ᵥC(═NR¹¹)NR¹⁰R¹¹.

In some embodiments of a compound of Formula (II) or (IIa)-(IId), each R^{7a} is independently —(CR¹²R¹³)ᵥNR¹⁰R¹¹, —(CR¹²R¹³)ᵥNR¹⁰C(═NR¹¹)NR¹⁰R¹¹, —(CR¹²R¹³)C(═NR¹⁰)R¹⁰, —(CR¹²R¹³)ᵥNR¹⁰C(═NR¹¹)R¹⁰, or —(CR¹²R¹³)ᵥC(═NR¹¹)NR¹⁰R¹¹.

In some embodiments of a compound of Formula (II) or (IIa)-(IId), each R^{7a} is independently —(CR¹²R¹³)ᵥNR¹⁰R¹¹, —(CR¹²R¹³)ᵥNR¹⁰C(═NR¹¹)NR¹⁰R¹¹, or —(CR¹²R¹³)ᵥC(═NR¹¹)NR¹⁰R¹¹.

In some embodiments of a compound of Formula (II) or (IIa)-(IId), each R^{7a} is independently —(CR¹²R¹³)ᵥNR¹⁰R¹¹ or —(CR¹²R¹³)ᵥNR¹⁰C(═NR¹¹)NR¹⁰R¹¹.

In some embodiments of a compound of Formula (II) or (IIa)-(IId), v is 0 or 1. In some embodiments of a compound of Formula (II) or (IIa)-(IId), v is 0. In some embodiments of a compound of Formula (II) or (IIa)-(IId), v is 1. In some embodiments of a compound of Formula (II) or (IIa)-(IId), v is 2. In some embodiments of a compound of Formula (II) or (IIa)-(IId), v is 3. In some embodiments of a compound of Formula (II) or (IIa)-(IId), v is 4. In some embodiments of a compound of Formula (II) or (IIa)-(IId), v is 0, 1, or 2. In some embodiments of a compound of Formula (II) or (IIa)-(IId), v is 1 or 2.

In some embodiments of a compound of Formula (II) or (IIa)-(IId), w is 2 or 3. In some embodiments of a compound of Formula (II) or (IIa)-(IId), w is 2. In some embodiments of a compound of Formula (II) or (IIa)-(IId), w is 3. In some embodiments of a compound of Formula (II) or (IIa)-(IId), w is 4.

In some embodiments of a compound of Formula (II) or (IIa)-(IId), each R¹⁰ and R¹¹ is independently hydrogen, C₁-C₆ alkyl, C₁-C₆ haloalkyl, C₃-C₈ cycloalkyl, C₂-C₈ heterocycloalkyl, aryl, heteroaryl, —(C₁-C₆ alkyl)(C₃-C₈ cycloalkyl), —(C₁-C₆ alkyl)(C₂-C₈ heterocycloalkyl), —(C₁-C₆ alkyl)(aryl), or —(C₁-C₆ alkyl)(heteroaryl). In some embodiments of a compound of Formula (II) or (IIa)-(IId), each R¹⁰ and R¹¹ is independently hydrogen, C₁-C₆ alkyl, C₁-C₆ haloalkyl, C₃-C₈ cycloalkyl, C₂-C₈ heterocycloalkyl, —(C₁-C₆ alkyl)(C₃-C₈ cycloalkyl), —(C₁-C₆ alkyl)(C₂-C₈ heterocycloalkyl), —(C₁-C₆ alkyl)(aryl), or —(C₁-C₆ alkyl)(heteroaryl). In some embodiments of a compound of Formula (II) or (IIa)-(IId), each R¹⁰ and R¹¹ is independently hydrogen, C₁-C₆ alkyl, C₁-C₆ haloalkyl, C₃-C₈ cycloalkyl, C₂-C₈ heterocycloalkyl, aryl, or heteroaryl. In some embodiments of a compound of Formula (II) or (IIa)-(IId), each R¹⁰ and R¹¹ is independently hydrogen, C₁-C₆ alkyl, or C₁-C₆ haloalkyl. In some embodiments of a compound of Formula (II) or (IIa)-(IId), each R¹⁰ and R¹¹ is independently hydrogen or C₁-C₆ alkyl. In some embodiments of a compound of Formula (II) or (IIa)-(IId), each R¹⁰ and R¹¹ is C₁-C₆ alkyl. In some embodiments of a compound of Formula (II) or (IIa)-(IId), each R¹⁰ and R¹¹ is hydrogen. In some embodiments of a compound of Formula (II) or (IIa)-(IId), R¹⁰ and R¹¹ are taken together with the nitrogen to which they are attached to form a C₂-C₈ heterocycloalkyl.

In some embodiments of a compound of Formula (II) or (IIa)-(IId), each R¹² and R¹³ is independently hydrogen, halogen, —CN, —OR^{a}, —NR^{c}R^{d}, C₁-C₆ alkyl, C₁-C₆ haloalkyl, C₃-C₈ cycloalkyl, C₂-C₈ heterocycloalkyl, aryl, or heteroaryl. In some embodiments of a compound of Formula (II) or (IIa)-(IId), each R¹² and R¹³ is independently hydrogen, halogen, —CN, —OR^{a}, —NR^{c}R^{d}, C₁-C₆ alkyl, C₁-C₆ haloalkyl, C₃-C₈ cycloalkyl, or C₂-C₈ heterocycloalkyl. In some embodiments of a compound of Formula (II) or (IIa)-(IId), each R¹² and R¹³ is independently hydrogen, halogen, —CN, —OR^{a}, —NR^{c}R^{d}, C₁-C₆ alkyl, or C₁-C₆ haloalkyl. In some embodiments of a compound of Formula (II) or (IIa)-(IId), each R¹² and R¹³ is independently hydrogen, halogen, C₁-C₆ alkyl, or C₁-C₆ haloalkyl. In some embodiments of a compound of Formula (II) or (IIa)-(IId), each R¹² and R¹³ is independently hydrogen, halogen, or C₁-C₆ alkyl. In some embodiments of a compound of Formula (II) or (IIa)-(IId), each R¹² and R¹³ is hydrogen.

In some embodiments of a compound of Formula (II) or (IIa)-(IId), each R^{a} is independently hydrogen, C₁-C₆ alkyl, C₁-C₆ haloalkyl, C₃-C₈ cycloalkyl, or C₂-C₈ heterocycloalkyl. In some embodiments of a compound of Formula (II) or (IIa)-(IId), each R^{a} is independently hydrogen, C₁-C₆ alkyl, or C₁-C₆ haloalkyl. In some embodiments of a compound of Formula (II) or (IIa)-(IId), each R^{a} is independently hydrogen or C₁-C₆ alkyl. In some embodiments of a compound of Formula (II) or (IIa)-(IId), each R^{a} is independently C₁-C₆ alkyl. In some embodiments of a compound of Formula (II) or (IIa)-(IId), each R^{a} is hydrogen.

In some embodiments of a compound of Formula (II) or (IIa)-(IId), each R^{b} is independently C₁-C₆ alkyl, C₁-C₆ haloalkyl, C₃-C₈ cycloalkyl, or C₂-C₈ heterocycloalkyl. In some embodiments of a compound of Formula (II) or (IIa)-(IId), each R^{b} is independently C₁-C₆ alkyl, or C₁-C₆ haloalkyl. In some embodiments of a compound of Formula (II) or (IIa)-(IId), each R^{b} is independently C₁-C₆ alkyl.

In some embodiments of a compound of Formula (II) or (IIa)-(IId), each R^{c} and R^{d} are independently hydrogen, C₁-C₆ alkyl, C₁-C₆ haloalkyl, C₃-C₈ cycloalkyl, or C₂-C₈ heterocycloalkyl. In some embodiments of a compound of Formula (II) or (IIa)-(IId), each R^{c} and R^{d} are independently hydrogen, C₁-C₆ alkyl, or C₁-C₆ haloalkyl. In some embodiments of a compound of Formula (II) or (IIa)-(IId), each R^{c} and R^{d} are independently hydrogen or C₁-C₆ alkyl. In some embodiments of a compound of Formula (II) or (IIa)-(IId), each R^{c} and R^{d} are independently C₁-C₆ alkyl. In some embodiments of a compound of Formula (II) or (IIa)-(IId), each R^{c} and R^{d} are hydrogen.

In some embodiments of a compound of Formula (II) or (IIa)-(IId), R^{c} and R^{d} are taken together with the nitrogen to which they are attached to form a C₂-C₈ heterocycloalkyl. In some embodiments of a compound of Formula (II) or (IIa)-(IId), R^{c} and R^{d} are taken together with the nitrogen to which they are attached to form a pyrrolidine, piperidine, morpholine, or piperazine.

Also disclosed herein is a compound of Formula (III), or a pharmaceutically acceptable salt, solvate, or stereoisomer thereof:

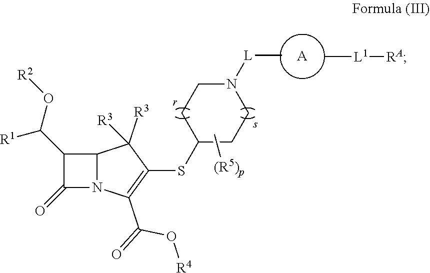

wherein
Ring A is cycloalkyl optionally substituted with one, two, or three oxo, halogen, C₁-C₆ alkyl, C₁-C₆ haloalkyl, —OR^{a}, —CN, —NO₂, —NR^{c}R^{d}, —S(═O)₂R^{b}, —S(═O)₂NR^{c}R^{d}, —C(═O)OR^{a}, —C(═O)R^{b}, —C(═O)NR^{c}R^{d}, —NR^{c}C(═NR^{c})NR^{c}R^{d}, or —NR^{c}C(═NR^{c})R^{b};
L¹ is a bond or C₁-C₆ alkylene optionally substituted with halogen, —OR^{a}, or —NR^{c}R^{d};
R^{A} is halogen, C₁-C₆ alkyl, C₂-C₆ alkenyl, C₂-C₆ alkynyl, C₁-C₆ haloalkyl, C₁-C₆ hydroxyalkyl, —OR¹⁰, —CN, —NO₂, —S(═O)₂R¹⁰, —S(═O)₂NR¹⁰R¹¹, —C(═O)OR¹⁰, —C(═O)NR¹⁰R¹¹, —NR¹⁰C(═NR¹¹)NR¹⁰R¹¹, —C(═NR¹⁰)R¹⁰, —NR¹⁰C(═NR¹¹)R¹⁰, —NR¹⁰(CR¹²R¹³)_{w}NR¹⁰R¹¹, —NR¹⁰C(═NR¹¹)NR¹⁰(CR¹²R¹³)_{w}NR¹⁰R¹¹, —C(═NR¹¹)NR¹⁰R¹¹, C₃-C₈ cycloalkyl, C₂-C₈ heterocycloalkyl, aryl, or heteroaryl; wherein each alkyl, alkenyl, alkynyl, cycloalkyl, heterocycloalkyl, aryl, and heteroaryl is independently optionally substituted with one, two, or three R^{A1};
each R^{A1} is independently halogen, C₁-C₆ alkyl, C₁-C₆ haloalkyl, —OR^{a}, —CN, —NO₂, —NR^{c}R^{d}, —S(═O)₂R^{b}, —S(═O)₂NR^{c}R^{d}, —C(═O)OR^{a}, —C(═O)R^{b}, —C(═O)NR^{c}R^{d}, —NR^{c}C(═NR)NR^{c}R^{d}, or —NR^{c}C(═NR^{c})R^{b};
L is a bond or C₁-C₆ alkylene optionally substituted with halogen, —OR^{a}, or —NR^{c}R^{d};
R¹ is C₁-C₆ alkyl, C₁-C₆ haloalkyl, C₂-C₆ alkenyl, or C₂-C₆ alkynyl;
R² is hydrogen, C₁-C₆ alkyl, C₁-C₆ haloalkyl, C₂-C₆ alkenyl, C₂-C₆ alkynyl, or —Si(R^{b})₃; each R³ is independently hydrogen, C₁-C₆ alkyl, C₁-C₆ haloalkyl, C₂-C₆ alkenyl, C₂-C₆ alkynyl, or —OR^{a};
R⁴ is hydrogen, C₁-C₆ alkyl, C₁-C₆ haloalkyl, C₂-C₆ alkenyl, C₂-C₆ alkynyl, C₃-C₈ cycloalkyl, C₂-C₈ heterocycloalkyl, aryl, heteroaryl, —(C₁-C₆ alkyl)(C₃-C₈ cycloalkyl), —(C₁-C₆ alkyl)(C₂-C₈ heterocycloalkyl), —(C₁-C₆ alkyl)(aryl), or —(C₁-C₆ alkyl)(heteroaryl);
each R⁵ is independently halogen, C₁-C₆ alkyl, C₁-C₆ haloalkyl, C₂-C₆ alkenyl, C₂-C₆ alkynyl, —OR^{a}, —CN, —NO₂, —S(═O)₂R^{b}, —S(═O)₂NR^{c}R^{d}, —C(═O)OR^{a}, —C(═O)R^{b}, —C(═O)NR^{c}R^{d}, —NR^{c}R^{d}, C₃-C₈ cycloalkyl, or C₂-C₈ heterocycloalkyl;
or two R⁵ on the same carbon are taken together to form an oxo;
each R¹⁰ and R¹¹ is independently hydrogen, C₁-C₆ alkyl, C₁-C₆ haloalkyl, C₂-C₆ alkenyl, C₂-C₆ alkynyl, C₃-C₈ cycloalkyl, C₂-C₈ heterocycloalkyl, aryl, heteroaryl, —(C₁-C₆ alkyl)(C₃-C₈ cycloalkyl), —(C₁-C₆ alkyl)(C₂-C₈ heterocycloalkyl), —(C₁-C₆ alkyl)(aryl), or —(C₁-C₆ alkyl)(heteroaryl);
or R¹⁰ and R¹¹ are taken together with the nitrogen to which they are attached to form a C₂-C₈ heterocycloalkyl;
each R¹² and R¹³ is independently hydrogen, halogen, —CN, —OR^{a}, —NR^{c}R^{d}, C₁-C₆ alkyl, C₁-C₆ haloalkyl, C₂-C₆ alkenyl, C₂-C₆ alkynyl, C₃-C₈ cycloalkyl, C₂-C₈ heterocycloalkyl, aryl, or heteroaryl;
each R^{a} is independently hydrogen, C₁-C₆ alkyl, C₁-C₆ haloalkyl, C₂-C₆ alkenyl, C₂-C₆ alkynyl, C₃-C₈ cycloalkyl, or C₂-C₈ heterocycloalkyl;
each R^{b} is independently C₁-C₆ alkyl, C₁-C₆ haloalkyl, C₂-C₆ alkenyl, C₂-C₆ alkynyl, C₃-C₈ cycloalkyl, or C₂-C₈ heterocycloalkyl;
each R^{c} and R^{d} are independently hydrogen, C₁-C₆ alkyl, C₁-C₆ haloalkyl, C₂-C₆ alkenyl, C₂-C₆ alkynyl, C₃-C₈ cycloalkyl, or C₂-C₈ heterocycloalkyl;
or R^{c} and R^{d} are taken together with the nitrogen to which they are attached to form a C₂-C₈ heterocycloalkyl;
w is 2-4;
r is 1-2;
s is 0-2; and
p is 0-5.

In some embodiments of a compound of Formula (III), R¹ is C₁-C₆ alkyl. In some embodiments of a compound of Formula (III), R¹ is methyl. In some embodiments of a compound of Formula (III), R¹ is C₂-C₆ alkenyl. In some embodiments of a compound of Formula (III), R¹ is C₂-C₆ alkynyl.

In some embodiments of a compound of Formula (III) R² is hydrogen. In some embodiments of a compound of Formula (III), R² is C₁-C₆ alkyl. In some embodiments of a compound of Formula (III), R² is methyl. In some embodiments of a compound of Formula (III), R² is C₂-C₆ alkenyl. In some embodiments of a compound of Formula (III), R² is C₂-C₆ alkynyl. In some embodiments of a compound of Formula (III), R² is —Si(R^{b})₃.

In some embodiments of a compound of Formula (III), each R³ is hydrogen. In some embodiments of a compound of Formula (III), each R³ is independently hydrogen or C₁-C₆ alkyl. In some embodiments of a compound of Formula (III), each R³ is independently hydrogen, C₁-C₆ alkyl, or OR^{a}. In some embodiments of a compound of Formula (III), each R³ is independently hydrogen, C₁-C₆ alkyl, or OH. In some embodiments of a compound of Formula (III), each R³ is independently C₁-C₆ alkyl. In some embodiments of a compound of Formula (III), one R³ is methyl and the other R³ is hydrogen. In some embodiments of a compound of Formula (III), each R³ is independently hydrogen or C₂-C₆ alkenyl. In some embodiments of a compound of Formula (III), each R³ is independently hydrogen or C₂-C₆ alkynyl.

In some embodiments of a compound of Formula (III), R⁴ is hydrogen. In some embodiments of a compound of Formula (III), R⁴ is C₁-C₆ alkyl. In some embodiments of a compound of Formula (III), R⁴ is C₂-C₆ alkenyl. In some embodiments of a compound of Formula (III), R⁴ is C₂-C₆ alkynyl. In some embodiments of a compound of Formula (III), R⁴ is —(C₁-C₆ alkyl)(aryl).

In some embodiments of a compound of Formula (III), the compound is of Formula (IIIa):

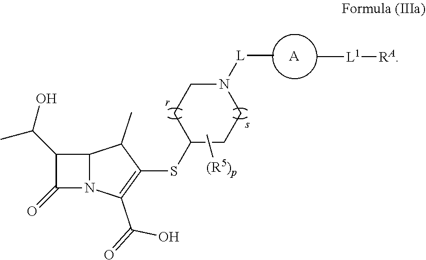

In some embodiments of a compound of Formula (III), the compound is of Formula (IIIb):

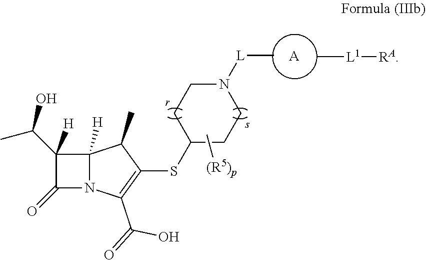

In some embodiments of a compound of Formula (III), (IIIa), or (IIIb), p is 0. In some embodiments of a compound of Formula (III), (IIIa), or (IIIb), p is 1. In some embodiments of a compound of Formula (III), (IIIa), or (IIIb), p is 2. In some embodiments of a compound of Formula (III), (IIIa), or (IIIb), p is 3. In some embodiments of a compound of Formula (III), (IIIa), or (IIIb), p is 4. In some embodiments of a compound of Formula (III), (IIIa), or (IIIb), p is 5. In some embodiments of a compound of Formula (III), (IIIa), or (IIIb), p is 0 or 1. In some embodiments of a compound of Formula (III), (IIIa), or (IIIb), p is 0, 1, or 2. In some embodiments of a compound of Formula (III), (IIIa), or (IIIb), p is 1 or 2. In some embodiments of a compound of Formula (III), (IIIa), or (IIIb), p is 2 or 3.

In some embodiments of a compound of Formula (III), (IIIa), or (IIIb), each R is independently halogen, C₁-C₆ alkyl, C₁-C₆ haloalkyl, —OR^{a}, —CN, —C(═O)R^{b}, —NR^{c}R^{d}, C₃-C₈ cycloalkyl, or C₂-C₈ heterocycloalkyl. In some embodiments of a compound of Formula (III), (IIIa), or (IIIb), each R⁵ is independently halogen, C₁-C₆ alkyl, C₁-C₆ haloalkyl, or —OR^{a}. In some embodiments of a compound of Formula (III), (IIIa), or (IIIb), each R⁵ is independently halogen or C₁-C₆ alkyl. In some embodiments of a compound of Formula (III), (IIIa), or (IIIb), two R⁵ on the same carbon are taken together to form an oxo.

In some embodiments of a compound of Formula (III), (IIIa), or (IIIb), r is 1 and s is 0. In some embodiments of a compound of Formula (III), (IIIa), or (IIIb), r is 2 and s is 0. In some embodiments of a compound of Formula (III), (IIIa), or (IIIb), r is 1 and s is 1. In some embodiments of a compound of Formula (III), (IIIa), or (IIIb), r is 1 and s is 2. In some embodiments of a compound of Formula (III), (IIIa), or (IIIb), r is 2 and s is 1. In some embodiments of a compound of Formula (III), (IIIa), or (IIIb), r is 2 and s is 2.

In some embodiments of a compound of Formula (III), (IIIa), or (IIIb), L is a bond. In some embodiments of a compound of Formula (III), (IIIa), or (IIIb), L is C₁-C₆ alkylene. In some embodiments of a compound of Formula (III), (IIIa), or (IIIb), L is C₁-C₂ alkylene. In some embodiments of a compound of Formula (III), (IIIa), or (IIIb), L is C₁ alkylene.

In some embodiments of a compound of Formula (III), (IIIa), or (IIIb), Ring A is cyclobutyl, cyclopentyl, or cyclohexyl. In some embodiments of a compound of Formula (III), (IIIa), or (IIIb), Ring A is cyclohexyl.

In some embodiments of a compound of Formula (III), (IIIa), or (IIIb), L¹ is a bond. In some embodiments of a compound of Formula (III), (IIIa), or (IIIb), L¹ is C₁-C₆ alkylene. In some embodiments of a compound of Formula (III), (IIIa), or (IIIb), L¹ is C₁-C₂ alkylene. In some embodiments of a compound of Formula (III), (IIIa), or (IIIb), L¹ is C₁ alkylene.

In some embodiments of a compound of Formula (III), (IIIa), or (IIIb), R^{A} is —S(═O)₂NR¹⁰R¹¹, —C(═O)NR¹⁰R¹¹, —NR¹⁰C(═NR¹¹)NR¹⁰R¹¹, —C(═NR¹⁰)R¹⁰, —NR¹⁰C(═NR¹¹)R¹⁰, —NR¹⁰(CR¹²R¹³)_{w}NR¹⁰R¹¹, —NR¹⁰C(═NR¹¹)NR¹⁰(CR¹²R¹³)_{w}NR¹⁰R¹¹, or —C(═NR¹¹)NR¹⁰R¹¹. In some embodiments of a compound of Formula (III), (IIIa), or (IIIb), R^{A} is —NR¹⁰C(═NR¹¹)NR¹⁰R¹¹, —C(═NR¹⁰)R¹⁰, or —NR¹⁰C(═NR¹¹)R¹⁰. In some embodiments of a compound of Formula (III), (IIIa), or (IIIb), R^{A} is NR¹⁰C(═NR¹¹)NR¹⁰R¹¹.

In some embodiments of a compound of Formula (III), (IIIa), or (IIIb), each R¹⁰ and R¹¹ is independently hydrogen, C₁-C₆ alkyl, C₁-C₆ haloalkyl, C₃-C₈ cycloalkyl, C₂-C₈ heterocycloalkyl, aryl, heteroaryl, —(C₁-C₆ alkyl)(C₃-C₈ cycloalkyl), —(C₁-C₆ alkyl)(C₂-C₈ heterocycloalkyl), —(C₁-C₆ alkyl)(aryl), or —(C₁-C₆ alkyl)(heteroaryl). In some embodiments of a compound of Formula (III), (IIIa), or (IIIb), each R¹⁰ and R¹¹ is independently hydrogen, C₁-C₆ alkyl, C₁-C₆ haloalkyl, C₃-C₈ cycloalkyl, C₂-C₈ heterocycloalkyl, —(C₁-C₆ alkyl)(C₃-C₈ cycloalkyl), —(C₁-C₆ alkyl)(C₂-C₈ heterocycloalkyl), —(C₁-C₆ alkyl)(aryl), or —(C₁-C₆ alkyl)(heteroaryl). In some embodiments of a compound of Formula (III), (IIIa), or (IIIb), each R¹⁰ and R¹¹ is independently hydrogen, C₁-C₆ alkyl, C₁-C₆ haloalkyl, C₃-C₈ cycloalkyl, C₂-C₈ heterocycloalkyl, aryl, or heteroaryl. In some embodiments of a compound of Formula (III), (IIIa), or (IIIb), each R¹⁰ and R¹¹ is independently hydrogen, C₁-C₆ alkyl, or C₁-C₆ haloalkyl. In some embodiments of a compound of Formula (III), (IIIa), or (IIIb), each R¹⁰ and R¹¹ is independently hydrogen or C₁-C₆ alkyl. In some embodiments of a compound of Formula (III), (IIIa), or (IIIb), each R¹⁰ and R¹¹ is C₁-C₆ alkyl. In some embodiments of a compound of Formula (III), (IIIa), or (IIIb), each R¹⁰ and R¹¹ is hydrogen. In some embodiments of a compound of Formula (III), (IIIa), or (IIIb), R¹⁰ and R¹¹ are taken together with the nitrogen to which they are attached to form a C₂-C₈ heterocycloalkyl.

In some embodiments of a compound of Formula (III), (IIIa), or (IIIb), each R¹² and R¹³ is independently hydrogen, halogen, —CN, —OR^{a}, —NR^{c}R^{d}, C₁-C₆ alkyl, C₁-C₆ haloalkyl, C₃-C₈ cycloalkyl, C₂-C₈ heterocycloalkyl, aryl, or heteroaryl. In some embodiments of a compound of Formula (III), (IIIa), or (IIIb), each R¹² and R¹³ is independently hydrogen, halogen, —CN, —OR^{a}, —NR^{c}R^{d}, C₁-C₆ alkyl, C₁-C₆ haloalkyl, C₃-C₈ cycloalkyl, or C₂-C₈ heterocycloalkyl. In some embodiments of a compound of Formula (III), (IIIa), or (IIIb), each R¹² and R¹³ is independently hydrogen, halogen, —CN, —OR^{a}, —NR^{c}R^{d}, C₁-C₆ alkyl, or C₁-C₆ haloalkyl. In some embodiments of a compound of Formula (III), (IIIa), or (IIIb), each R¹² and R¹³ is independently hydrogen, halogen, C₁-C₆ alkyl, or C₁-C₆ haloalkyl. In some embodiments of a compound of Formula (III), (IIIa), or (IIIb), each R¹² and R¹³ is independently hydrogen, halogen, or C₁-C₆ alkyl. In some embodiments of a compound of Formula (III), (IIIa), or (IIIb), each R¹² and R¹³ is hydrogen.

In some embodiments of a compound of Formula (III), (IIIa), or (IIIb), each R^{a} is independently hydrogen, C₁-C₆ alkyl, C₁-C₆ haloalkyl, C₃-C₈ cycloalkyl, or C₂-C₈ heterocycloalkyl. In some embodiments of a compound of Formula (III), (IIIa), or (IIIb), each R^{a} is independently hydrogen, C₁-C₆ alkyl, or C₁-C₆ haloalkyl. In some embodiments of a compound of Formula (III), (IIIa), or (IIIb), each R^{a} is independently hydrogen or C₁-C₆ alkyl. In some embodiments of a compound of Formula (III), (IIIa), or (IIIb), each R^{a} is independently C₁-C₆ alkyl. In some embodiments of a compound of Formula (III), (IIIa), or (IIIb), each R^{a} is hydrogen.

In some embodiments of a compound of Formula (III), (IIIa), or (IIIb), each R^{b} is independently C₁-C₆ alkyl, C₁-C₆ haloalkyl, C₃-C₈ cycloalkyl, or C₂-C₈ heterocycloalkyl. In some embodiments of a compound of Formula (III), (IIIa), or (IIIb), each R^{b} is independently C₁-C₆ alkyl, or C₁-C₆ haloalkyl. In some embodiments of a compound of Formula (III), (IIIa), or (IIIb), each R^{b} is independently C₁-C₆ alkyl.

In some embodiments of a compound of Formula (III), (IIIa), or (IIIb), each R^{c} and R^{d} are independently hydrogen, C₁-C₆ alkyl, C₁-C₆ haloalkyl, C₃-C₈ cycloalkyl, or C₂-C₈ heterocycloalkyl. In some embodiments of a compound of Formula (III), (IIIa), or (IIIb), each R^{c} and R^{d} are independently hydrogen, C₁-C₆ alkyl, or C₁-C₆ haloalkyl. In some embodiments of a compound of Formula (III), (IIIa), or (IIIb), each R^{c} and R^{d} are independently hydrogen or C₁-C₆ alkyl. In some embodiments of a compound of Formula (III), (IIIa), or (IIIb), each R and R^{a} are independently C₁-C₆ alkyl. In some embodiments of a compound of Formula (III), (IIIa), or (IIIb), each R^{c} and R^{d} are hydrogen.

In some embodiments of a compound of Formula (III), (IIIa), or (IIIb), R^{c} and R^{d} are taken together with the nitrogen to which they are attached to form a C₂-C₈ heterocycloalkyl. In some embodiments of a compound of Formula (III), (IIIa), or (IIIb), R^{c} and R^{d} are taken together with the nitrogen to which they are attached to form a pyrrolidine, piperidine, morpholine, or piperazine.

Described herein are compounds, or a pharmaceutically acceptable salt, solvate, or stereoisomer thereof, selected from the group consisting of a compound found in table 1.

TABLE 1

| | | | ESI-MS (m/z) |
|---|---|---|---|
| Example | Structure | MW | for [M + H]⁺ |
| 1 | 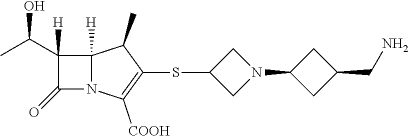 [SMILES: C[C@@H](O)[C@H]1C(=O)N2C(C(=O)O)=C(SC3CN([C@H]4C[C@@H](CN)C4)C3)[C@H](C)[C@H]12] | 381.49 | 382 |
| 2 | 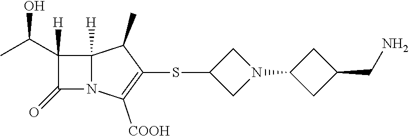 [SMILES: C[C@@H](O)[C@H]1C(=O)N2C(C(=O)O)=C(SC3CN([C@H]4C[C@H](CN)C4)C3)[C@H](C)[C@H]12] | 381.49 | 382 |
| 3 | 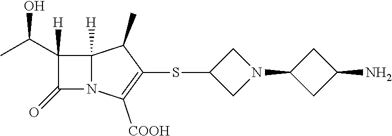 [SMILES: C[C@@H](O)[C@H]1C(=O)N2C(C(=O)O)=C(SC3CN([C@H]4C[C@@H](N)C4)C3)[C@H](C)[C@H]12] | 367.46 | 368 |
| 4 | 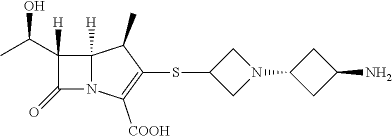 [SMILES: C[C@@H](O)[C@H]1C(=O)N2C(C(=O)O)=C(SC3CN([C@H]4C[C@H](N)C4)C3)[C@H](C)[C@H]12] | 367.46 | 368 |
| 5 | 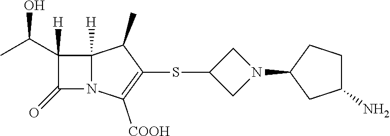 [SMILES: C[C@@H](O)[C@H]1C(=O)N2C(C(=O)O)=C(SC3CN([C@H]4CC[C@H](N)C4)C3)[C@H](C)[C@H]12] | 381.49 | 382 |
| 6 | 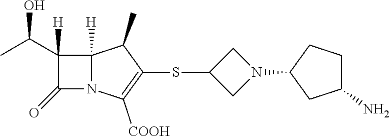 [SMILES: C[C@@H](O)[C@H]1C(=O)N2C(C(=O)O)=C(SC3CN([C@@H]4CC[C@H](N)C4)C3)[C@H](C)[C@H]12] | 381.49 | 382 |
| 7 | 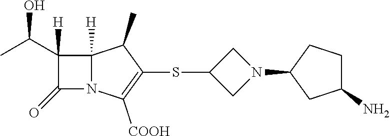 [SMILES: C[C@@H](O)[C@H]1C(=O)N2C(C(=O)O)=C(SC3CN([C@H]4CC[C@@H](N)C4)C3)[C@H](C)[C@H]12] | 381.49 | 382 |
| 8 | 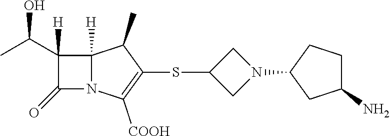 [SMILES: C[C@@H](O)[C@H]1C(=O)N2C(C(=O)O)=C(SC3CN([C@@H]4CC[C@@H](N)C4)C3)[C@H](C)[C@H]12] | 381.49 | 382 |
| 9 | 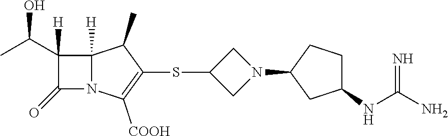 [SMILES: C[C@@H](O)[C@H]1C(=O)N2C(C(=O)O)=C(SC3CN([C@H]4CC[C@@H](NC(=N)N)C4)C3)[C@H](C)[C@H]12] | 423.53 | 424 |
| 10 | 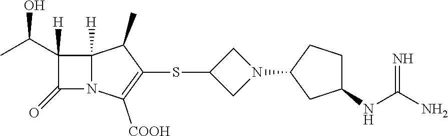 [SMILES: C[C@@H](O)[C@H]1C(=O)N2C(C(=O)O)=C(SC3CN([C@@H]4CC[C@@H](NC(=N)N)C4)C3)[C@H](C)[C@H]12] | 423.53 | 424 |
| 11 | 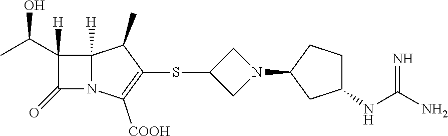 [SMILES: C[C@@H](O)[C@H]1C(=O)N2C(C(=O)O)=C(SC3CN([C@H]4CC[C@H](NC(=N)N)C4)C3)[C@H](C)[C@H]12] | 423.53 | 424 |
| 12 | 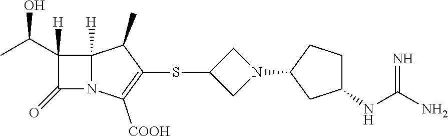 [SMILES: C[C@@H](O)[C@H]1C(=O)N2C(C(=O)O)=C(SC3CN([C@@H]4CC[C@H](NC(=N)N)C4)C3)[C@H](C)[C@H]12] | 423.53 | 424 |
| 13 | 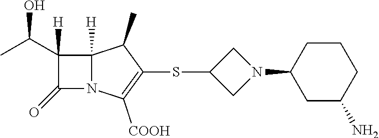 [SMILES: C[C@@H](O)[C@H]1C(=O)N2C(C(=O)O)=C(SC3CN([C@H]4CCC[C@H](N)C4)C3)[C@H](C)[C@H]12] | 395.52 | 396 |
| 14 | 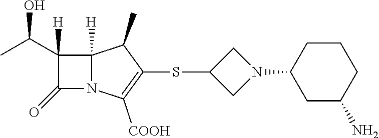 [SMILES: C[C@@H](O)[C@H]1C(=O)N2C(C(=O)O)=C(SC3CN([C@@H]4CCC[C@H](N)C4)C3)[C@H](C)[C@H]12] | 395.52 | 396 |
| 15 | 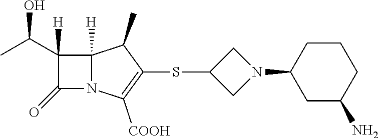 [SMILES: C[C@@H](O)[C@H]1C(=O)N2C(C(=O)O)=C(SC3CN([C@H]4CCC[C@@H](N)C4)C3)[C@H](C)[C@H]12] | 395.52 | 396 |
| 16 | 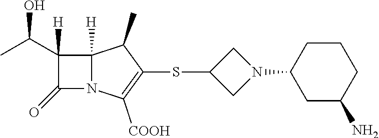 [SMILES: C[C@@H](O)[C@H]1C(=O)N2C(C(=O)O)=C(SC3CN([C@@H]4CCC[C@@H](N)C4)C3)[C@H](C)[C@H]12] | 395.52 | 396 |
| 17 | 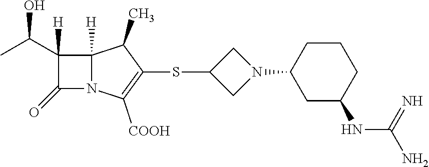 [SMILES: C[C@@H](O)[C@H]1C(=O)N2C(C(=O)O)=C(SC3CN([C@@H]4CCC[C@@H](NC(=N)N)C4)C3)[C@H](C)[C@H]12] | 437.56 | 438 |
| 18 | 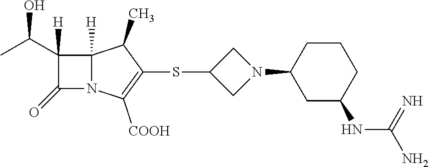 [SMILES: C[C@@H](O)[C@H]1C(=O)N2C(C(=O)O)=C(SC3CN([C@H]4CCC[C@@H](NC(=N)N)C4)C3)[C@H](C)[C@H]12] | 437.56 | 438 |
| 19 | 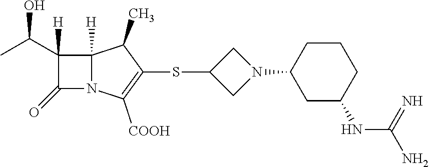 [SMILES: C[C@@H](O)[C@H]1C(=O)N2C(C(=O)O)=C(SC3CN([C@@H]4CCC[C@H](NC(=N)N)C4)C3)[C@H](C)[C@H]12] | 437.56 | 438 |
| 20 | 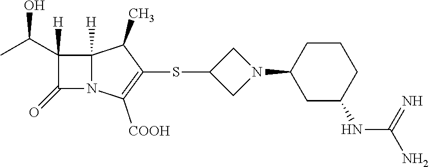 [SMILES: C[C@@H](O)[C@H]1C(=O)N2C(C(=O)O)=C(SC3CN([C@H]4CCC[C@H](NC(=N)N)C4)C3)[C@H](C)[C@H]12] | 437.56 | 438 |
| 21 | 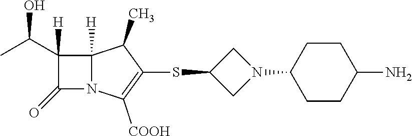 | 395.52 | |
| 22 | 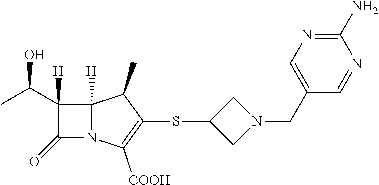 [SMILES: C[C@@H](O)[C@H]1C(=O)N2C(C(=O)O)=C(SC3CN(Cc4cnc(N)nc4)C3)[C@H](C)[C@H]12] | 405.47 | 406 |
| 23 | 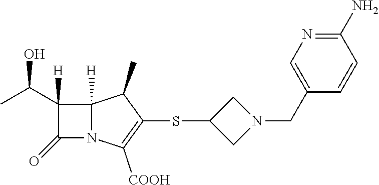 [SMILES: C[C@@H](O)[C@H]1C(=O)N2C(C(=O)O)=C(SC3CN(Cc4ccc(N)nc4)C3)[C@H](C)[C@H]12] | 405.47 | 406 |
| 24 | 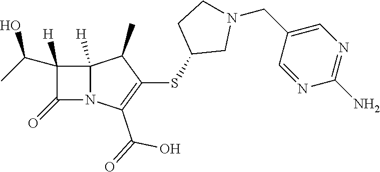 [SMILES: C[C@@H](O)[C@H]1C(=O)N2C(C(=O)O)=C(S[C@@H]3CCN(Cc4cnc(N)nc4)C3)[C@H](C)[C@H]12] | 419.50 | 420 |
| 25 | 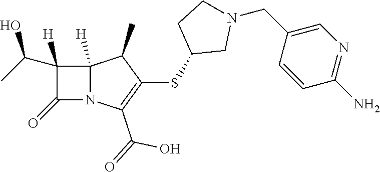 [SMILES: C[C@@H](O)[C@H]1C(=O)N2C(C(=O)O)=C(S[C@@H]3CCN(Cc4ccc(N)nc4)C3)[C@H](C)[C@H]12] | 418.51 | 419 |
| 26 | 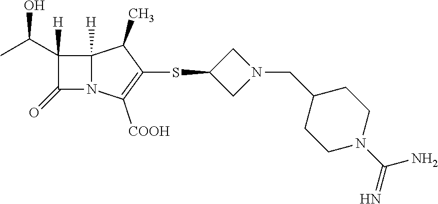 | 437.56 | 438 |
| 27 | 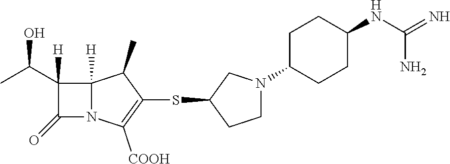 [SMILES: C[C@@H](O)[C@H]1C(=O)N2C(C(=O)O)=C(S[C@@H]3CCN([C@H]4CC[C@H](NC(=N)N)CC4)C3)[C@H](C)[C@H]12] | 451.59 | 452 |
| 28 | 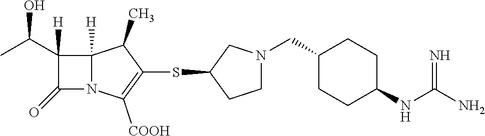 [SMILES: C[C@@H](O)[C@H]1C(=O)N2C(C(=O)O)=C(S[C@@H]3CCN(C[C@H]4CC[C@H](NC(=N)N)CC4)C3)[C@H](C)[C@H]12] | 465.61 | 466 |
| 29 | 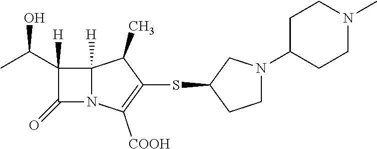 [SMILES: C[C@@H](O)[C@H]1C(=O)N2C(C(=O)O)=C(S[C@@H]3CCN(C4CCN(C)CC4)C3)[C@H](C)[C@H]12] | 409.55 | 410 |
| 30 | 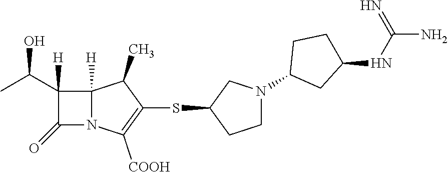 [SMILES: C[C@@H](O)[C@H]1C(=O)N2C(C(=O)O)=C(S[C@@H]3CCN([C@@H]4CC[C@@H](NC(=N)N)C4)C3)[C@H](C)[C@H]12] | 437.56 | 438 |
| 31 | 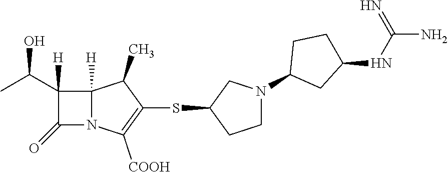 [SMILES: C[C@@H](O)[C@H]1C(=O)N2C(C(=O)O)=C(S[C@@H]3CCN([C@H]4CC[C@@H](NC(=N)N)C4)C3)[C@H](C)[C@H]12] | 437.56 | 438 |
| 32 | 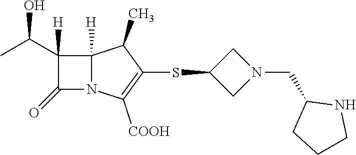 | 381.49 | 382 |
| 33 | 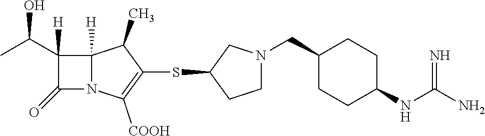 [SMILES: C[C@@H](O)[C@H]1C(=O)N2C(C(=O)O)=C(S[C@@H]3CCN(C[C@H]4CC[C@@H](NC(=N)N)CC4)C3)[C@H](C)[C@H]12] | 465.61 | 466 |
| 34 | 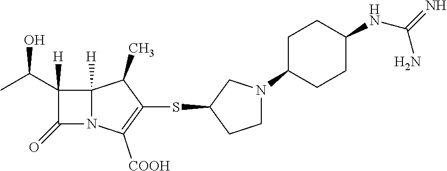 [SMILES: C[C@@H](O)[C@H]1C(=O)N2C(C(=O)O)=C(S[C@@H]3CCN([C@H]4CC[C@@H](NC(=N)N)CC4)C3)[C@H](C)[C@H]12] | 451.59 | 452 |
| 35 | 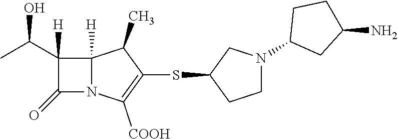 [SMILES: C[C@@H](O)[C@H]1C(=O)N2C(C(=O)O)=C(S[C@@H]3CCN([C@@H]4CC[C@@H](N)C4)C3)[C@H](C)[C@H]12] | 395.52 | 396 |
| 36 | 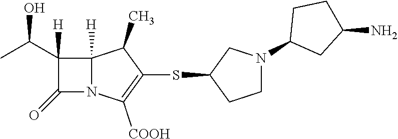 [SMILES: C[C@@H](O)[C@H]1C(=O)N2C(C(=O)O)=C(S[C@@H]3CCN([C@H]4CC[C@@H](N)C4)C3)[C@H](C)[C@H]12] | 395.52 | 396 |
| 37 | 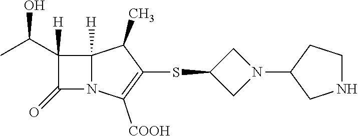 | 367.46 | 368 |
| 38 | 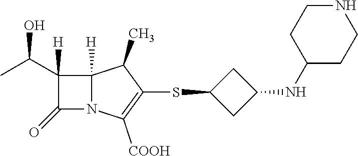 [SMILES: C[C@@H](O)[C@H]1C(=O)N2C(C(=O)O)=C(S[C@H]3C[C@H](NC4CCNCC4)C3)[C@H](C)[C@H]12] | 395.52 | 396 |
| 39 | 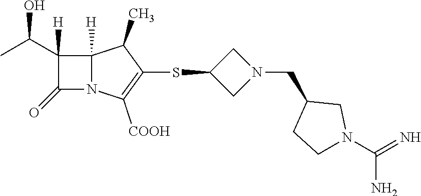 | 423.53 | 424 |
| 40 | 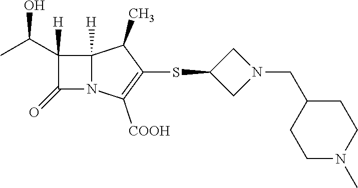 | 409.55 | 410 |
| 41 | 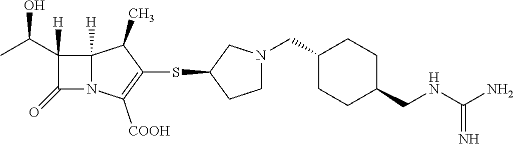 [SMILES: C[C@@H](O)[C@H]1C(=O)N2C(C(=O)O)=C(S[C@@H]3CCN(C[C@H]4CC[C@H](CNC(=N)N)CC4)C3)[C@H](C)[C@H]12] | 479.64 | 480 |
| 42 | 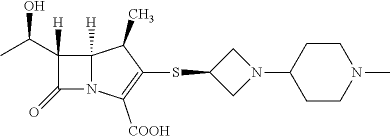 | 395.52 | 396 |
| 43 | 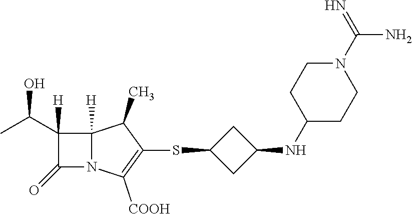 [SMILES: C[C@@H](O)[C@H]1C(=O)N2C(C(=O)O)=C(S[C@H]3C[C@@H](NC4CCN(C(=N)N)CC4)C3)[C@H](C)[C@H]12] | 437.56 | 438 |
| 44 | 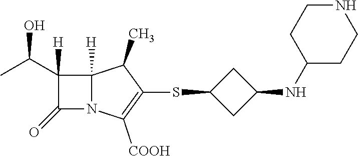 [SMILES: C[C@@H](O)[C@H]1C(=O)N2C(C(=O)O)=C(S[C@H]3C[C@@H](NC4CCNCC4)C3)[C@H](C)[C@H]12] | 395.52 | 396 |
| 45 | 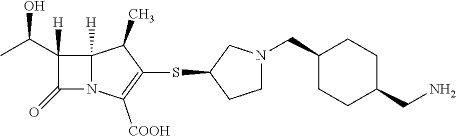 [SMILES: C[C@@H](O)[C@H]1C(=O)N2C(C(=O)O)=C(S[C@@H]3CCN(C[C@H]4CC[C@@H](CN)CC4)C3)[C@H](C)[C@H]12] | 437.60 | 438 |
| 46 | 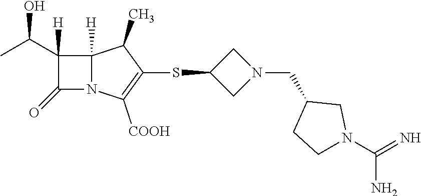 | 423.53 | 424 |
| 47 | 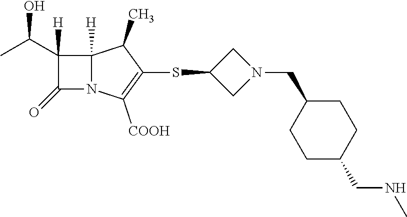 | 437.60 | 438 |
| 48 | 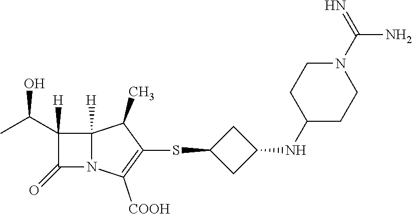 [SMILES: C[C@@H](O)[C@H]1C(=O)N2C(C(=O)O)=C(S[C@H]3C[C@H](NC4CCN(C(=N)N)CC4)C3)[C@H](C)[C@H]12] | 437.56 | 438 |
| 49 | 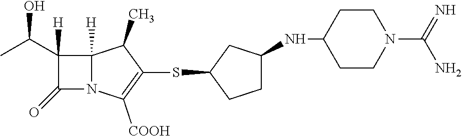 [SMILES: C[C@@H](O)[C@H]1C(=O)N2C(C(=O)O)=C(S[C@@H]3CC[C@H](NC4CCN(C(=N)N)CC4)C3)[C@H](C)[C@H]12] | 451.59 | 452 |
| 50 | 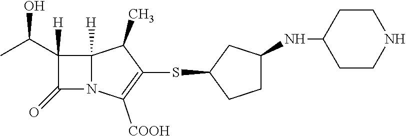 [SMILES: C[C@@H](O)[C@H]1C(=O)N2C(C(=O)O)=C(S[C@@H]3CC[C@H](NC4CCNCC4)C3)[C@H](C)[C@H]12] | 409.55 | 410 |
| 51 | 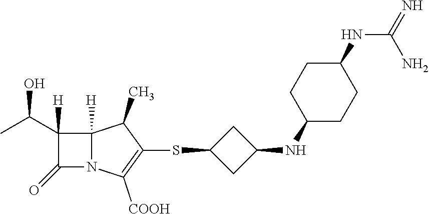 [SMILES: C[C@@H](O)[C@H]1C(=O)N2C(C(=O)O)=C(S[C@H]3C[C@@H](N[C@H]4CC[C@@H](NC(=N)N)CC4)C3)[C@H](C)[C@H]12] | 451.59 | 452 |
| 52 | 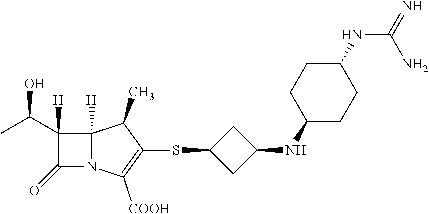 [SMILES: C[C@@H](O)[C@H]1C(=O)N2C(C(=O)O)=C(S[C@H]3C[C@@H](N[C@H]4CC[C@H](NC(=N)N)CC4)C3)[C@H](C)[C@H]12] | 451.59 | 452 |
| 53 | 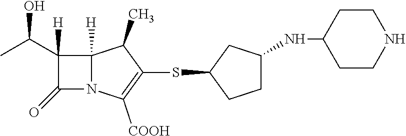 [SMILES: C[C@@H](O)[C@H]1C(=O)N2C(C(=O)O)=C(S[C@@H]3CC[C@@H](NC4CCNCC4)C3)[C@H](C)[C@H]12] | 409.55 | 410 |
| 54 | 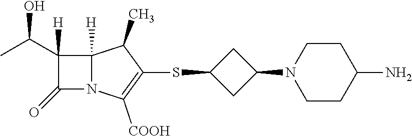 [SMILES: C[C@@H](O)[C@H]1C(=O)N2C(C(=O)O)=C(S[C@H]3C[C@@H](N4CCC(N)CC4)C3)[C@H](C)[C@H]12] | 395.52 | 396 |
| 55 | 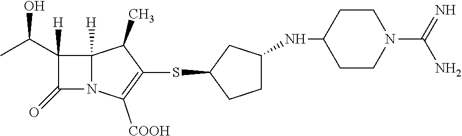 [SMILES: C[C@@H](O)[C@H]1C(=O)N2C(C(=O)O)=C(S[C@@H]3CC[C@@H](NC4CCN(C(=N)N)CC4)C3)[C@H](C)[C@H]12] | 451.59 | 452 |
| 56 | 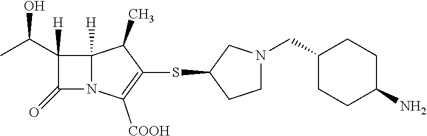 [SMILES: C[C@@H](O)[C@H]1C(=O)N2C(C(=O)O)=C(S[C@@H]3CCN(C[C@H]4CC[C@H](N)CC4)C3)[C@H](C)[C@H]12] | 423.57 | 424 |
| 57 | 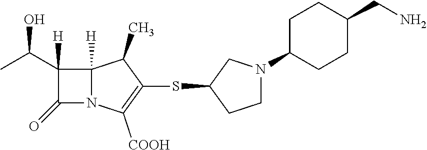 [SMILES: C[C@@H](O)[C@H]1C(=O)N2C(C(=O)O)=C(S[C@@H]3CCN([C@H]4CC[C@@H](CN)CC4)C3)[C@H](C)[C@H]12] | 423.57 | 424 |
| 58 | 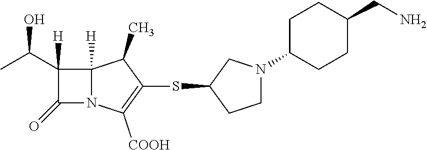 [SMILES: C[C@@H](O)[C@H]1C(=O)N2C(C(=O)O)=C(S[C@@H]3CCN([C@H]4CC[C@H](CN)CC4)C3)[C@H](C)[C@H]12] | 423.57 | 424 |
| 59 | 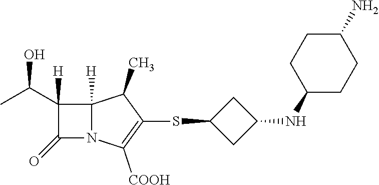 [SMILES: C[C@@H](O)[C@H]1C(=O)N2C(C(=O)O)=C(S[C@H]3C[C@H](N[C@H]4CC[C@H](N)CC4)C3)[C@H](C)[C@H]12] | 409.55 | 410 |
| 60 | 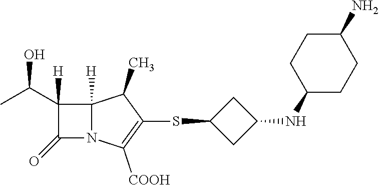 [SMILES: C[C@@H](O)[C@H]1C(=O)N2C(C(=O)O)=C(S[C@H]3C[C@H](N[C@H]4CC[C@@H](N)CC4)C3)[C@H](C)[C@H]12] | 409.55 | 410 |
| 61 | 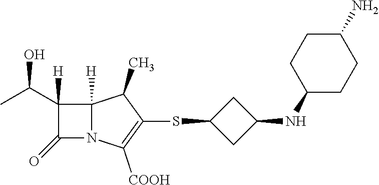 [SMILES: C[C@@H](O)[C@H]1C(=O)N2C(C(=O)O)=C(S[C@H]3C[C@@H](N[C@H]4CC[C@H](N)CC4)C3)[C@H](C)[C@H]12] | 409.55 | 410 |
| 62 | 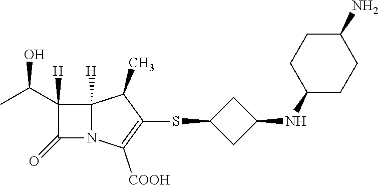 [SMILES: C[C@@H](O)[C@H]1C(=O)N2C(C(=O)O)=C(S[C@H]3C[C@@H](N[C@H]4CC[C@@H](N)CC4)C3)[C@H](C)[C@H]12] | 409.55 | 410 |
| 63 | 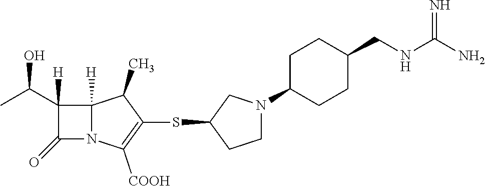 [SMILES: C[C@@H](O)[C@H]1C(=O)N2C(C(=O)O)=C(S[C@@H]3CCN([C@H]4CC[C@@H](CNC(=N)N)CC4)C3)[C@H](C)[C@H]12] | 465.61 | 466 |
| 64 | 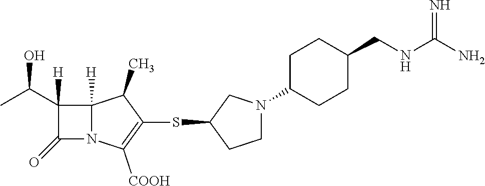 | 465.61 | 466 |
| 65 | 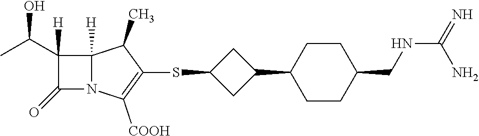 | 451.59 | 452 |
| 66 | 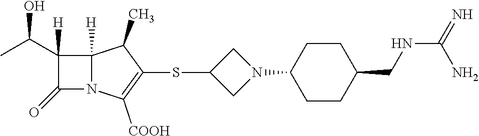 [SMILES: C[C@@H](O)[C@H]1C(=O)N2C(C(=O)O)=C(SC3CN([C@H]4CC[C@H](CNC(=N)N)CC4)C3)[C@H](C)[C@H]12] | 451.59 | 452 |
| 67 | 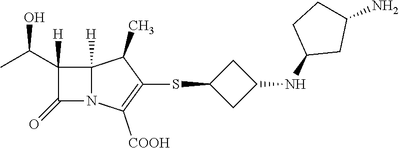 | 395.52 | 396 |
| 68 | 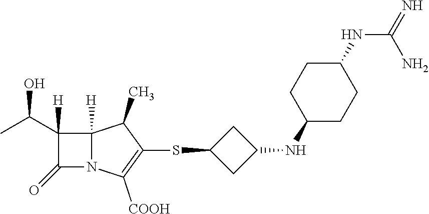 | 451.59 | 452 |
| 69 | 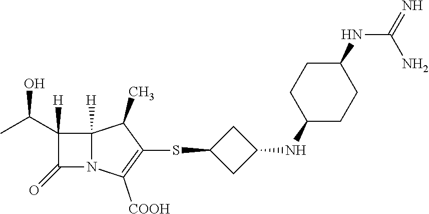 [SMILES: C[C@@H](O)[C@H]1C(=O)N2C(C(=O)O)=C(S[C@H]3C[C@H](N[C@H]4CC[C@@H](NC(=N)N)CC4)C3)[C@H](C)[C@H]12] | 451.59 | 452 |
| 70 | 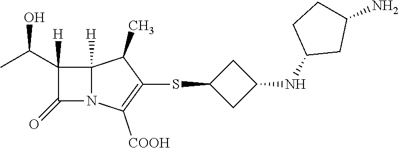 [SMILES: C[C@@H](O)[C@H]1C(=O)N2C(C(=O)O)=C(S[C@H]3C[C@H](N[C@@H]4CC[C@H](N)C4)C3)[C@H](C)[C@H]12] | 395.52 | 396 |
| 71 | 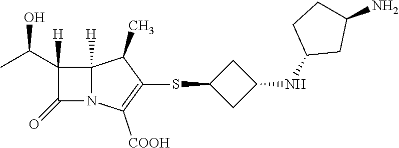 [SMILES: C[C@@H](O)[C@H]1C(=O)N2C(C(=O)O)=C(S[C@H]3C[C@H](N[C@@H]4CC[C@@H](N)C4)C3)[C@H](C)[C@H]12] | 395.52 | 396 |
| 72 | 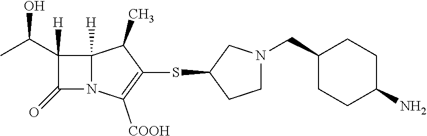 [SMILES: C[C@@H](O)[C@H]1C(=O)N2C(C(=O)O)=C(S[C@@H]3CCN(C[C@H]4CC[C@@H](N)CC4)C3)[C@H](C)[C@H]12] | 423.57 | 424 |
| 73 | 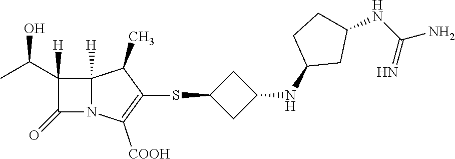 [SMILES: C[C@@H](O)[C@H]1C(=O)N2C(C(=O)O)=C(S[C@H]3C[C@H](N[C@H]4CC[C@H](NC(=N)N)C4)C3)[C@H](C)[C@H]12] | 437.56 | 438 |
| 74 | 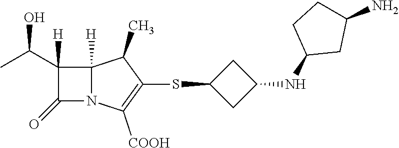 [SMILES: C[C@@H](O)[C@H]1C(=O)N2C(C(=O)O)=C(S[C@H]3C[C@H](N[C@H]4CC[C@@H](N)C4)C3)[C@H](C)[C@H]12] | 395.52 | 396 |
| 75 | 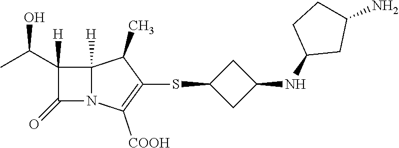 [SMILES: C[C@@H](O)[C@H]1C(=O)N2C(C(=O)O)=C(S[C@H]3C[C@@H](N[C@H]4CC[C@H](N)C4)C3)[C@H](C)[C@H]12] | 395.52 | 396 |
| 76 | 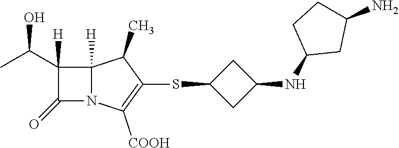 [SMILES: C[C@@H](O)[C@H]1C(=O)N2C(C(=O)O)=C(S[C@H]3C[C@@H](N[C@H]4CC[C@@H](N)C4)C3)[C@H](C)[C@H]12] | 395.52 | 396 |
| 77 | 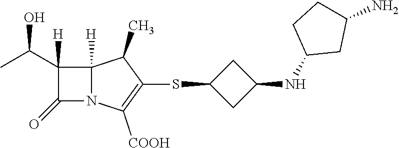 [SMILES: C[C@@H](O)[C@H]1C(=O)N2C(C(=O)O)=C(S[C@H]3C[C@@H](N[C@@H]4CC[C@H](N)C4)C3)[C@H](C)[C@H]12] | 395.52 | 396 |
| 78 | 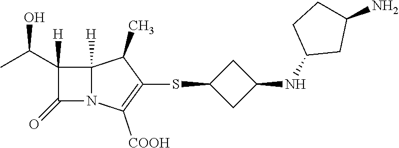 [SMILES: C[C@@H](O)[C@H]1C(=O)N2C(C(=O)O)=C(S[C@H]3C[C@@H](N[C@@H]4CC[C@@H](N)C4)C3)[C@H](C)[C@H]12] | 395.52 | 396 |
| 79 | 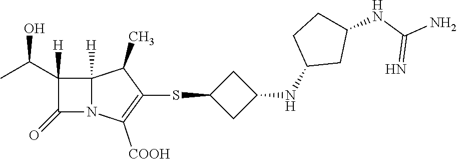 [SMILES: C[C@@H](O)[C@H]1C(=O)N2C(C(=O)O)=C(S[C@H]3C[C@H](N[C@@H]4CC[C@H](NC(=N)N)C4)C3)[C@H](C)[C@H]12] | 437.56 | 438 |

Described herein are compounds, or a pharmaceutically acceptable salt, solvate, or stereoisomer thereof, selected from the group consisting of:

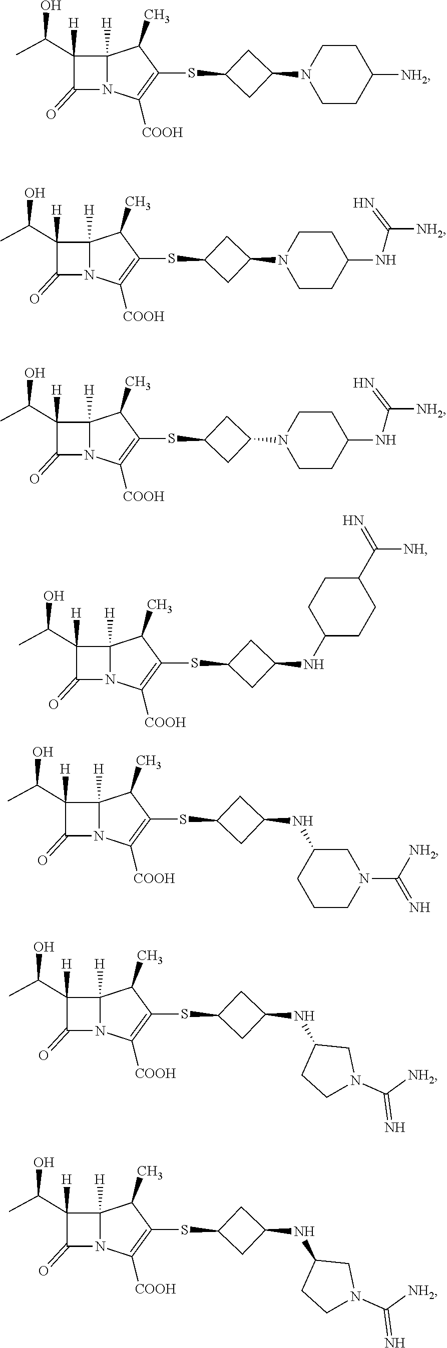

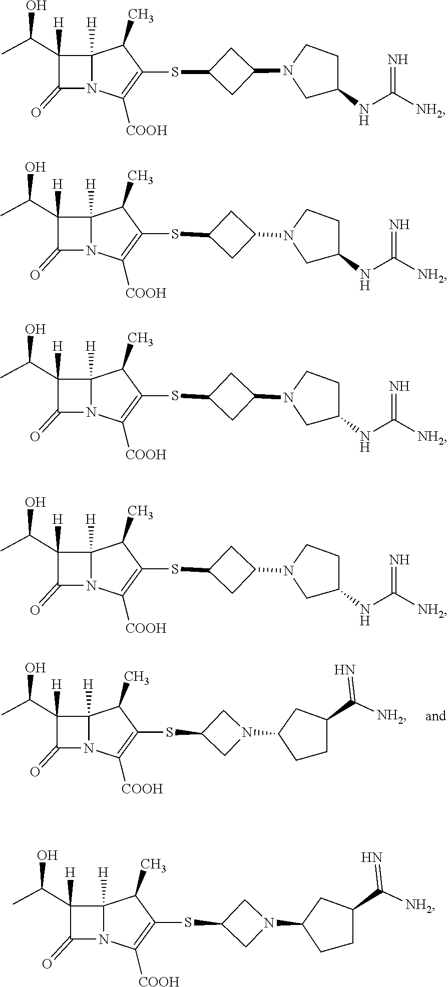

### Further Forms of Compounds Disclosed Herein

### Isomers Stereoisomers

In some embodiments, the compounds described herein exist as geometric isomers. In some embodiments, the compounds described herein possess one or more double bonds. The compounds presented herein include all cis, trans, syn, anti, entgegen (E), and zusammen (Z) isomers as well as the corresponding mixtures thereof. In some situations, the compounds described herein possess one or more chiral centers and each center exists in the R configuration, or S configuration. The compounds described herein include all diastereomeric, enantiomeric, and epimeric forms as well as the corresponding mixtures thereof. In additional embodiments of the compounds and methods provided herein, mixtures of enantiomers and/or diastereoisomers, resulting from a single preparative step, combination, or interconversion are useful for the applications described herein. In some embodiments, the compounds described herein are prepared as their individual stereoisomers by reacting a racemic mixture of the compound with an optically active resolving agent to form a pair of diastereoisomeric compounds, separating the diastereomers and recovering the optically pure enantiomers. In some embodiments, dissociable complexes are preferred. In some embodiments, the diastereomers have distinct physical properties (e.g., melting points, boiling points, solubilities, reactivity, etc.) and are separated by taking advantage of these dissimilarities. In some embodiments, the diastereomers are separated by chiral chromatography, or preferably, by separation/resolution techniques based upon differences in solubility. In some embodiments, the optically pure enantiomer is then recovered, along with the resolving agent, by any practical means that would not result in racemization.

### Labeled Compounds

In some embodiments, the compounds described herein exist in their isotopically-labeled forms. In some embodiments, the methods disclosed herein include methods of treating diseases by administering such isotopically-labeled compounds. In some embodiments, the methods disclosed herein include methods of treating diseases by administering such isotopically-labeled compounds as pharmaceutical compositions. Thus, in some embodiments, the compounds disclosed herein include isotopically-labeled compounds, which are identical to those recited herein, but for the fact that one or more atoms are replaced by an atom having an atomic mass or mass number different from the atomic mass or mass number usually found in nature. Examples of isotopes that can be incorporated into compounds disclosed herein, or a pharmaceutically acceptable salt, solvate, or stereoisomer thereof, include isotopes of hydrogen, carbon, nitrogen, oxygen, phosphorous, sulfur, fluorine and chloride, such as ²H, ³H, ¹³C, ¹⁴C, ¹⁵N, ¹⁸O, ¹⁷O, ³¹P, ³²P, ³⁵S, ¹⁸F, and ³⁶Cl, respectively. Compounds described herein, and pharmaceutically acceptable salts, solvate, or stereoisomers thereof which contain the aforementioned isotopes and/or other isotopes of other atoms are within the scope of this invention. Certain isotopically-labeled compounds, for example those into which radioactive isotopes such as 3H and ¹⁴C are incorporated, are useful in drug and/or substrate tissue distribution assays. Tritiated, i.e., ³H and carbon-14, i.e., ¹⁴C, isotopes are particularly preferred for their ease of preparation and detectability. Further, substitution with heavy isotopes such as deuterium, i.e., ²H, produces certain therapeutic advantages resulting from greater metabolic stability, for example increased in vivo half-life or reduced dosage requirements. In some embodiments, the isotopically labeled compounds, pharmaceutically acceptable salt, ester, prodrug, solvate, hydrate, or derivative thereof is prepared by any suitable method.

In some embodiments, the compounds described herein are labeled by other means, including, but not limited to, the use of chromophores or fluorescent moieties, bioluminescent labels, or chemiluminescent labels.

### Pharmaceutically Acceptable Salts

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefore react with any of a number of inorganic or organic bases, and inorganic and organic acids, to form a pharmaceutically acceptable salt. In some embodiments, these salts are prepared in situ during the final isolation and purification of the compounds disclosed herein or by separately reacting a purified compound in its free form with a suitable acid or base, and isolating the salt thus formed.

Examples of pharmaceutically acceptable salts include those salts prepared by reaction of the compounds described herein with a mineral, organic acid, or inorganic base, such salts including, acetate, acrylate, adipate, alginate, aspartate, benzoate, benzenesulfonate, bisulfate, bisulfite, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, γ-hydroxybutyrate, hydrochloride, hydrobromide, hydroiodide, 2-hydroxyethanesulfonate, iodide, isobutyrate, lactate, maleate, malonate, methanesulfonate, mandelate metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylateundeconate, and xylenesulfonate.

Further, the compounds described herein can be prepared as pharmaceutically acceptable salts formed by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, p-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid and muconic acid. In some embodiments, other acids, such as oxalic, while not in themselves pharmaceutically acceptable, are employed in the preparation of salts useful as intermediates in obtaining the compounds disclosed herein and their pharmaceutically acceptable acid addition salts.

In some embodiments, those compounds described herein which comprise a free acid group react with a suitable base, such as the hydroxide, carbonate, bicarbonate, sulfate, of a pharmaceutically acceptable metal cation, with ammonia, or with a pharmaceutically acceptable organic primary, secondary, tertiary, or quaternary amine. Representative salts include the alkali or alkaline earth salts, like lithium, sodium, potassium, calcium, and magnesium, and aluminum salts and the like. Illustrative examples of bases include sodium hydroxide, potassium hydroxide, choline hydroxide, sodium carbonate, N⁺(C₁₋₄ alkyl)₄, and the like.

Representative organic amines useful for the formation of base addition salts include ethylamine, diethylamine, ethylenediamine, ethanolamine, diethanolamine, piperazine, and the like. It should be understood that the compounds described herein also include the quaternization of any basic nitrogen-containing groups they contain. In some embodiments, water or oil-soluble or dispersible products are obtained by such quaternization.

### Solvates

In some embodiments, the compounds described herein exist as solvates. The invention provides for methods of treating diseases by administering such solvates. The invention further provides for methods of treating diseases by administering such solvates as pharmaceutical compositions.

Solvates contain either stoichiometric or non-stoichiometric amounts of a solvent, and, in some embodiments, are formed during the process of crystallization with pharmaceutically acceptable solvents such as water, ethanol, and the like. Hydrates are formed when the solvent is water, or alcoholates are formed when the solvent is alcohol. Solvates of the compounds described herein can be conveniently prepared or formed during the processes described herein. By way of example only, hydrates of the compounds described herein can be conveniently prepared by recrystallization from an aqueous/organic solvent mixture, using organic solvents including, but not limited to, dioxane, tetrahydrofuran or methanol. In addition, the compounds provided herein can exist in unsolvated as well as solvated forms. In general, the solvated forms are considered equivalent to the unsolvated forms for the purposes of the compounds and methods provided herein.

### Tautomers

In some situations, compounds exist as tautomers. The compounds described herein include all possible tautomers within the formulas described herein. Tautomers are compounds that are interconvertible by migration of a hydrogen atom, accompanied by a switch of a single bond and adjacent double bond. In bonding arrangements where tautomerization is possible, a chemical equilibrium of the tautomers will exist. All tautomeric forms of the compounds disclosed herein are contemplated. The exact ratio of the tautomers depends on several factors, including temperature, solvent, and pH.

### Preparation of Compounds

Described herein are compounds, or a pharmaceutically acceptable salt, solvate, or stereoisomer thereof useful in the treatment of bacterial infections and processes for their preparation. In some embodiments, compounds described herein, or a pharmaceutically acceptable salt, solvate, or stereoisomer thereof are synthesized using standard synthetic reactions known to those of skill in the art or using methods known in the art. The reactions can be employed in a linear sequence to provide the compounds or they may be used to synthesize fragments which are subsequently joined by the methods known in the art. In some embodiments, the starting material used for the synthesis of the compounds described herein are synthesized or are obtained from commercial sources, such as, but not limited to, Aldrich Chemical Co. (Milwaukee, Wis.), Bachem (Torrance, Calif.), or Sigma Chemical Co. (St. Louis, Mo.). In some embodiments, the compounds described herein, and other related compounds having different substituents are synthesized using techniques and materials known to those of skill in the art, such as described, for example, in March, ADVANCED ORGANIC CHEMISTRY 4^{th} Ed., (Wiley 1992); Carey and Sundberg, ADVANCED ORGANIC CHEMISTRY 4^{th} Ed., Vols. A and B (Plenum 2000, 2001); Green and Wuts, PROTECTIVE GROUPS IN ORGANIC SYNTHESIS 3^{rd} Ed., (Wiley 1999); Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); and Larock's Comprehensive Organic Transformations (VCH Publishers Inc., 1989), all of which are incorporated by reference for such disclosures. In some embodiments, general methods for the preparation of compound as disclosed herein are derived from known reactions in the field, and the reactions are modified by the use of appropriate reagents and conditions, as would be recognized by the skilled person, for the introduction of the various moieties found in the formulae as provided herein.

In some embodiments, the products of the reactions are isolated and purified, if desired, using conventional techniques, including, but not limited to, filtration, distillation, crystallization, chromatography, and the like. In some embodiments, such materials are characterized using conventional means, including physical constants and spectral data.

### Pharmaceutical Compositions/Formulations

In another aspect, provided herein are pharmaceutical compositions comprising a compound describe herein, or a pharmaceutically acceptable salt, solvate, or stereoisomer thereof, and at least one pharmaceutically acceptable excipient. Pharmaceutical compositions are formulated in a conventional manner using one or more pharmaceutically acceptable excipients that facilitate processing of the active compounds into preparations that can be used pharmaceutically. Proper formulation is dependent upon the route of administration chosen. A summary of pharmaceutical compositions described herein can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

In some embodiments, the pharmaceutically acceptable excipient is selected from carriers, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, and any combinations thereof.

The pharmaceutical compositions described herein are administered to a subject by appropriate administration routes, including, but not limited to, oral, parenteral (e.g., intravenous, subcutaneous, intramuscular), intranasal, buccal, topical, rectal, or transdermal administration routes. The pharmaceutical formulations described herein include, but are not limited to, aqueous liquid dispersions, liquids, gels, syrups, elixirs, slurries, suspensions, self-emulsifying dispersions, solid solutions, liposomal dispersions, aerosols, solid oral dosage forms, powders, immediate release formulations, controlled release formulations, fast melt formulations, tablets, capsules, pills, powders, dragees, effervescent formulations, lyophilized formulations, delayed release formulations, extended release formulations, pulsatile release formulations, multiparticulate formulations, and mixed immediate and controlled release formulations.

Pharmaceutical compositions including compounds described herein, or a pharmaceutically acceptable salt, solvate, or stereoisomer thereof are manufactured in a conventional manner, such as, by way of example only, by means of conventional mixing, dissolving, granulating, dragee-making, levigating, emulsifying, encapsulating, entrapping, or compression processes.

Pharmaceutical compositions for oral use are obtained by mixing one or more solid excipient with one or more of the compounds described herein, optionally grinding the resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose; or others such as polyvinylpyrrolidone (PVP or povidone) or calcium phosphate. If desired, disintegrating agents are added, such as the cross-linked croscarmellose sodium, polyvinylpyrrolidone, agar, or alginic acid or a salt thereof such as sodium alginate. In some embodiments, dyestuffs or pigments are added to the tablets or dragee coatings for identification or to characterize different combinations of active compound doses.

Pharmaceutical compositions that are administered orally include push-fit capsules made of gelatin, as well as soft, sealed capsules made of gelatin and a plasticizer, such as glycerol or sorbitol. The push-fit capsules contain the active ingredients in admixture with filler such as lactose, binders such as starches, and/or lubricants such as talc or magnesium stearate and, optionally, stabilizers. In soft capsules, the active compounds are dissolved or suspended in suitable liquids, such as fatty oils, liquid paraffin, or liquid polyethylene glycols. In some embodiments, stabilizers are added.

Pharmaceutical compositions for parental use are formulated as infusions or injections. In some embodiments, the pharmaceutical composition suitable for injection or infusion includes sterile aqueous solutions, or dispersions, or sterile powders comprising a compound described herein, or a pharmaceutically acceptable salt, solvate, or stereoisomer thereof. In some embodiments, the pharmaceutical composition comprises a liquid carrier. In some embodiments, the liquid carrier is a solvent or liquid dispersion medium comprising, for example, water, saline, ethanol, a polyol (for example, glycerol, propylene glycol, liquid polyethylene glycols, and the like), vegetable oils, nontoxic glyceryl esters, and any combinations thereof. In some embodiments, the pharmaceutical compositions further comprise a preservative to prevent growth of microorganisms.

### Combination Treatment

Disclosed herein are combinations of compounds described herein, or a pharmaceutically acceptable salt, solvate, or stereoisomer thereof with one or more antibiotics for the treatment of bacterial infections. In some embodiments, the antibiotic is administered by a route and in an amount commonly used, therefore, contemporaneously or sequentially with a compound described herein, or a pharmaceutically acceptable salt, solvate, or stereoisomer thereof. When a compound described herein, or a pharmaceutically acceptable salt, solvate, or stereoisomer thereof is used contemporaneously with one or more antibiotics, a pharmaceutical composition in unit dosage form containing such other drugs and the compound of the present disclosure is optionally used. In some embodiments, the combination therapy also includes therapies in which the compound described herein, or a pharmaceutically acceptable salt, solvate, or stereoisomer thereof and one or more antibiotic are administered on different overlapping schedules. In some embodiments, when used in combination with one or more antibiotics, the compound described herein, or a pharmaceutically acceptable salt, solvate, or stereoisomer thereof is used in lower doses than when each is used singly.

In some embodiments, the one or more antibiotics are beta-lactam antibiotics. In certain embodiments, the beta-lactam antibiotic is a penicillin, a penem, a carbapenem, a cephalosporin, a cephamycin, a monobactam, or combinations thereof. Penicillins include, but are not limited to, amoxicillin, ampicillin, azidocillin, azlocillin, bacampicillin, benzathinebenzylpenicillin, benzathinephenoxymethylpenicillin, benzylpenicillin (G), carbenicillin, carindacillin, clometocillin, cloxacillin, dicloxacillin, epicillin, flucloxacillin, hetacillin, mecillinam, metampicillin, meticillin, mezlocillin, nafcillin, oxacillin, penamecillin, pheneticillin, phenoxymethylpenicillin (V), piperacillin, pivampicillin, pivmecillinam, procaine benzylpenicillin, propicillin, sulbenicillin, talampicillin, temocillin, ticarcillin, or any combinations thereof. Penems include, but are not limited to, faropenem. Carbapenems include, but are not limited to, biapenem, ertapenem, doripenem, imipenem, meropenem, panipenem, or any combinations thereof. Cephalosprins/cephamycins include, but are not limited to, cefacetrile, cefaclor, cefadroxil, cefalexin, cefaloglycin, cefalonium, cefaloridine, cefalotin, cefamandole, cefapirin, cefatrizine, cefazaflur, cefazedone, cefazolin, cefbuperazone, cefcapene, cefdaloxime, cefdinir, cefditoren, cefepime, cefetamet, cefixime, cefinenoxime, cefinetazole, cefminox, cefodizime, cefonicid, cefoperazone, ceforanide, cefotaxime, cefotetan, cefotiam, cefovecin, cefoxitin, cefozopran, cefpimizole, cefpiramide, cefpirome, cefpodoxime, cefprozil, cefquinome, cefquinome, cefradine, cefroxadine, cefsulodin, ceftarolinefosamil, ceftazidime, cefteram, ceftezole, ceftibuten, ceftiofur, ceftiolene, ceftizoxime, ceftobiprole, ceftriaxone, cefuroxime, cefuzonam, flomoxef, latamoxef, loracarbef, or any combinations thereof. Monobactams include, but are not limited to, aztreonam, carumonam, nocardicin A, tigemonam, or any combinations thereof.

Also disclosed herein, are combination of compounds described herein, or a pharmaceutically acceptable salt, solvate, or stereoisomer thereof with one or more beta-lactamase inhibitors for the treatment of bacterial infections. In some embodiments, the beta-lactamase inhibitor is VNRX-5133 (taniborbactam), clavulanic acid, sulbactam, tazobactam, or any combinations thereof. In some embodiments, the beta-lactamase inhibitor is VNRX-5133 (taniborbactam).

Also disclosed herein, are combination of compounds described herein, or a pharmaceutically acceptable salt, solvate, or stereoisomer thereof with one or more non beta-lactam beta-lactamase inhibitors for the treatment of bacterial infections. In some embodiments, the non beta-lactam beta-lactamase inhibitor is avibactam, relebactam, or any combinations thereof.

### Administration of Pharmaceutical Composition

Suitable routes of administration include, but are not limited to, oral, intravenous, rectal, aerosol, parenteral, ophthalmic, pulmonary, transmucosal, transdermal, vaginal, otic, nasal, and topical administration. In addition, by way of example only, parenteral delivery includes intramuscular, subcutaneous, intravenous, intramedullary injections, as well as intrathecal, direct intraventricular, intraperitoneal, intralymphatic, and intranasal injections.

In some embodiments, compounds described herein, or a pharmaceutically acceptable salt, solvate, or stereoisomer thereof and compositions thereof are administered in any suitable manner. The manner of administration can be chosen based on, for example, whether local or systemic treatment is desired, and on the area to be treated. For example, the compositions can be administered orally, parenterally (e.g., intravenous, subcutaneous, intraperitoneal, or intramuscular injection), by inhalation, extracorporeally, topically (including transdermally, ophthalmically, vaginally, rectally, intranasally) or the like.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained.

### Assays for Antibacterial Activity

Assays for the inhibition of bacterial growth are well known in the art.

### Methods

Disclosed herein are methods of treating a bacterial infection in a subject, comprising administering to the subject an effective amount of a compound described herein, or a pharmaceutically acceptable salt, solvate, or stereoisomer thereof. Also disclosed herein are methods of treating a bacterial infection in a subject, comprising administering to the subject a pharmaceutical composition comprising an effective amount of a compound described herein, or a pharmaceutically acceptable salt, solvate, or stereoisomer thereof. In some embodiments, the bacterial infection is caused by gram-negative bacteria. In some embodiments, the bacterial infection is caused by multidrug-resistant (MDR) bacteria. In some embodiments, the bacterial infection is caused by carbapenem resistant Enterobacteriaceae (CRE). In some embodiments, the bacterial infection is caused by an aerobic bacteria. In some embodiments, the bacterial infection is caused by an anaerobic bacteria. In some embodiments, the bacterial infection is an upper or lower respiratory tract infection, a urinary tract infection, an intra-abdominal infection, a skin infection, or septicemia.

In some embodiments, the bacterial infection is caused by a bacteria that include Pseudomonas aeruginosa, Pseudomonas fluorescens, Pseudomonas acidovorans, Pseudomonas alcaligenes, Pseudomonas putida, Stenotrophomonas maltophilia, Burkholderia cepacia, Aeromonas hydrophilia, Escherichia coli, Citrobacter freundii, Salmonella typhimurium, Salmonella typhi, Salmonella paratyphi, Salmonella enteritidis, Shigella dysenteriae, Shigella flexneri, Shigella sonnei, Enterobacter cloacae, Enterobacter aerogenes, Klebsiella pneumoniae, Klebsiella oxytoca, Serratia marcescens, Francisella tularensis, Morganella morganii, Proteus mirabilis, Proteus vulgaris, Providencia alcalifaciens, Providencia rettgeri, Providencia stuartii, Acinetobacter baumannii, Acinetobacter calcoaceticus, Acinetobacter haemolyticus, Yersinia enterocolitica, Yersinia pestis, Yersinia pseudotuberculosis, Yersinia intermedia, Bordetella pertussis, Bordetella parapertussis, Bordetella bronchiseptica, Haemophilus influenzae, Haemophilus parainfluenzae, Haemophilus haemolyticus, Haemophilus parahaemolyticus, Haemophilus ducreyi, Pasteurella multocida, Pasteurella haemolytica, Branhamella catarrhalis, Helicobacter pylori, Campylobacter fetus, Campylobacter jejuni, Campylobacter coli, Borrelia burgdorferi, Vibrio cholerae, Vibrio parahaemolyticus, Legionella pneumophila, Listeria monocytogenes, Neisseria gonorrhoeae, Neisseria meningitidis, Kingella, Moraxella, Gardnerella vaginalis, Bacteroides fragilis, Bacteroides distasonis, Bacteroides 3452A homology group, Bacteroides vulgatus, Bacteroides ovalus, Bacteroides thetaiotaomicron, Bacteroides uniformis, Bacteroides eggerthii, Bacteroides splanchnicus, Clostridium difficile, Mycobacterium tuberculosis, Mycobacterium avium, Mycobacterium intracellulare, Mycobacterium leprae, Corynebacterium diphtheriae, Corynebacterium ulcerans, Streptococcus pneumoniae, Streptococcus agalactiae, Streptococcus pyogenes, Enterococcus faecalis, Enterococcus faecium, Staphylococcus aureus, Staphylococcus epidermidis, Staphylococcus saprophyticus, Staphylococcus intermedius, Staphylococcus hyicus subsp. hyicus, Staphylococcus haemolyticus, Staphylococcus hominis, or Staphylococcus saccharolyticus.

In some embodiments, the infection that is treated is caused by a bacteria that includes Pseudomonas aeruginosa, Pseudomonas fluorescens, Stenotrophomonas maltophilia, Escherichia coli, Citrobacter freundii, Salmonella typhimurium, Salmonella typhi, Salmonella paratyphi, Salmonella enteritidis, Shigella dysenteriae, Shigella flexneri, Shigella sonnei, Enterobacter cloacae, Enterobacter aerogenes, Klebsiella pneumoniae, Klebsiella oxytoca, Serratia marcescens, Acinetobacter calcoaceticus, Acinetobacter haemolyticus, Yersinia enterocolitica, Yersinia pestis, Yersinia pseudotuberculosis, Yersinia intermedia, Haemophilus influenzae, Haemophilus parainfluenzae, Haemophilus haemolyticus, Haemophilus parahaemolyticus, Helicobacter pylori, Campylobacter fetus, Campylobacter jejuni, Campylobacter coli, Vibrio cholerae, Vibrio parahaemolyticus, Legionella pneumophila, Listeria monocytogenes, Neisseria gonorrhoeae, Neisseria meningitidis, Moraxella, Bacteroides fragilis, Bacteroides vulgatus, Bacteroides ovalus, Bacteroides thetaiotaomicron, Bacteroides uniformis, Bacteroides eggerthii, or Bacteroides splanchnicus.

Also disclosed herein are methods for inhibiting bacterial growth, such methods comprising contacting a bacterial cell culture, or a bacterially infected cell culture, tissue, or organism, with a carbapenem derivative described herein. Preferably, the bacteria to be inhibited by administration of a compound described herein, or a pharmaceutically acceptable salt, solvate, or stereoisomer thereof are bacteria that are resistant to beta-lactam antibiotics. The term “resistant” is well-understood by those of ordinary skill in the art (see, e g Payne et al., Antimicrobial Agents and Chemotherapy 38 767-772 (1994), Hanaki et al., Antimicrobial Agents and Chemotherapy 30 1120-1126 (1995)).

These methods are useful for inhibiting bacterial growth in a variety of contexts. In certain embodiments, a compound described herein, or a pharmaceutically acceptable salt, solvate, or stereoisomer thereof is administered to an experimental cell culture in vitro to prevent the growth of beta-lactam resistant bacteria. In certain some embodiments, a compound described herein, or a pharmaceutically acceptable salt, solvate, or stereoisomer thereof is administered to a mammal, including a human to prevent the growth of beta-lactam resistant bacteria in vivo. The method according to this embodiment comprises administering a therapeutically effective amount of a compound described herein, or a pharmaceutically acceptable salt, solvate, or stereoisomer thereof for a therapeutically effective period of time to a mammal, including a human.

### EXAMPLES

### General Examples for the Preparation of Compounds

The starting materials and intermediates for the compounds of this invention may be prepared by the application or adaptation of the methods described below, their obvious chemical equivalents, or, for example, as described in literature such as The Science of Synthesis, Volumes 1-8. Editors E. M. Carreira et al. Thieme publishers (2001-2008). The use of protective groups may be as described in methodology compendia such as Greene's Protective Groups in Organic Synthesis, Fourth Edition. John Wiley & Sons, Inc. 2006.

Certain compounds described herein, or a pharmaceutically acceptable salt, solvate, or stereoisomer thereof (SCHEME 1) are prepared from the corresponding functional-group-protected beta-lactams 1.1 or 1.2 by hydrogenation in the presence of a catalyst such as platinum on carbon, at a temperature between 0° C. and 10° C., followed by purification of the crude products by flash chromatography on MCI GEL CHP20P.

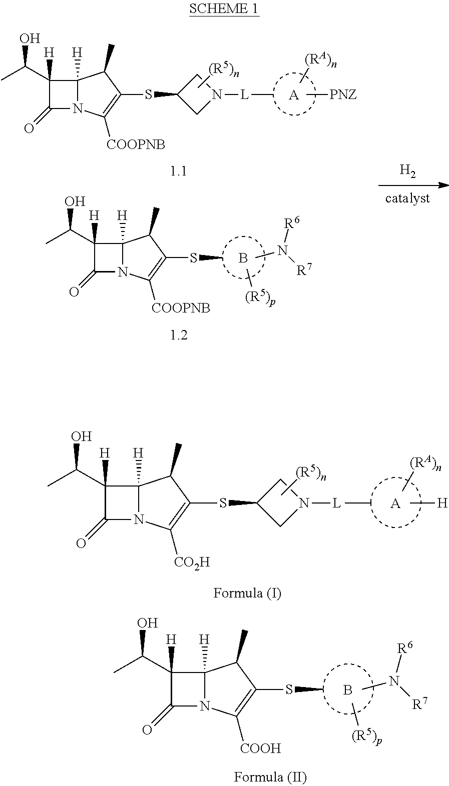

The functional-group-protected intermediates may be prepared according to the route outlined in SCHEME 2. Amino-thiols 2.1, 5, and 6 were each coupled with carbonyl compounds 3 in the presence of a reducing reagent such as sodium triacetoxyborohydride to give the thiols 4. Thiols 4 were then utilized for substitution reactions with the known and commercially available enol phosphate 7 in the presence of a base such as diisopropylethylamine (DIEA). Amino-thiols 2.1, 5 and 6, and the carbonyl compounds 3 may be obtained from commercial sources, prepared according to known methods in the literature, or prepared by a number of different reaction sequences such as those outlined in the Synthetic Examples below.

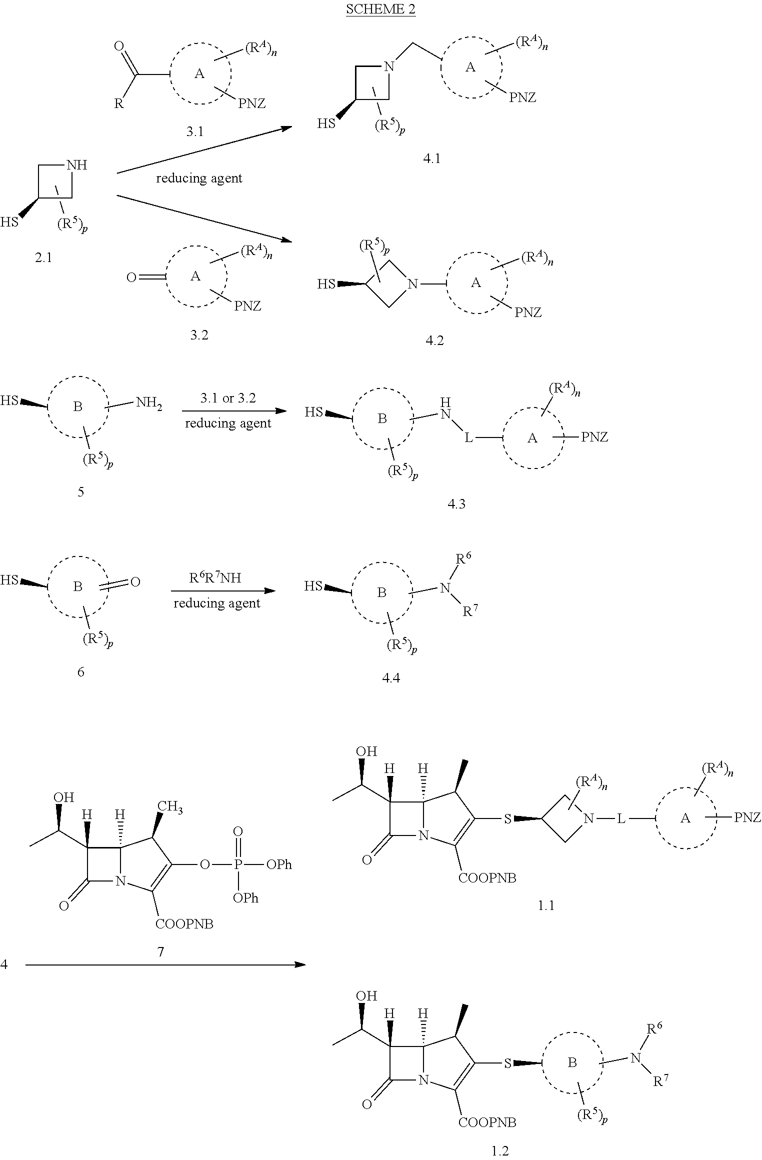

### Synthetic Examples

The following preparations of compounds described herein, or a pharmaceutically acceptable salt, solvate, or stereoisomer thereof and intermediates are given to enable those of skill in the art to more clearly understand and to practice the present invention. They should not be considered as limiting the scope of the invention, but merely as illustrative and representative thereof.

### Example 1: (4R,5S,6S)-3-((1-((1s,3S)-3-(aminomethyl)cyclobutyl)azetidin-3-yl)thio)-6-((R)-1-hydroxyethyl)-4-methyl-7-oxo-1-azabicyclo[3.2.0]hept-2-ene-2-carboxylic acid

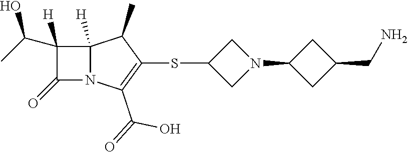 [SMILES: C[C@@H](O)[C@H]1C(=O)N2C(C(=O)O)=C(SC3CN([C@H]4C[C@@H](CN)C4)C3)[C@H](C)[C@H]12]

### Step 1. Synthesis of tert-butyl 3-hydroxyazetidine-1-carboxylate

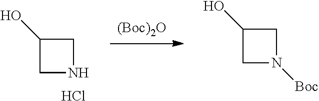 [SMILES: "(Boc)2O" ; OC1CNC1 ; CC(C)(C)OC(=O)N1CC(O)C1 ; Cl]

3-Hydroxyazetidine hydrochloride (30 g, 273.9 mmol) was dissolved in MeOH (600 mL). Then, TEA (118 mL, 83 g, 821.9 mmol) was added dropwise, and di-tert-butyl decarbonate (59.7 g, 273.9 mmol) was added in portions at 0° C. The reaction mixture was stirred at room temperature for 16 hrs. The resulting mixture was diluted with DCM, washed with water. The organic layer was dried over Na₂SO₄, concentrated under vacuum to give a yellow oil, 37 g, yield: 87.2%. ESI-MS m/z 174 (M+H)⁺.

### Step 2. Synthesis of tert-butyl 3-((methylsulfonyl)oxy)azetidine-1-carboxylate

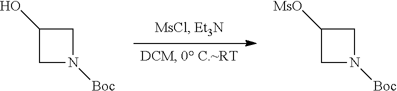

The resulting product from Step 1 (37 g, 212.6 mmol) was dissolved in dichloromethane (400 mL) under N₂, then triethylamine (46 mL, 32 g, 318.9 mmol) was added, the resulting solution was cooled in an ice-bath, and methylsulfonyl chloride (25.4 g, 223.2 mmol) was added dropwise over 15 min. The reaction was stirred at 0° C. for 0.5 hr, then the ice bath was removed, and the mixture was allowed to warm to room temperature and stir for 5 hrs. The resulting mixture was diluted with DCM, washed with NaHCO₃(aq), dried over Na₂SO₄, concentrated under vacuum to give a yellow oil, 50 g, yield: 89.1%. ESI-MS m/z 252 (M+H)⁺.

### Step 3. Synthesis of tert-butyl 3-(acetylthio)azetidine-1-carboxylate

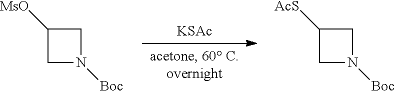

The resulting product from Step 2 (50 g, 198.4 mmol) was dissolved in DMF (800 mL), potassium thioacetate (45.2 g, 396.8 mmol) was added, and the reaction mixture was heated at 60° C. for 30 hrs. The resulting mixture was filtered. The filtrate was diluted with DCM, washed with water, dried over Na₂SO₄, concentrated under vacuum. The residue was purified by flash chromatography on silica gel (PE/EA=1:1) to give a yellow oil, 34 g, 45.9%. ESI-MS m/z 232 (M+H)⁺.

### Step 4. Synthesis of tert-butyl 3-mercaptoazetidine-1-carboxylate

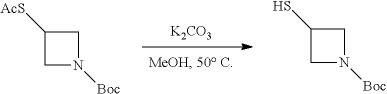

To a solution of the above product from Step 3 (34 g, 146.5 mmol) in methanol (300 mL) was added potassium carbonate (40.4 g, 293 mmol). The reaction mixture was stirred at 50° C. under Argon for 2 h, then cooled in ice-bath, acidified with 1 N HCl to pH 4-5, extracted with diethyl ether. The organic extracts were combined, washed with water, brine, dried over Na₂SO₄, and concentrated in vacuo to give the crude thiol, which was used directly for the next step without further purification, 33 g crude product. ESI-MS m/z 190 (M+H)⁺.

### Step 5. Synthesis of azetidine-3-thiol, trifluoroacetic acid salt

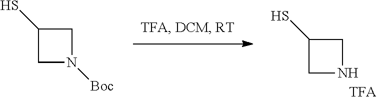 [SMILES: CC(C)(C)OC(=O)N1CC(S)C1 ; SC1CNC1]

The above crude product from Step 4 was dissolved in DCM (600 mL), treated with TFA (150 mL) at 0° C. for 1 h, and then was warmed to room temperature and stir for 5 hrs. The resulting mixture was concentrated in vacuo to give the crude product as a TFA salt, which was used directly for the next step without further purification. 40 g, crude product. ESI-MS m/z 90 (M+H)⁺.

### Step 6. Synthesis of 4-nitrobenzyl (((1r,3r)-3-hydroxycyclobutyl)methyl)carbamate

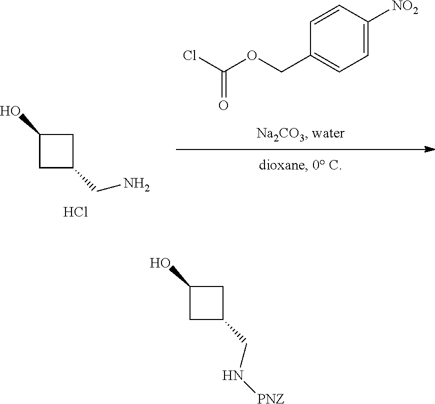

To a solution of (1r,3r)-3-(aminomethyl)cyclobutan-1-ol hydrochloride (2 g, 14.5 mmol) in water (15 mL) and dioxane (30 mL) was added sodium carbonate (3.1 g, 29 mmol), and the resulting solution was cooled to 0° C. (N₂) and then treated with 4-nitrobenzyl carbonochloridate (3.1 g, 14.5 mmol). The resulting mixture was stirred at 0° C. for 0.5 hr, then warmed to room temperature and stirred for 12 hrs. The resulting mixture was quenched by water, extracted with EA, dried over Na₂SO₄, concentrated under vacuum to give a white solid, 3.5 g, yield: 85.7%. ESI-MS m/z 281 (M+H)⁺.

### Step 7. Synthesis of 4-nitrobenzyl ((3-oxocyclobutyl)methyl)carbamate

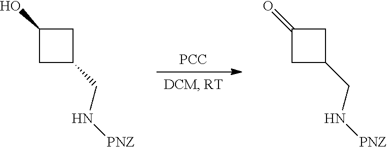

The product of 4-nitrobenzyl (((1r,3r)-3-hydroxycyclobutyl)methyl)carbamate from Step 6 (3.5 g, 12.4 mmol) was oxidized with PCC (5.37 g, 24.9 mmol) in DCM (60 mL) at rt for 5 h, diluted with DCM, filtered through a Florisil column, the filtrate was concentrated under vacuum. The residue was purified by flash chromatography on silica gel (PE/EA=1:3) to afford a white solid, 2.8 g, yield: 80.7%. ESI-MS m/z 279 (M+H)⁺.

### Step 8. Synthesis of 4-nitrobenzyl ((3-oxocyclobutyl)methyl)carbamate

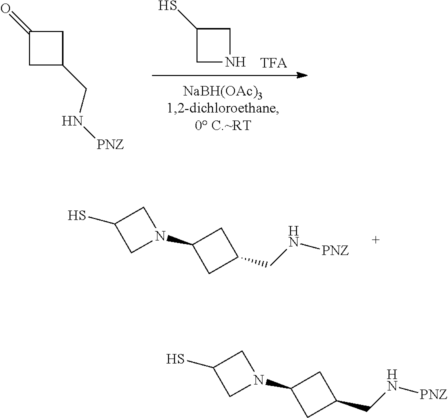

To a solution of 4-nitrobenzyl ((3-oxocyclobutyl)methyl)carbamate (2.8 g, 10.0 mmol), azetidine-3-thiol, trifluoroacetic acid salt from Step 5 (crude, 20.0 mmol) in 1,2-dichloroethane (50 mL) was added sodium triacetoxyborohydride (4.24 g, 20.0 mmol). The reaction mixture was stirred at rt overnight, diluted with DCM, washed with saturated aqueous NaHCO₃, dried over Na₂SO₄, concentrated in vacuo. The residue was purified by flash chromatography on silica gel (EA/MeOH=10:1) to afford two isomeric products, a less polar one (0.9 g) and more polar one (1.0 g). ESI-MS m/z 352 (M+H)⁺ for both isolated isomers.

### Step 9. Synthesis of (4R,5S,6S)-6-((R)-1-hydroxyethyl)-4-methyl-3-((1-((1r,3R)-3-(((((4-nitrobenzyl)oxy)carbonyl)amino)methyl)cyclobutyl)azetidin-3-yl)thio)-7-oxo-1-azabicyclo[3.2.0]hept-2-ene-2-carboxylic 4-nitrobenzoic anhydride

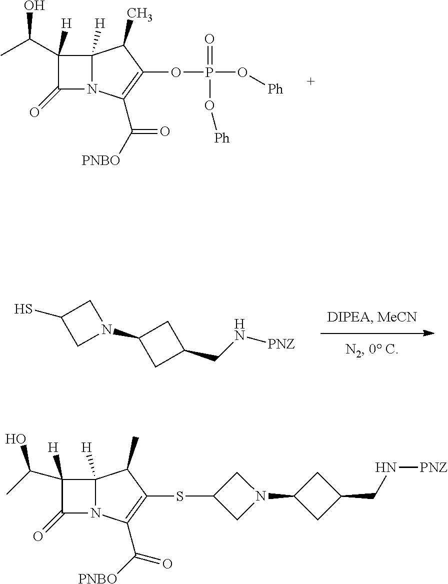

To 4-nitrobenzyl (((1s,3s)-3-(3-mercaptoazetidin-1-yl)cyclobutyl)methyl)carbamate (more polar isomer, 1.0 g, 2.84 mmol), and 4-nitrobenzyl (4R,5R,6S)-3-((diphenoxyphosphoryl)oxy)-6-((R)-1-hydroxyethyl)-4-methyl-7-oxo-1-azabicyclo[3.2.0]hept-2-ene-2-carboxylate (1.68 g, 2.84 mmol) was add dry acetonitrile (20 mL) under Argon at 0° C., followed by diisopropylethylamine (1.05 mL, 5.68 mmol). The reaction mixture was stirred at room temperature for 12 h, then concentrated. The residue was purified twice by flash chromatography on silica gel (EA/MeOH=10:1) to yield the product (800 mg, 40.5%). ESI-MS m/z 696 (M+H)⁺.

### Step 10. Synthesis of (4R,5S,6S)-3-((1-((1s,3S)-3-(aminomethyl)cyclobutyl)azetidin-3-yl)thio)-6-((R)-1-hydroxyethyl)-4-methyl-7-oxo-1-azabicyclo[3.2.0]hept-2-ene-2-carboxylic acid

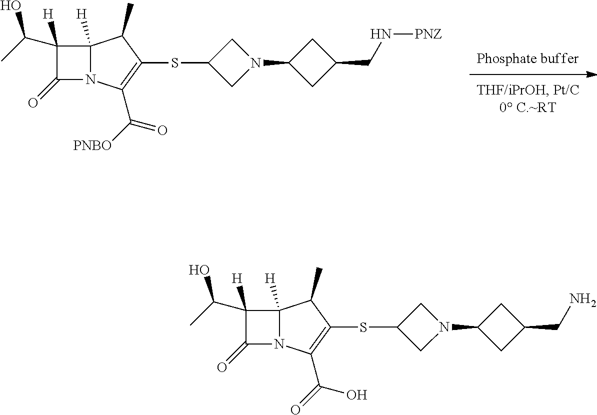

The above product (400 mg, 0.57 mmol) in THF (6 mL), iPrOH (6 mL), and phosphate buffer (pH 7, 12 mL) was hydrogenated in the presence of 5% Pt/C (200 mg) in ice-bath for 1 h. Then, the reaction mixture was stirred at room temperature for 12 hrs. The reaction mixture was filtered through a pad of celite, washed with small amount of water and EtOAc. The aqueous layer was separated, washed with EtOAc, then purified by flash chromatography on MCI GEL CHP20P (0-10% aqueous THF) followed by lyophilized to yield the product (60 mg). ESI-MS m/z 382 (M+H)⁺.

### Example 2: (4R,5S,6S)-3-((1-((1r,3R)-3-(aminomethyl)cyclobutyl)azetidin-3-yl)thio)-6-((R)-1-hydroxyethyl)-4-methyl-7-oxo-1-azabicyclo[3.2.0]hept-2-ene-2-carboxylic acid

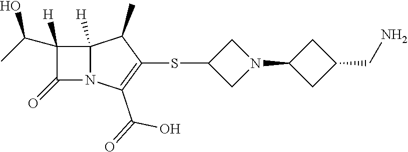 [SMILES: C[C@@H](O)[C@H]1C(=O)N2C(C(=O)O)=C(SC3CN([C@H]4C[C@H](CN)C4)C3)[C@H](C)[C@H]12]

By following the same reaction procedures as described in Steps 9 and 10 of Example 1, 4-nitrobenzyl (((1r,3r)-3-(3-mercaptoazetidin-1-yl)cyclobutyl)methyl)carbamate (less polar isomer from Example 1, Step 8) was converted to the target compound. ESI-MS m/z 382 (M+H)⁺.

### Example 3: (4R,5S,6S)-3-((1-((1s,3S)-3-aminocyclobutyl)azetidin-3-yl)thio)-6-((R)-1-hydroxyethyl)-4-methyl-7-oxo-1-azabicyclo[3.2.0]hept-2-ene-2-carboxylic acid

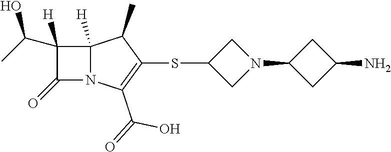 [SMILES: C[C@@H](O)[C@H]1C(=O)N2C(C(=O)O)=C(SC3CN([C@H]4C[C@@H](N)C4)C3)[C@H](C)[C@H]12]

By following the same reaction procedures as described in Steps 9 and 10 of Example 1, 4-nitrobenzyl ((1s,3s)-3-(3-mercaptoazetidin-1-yl)cyclobutyl)carbamate (more polar isomer prepared from 3-aminocyclobutan-1-ol via Steps 6, 7 and 8 of Example 1) was converted to the target compound. ESI-MS m/z 368 (M+H)⁺.

### Example 4: (4R,5S,6S)-3-((1-((1r,3R)-3-aminocyclobutyl)azetidin-3-yl)thio)-6-((R)-1-hydroxyethyl)-4-methyl-7-oxo-1-azabicyclo[3.2.0]hept-2-ene-2-carboxylic acid

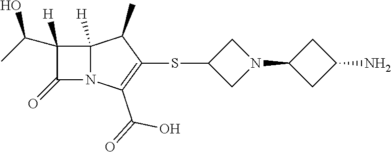 [SMILES: C[C@@H](O)[C@H]1C(=O)N2C(C(=O)O)=C(SC3CN([C@H]4C[C@H](N)C4)C3)[C@H](C)[C@H]12]

By following the same reaction procedures as described in Steps 9 and 10 of Example 1, 4-nitrobenzyl ((1r,3r)-3-(3-mercaptoazetidin-1-yl)cyclobutyl)carbamate (less polar isomer from Example 3) was converted to the target compound. ESI-MS m/z 368 (M+H)⁺.

### Example 5: (4R,5S,6S)-3-((1-((1S,3S)-3-aminocyclopentyl)azetidin-3-yl)thio)-6-((R)-1-hydroxyethyl)-4-methyl-7-oxo-1-azabicyclo[3.2.0]hept-2-ene-2-carboxylic acid

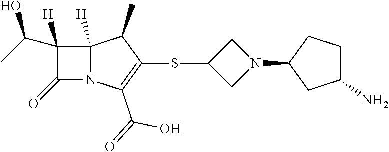 [SMILES: C[C@@H](O)[C@H]1C(=O)N2C(C(=O)O)=C(SC3CN([C@H]4CC[C@H](N)C4)C3)[C@H](C)[C@H]12]

By following the same reaction procedures as described in Steps 9 and 10 of Example 1, 4-nitrobenzyl ((1S,3S)-3-(3-mercaptoazetidin-1-yl)cyclopentyl)carbamate (less polar isomer prepared from (1S,3S)-3-aminocyclopentan-1-ol via Steps 6, 7 and 8 of Example 1) was converted to the target compound. ESI-MS m/z 382 (M+H)⁺.

### Example 6: (4R,5S,6S)-3-((1-((1R,3S)-3-aminocyclopentyl)azetidin-3-yl)thio)-6-((R)-1-hydroxyethyl)-4-methyl-7-oxo-1-azabicyclo[3.2.0]hept-2-ene-2-carboxylic acid

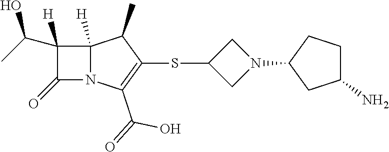 [SMILES: C[C@@H](O)[C@H]1C(=O)N2C(C(=O)O)=C(SC3CN([C@@H]4CC[C@H](N)C4)C3)[C@H](C)[C@H]12]

By following the same reaction procedures as described in Steps 9 and 10 of Example 1, 4-nitrobenzyl ((1R,3S)-3-(3-mercaptoazetidin-1-yl)cyclopentyl)carbamate (more polar isomer from Example 5) was converted to the target compound. ESI-MS m/z 382 (M+H)⁺.

### Example 7: (4R,5S,6S)-3-((1-((1S,3R)-3-aminocyclopentyl)azetidin-3-yl)thio)-6-((R)-1-hydroxyethyl)-4-methyl-7-oxo-1-azabicyclo[3.2.0]hept-2-ene-2-carboxylic acid

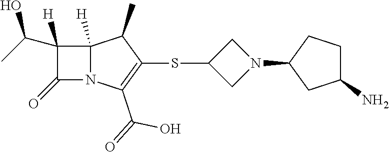 [SMILES: C[C@@H](O)[C@H]1C(=O)N2C(C(=O)O)=C(SC3CN([C@H]4CC[C@@H](N)C4)C3)[C@H](C)[C@H]12]

By following the same reaction procedures as described in Steps 9 and 10 of Example 1, 4-nitrobenzyl ((1S,3R)-3-(3-mercaptoazetidin-1-yl)cyclopentyl)carbamate (more polar isomer prepared from (1S,3R)-3-aminocyclopentan-1-ol via Steps 6, 7 and 8 of Example 1) was converted to the target compound. ESI-MS m/z 382 (M+H)⁺.

### Example 8: (4R,5S,6S)-3-((1-((1R,3R)-3-aminocyclopentyl)azetidin-3-yl)thio)-6-((R)-1-hydroxyethyl)-4-methyl-7-oxo-1-azabicyclo[3.2.0]hept-2-ene-2-carboxylic acid

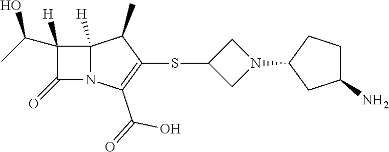 [SMILES: C[C@@H](O)[C@H]1C(=O)N2C(C(=O)O)=C(SC3CN([C@@H]4CC[C@@H](N)C4)C3)[C@H](C)[C@H]12]

By following the same reaction procedures as described in Steps 9 and 10 of Example 1, 4-nitrobenzyl ((1R,3R)-3-(3-mercaptoazetidin-1-yl)cyclopentyl)carbamate (less polar isomer from Example 7) was converted to the target compound. ESI-MS m/z 382 (M+H)⁺.

### Example 9: (4R,5S,6S)-3-((1-((1S,3R)-3-guanidinocyclopentyl)azetidin-3-yl)thio)-6-((R)-1-hydroxyethyl)-4-methyl-7-oxo-1-azabicyclo[3.2.0]hept-2-ene-2-carboxylic acid

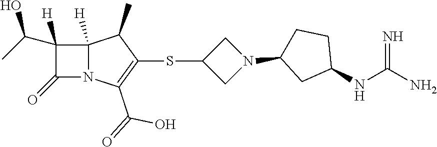 [SMILES: C[C@@H](O)[C@H]1C(=O)N2C(C(=O)O)=C(SC3CN([C@H]4CC[C@@H](NC(=N)N)C4)C3)[C@H](C)[C@H]12]

### Step 1. Synthesis of 1-((1R,3S)-3-hydroxycyclopentyl)guanidine, bis(4-nitrophenylmethyl) ester

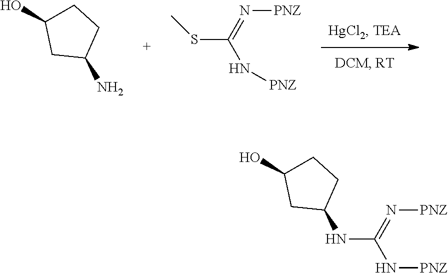

To a solution of (1S,3R)-3-aminocyclopentan-1-ol (1 g, 7.27 mmol) in DCM (60 mL) was added 2-methyl-2-thiopseudourea protected by PNZ (3.26 g, 7.27 mmol), thiethylamine (2.2 g, 21.78 mmol), and HgCl₂ (2 g, 7.27 mmol). The reaction mixture was stirred at room temperature overnight, filtered, the filtrate was washed with saturated aqueous NH₄Cl, water and brine, dried over Na₂SO₄, concentrated in vacuo. The residue was purified by flash chromatography on silica gel (PE/EA=10:1-2:1) to afford the product, 2.5 g, 69.4%. ESI-MS m/z 502 (M+H)⁺.

### Step 2. Synthesis of (4R,5S,6S)-3-((1-((1S,3R)-3-guanidinocyclopentyl)azetidin-3-yl)thio)-6-((R)-1-hydroxyethyl)-4-methyl-7-oxo-1-azabicyclo[3.2.0]hept-2-ene-2-carboxylic acid

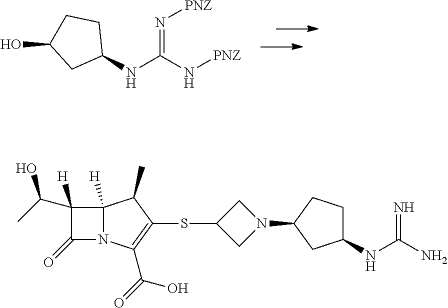

By following the same reaction procedures as described in Steps 9 and 10 of Example 1, 1-((1R,3S)-3-(3-mercaptoazetidin-1-yl)cyclopentyl)guanidine, bis(4-nitrophenylmethyl) ester (more polar isomer prepared from 1-((1R,3S)-3-hydroxycyclopentyl)guanidine, bis(4-nitrophenylmethyl) ester via Step 7, 8 of Example 1) was converted to the target compound. ESI-MS m/z 424 (M+H)⁺.

### Example 10: (4R,5S,6S)-3-((1-((1R,3R)-3-guanidinocyclopentyl)azetidin-3-yl)thio)-6-((R)-1-hydroxyethyl)-4-methyl-7-oxo-1-azabicyclo[3.2.0]hept-2-ene-2-carboxylic acid

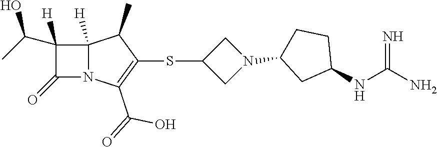 [SMILES: C[C@@H](O)[C@H]1C(=O)N2C(C(=O)O)=C(SC3CN([C@@H]4CC[C@@H](NC(=N)N)C4)C3)[C@H](C)[C@H]12]

By following the same reaction procedures as described in Steps 9 and 10 of Example 1, 1-((1R,3R)-3-(3-mercaptoazetidin-1-yl)cyclopentyl)guanidine, bis(4-nitrophenylmethyl) ester (less polar isomer from Example 9) was converted to the target compound. ESI-MS m/z 424 (M+H)⁺.

### Example 11: (4R,5S,6S)-3-((1-((1S,3S)-3-guanidinocyclopentyl)azetidin-3-yl)thio)-6-((R)-1-hydroxyethyl)-4-methyl-7-oxo-1-azabicyclo[3.2.0]hept-2-ene-2-carboxylic acid

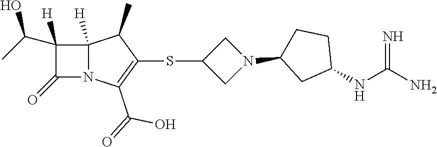 [SMILES: C[C@@H](O)[C@H]1C(=O)N2C(C(=O)O)=C(SC3CN([C@H]4CC[C@H](NC(=N)N)C4)C3)[C@H](C)[C@H]12]

### Step 1. Synthesis of 1-((1S,3S)-3-hydroxycyclopentyl)guanidine, bis(4-nitrophenylmethyl) ester

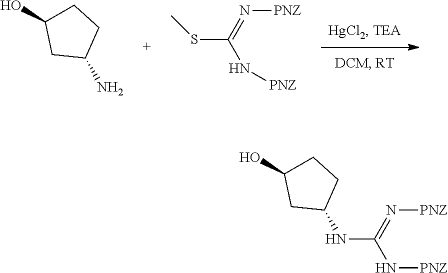

By following the same reaction procedures as described in Step 1 of Example 9, 1-((1S,3S)-3-hydroxycyclopentyl)guanidine, bis(4-nitrophenylmethyl) ester was obtained, 2.4 g. ESI-MS m/z 502 (M+H)⁺.

### Step 2. Synthesis of (4R,5S,6S)-3-((1-((1S,3S)-3-guanidinocyclopentyl)azetidin-3-yl)thio)-6-((R)-1-hydroxyethyl)-4-methyl-7-oxo-1-azabicyclo[3.2.0]hept-2-ene-2-carboxylic acid

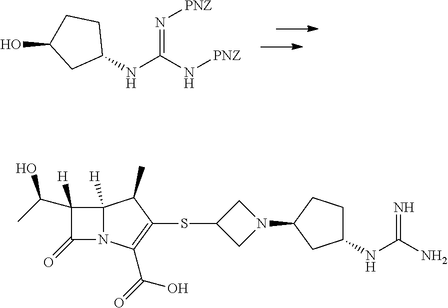

By following the same reaction procedures as described in Steps 9 and 10 of Example 1, 1-((1S,3S)-3-(3-mercaptoazetidin-1-yl)cyclopentyl)guanidine, bis(4-nitrophenylmethyl) ester (less polar isomer prepared from 1-((1S,3S)-3-hydroxycyclopentyl)guanidine, bis(4-nitrophenylmethyl) ester via Step 7, 8 of Example 1) was converted to the target compound. ESI-MS m/z 424 (M+H)⁺.

### Example 12: (4R,5S,6S)-3-((1-((1R,3S)-3-guanidinocyclopentyl)azetidin-3-yl)thio)-6-((R)-1-hydroxyethyl)-4-methyl-7-oxo-1-azabicyclo[3.2.0]hept-2-ene-2-carboxylic acid

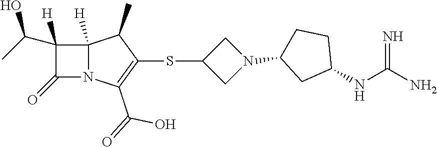 [SMILES: C[C@@H](O)[C@H]1C(=O)N2C(C(=O)O)=C(SC3CN([C@@H]4CC[C@H](NC(=N)N)C4)C3)[C@H](C)[C@H]12]

By following the same reaction procedures as described in Steps 9 and 10 of Example 1, 1-((1S,3R)-3-(3-mercaptoazetidin-1-yl)cyclopentyl)guanidine, bis(4-nitrophenylmethyl) ester (more polar isomer from Example 11) was converted to the target compound. ESI-MS m/z 424 (M+H)⁺.

### Example 13: (4R,5S,6S)-3-((1-((1S,3S)-3-aminocyclohexyl)azetidin-3-yl)thio)-6-((R)-1-hydroxyethyl)-4-methyl-7-oxo-1-azabicyclo[3.2.0]hept-2-ene-2-carboxylic acid

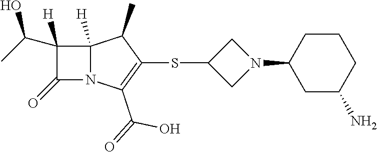 [SMILES: C[C@@H](O)[C@H]1C(=O)N2C(C(=O)O)=C(SC3CN([C@H]4CCC[C@H](N)C4)C3)[C@H](C)[C@H]12]

By following the same reaction procedures as described in Steps 9 and 10 of Example 1, 4-nitrobenzyl ((1S,3S)-3-(3-mercaptoazetidin-1-yl)cyclohexyl)carbamate (less polar isomer prepared from (1S,3S)-3-aminocyclohexan-1-ol via Steps 6, 7 and 8 of Example 1) was converted to the target compound. ESI-MS m/z 396 (M+H)⁺.

### Example 14: (4R,5S,6S)-3-((1-((1R,3S)-3-aminocyclohexyl)azetidin-3-yl)thio)-6-((R)-1-hydroxyethyl)-4-methyl-7-oxo-1-azabicyclo[3.2.0]hept-2-ene-2-carboxylic acid

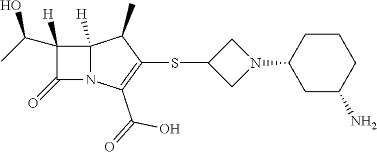 [SMILES: C[C@@H](O)[C@H]1C(=O)N2C(C(=O)O)=C(SC3CN([C@@H]4CCC[C@H](N)C4)C3)[C@H](C)[C@H]12]

By following the same reaction procedures as described in Steps 9 and 10 of Example 1, 4-nitrobenzyl ((1S,3R)-3-(3-mercaptoazetidin-1-yl)cyclohexyl)carbamate (more polar isomer from Example 13) was converted to the target compound ESI-MS m/z 396 (M+H)⁺.

### Example 15: (4R,5S,6S)-3-((1-((1S,3R)-3-aminocyclohexyl)azetidin-3-yl)thio)-6-((R)-1-hydroxyethyl)-4-methyl-7-oxo-1-azabicyclo[3.2.0]hept-2-ene-2-carboxylic acid

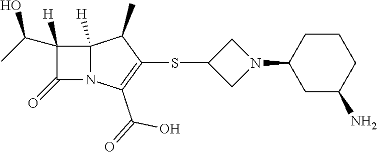 [SMILES: C[C@@H](O)[C@H]1C(=O)N2C(C(=O)O)=C(SC3CN([C@H]4CCC[C@@H](N)C4)C3)[C@H](C)[C@H]12]

By following the same reaction procedures as described in Steps 9 and 10 of Example 1, 4-nitrobenzyl ((1R,3S)-3-(3-mercaptoazetidin-1-yl)cyclohexyl)carbamate (more polar isomer prepared from (1S,3R)-3-aminocyclohexan-1-ol via Steps 6, 7 and 8 of Example 1) was converted to the target compound. ESI-MS m/z 396 (M+H)⁺.

### Example 16: (4R,5S,6S)-3-((1-((1R,3R)-3-aminocyclohexyl)azetidin-3-yl)thio)-6-((R)-1-hydroxyethyl)-4-methyl-7-oxo-1-azabicyclo[3.2.0]hept-2-ene-2-carboxylic acid

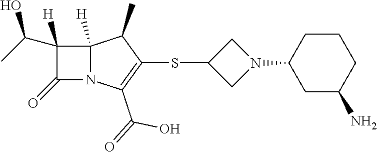 [SMILES: C[C@@H](O)[C@H]1C(=O)N2C(C(=O)O)=C(SC3CN([C@@H]4CCC[C@@H](N)C4)C3)[C@H](C)[C@H]12]

By following the same reaction procedures as described in Steps 9 and 10 of Example 1, 4-nitrobenzyl ((1R,3R)-3-(3-mercaptoazetidin-1-yl)cyclohexyl)carbamate (more polar isomer from Example 15) was converted to the target compound. ESI-MS m/z 396 (M+H)⁺.

### Example 17: (4R,5S,6S)-3-((1-((1R,3R)-3-guanidinocyclohexyl)azetidin-3-yl)thio)-6-((R)-1-hydroxyethyl)-4-methyl-7-oxo-1-azabicyclo[3.2.0]hept-2-ene-2-carboxylic acid

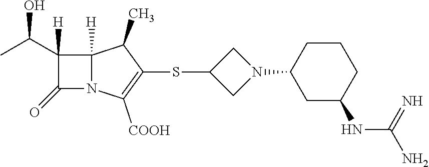 [SMILES: C[C@@H](O)[C@H]1C(=O)N2C(C(=O)O)=C(SC3CN([C@@H]4CCC[C@@H](NC(=N)N)C4)C3)[C@H](C)[C@H]12]

### Example 18: (4R,5S,6S)-3-((1-((1S,3R)-3-guanidinocyclohexyl)azetidin-3-yl)thio)-6-((R)-1-hydroxyethyl)-4-methyl-7-oxo-1-azabicyclo[3.2.0]hept-2-ene-2-carboxylic acid

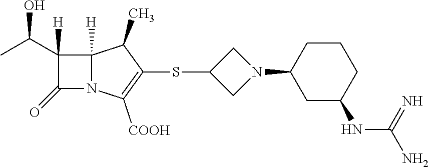 [SMILES: C[C@@H](O)[C@H]1C(=O)N2C(C(=O)O)=C(SC3CN([C@H]4CCC[C@@H](NC(=N)N)C4)C3)[C@H](C)[C@H]12]

### Step 1. Synthesis of 1-((1R,3S)-3-hydroxycyclohexyl)guanidine, bis(4-nitrophenylmethyl) ester

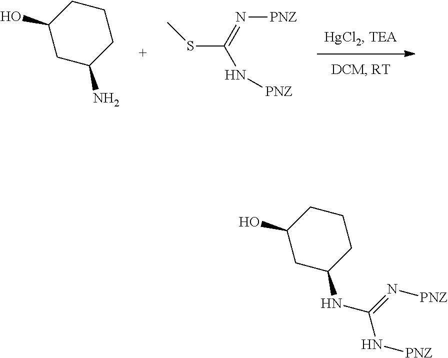

By following the same reaction procedures as described in Step 1 of Example 9, 1-((1R,3S)-3-hydroxycyclohexyl)guanidine, bis(4-nitrophenylmethyl) ester was obtained, 2.4 g. ESI-MS m/z 516 (M+H)⁺.

### Step 2. Synthesis of (4R,5S,6S)-3-((1-((1S,3R)-3-guanidinocyclohexyl)azetidin-3-yl)thio)-6-((R)-1-hydroxyethyl)-4-methyl-7-oxo-1-azabicyclo[3.2.0]hept-2-ene-2-carboxylic acid

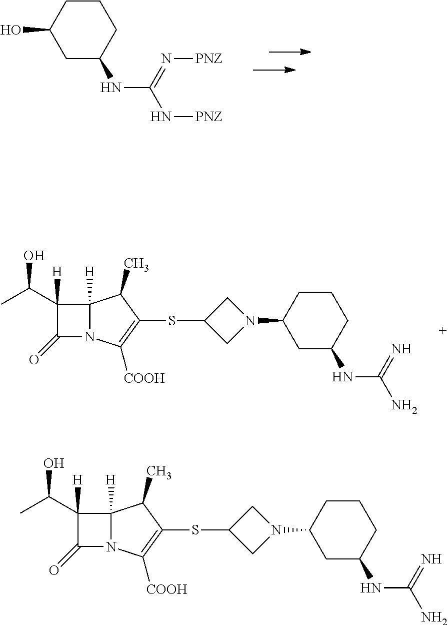

By following the same reaction procedures as described in Steps 9 and 10 of Example 1, 1-((1R)-3-(3-mercaptoazetidin-1-yl)cyclohexyl)guanidine, bis(4-nitrophenylmethyl) ester (more polar isomer and less polar isomer could not be separated by prep-TLC or chiral-HPLC, prepared from 1-((1R,3S)-3-hydroxycyclohexyl)guanidine, bis(4-nitrophenylmethyl) ester via Step 7, 8 of Example 1) was converted to a 1:1 mixture of Example 17 and Example 18. ESI-MS m/z 438 (M+H)⁺.

### Example 19: (4R,5S,6S)-3-((1-((1R,3S)-3-guanidinocyclohexyl)azetidin-3-yl)thio)-6-((R)-1-hydroxyethyl)-4-methyl-7-oxo-1-azabicyclo[3.2.0]hept-2-ene-2-carboxylic acid

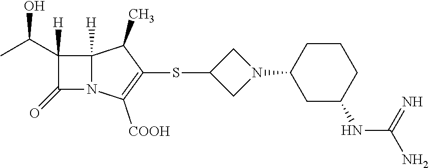 [SMILES: C[C@@H](O)[C@H]1C(=O)N2C(C(=O)O)=C(SC3CN([C@@H]4CCC[C@H](NC(=N)N)C4)C3)[C@H](C)[C@H]12]

### Example 20: (4R,5S,6S)-3-((1-((1S,3S)-3-guanidinocyclohexyl)azetidin-3-yl)thio)-6-((R)-1-hydroxyethyl)-4-methyl-7-oxo-1-azabicyclo[3.2.0]hept-2-ene-2-carboxylic acid

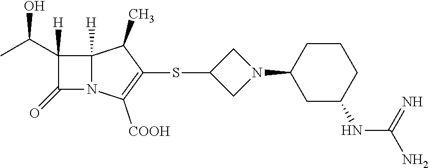 [SMILES: C[C@@H](O)[C@H]1C(=O)N2C(C(=O)O)=C(SC3CN([C@H]4CCC[C@H](NC(=N)N)C4)C3)[C@H](C)[C@H]12]

### Step 1. Synthesis of 1-((1S,3S)-3-hydroxycyclohexyl)guanidine, bis(4-nitrophenylmethyl) ester

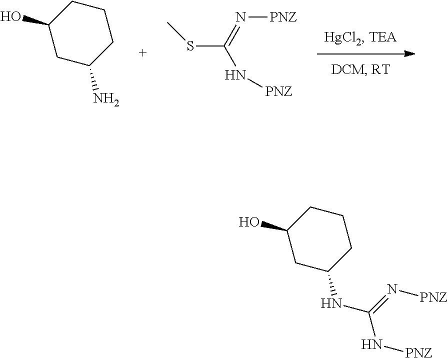

By following the same reaction procedures as described in Step 1 of Example 9, 1-((1S,3S)-3-hydroxycyclohexyl)guanidine, bis(4-nitrophenylmethyl) ester was obtained, 2.1 g. ESI-MS m/z 516 (M+H)⁺.

### Step 2. Synthesis of (4R,5S,6S)-3-((1-((1S,3S)-3-guanidinocyclohexyl)azetidin-3-yl)thio)-6-((R)-1-hydroxyethyl)-4-methyl-7-oxo-1-azabicyclo[3.2.0]hept-2-ene-2-carboxylic acid and (4R,5S,6S)-3-((1-((1R,3S)-3-guanidinocyclohexyl)azetidin-3-yl)thio)-6-((R)-1-hydroxyethyl)-4-methyl-7-oxo-1-azabicyclo[3.2.0]hept-2-ene-2-carboxylic acid

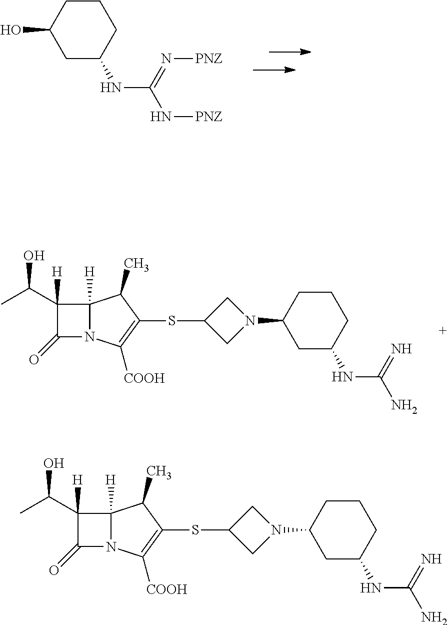

By following the same reaction procedures as described in Steps 9 and 10 of Example 1, 1-((1S)-3-(3-mercaptoazetidin-1-yl)cyclohexyl)guanidine, bis(4-nitrophenylmethyl) ester (more polar isomer and less polar isomer could not be separated by prep-TLC or chiral HPLC, prepared from 1-((1S,3S)-3-hydroxycyclohexyl)guanidine, bis(4-nitrophenylmethyl) ester via Step 7, 8 of Example 1) was converted to a 1:1 mixture of Example 19 and Example 20. ESI-MS m/z 438 (M+H)⁺.

### Example 21: (4R,5S,6S)-3-(((1r,3R)-3-(4-aminopiperidin-1-yl)cyclobutyl)thio)-6-((R)-1-hydroxyethyl)-4-methyl-7-oxo-1-azabicyclo[3.2.0]hept-2-ene-2-carboxylic acid

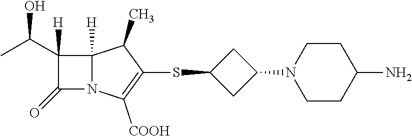 [SMILES: C[C@@H](O)[C@H]1C(=O)N2C(C(=O)O)=C(S[C@H]3C[C@H](N4CCC(N)CC4)C3)[C@H](C)[C@H]12]

### Step 1. Synthesis of tert-butyl (1-((1r,3r)-3-mercaptocyclobutyl)piperidin-4-yl)carbamate

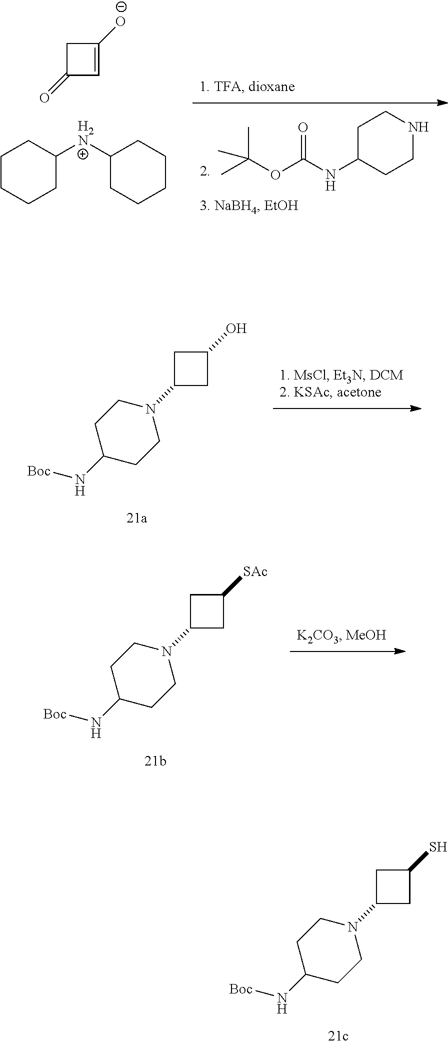

The procedures described by Denonne, F, et al. (WO 2009/092764) is adapted to prepare alcohol 21a, and the procedures of Example 1, Steps 2, 3, and 4 are used to prepare intermediate 21c.

### Step 2. Synthesis of (4R,5S,6S)-3-(((1r,3R)-3-(4-aminopiperidin-1-yl)cyclobutyl)thio)-6-((R)-1-hydroxyethyl)-4-methyl-7-oxo-1-azabicyclo[3.2.0]hept-2-ene-2-carboxylic acid

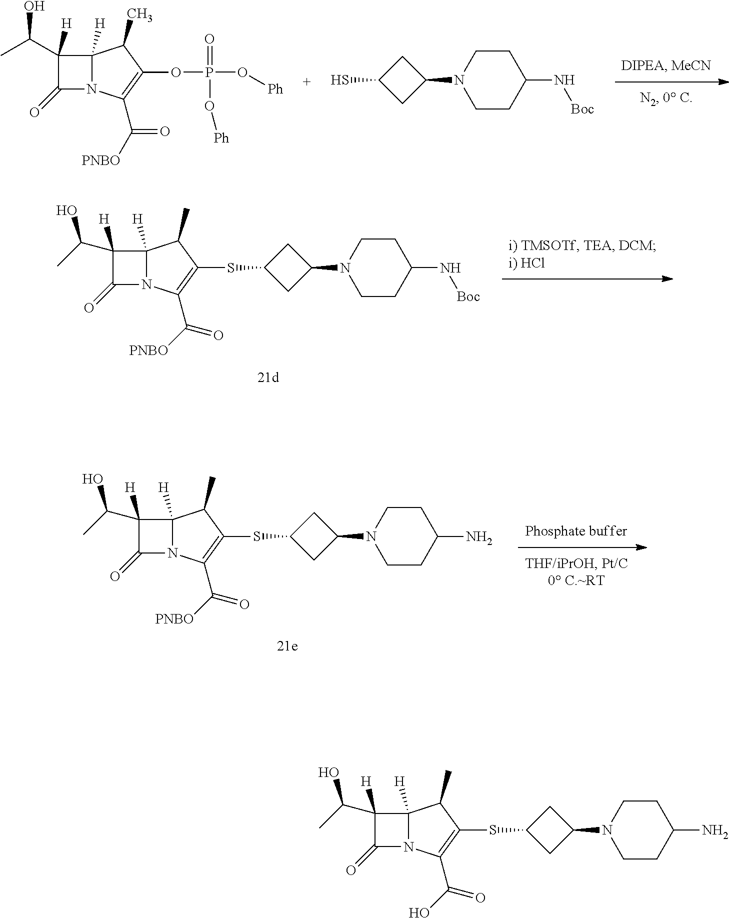

The procedure of Example 1, Step 9 is utilized to prepare carbamate 21d. The carbamate is deprotected using trimethylsilyl trifluoromethanesulfonate in DCM to provide amine 21e. The procedure of Example 1, Step 10 is used to prepare the target compound from 21e.

### Example 22: (4R,5S,6S)-3-((1-((2-aminopyrimidin-5-yl)methyl)azetidin-3-yl)thio)-6-((R)-1-hydroxyethyl)-4-methyl-7-oxo-1-azabicyclo[3.2.0]hept-2-ene-2-carboxylic acid

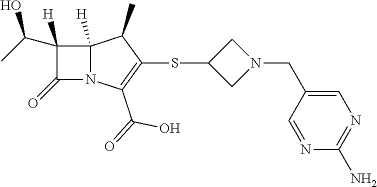 [SMILES: C[C@@H](O)[C@H]1C(=O)N2C(C(=O)O)=C(SC3CN(Cc4cnc(N)nc4)C3)[C@H](C)[C@H]12]

### Step 1: Synthesis of 1-((2-aminopyrimidin-5-yl)methyl)azetidine-3-thiol

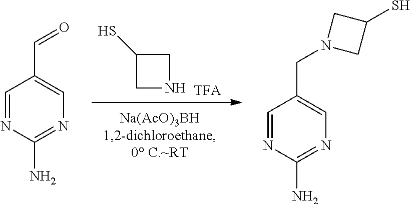 [SMILES: Nc1ncc(C=O)cn1 ; SC1CNC1 ; Nc1ncc(CN2CC(S)C2)cn1 ; "TFA"]

By using the same reaction procedures as described in Step 8 of Example 1, using 2-aminopyrimidine-5-carbaldehyde instead of 4-nitrobenzyl 4-oxopiperidine-1-carboxylate, the target compound was prepared, 1 g. ESI-MS m/z 197 (MH)⁺.

### Step 2: Synthesis of (4R,5S,6S)-3-((1-((2-aminopyrimidin-5-yl)methyl)azetidin-3-yl)thio)-6-((R)-1-hydroxyethyl)-4-methyl-7-oxo-1-azabicyclo[3.2.0]hept-2-ene-2-carboxylic acid

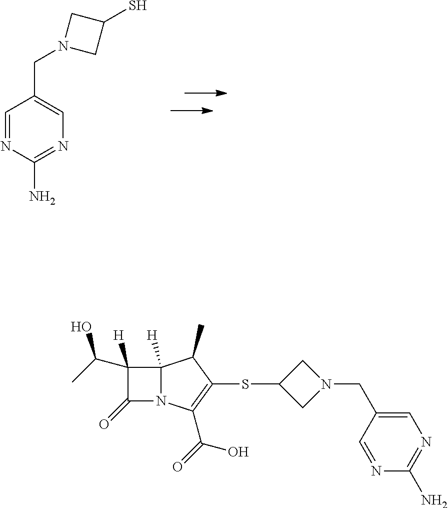 [SMILES: Nc1ncc(CN2CC(S)C2)cn1 ; C[C@@H](O)[C@H]1C(=O)N2C(C(=O)O)=C(SC3CN(Cc4cnc(N)nc4)C3)[C@H](C)[C@H]12]

By following the same reaction procedures as described in Steps 9 and 10 of Example 1, the above product was converted to the target compound. ESI-MS m/z 406 (MH)⁺.

### Example 23: (4R,5S,6S)-3-((1-((6-aminopyridin-3-yl)methyl)azetidin-3-yl)thio)-6-((R)-1-hydroxyethyl)-4-methyl-7-oxo-1-azabicyclo[3.2.0]hept-2-ene-2-carboxylic acid

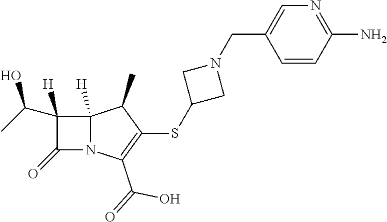 [SMILES: C[C@@H](O)[C@H]1C(=O)N2C(C(=O)O)=C(SC3CN(Cc4ccc(N)nc4)C3)[C@H](C)[C@H]12]

By following the same reaction procedures as described in Steps 1 and 2 of Example 22, 6-aminonicotinaldehyde was converted to the target compound. ESI-MS m/z 405 (MH)⁺.

### Example 24: (4R,5S,6S)-3-(((R)-1-((2-aminopyrimidin-5-yl)methyl)pyrrolidin-3-yl)thio)-6-((R)-1-hydroxyethyl)-4-methyl-7-oxo-1-azabicyclo[3.2.0]hept-2-ene-2-carboxylic acid

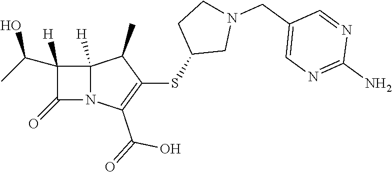 [SMILES: C[C@@H](O)[C@H]1C(=O)N2C(C(=O)O)=C(S[C@@H]3CCN(Cc4cnc(N)nc4)C3)[C@H](C)[C@H]12]

By following the same reaction procedures as described in Steps 1 and 2 of Example 22, (R)-pyrrolidine-3-thiol 2,2,2-trifluoroacetic acid was converted to the target compound. ESI-MS m/z 420 (MH)⁺.

### Example 25: (4R,5S,6S)-3-(((R)-1-((6-aminopyridin-3-yl)methyl)pyrrolidin-3-yl)thio)-6-((R)-1-hydroxyethyl)-4-methyl-7-oxo-1-azabicyclo[3.2.0]hept-2-ene-2-carboxylic acid

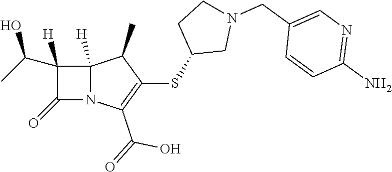 [SMILES: C[C@@H](O)[C@H]1C(=O)N2C(C(=O)O)=C(S[C@@H]3CCN(Cc4ccc(N)nc4)C3)[C@H](C)[C@H]12]

By following the same reaction procedures as described in Steps 1 and 2 of Example 11, (R)-pyrrolidine-3-thiol 2,2,2-trifluoroacetic acid and 6-aminonicotinaldehyde were converted to the target compound. ESI-MS m/z 419 (MH)⁺.

### Example 26: (4R,5S,6S)-3-((1-((1-carbamimidoylpiperidin-4-yl)methyl)azetidin-3-yl)thio)-6-((R)-1-hydroxyethyl)-4-methyl-7-oxo-1-azabicyclo[3.2.0]hept-2-ene-2-carboxylic acid

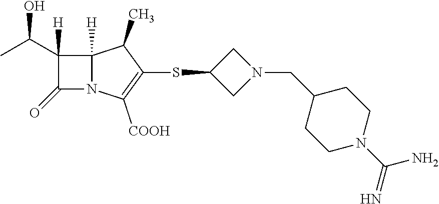

### Step 1. Synthesis of 4-nitrobenzyl (E)-((4-(hydroxymethyl)piperidin-1-yl)((((4-nitrobenzyl)oxy)carbonyl)imino)methyl)carbamate

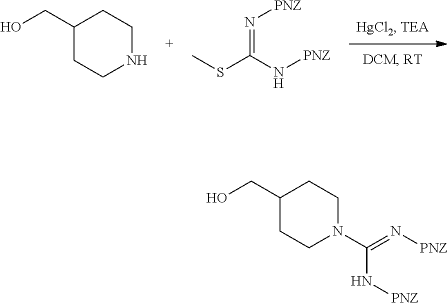

By following the same reaction procedures as described in Step 1 of Example 9, 4-nitrobenzyl (E)-((4-(hydroxymethyl)piperidin-1-yl)((((4-nitrobenzyl)oxy)carbonyl)imino)methyl)carbamate was obtained. ESI-MS m/z 516 (M+H)⁺.

### Step 2. Synthesis of (4R,5S,6S)-3-((1-((1-carbamimidoylpiperidin-4-yl)methyl)azetidin-3-yl)thio)-6-((R)-1-hydroxyethyl)-4-methyl-7-oxo-1-azabicyclo[3.2.0]hept-2-ene-2-carboxylic acid

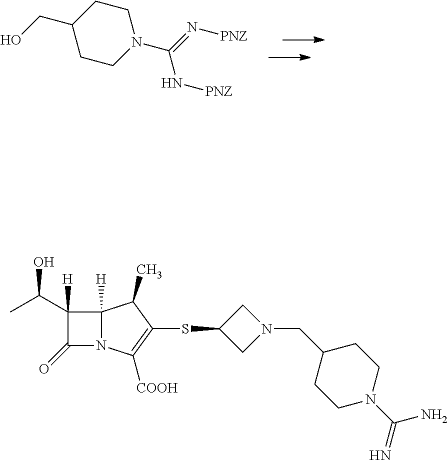

By following the same reaction procedures as described in Steps 7, 8, 9 and 10 of Example 1. 4-Nitrobenzyl (E)-((4-(hydroxymethyl)piperidin-1-yl)((((4-nitrobenzyl)oxy)carbonyl)imino)methyl)carbamate was converted to the target compound. ESI-MS m/z 438 (M+H)⁺.

### Example 27: (4R,5S,6S)-3-(((R)-1-((1R,4R)-4-guanidinocyclohexyl)pyrrolidin-3-yl)thio)-6-((R)-1-hydroxyethyl)-4-methyl-7-oxo-1-azabicyclo[3.2.0]hept-2-ene-2-carboxylic acid

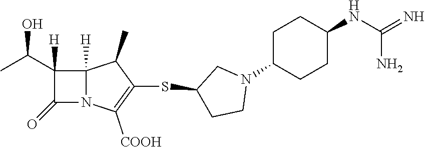 [SMILES: C[C@@H](O)[C@H]1C(=O)N2C(C(=O)O)=C(S[C@@H]3CCN([C@H]4CC[C@H](NC(=N)N)CC4)C3)[C@H](C)[C@H]12]

### Step 1. Synthesis of pyrrolidine-3-thiol

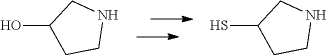 [SMILES: OC1CCNC1 ; SC1CCNC1]

By following the same reaction procedures as described in Steps 1, 2, 3, 4 and 5 of Example 1, pyrrolidin-3-ol was converted to the target compound. ESI-MS m/z 104 (M+H)⁺.

### Step 2. Synthesis of 1-((1S,4S)-4-hydroxycyclohexyl)-1,3-bis(4-nitrobenzyl)guanidine ester

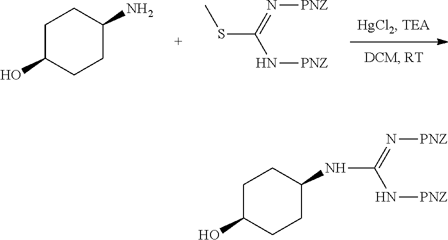

By following the same reaction procedures as described in Step 1 of Example 9, 1-((1S,4S)-4-hydroxycyclohexyl)-1,3-bis(4-nitrobenzyl)guanidine ester was obtained. ESI-MS m/z 516 (M+H)⁺.

### Step 3. Synthesis of (4R,5S,6S)-3-(((R)-1-((1R,4R)-4-guanidinocyclohexyl)pyrrolidin-3-yl)thio)-6-((R)-1-hydroxyethyl)-4-methyl-7-oxo-1-azabicyclo[3.2.0]hept-2-ene-2-carboxylic acid

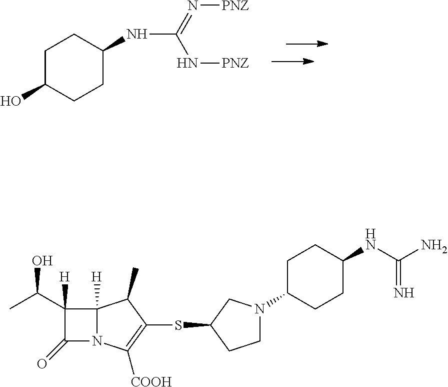

By following the same reaction procedures as described in Steps 9 and 10 of Example 1, 1-((1S,4S)-4-((R)-3-mercaptopyrrolidin-1-yl)cyclohexyl)-1,3-bis(4-nitrobenzyl)guanidine ester (less polar isomer prepared from synthesis of 1-((1S,4S)-4-hydroxycyclohexyl)-1,3-bis(4-nitrobenzyl)guanidine ester via Step 7, 8 of Example 1) was converted to the target compound. ESI-MS m/z 452 (M+H)⁺.

### Example 28: (4R,5S,6S)-3-(((R)-1-(((1R,4R)-4-guanidinocyclohexyl)methyl)pyrrolidin-3-yl)thio)-6-((R)-1-hydroxyethyl)-4-methyl-7-oxo-1-azabicyclo[3.2.0]hept-2-ene-2-carboxylic acid

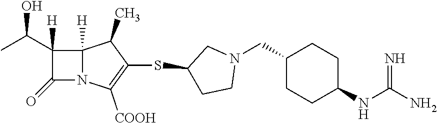 [SMILES: C[C@@H](O)[C@H]1C(=O)N2C(C(=O)O)=C(S[C@@H]3CCN(C[C@H]4CC[C@H](NC(=N)N)CC4)C3)[C@H](C)[C@H]12]

### Step 1. Synthesis of 1-((1R,4R)-4-(hydroxymethyl)cyclohexyl)-1,3-bis(4-nitrobenzyl)guanidine ester

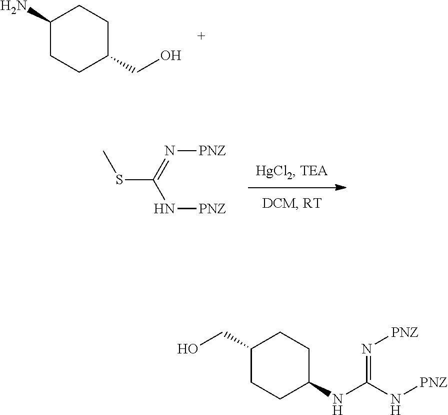

By following the same reaction procedures as described in Step 1 of Example 9, 1-((1R,4R)-4-(hydroxymethyl)cyclohexyl)-1,3-bis(4-nitrobenzyl)guanidine ester was obtained. ESI-MS m/z 530 (M+H)⁺.

### Step 2. Synthesis of (4R,5S,6S)-3-(((R)-1-(((1R,4R)-4-guanidinocyclohexyl)methyl)pyrrolidin-3-yl)thio)-6-((R)-1-hydroxyethyl)-4-methyl-7-oxo-1-azabicyclo[3.2.0]hept-2-ene-2-carboxylic acid

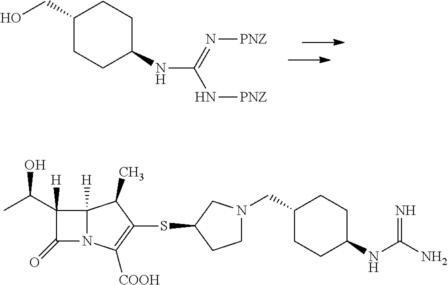

By following the same reaction procedures as described in Steps 7, 8, 9 and 10 of Example 1, 1-((1R,4R)-4-(hydroxymethyl)cyclohexyl)-1,3-bis(4-nitrobenzyl)guanidine ester was converted to the target compound. ESI-MS m/z 466 (M+H)⁺.

### Example 29: (4R,5S,6S)-6-((R)-1-hydroxyethyl)-4-methyl-3-(((R)-1-(1-methylpiperidin-4-yl)pyrrolidin-3-yl)thio)-7-oxo-1-azabicyclo[3.2.0]hept-2-ene-2-carboxylic acid

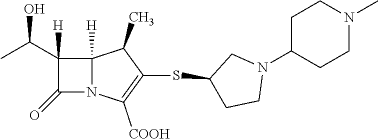 [SMILES: C[C@@H](O)[C@H]1C(=O)N2C(C(=O)O)=C(S[C@@H]3CCN(C4CCN(C)CC4)C3)[C@H](C)[C@H]12]

By following the same reaction procedures as described in Steps 8, 9 and 10 of Example 1, 1-methylpiperidin-4-one was converted to the target compound. ESI-MS m/z 410 (M+H)⁺.

### Example 30: (4R,5S,6S)-3-(((R)-1-((1R,3R)-3-guanidinocyclopentyl)pyrrolidin-3-yl)thio)-6-((R)-1-hydroxyethyl)-4-methyl-7-oxo-1-azabicyclo[3.2.0]hept-2-ene-2-carboxylic acid

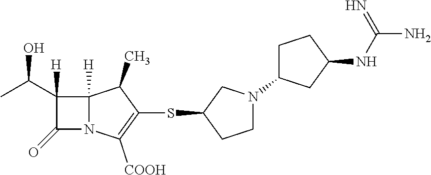 [SMILES: C[C@@H](O)[C@H]1C(=O)N2C(C(=O)O)=C(S[C@@H]3CCN([C@@H]4CC[C@@H](NC(=N)N)C4)C3)[C@H](C)[C@H]12]

### Step 1. Synthesis of 1-((1R,3S)-3-hydroxycyclopentyl)-1,3-bis(4-nitrobenzyl)guanidine ester

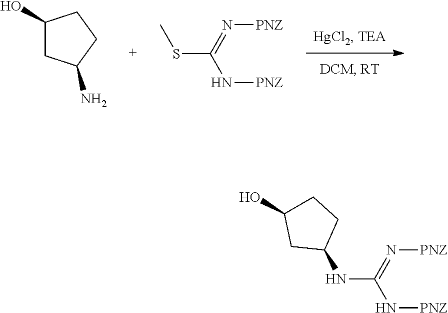

By following the same reaction procedures as described in Step 1 of Example 9, 1-((1R,3S)-3-hydroxycyclopentyl)-1,3-bis(4-nitrobenzyl)guanidine ester was obtained. ESI-MS m/z 502 (M+H)⁺.

### Step 2. Synthesis of (4R,5S,6S)-3-(((R)-1-((1R,3R)-3-guanidinocyclopentyl)pyrrolidin-3-yl)thio)-6-((R)-1-hydroxyethyl)-4-methyl-7-oxo-1-azabicyclo[3.2.0]hept-2-ene-2-carboxylic acid

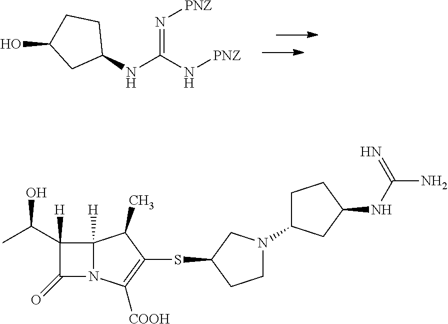

By following the same reaction procedures as described in Steps 9 and 10 of Example 1, 1-((1R,3R)-3-((R)-3-mercaptopyrrolidin-1-yl)cyclopentyl)-1,3-bis(4-nitrobenzyl)guanidine ester (less polar isomer prepared from 1-((1R,3S)-3-hydroxycyclopentyl)-1,3-bis(4-nitrobenzyl)guanidine ester via Step 7, 8 of Example 1) was converted to the target compound. ESI-MS m/z 438 (M+H)⁺.

### Example 31: (4R,5S,6S)-3-(((R)-1-((1S,3R)-3-guanidinocyclopentyl)pyrrolidin-3-yl)thio)-6-((R)-1-hydroxyethyl)-4-methyl-7-oxo-1-azabicyclo[3.2.0]hept-2-ene-2-carboxylic acid

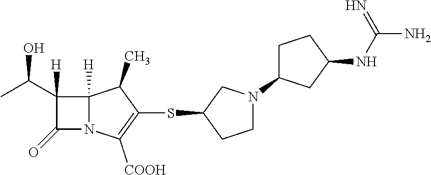 [SMILES: C[C@@H](O)[C@H]1C(=O)N2C(C(=O)O)=C(S[C@@H]3CCN([C@H]4CC[C@@H](NC(=N)N)C4)C3)[C@H](C)[C@H]12]

By following the same reaction procedures as described in Steps 9 and 10 of Example 1, 1-((1R,3S)-3-((R)-3-mercaptopyrrolidin-1-yl)cyclopentyl)-1,3-bis(4-nitrobenzyl)guanidine ester (more polar isomer from Example 30) was converted to the target compound. ESI-MS m/z 438 (M+H)⁺.

### Example 32: (4R,5S,6S)-6-((R)-1-hydroxyethyl)-4-methyl-7-oxo-3-((1-(((R)-pyrrolidin-2-yl)methyl)azetidin-3-yl)thio)-1-azabicyclo[3.2.0]hept-2-ene-2-carboxylic acid

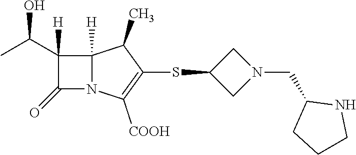

By following the same reaction procedures as described in Steps 6, 7, 8, 9, and 10 of Example 1, from (R)-pyrrolidin-2-ylmethanol, was converted to the target compound. ESI-MS m/z 382 (M+H)⁺.

### Example 33: (4R,5S,6S)-3-(((R)-1-(((1S,4S)-4-guanidinocyclohexyl)methyl)pyrrolidin-3-yl)thio)-6-((R)-1-hydroxyethyl)-4-methyl-7-oxo-1-azabicyclo[3.2.0]hept-2-ene-2-carboxylic acid

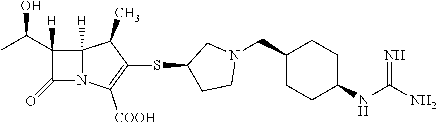 [SMILES: C[C@@H](O)[C@H]1C(=O)N2C(C(=O)O)=C(S[C@@H]3CCN(C[C@H]4CC[C@@H](NC(=N)N)CC4)C3)[C@H](C)[C@H]12]

### Step 1. Synthesis of 1-((1S,4S)-4-(hydroxymethyl)cyclohexyl)-1,3-bis(4-nitrobenzyl)guanidine ester

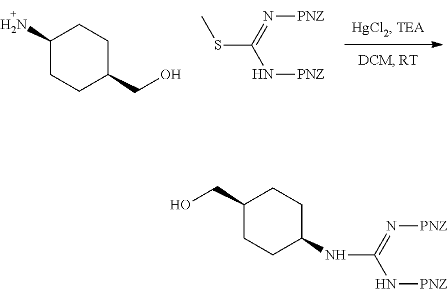

By following the same reaction procedures as described in Step 1 of Example 9, 1-((1S,4S)-4-(hydroxymethyl)cyclohexyl)-1,3-bis(4-nitrobenzyl)guanidine ester was obtained. ESI-MS m/z 530 (M+H)⁺.

### Step 2. Synthesis of: (4R,5S,6S)-3-(((R)-1-(((1S,4S)-4-guanidinocyclohexyl)methyl)pyrrolidin-3-yl)thio)-6-((R)-1-hydroxyethyl)-4-methyl-7-oxo-1-azabicyclo[3.2.0]hept-2-ene-2-carboxylic acid

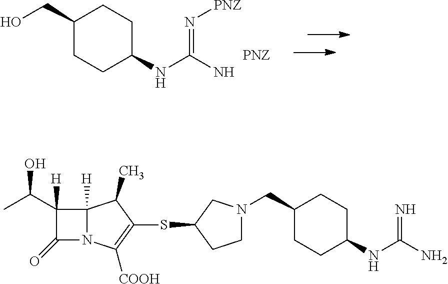

By following the same reaction procedures as described in Steps 7, 8, 9, and 10 of Example 1, 1-((1S,4S)-4-(hydroxymethyl)cyclohexyl)-1,3-bis(4-nitrobenzyl)guanidine ester was converted to the target compound. ESI-MS m/z 466 (M+H)⁺.

### Example 34: (4R,5S,6S)-3-(((R)-1-((1S,4S)-4-guanidinocyclohexyl)pyrrolidin-3-yl)thio)-6-((R)-1-hydroxyethyl)-4-methyl-7-oxo-1-azabicyclo[3.2.0]hept-2-ene-2-carboxylic acid

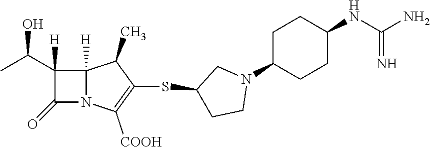 [SMILES: C[C@@H](O)[C@H]1C(=O)N2C(C(=O)O)=C(S[C@@H]3CCN([C@H]4CC[C@@H](NC(=N)N)CC4)C3)[C@H](C)[C@H]12]

By following the same reaction procedures as described in Steps 9 and 10 of Example 11-((1R,4R)-4-((R)-3-mercaptopyrrolidin-1-yl)cyclohexyl)-1,3-bis(4-nitrobenzyl)guanidine ester (more polar isomer from Example 27) was converted to the target compound. ESI-MS m/z 452 (M+H)⁺.

### Example 35: (4R,5S,6S)-3-(((R)-1-((1R,3R)-3-aminocyclopentyl)pyrrolidin-3-yl)thio)-6-((R)-1-hydroxyethyl)-4-methyl-7-oxo-1-azabicyclo[3.2.0]hept-2-ene-2-carboxylic acid

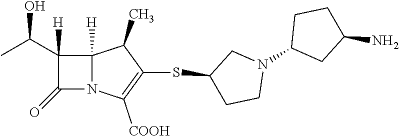 [SMILES: C[C@@H](O)[C@H]1C(=O)N2C(C(=O)O)=C(S[C@@H]3CCN([C@@H]4CC[C@@H](N)C4)C3)[C@H](C)[C@H]12]

By following the same reaction procedures as described in Steps 9 and 10 of Example 1, 4-nitrobenzyl ((1R,3R)-3-((R)-3-mercaptopyrrolidin-1-yl)cyclopentyl)carbamate (less polar isomer prepared from (1S,3R)-3-aminocyclopentan-1-ol via Steps 6, 7 and 8 of Example 1) was converted to the target compound. ESI-MS m/z 396 (M+H)⁺.

### Example 36: (4R,5S,6S)-3-(((R)-1-((1S,3R)-3-aminocyclopentyl)pyrrolidin-3-yl)thio)-6-((R)-1-hydroxyethyl)-4-methyl-7-oxo-1-azabicyclo[3.2.0]hept-2-ene-2-carboxylic acid

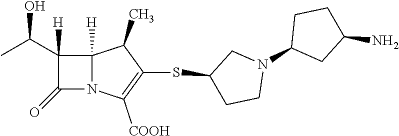 [SMILES: C[C@@H](O)[C@H]1C(=O)N2C(C(=O)O)=C(S[C@@H]3CCN([C@H]4CC[C@@H](N)C4)C3)[C@H](C)[C@H]12]

By following the same reaction procedures as described in Steps 9 and 10 of Example 1, 4-nitrobenzyl ((1R,3S)-3-((R)-3-mercaptopyrrolidin-1-yl)cyclopentyl)carbamate (more polar isomer from Example 35) was converted to the target compound ESI-MS m/z 396 (M+H)⁺.

### Example 37: (4R,5S,6S)-6-((R)-1-hydroxyethyl)-4-methyl-7-oxo-3-((1-(pyrrolidin-3-yl)azetidin-3-yl)thio)-1-azabicyclo[3.2.0]hept-2-ene-2-carboxylic acid

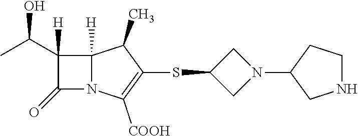

By following the same reaction procedures as described in Steps 6, 7, 8, 9, and 10 of Example 1, pyrrolidin-3-ol was converted to the target compound. ESI-MS m/z 368 (M+H)⁺.

### Example 38: (4R,5S,6S)-6-((R)-1-hydroxyethyl)-4-methyl-7-oxo-3-(((1R,3R)-3-(piperidin-4-ylamino)cyclobutyl)thio)-1-azabicyclo[3.2.0]hept-2-ene-2-carboxylic acid

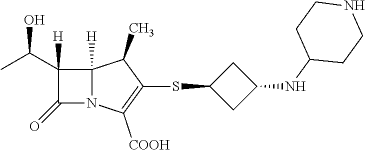 [SMILES: C[C@@H](O)[C@H]1C(=O)N2C(C(=O)O)=C(S[C@H]3C[C@H](NC4CCNCC4)C3)[C@H](C)[C@H]12]

### Step 1. Synthesis of (1r,3r-3-aminocyclobutane-1-thiol

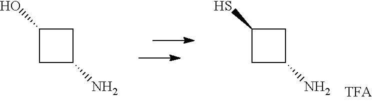 [SMILES: N[C@H]1C[C@@H](O)C1 ; N[C@H]1C[C@H](S)C1]

By following the same reaction procedures as described in Steps 1, 2, 3, 4, and 5 of Example 1, (1s,3s)-3-aminocyclobutan-1-ol was converted to the target compound. ESI-MS m/z 104 (M+H)⁺.

### Step 2. Synthesis of (4R,5S,6S)-6-((R)-1-hydroxyethyl)-4-methyl-7-oxo-3-(((1R,3R)-3-(piperidin-4-ylamino)cyclobutyl)thio)-1-azabicyclo[3.2.0]hept-2-ene-2-carboxylic acid

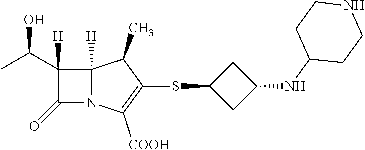 [SMILES: C[C@@H](O)[C@H]1C(=O)N2C(C(=O)O)=C(S[C@H]3C[C@H](NC4CCNCC4)C3)[C@H](C)[C@H]12]

By following the same reaction procedures as described in Steps 6, 8, 9, and 10 of Example 1, piperidin-4-one was converted to the target compound. ESI-MS m/z 396 (M+H)⁺.

### Example 39: (4R,5S,6S)-3-((1-(((S)-1-carbamimidoylpyrrolidin-3-yl)methyl)azetidin-3-yl)thio)-6-((R)-1-hydroxyethyl)-4-methyl-7-oxo-1-azabicyclo[3.2.0]hept-2-ene-2-carboxylic acid

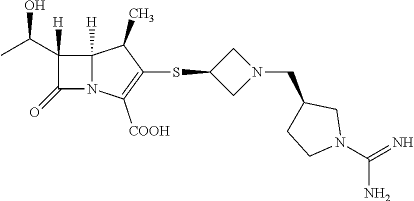

### Step 1. Synthesis of 4-nitrobenzyl (R,E)-((3-(hydroxymethyl)pyrrolidin-1-yl)((((4-nitrobenzyl)oxy)carbonyl)imino)methyl)carbamate ester

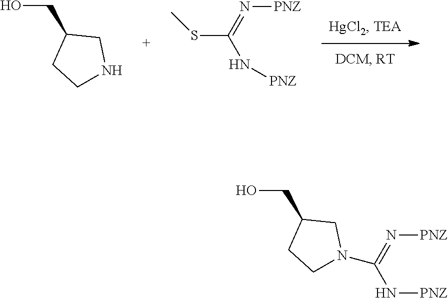

By following the same reaction procedures as described in Step 1 of Example 9, 4-nitrobenzyl (R,E)-((3-(hydroxymethyl)pyrrolidin-1-yl)((((4-nitrobenzyl)oxy)carbonyl)imino)methyl)carbamate ester was obtained. ESI-MS m/z 502 (M+H)⁺.

### Step 2. Synthesis of (4R,5S,6S)-3-((1-(((S)-1-carbamimidoylpyrrolidin-3-yl)methyl)azetidin-3-yl)thio)-6-((R)-1-hydroxyethyl)-4-methyl-7-oxo-1-azabicyclo[3.2.0]hept-2-ene-2-carboxylic acid

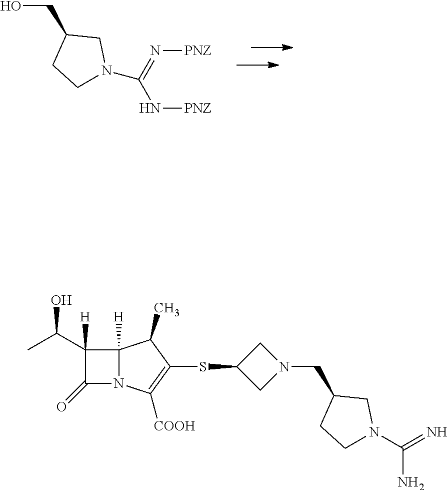

By following the same reaction procedures as described in Steps 7, 8, 9, and 10 of Example 1, 4-nitrobenzyl (R,E)-((3-(hydroxymethyl)pyrrolidin-1-yl)((((4-nitrobenzyl)oxy)carbonyl)imino) methyl)carbamate ester was converted to the target compound. ESI-MS m/z 424 (M+H)⁺.

### Example 40: (4R,5S,6S)-6-((R)-1-hydroxyethyl)-4-methyl-3-((1-((1-methylpiperidin-4-yl)methyl)azetidin-3-yl)thio)-7-oxo-1-azabicyclo[3.2.0]hept-2-ene-2-carboxylic acid

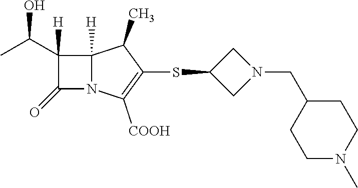

By following the same reaction procedures as described in Steps 8, 9, and 10 of Example 1, 1-methylpiperidine-4-carbaldehyde was converted to the target compound. ESI-MS m/z 410 (M+H)⁺.

### Example 41: (4R,5S,6S)-3-(((R)-1-(((1R,4R)-4-(guanidinomethyl)cyclohexyl)methyl)pyrrolidin-3-yl)thio)-6-((R)-1-hydroxyethyl)-4-methyl-7-oxo-1-azabicyclo[3.2.0]hept-2-ene-2-carboxylic acid

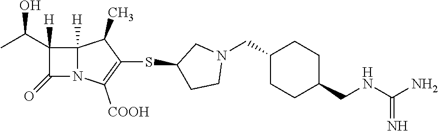 [SMILES: C[C@@H](O)[C@H]1C(=O)N2C(C(=O)O)=C(S[C@@H]3CCN(C[C@H]4CC[C@H](CNC(=N)N)CC4)C3)[C@H](C)[C@H]12]

### Step 1. Synthesis of 1-(((1R,4R)-4-(hydroxymethyl)cyclohexyl)methyl)-1,3-bis(4-nitrobenzyl)guanidine ester

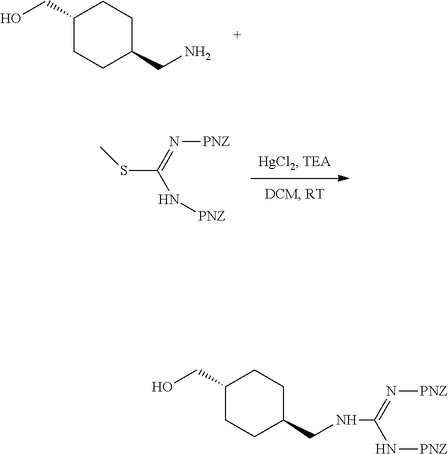

By following the same reaction procedures as described in Step 1 of Example 9, 1-(((1R,4R)-4-(hydroxymethyl)cyclohexyl)methyl)-1,3-bis(4-nitrobenzyl)guanidine ester was obtained. ESI-MS m/z 544 (M+H)⁺.

### Step 2. Synthesis of (4R,5S,6S)-3-(((R)-1-(((1R,4R)-4-(guanidinomethyl)cyclohexyl)methyl)pyrrolidin-3-yl)thio)-6-((R)-1-hydroxyethyl)-4-methyl-7-oxo-1-azabicyclo[3.2.0]hept-2-ene-2-carboxylic acid

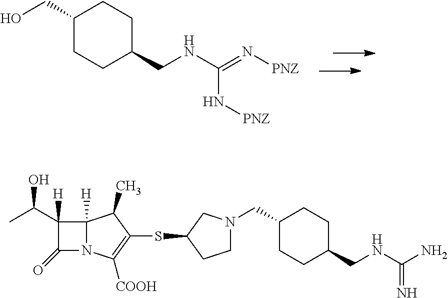

By following the same reaction procedures as described in Steps 7, 8, 9, and 10 of Example 1, 1-(((1R,4R)-4-(hydroxymethyl)cyclohexyl)methyl)-1,3-bis(4-nitrobenzyl)guanidine ester was converted to the target compound. ESI-MS m/z 480 (M+H)⁺.

### Example 42: (4R,5S,6S)-6-((R)-1-hydroxyethyl)-4-methyl-3-((1-(1-methylpiperidin-4-yl)azetidin-3-yl)thio)-7-oxo-1-azabicyclo[3.2.0]hept-2-ene-2-carboxylic acid

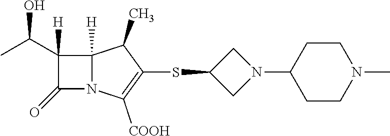

By following the same reaction procedures as described in Steps 8, 9, and 10 of Example 1, 1-methylpiperidin-4-one was converted to the target compound. ESI-MS m/z 396 (M+H)⁺.

### Example 43: (4R,5S,6S)-3-(((1S,3S)-3-((1-carbamimidoylpiperidin-4-yl)amino)cyclobutyl)thio)-6-((R)-1-hydroxyethyl)-4-methyl-7-oxo-1-azabicyclo[3.2.0]hept-2-ene-2-carboxylic acid

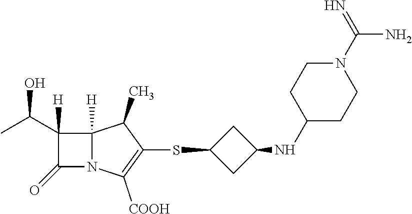 [SMILES: C[C@@H](O)[C@H]1C(=O)N2C(C(=O)O)=C(S[C@H]3C[C@@H](NC4CCN(C(=N)N)CC4)C3)[C@H](C)[C@H]12]

### Step 1. Synthesis of (1s,3s)-3-aminocyclobutane-1-thiol

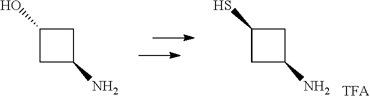 [SMILES: N[C@H]1C[C@H](O)C1 ; N[C@H]1C[C@@H](S)C1]

By following the same reaction procedures as described in Steps 1, 2, 3, 4, and 5 of Example 1, (1r,3r)-3-aminocyclobutan-1-ol was converted to the target compound. ESI-MS m/z 104 (M+H)⁺.

### Step 2. Synthesis of 4-nitrobenzyl (E)-(((((4-nitrobenzyl)oxy)carbonyl)imino)(4-oxopiperidin-1-yl)methyl)carbamate

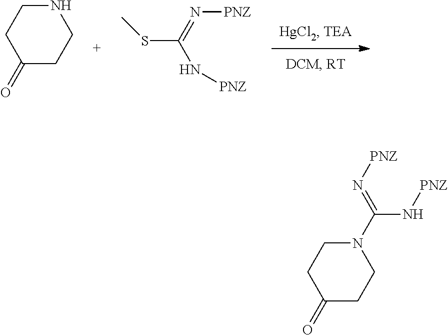

By following the same reaction procedures as described in Step 1 of Example 9, 4-nitrobenzyl (E)-(((((4-nitrobenzyl)oxy)carbonyl)imino)(4-oxopiperidin-1-yl)methyl)carbamate was obtained. ESI-MS m/z 500 (M+H)⁺.

### Step 3. Synthesis of (4R,5S,6S)-3-(((1S,3S)-3-((1-carbamimidoylpiperidin-4-yl)amino)cyclobutyl)thio)-6-((R)-1-hydroxyethyl)-4-methyl-7-oxo-1-azabicyclo[3.2.0]hept-2-ene-2-carboxylic acid

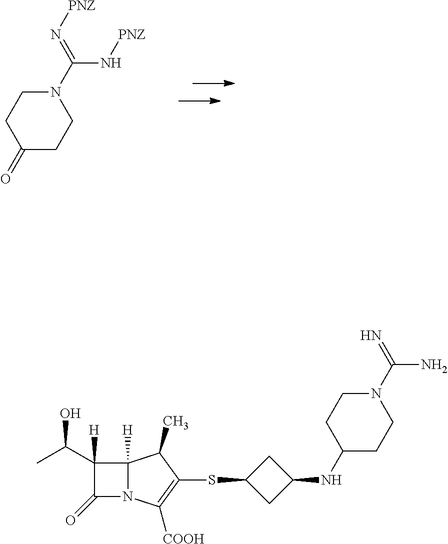

By following the same reaction procedures as described in Steps 8, 9, and 10 of Example 1, 4-nitrobenzyl (E)-(((((4-nitrobenzyl)oxy)carbonyl)imino)(4-oxopiperidin-1-yl)methyl)carbamate was converted to the target compound. ESI-MS m/z 438 (M+H)⁺.

### Example 44: (4R,5S,6S)-6-((R)-1-hydroxyethyl)-4-methyl-7-oxo-3-(((1S,3S)-3-(piperidin-4-ylamino)cyclobutyl)thio)-1-azabicyclo[3.2.0]hept-2-ene-2-carboxylic acid

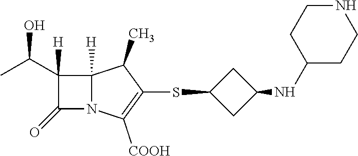 [SMILES: C[C@@H](O)[C@H]1C(=O)N2C(C(=O)O)=C(S[C@H]3C[C@@H](NC4CCNCC4)C3)[C@H](C)[C@H]12]

By following the same reaction procedures as described in Steps 6, 8, 9, and 10 of Example 1, piperidin-4-one was converted to the target compound. ESI-MS m/z 396 (M+H)⁺.

### Example 45: (4R,5S,6S)-3-(((R)-1-(((1S,4S)-4-(aminomethyl)cyclohexyl)methyl)pyrrolidin-3-yl)thio)-6-((R)-1-hydroxyethyl)-4-methyl-7-oxo-1-azabicyclo[3.2.0]hept-2-ene-2-carboxylic acid

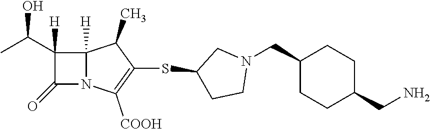 [SMILES: C[C@@H](O)[C@H]1C(=O)N2C(C(=O)O)=C(S[C@@H]3CCN(C[C@H]4CC[C@@H](CN)CC4)C3)[C@H](C)[C@H]12]

By following the same reaction procedures as described in Steps 6, 7, 8, 9, and 10 of Example 1, ((1S,4S)-4-(aminomethyl)cyclohexyl)methanol was converted to the target compound. ESI-MS m/z 438 (M+H)⁺.

### Example 46: (4R,5S,6S)-3-((1-(((R)-1-carbamimidoylpyrrolidin-3-yl)methyl)azetidin-3-yl)thio)-6-((R)-1-hydroxyethyl)-4-methyl-7-oxo-1-azabicyclo[3.2.0]hept-2-ene-2-carboxylic acid

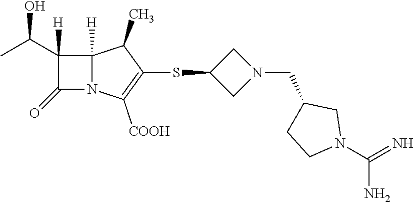

### Step 1. Synthesis of 4-nitrobenzyl (S,E)-((3-(hydroxymethyl)pyrrolidin-1-yl)((((4-nitrobenzyl)oxy)carbonyl)imino)methyl)carbamate

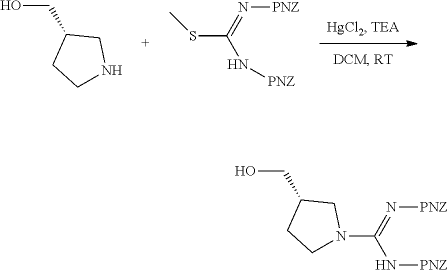

By following the same reaction procedures as described in Step 1 of Example 9, 4-nitrobenzyl (S,E)-((3-(hydroxymethyl)pyrrolidin-1-yl)((((4-nitrobenzyl)oxy)carbonyl)imino)methyl)carbamate was obtained. ESI-MS m/z 502 (M+H)⁺.

### Step 2. Synthesis of (4R,5S,6S)-3-((1-(((R)-1-carbamimidoylpyrrolidin-3-yl)methyl)azetidin-3-yl)thio)-6-((R)-1-hydroxyethyl)-4-methyl-7-oxo-1-azabicyclo[3.2.0]hept-2-ene-2-carboxylic acid

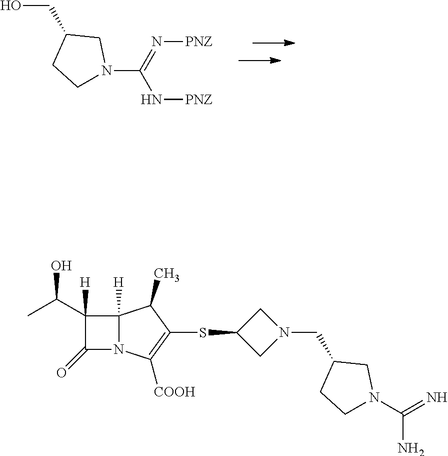

By following the same reaction procedures as described in Steps 7, 8, 9, and 10 of Example 1, 4-nitrobenzyl (S,E)-((3-(hydroxymethyl)pyrrolidin-1-yl)((((4-nitrobenzyl)oxy)carbonyl)imino) methyl)carbamate was converted to the target compound. ESI-MS m/z 424 (M+H)⁺.

### Example 47: (4R,5S,6S)-6-((R)-1-hydroxyethyl)-4-methyl-3-((1-(((1r,4R)-4-((methylamino)methyl)cyclohexyl)methyl)azetidin-3-yl)thio)-7-oxo-1-azabicyclo[3.2.0]hept-2-ene-2-carboxylic acid

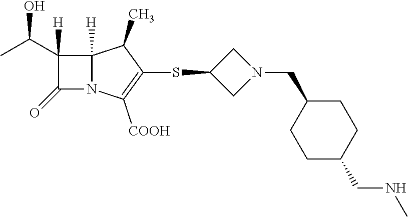

By following the same reaction procedures as described in Steps 6, 7, 8, 9, and 10 of Example 1, ((1r,4r)-4-((methylamino)methyl)cyclohexyl)methanol was converted to the target compound. ESI-MS m/z 438 (M+H)⁺.

### Example 48: (4R,5S,6S)-3-(((1R,3R)-3-((1-carbamimidoylpiperidin-4-yl)amino)cyclobutyl)thio)-6-((R)-1-hydroxyethyl)-4-methyl-7-oxo-1-azabicyclo[3.2.0]hept-2-ene-2-carboxylic acid

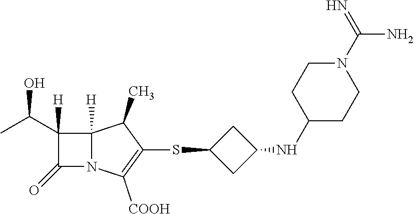 [SMILES: C[C@@H](O)[C@H]1C(=O)N2C(C(=O)O)=C(S[C@H]3C[C@H](NC4CCN(C(=N)N)CC4)C3)[C@H](C)[C@H]12]

### Step 1. Synthesis of 4-nitrobenzyl (E)-(((((4-nitrobenzyl)oxy)carbonyl)imino)(4-oxopiperidin-1-yl)methyl)carbamate

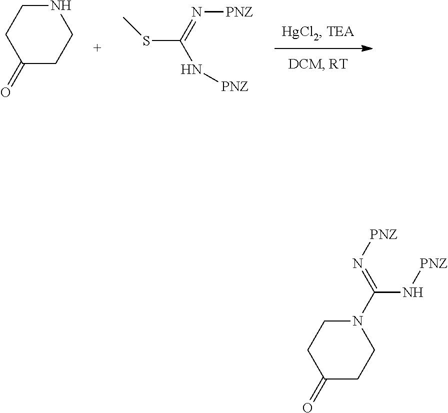

By following the same reaction procedures as described in Step 1 of Example 9, 4-nitrobenzyl (E)-(((((4-nitrobenzyl)oxy)carbonyl)imino)(4-oxopiperidin-1-yl)methyl)carbamate was obtained. ESI-MS m/z 500 (M+H)⁺.

### Step 2. Synthesis of (4R,5S,6S)-3-(((1R,3R)-3-((1-carbamimidoylpiperidin-4-yl)amino)cyclobutyl)thio)-6-((R)-1-hydroxyethyl)-4-methyl-7-oxo-1-azabicyclo[3.2.0]hept-2-ene-2-carboxylic acid

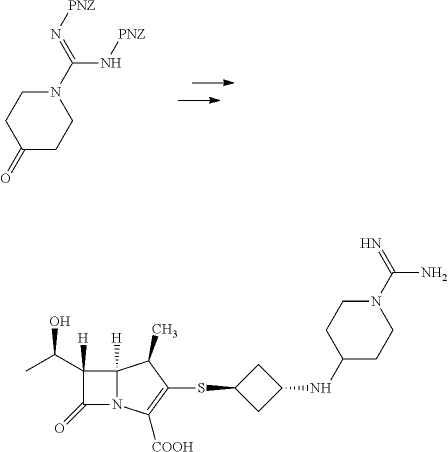

By following the same reaction procedures as described in Steps 8, 9, and 10 of Example 1, 4-nitrobenzyl (E)-(((((4-nitrobenzyl)oxy)carbonyl)imino)(4-oxopiperidin-1-yl)methyl)carbamate was converted to the target compound. ESI-MS m/z 438 (M+H)⁺.

### Example 49: (4R,5S,6S)-3-(((1R,3S)-3-((1-carbamimidoylpiperidin-4-yl)amino)cyclopentyl)thio)-6-((R)-1-hydroxyethyl)-4-methyl-7-oxo-1-azabicyclo[3.2.0]hept-2-ene-2-carboxylic acid

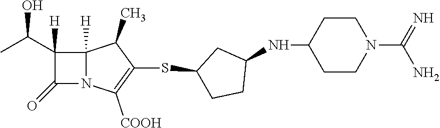 [SMILES: C[C@@H](O)[C@H]1C(=O)N2C(C(=O)O)=C(S[C@@H]3CC[C@H](NC4CCN(C(=N)N)CC4)C3)[C@H](C)[C@H]12]

### Step 1. Synthesis of (1R,3S)-3-aminocyclopentane-1-thiol

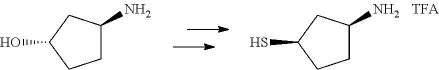 [SMILES: N[C@H]1CC[C@H](O)C1 ; N[C@H]1CC[C@@H](S)C1]

By following the same reaction procedures as described in Steps 1, 2, 3, 4, and 5 of Example 1, (1S,3S)-3-aminocyclopentan-1-ol was converted to the target compound. ESI-MS m/z 118 (M+H)⁺.

### Step 2. Synthesis of 4-nitrobenzyl (E)-(((((4-nitrobenzyl)oxy)carbonyl)imino)(4-oxopiperidin-1-yl)methyl)carbamate

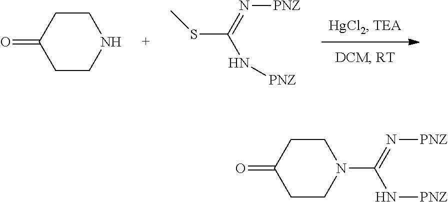

By following the same reaction procedures as described in Step 1 of Example 9, 4-nitrobenzyl (E)-(((((4-nitrobenzyl)oxy)carbonyl)imino)(4-oxopiperidin-1-yl)methyl)carbamate was obtained. ESI-MS m/z 500 (M+H)⁺.

### Step 3. Synthesis of (4R,5S,6S)-3-(((1R,3S)-3-((1-carbamimidoylpiperidin-4-yl)amino)cyclopentyl)thio)-6-((R)-1-hydroxyethyl)-4-methyl-7-oxo-1-azabicyclo[3.2.0]hept-2-ene-2-carboxylic acid

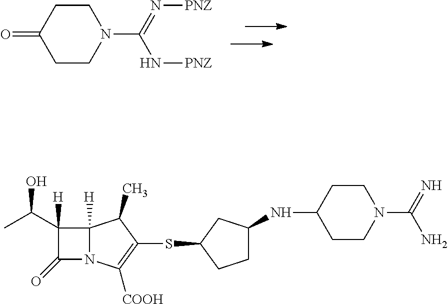

By following the same reaction procedures as described in Steps 8, 9, and 10 of Example 1, 4-nitrobenzyl (E)-(((((4-nitrobenzyl)oxy)carbonyl)imino)(4-oxopiperidin-1-yl)methyl)carbamate was converted to the target compound. ESI-MS m/z 452 (M+H)⁺.

### Example 50: (4R,5S,6S)-3-(((1R,3S)-3-((1-carbamimidoylpiperidin-4-yl)amino)cyclopentyl)thio)-6-((R)-1-hydroxyethyl)-4-methyl-7-oxo-1-azabicyclo[3.2.0]hept-2-ene-2-carboxylic acid

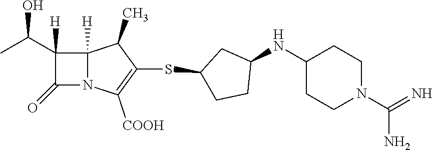 [SMILES: C[C@@H](O)[C@H]1C(=O)N2C(C(=O)O)=C(S[C@@H]3CC[C@H](NC4CCN(C(=N)N)CC4)C3)[C@H](C)[C@H]12]

### Step 1. Synthesis of (1R,3S)-3-aminocyclopentane-1-thiol

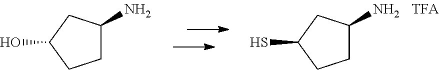 [SMILES: N[C@H]1CC[C@H](O)C1 ; N[C@H]1CC[C@@H](S)C1]

By following the same reaction procedures as described in Steps 1, 2, 3, 4, and 5 of Example 1, (1S,3S)-3-aminocyclopentan-1-ol was converted to the target compound. ESI-MS m/z 118 (M+H)⁺.

### Step 2. Synthesis of 4-nitrobenzyl (E)-(((((4-nitrobenzyl)oxy)carbonyl)imino)(4-oxopiperidin-1-yl)methyl)carbamate

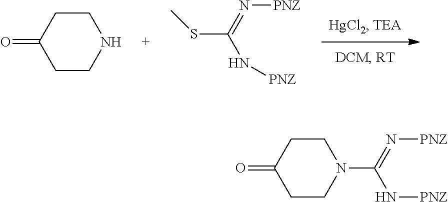

By following the same reaction procedures as described in Step 1 of Example 9, 4-nitrobenzyl (E)-(((((4-nitrobenzyl)oxy)carbonyl)imino)(4-oxopiperidin-1-yl)methyl)carbamate was obtained. ESI-MS m/z 500 (M+H)⁺.

### Step 3. Synthesis of (4R,5S,6S)-3-(((1R,3S)-3-((1-carbamimidoylpiperidin-4-yl)amino)cyclopentyl)thio)-6-((R)-1-hydroxyethyl)-4-methyl-7-oxo-1-azabicyclo[3.2.0]hept-2-ene-2-carboxylic acid

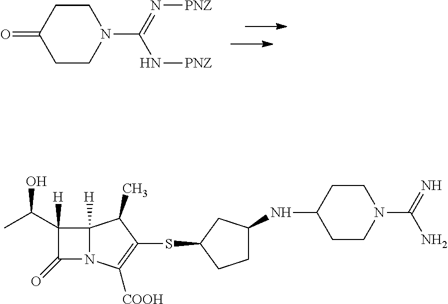

By following the same reaction procedures as described in Steps 8, 9, and 10 of Example 1, 4-nitrobenzyl (E)-(((((4-nitrobenzyl)oxy)carbonyl)imino)(4-oxopiperidin-1-yl)methyl)carbamate was converted to the target compound. ESI-MS m/z 452 (M+H)⁺.

### Example 50: (4R,5S,6S)-6-((R)-1-hydroxyethyl)-4-methyl-7-oxo-3-(((1R,3S)-3-(piperidin-4-ylamino)cyclopentyl)thio)-1-azabicyclo[3.2.0]hept-2-ene-2-carboxylic acid

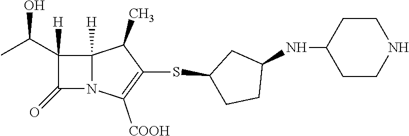 [SMILES: C[C@@H](O)[C@H]1C(=O)N2C(C(=O)O)=C(S[C@@H]3CC[C@H](NC4CCNCC4)C3)[C@H](C)[C@H]12]

By following the same reaction procedures as described in Steps 6, 8, 9, and 10 of Example 1, piperidin-4-one was converted to the target compound. ESI-MS m/z 410 (M+H)⁺.

### Example 51: (4R,5S,6S)-3-(((1S,3S)-3-(((1S,4R)-4-guanidinocyclohexyl)amino)cyclobutyl)thio)-6-((R)-1-hydroxyethyl)-4-methyl-7-oxo-1-azabicyclo[3.2.0]hept-2-ene-2-carboxylic acid

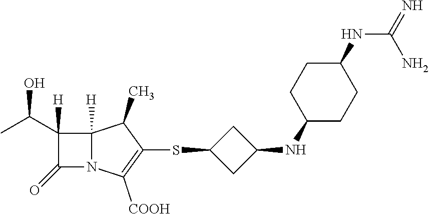 [SMILES: C[C@@H](O)[C@H]1C(=O)N2C(C(=O)O)=C(S[C@H]3C[C@@H](N[C@H]4CC[C@@H](NC(=N)N)CC4)C3)[C@H](C)[C@H]12]

By following the same reaction procedures as described in Steps 9 and 10 of Example 1, 1-((1R,4R)-4-(((1S,3S)-3-mercaptocyclobutyl)amino)cyclohexyl)-1,3-bis(4-nitrobenzyl)guanidine ester (more polar isomer from Example 53) was converted to the target compound. ESI-MS m/z 452 (M+H)⁺.

### Example 52: (4R,5S,6S)-3-(((1S,3S)-3-(((1R,4S)-4-guanidinocyclohexyl)amino)cyclobutyl)thio)-6-((R)-1-hydroxyethyl)-4-methyl-7-oxo-1-azabicyclo[3.2.0]hept-2-ene-2-carboxylic acid

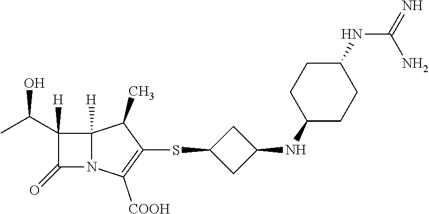 [SMILES: C[C@@H](O)[C@H]1C(=O)N2C(C(=O)O)=C(S[C@H]3C[C@@H](N[C@H]4CC[C@H](NC(=N)N)CC4)C3)[C@H](C)[C@H]12]

### Step 1. Synthesis of 1-((1s,4s)-4-hydroxycyclohexyl)-1,3-bis(4-nitrobenzyl)guanidine ester

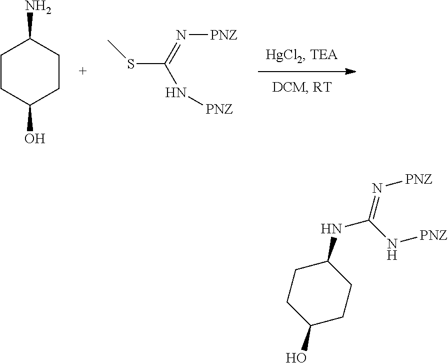

By following the same reaction procedures as described in Step 1 of Example 9, 1-((1S,4S)-4-hydroxycyclohexyl)-1,3-bis(4-nitrobenzyl)guanidine ester was obtained. ESI-MS m/z 516 (M+H)⁺.

### Step 2. Synthesis of (4R,5S,6S)-3-(((1S,3S)-3-(((1R,4S)-4-guanidinocyclohexyl)amino)cyclobutyl)thio)-6-((R)-1-hydroxyethyl)-4-methyl-7-oxo-1-azabicyclo[3.2.0]hept-2-ene-2-carboxylic acid

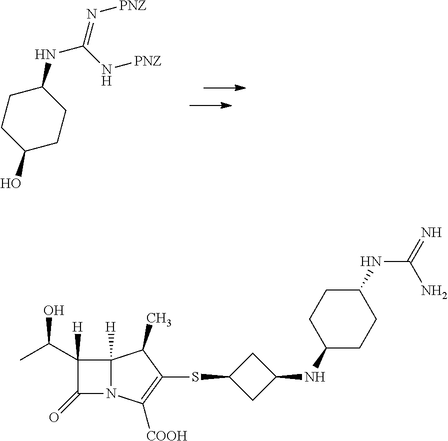

By following the same reaction procedures as described in Steps 9 and 10 of Example 1, 1-((1S,4S)-4-(((1S,3S)-3-mercaptocyclobutyl)amino)cyclohexyl)-1,3-bis(4-nitrobenzyl)guanidine ester (less polar isomer prepared from 1-((1s,4s)-4-hydroxycyclohexyl)-1,3-bis(4-nitrobenzyl)guanidine ester via Step 7, 8 of Example 1) was converted to the target compound. ESI-MS m/z 452 (M+H)⁺.

### Example 53: (4R,5S,6S)-6-((R)-1-hydroxyethyl)-4-methyl-7-oxo-3-(((1R,3R)-3-(piperidin-4-ylamino)cyclopentyl)thio)-1-azabicyclo[3.2.0]hept-2-ene-2-carboxylic acid

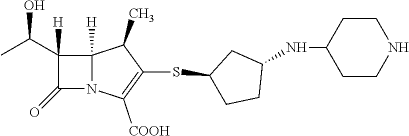 [SMILES: C[C@@H](O)[C@H]1C(=O)N2C(C(=O)O)=C(S[C@@H]3CC[C@@H](NC4CCNCC4)C3)[C@H](C)[C@H]12]

### Step 1. Synthesis of (1R,3R)-3-aminocyclopentane-1-thiol

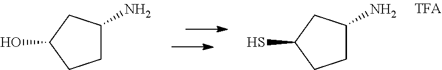 [SMILES: N[C@@H]1CC[C@H](O)C1 ; N[C@@H]1CC[C@@H](S)C1]

By following the same reaction procedures as described in Steps 1, 2, 3, 4, and 5 of Example 1, (1S,3R)-3-aminocyclopentan-1-ol was converted to the target compound. ESI-MS m/z 118 (M+H)⁺.

### Step 2. Synthesis of (4R,5S,6S)-6-((R)-1-hydroxyethyl)-4-methyl-7-oxo-3-(((1R,3R)-3-(piperidin-4-ylamino)cyclopentyl)thio)-1-azabicyclo[3.2.0]hept-2-ene-2-carboxylic acid

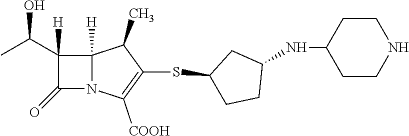 [SMILES: C[C@@H](O)[C@H]1C(=O)N2C(C(=O)O)=C(S[C@@H]3CC[C@@H](NC4CCNCC4)C3)[C@H](C)[C@H]12]

By following the same reaction procedures as described in Steps 6, 8, 9, and 10 of Example 1, piperidin-4-one was converted to the target compound. ESI-MS m/z 410 (M+H)⁺.

### Example 55: (4R,5S,6S)-3-(((1R,3R)-3-((1-carbamimidoylpiperidin-4-yl)amino)cyclopentyl)thio)-6-((R)-1-hydroxyethyl)-4-methyl-7-oxo-1-azabicyclo[3.2.0]hept-2-ene-2-carboxylic acid

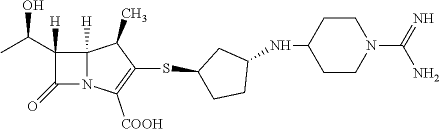 [SMILES: C[C@@H](O)[C@H]1C(=O)N2C(C(=O)O)=C(S[C@@H]3CC[C@@H](NC4CCN(C(=N)N)CC4)C3)[C@H](C)[C@H]12]

### Step 1. Synthesis of 4-nitrobenzyl (E)-(((((4-nitrobenzyl)oxy)carbonyl)imino)(4-oxopiperidin-1-yl)methyl)carbamate

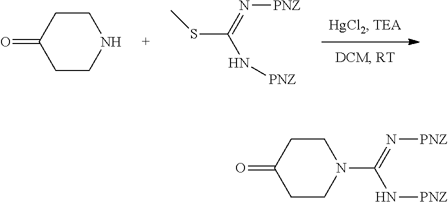

By following the same reaction procedures as described in Step 1 of Example 9, 4-nitrobenzyl (E)-(((((4-nitrobenzyl)oxy)carbonyl)imino)(4-oxopiperidin-1-yl)methyl)carbamate was obtained. ESI-MS m/z 500 (M+H)⁺.

### Step 2. Synthesis of (4R,5S,6S)-3-(((1R,3R)-3-((1-carbamimidoylpiperidin-4-yl)amino)cyclopentyl)thio)-6-((R)-1-hydroxyethyl)-4-methyl-7-oxo-1-azabicyclo[3.2.0]hept-2-ene-2-carboxylic acid

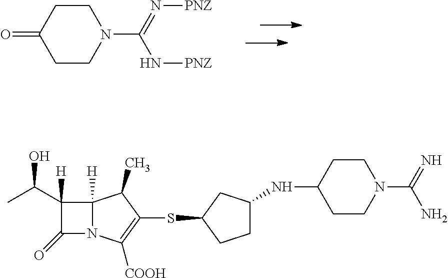

By following the same reaction procedures as described in Steps 8, 9, and 10 of Example 1, 4-nitrobenzyl (E)-(((((4-nitrobenzyl)oxy)carbonyl)imino)(4-oxopiperidin-1-yl)methyl)carbamate was converted to the target compound. ESI-MS m/z 452 (M+H)⁺.

### Example 56: (4R,5S,6S)-3-(((R)-1-(((1R,4R)-4-aminocyclohexyl)methyl)pyrrolidin-3-yl)thio)-6-((R)-1-hydroxyethyl)-4-methyl-7-oxo-1-azabicyclo[3.2.0]hept-2-ene-2-carboxylic acid

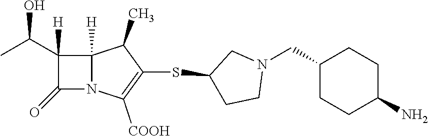 [SMILES: C[C@@H](O)[C@H]1C(=O)N2C(C(=O)O)=C(S[C@@H]3CCN(C[C@H]4CC[C@H](N)CC4)C3)[C@H](C)[C@H]12]

By following the same reaction procedures as described in Steps 6, 7, 8, 9, and 10 of Example 1, ((1R,4R)-4-aminocyclohexyl)methanol was converted to the target compound. ESI-MS m/z 424 (M+H)⁺.

### Example 57: (4R,5S,6S)-3-(((R)-1-((1S,4S)-4-(aminomethyl)cyclohexyl)pyrrolidin-3-yl)thio)-6-((R)-1-hydroxyethyl)-4-methyl-7-oxo-1-azabicyclo[3.2.0]hept-2-ene-2-carboxylic acid

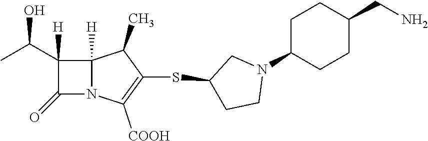 [SMILES: C[C@@H](O)[C@H]1C(=O)N2C(C(=O)O)=C(S[C@@H]3CCN([C@H]4CC[C@@H](CN)CC4)C3)[C@H](C)[C@H]12]

By following the same reaction procedures as described in Steps 9 and 10 of Example 1, 4-nitrobenzyl (((1S,4S)-4-((R)-3-mercaptopyrrolidin-1-yl)cyclohexyl)methyl)carbamate (more polar isomer from Example 58) was converted to the target compound. ESI-MS m/z 424 (M+H)⁺.

### Example 58: (4R,5S,6S)-3-(((R)-1-((1R,4R)-4-(aminomethyl)cyclohexyl)pyrrolidin-3-yl)thio)-6-((R)-1-hydroxyethyl)-4-methyl-7-oxo-1-azabicyclo[3.2.0]hept-2-ene-2-carboxylic acid

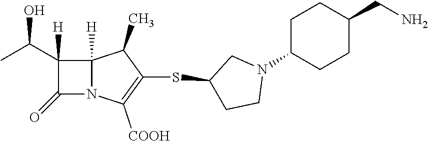 [SMILES: C[C@@H](O)[C@H]1C(=O)N2C(C(=O)O)=C(S[C@@H]3CCN([C@H]4CC[C@H](CN)CC4)C3)[C@H](C)[C@H]12]

By following the same reaction procedures as described in Steps 9 and 10 of Example 1, 4-nitrobenzyl (((1R,4R)-4-((R)-3-mercaptopyrrolidin-1-yl)cyclohexyl)methyl)carbamate (less polar isomer prepared from (1R,4R)-4-(aminomethyl)cyclohexan-1-ol via Steps 6, 7, and 8 of Example 1) was converted to the target compound. ESI-MS m/z 424 (M+H)⁺.

### Example 59: (4R,5S,6S)-3-(((1R,3R)-3-(((1R,4R)-4-aminocyclohexyl)amino)cyclobutyl)thio)-6-((R)-1-hydroxyethyl)-4-methyl-7-oxo-1-azabicyclo[3.2.0]hept-2-ene-2-carboxylic acid

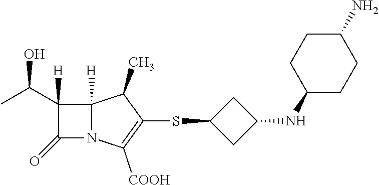 [SMILES: C[C@@H](O)[C@H]1C(=O)N2C(C(=O)O)=C(S[C@H]3C[C@H](N[C@H]4CC[C@H](N)CC4)C3)[C@H](C)[C@H]12]

By following the same reaction procedures as described in Steps 9 and 10 of Example 1, 4-nitrobenzyl ((1R,4R)-4-(((1R,3R)-3-mercaptocyclobutyl)amino)cyclohexyl)carbamate (less polar isomer prepared from (1r,4r)-4-aminocyclohexan-1-ol via Steps 6, 7, and 8 of Example 1) was converted to the target compound. ESI-MS m/z 410 (M+H)⁺.

### Example 60: (4R,5S,6S)-3-(((1R,3R)-3-(((1S,4S)-4-aminocyclohexyl)amino)cyclobutyl)thio)-6-((R)-1-hydroxyethyl)-4-methyl-7-oxo-1-azabicyclo[3.2.0]hept-2-ene-2-carboxylic acid

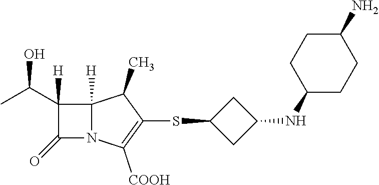 [SMILES: C[C@@H](O)[C@H]1C(=O)N2C(C(=O)O)=C(S[C@H]3C[C@H](N[C@H]4CC[C@@H](N)CC4)C3)[C@H](C)[C@H]12]

By following the same reaction procedures as described in Steps 9 and 10 of Example 1, 4-nitrobenzyl ((1S,4S)-4-(((1R,3R)-3-mercaptocyclobutyl)amino)cyclohexyl)carbamate (more polar isomer from Example 59) was converted to the target compound. ESI-MS m/z 410 (M+H)⁺.

### Example 61: (4R,5S,6S)-3-(((1S,3S)-3-(((1R,4S)-4-aminocyclohexyl)amino)cyclobutyl)thio)-6-((R)-1-hydroxyethyl)-4-methyl-7-oxo-1-azabicyclo[3.2.0]hept-2-ene-2-carboxylic acid

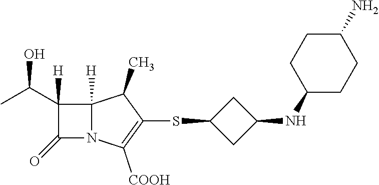 [SMILES: C[C@@H](O)[C@H]1C(=O)N2C(C(=O)O)=C(S[C@H]3C[C@@H](N[C@H]4CC[C@H](N)CC4)C3)[C@H](C)[C@H]12]

By following the same reaction procedures as described in Steps 9 and 10 of Example 1, 4-nitrobenzyl ((1R,4S)-4-(((1S,3S)-3-mercaptocyclobutyl)amino)cyclohexyl)carbamate (less polar isomer prepared from (1R,4R)-4-aminocyclohexan-1-ol via Steps 6, 7, and 8 of Example 1) was converted to the target compound. ESI-MS m/z 410 (M+H)⁺.

### Example 62: (4R,5S,6S)-3-(((1S,3S)-3-(((1S,4R)-4-aminocyclohexyl)amino)cyclobutyl)thio)-6-((R)-1-hydroxyethyl)-4-methyl-7-oxo-1-azabicyclo[3.2.0]hept-2-ene-2-carboxylic acid

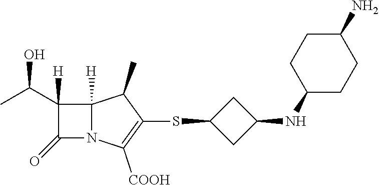 [SMILES: C[C@@H](O)[C@H]1C(=O)N2C(C(=O)O)=C(S[C@H]3C[C@@H](N[C@H]4CC[C@@H](N)CC4)C3)[C@H](C)[C@H]12]

By following the same reaction procedures as described in Steps 9 and 10 of Example 1, 4-nitrobenzyl ((1S,4R)-4-(((1S,3S)-3-mercaptocyclobutyl)amino)cyclohexyl)carbamate (more polar isomer from Example 61) was converted to the target compound ESI-MS m/z 410 (M+H)⁺.

### Example 63: (4R,5S,6S)-3-(((R)-1-((1S,4S)-4-(guanidinomethyl)cyclohexyl)pyrrolidin-3-yl)thio)-6-((R)-1-hydroxyethyl)-4-methyl-7-oxo-1-azabicyclo[3.2.0]hept-2-ene-2-carboxylic acid

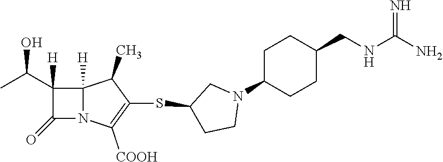 [SMILES: C[C@@H](O)[C@H]1C(=O)N2C(C(=O)O)=C(S[C@@H]3CCN([C@H]4CC[C@@H](CNC(=N)N)CC4)C3)[C@H](C)[C@H]12]

By following the same reaction procedures as described in Steps 9 and 10 of Example 1, 1-(((1S,4S)-4-((R)-3-mercaptopyrrolidin-1-yl)cyclohexyl)methyl)-1,3-bis(4-nitrobenzyl)guanidine ester (more polar isomer from Example 64) was converted to the target compound. ESI-MS m/z 466 (M+H)⁺.

### Example 64: (4R,5S,6S)-3-(((R)-1-((1R,4R)-4-(guanidinomethyl)cyclohexyl)pyrrolidin-3-yl)thio)-6-((R)-1-hydroxyethyl)-4-methyl-7-oxo-1-azabicyclo[3.2.0]hept-2-ene-2-carboxylic acid

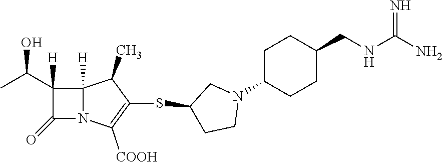 [SMILES: C[C@@H](O)[C@H]1C(=O)N2C(C(=O)O)=C(S[C@@H]3CCN([C@H]4CC[C@H](CNC(=N)N)CC4)C3)[C@H](C)[C@H]12]

### Step 1, Synthesis of 1,3-bis(4-nitrobenzyl)-1-((4-oxocyclohexyl)methyl)guanidine ester

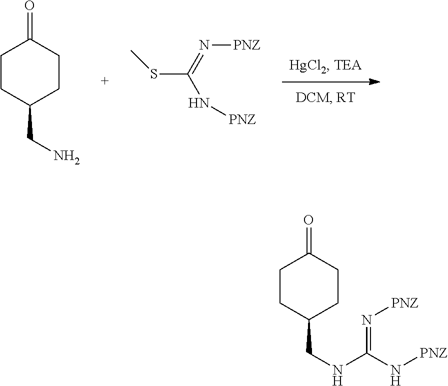

By following the same reaction procedures as described in Step 1 of Example 9, 1,3-bis(4-nitrobenzyl)-1-((4-oxocyclohexyl)methyl)guanidine ester was obtained. ESI-MS m/z 528 (M+H)⁺.

### Step 2. Synthesis of (4R,5S,6S)-3-(((R)-1-((1R,4R)-4-(guanidinomethyl)cyclohexyl)pyrrolidin-3-yl)thio)-6-((R)-1-hydroxyethyl)-4-methyl-7-oxo-1-azabicyclo[3.2.0]hept-2-ene-2-carboxylic acid

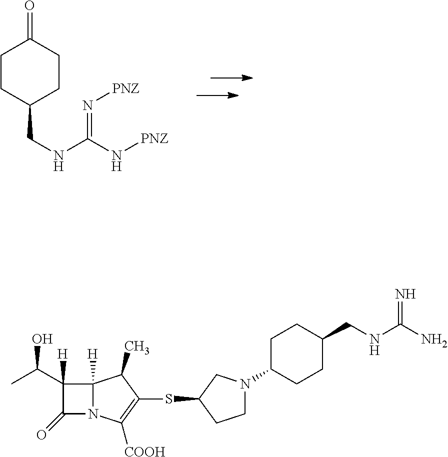

By following the same reaction procedures as described in Steps 9 and 10 of Example 1, 1-(((1R,4R)-4-((R)-3-mercaptopyrrolidin-1-yl)cyclohexyl)methyl)-1,3-bis(4-nitrobenzyl)guanidine ester (less polar isomer prepared from 1,3-bis(4-nitrobenzyl)-1-((4-oxocyclohexyl)methyl)guanidine ester via Step 8 of Example 1) was converted to the target compound. ESI-MS m/z 466 (M+H)⁺.

### Example 65: (4R,5S,6S)-3-((1-((1S,4S)-4-(guanidinomethyl)cyclohexyl)azetidin-3-yl)thio)-6-((R)-1-hydroxyethyl)-4-methyl-7-oxo-1-azabicyclo[3.2.0]hept-2-ene-2-carboxylic acid

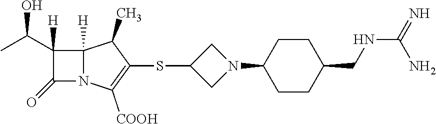 [SMILES: C[C@@H](O)[C@H]1C(=O)N2C(C(=O)O)=C(SC3CN([C@H]4CC[C@@H](CNC(=N)N)CC4)C3)[C@H](C)[C@H]12]

By following the same reaction procedures as described in Steps 9 and 10 of Example 1, 1-(((1S,4S)-4-(3-mercaptoazetidin-1-yl)cyclohexyl)methyl)-1,3-bis(4-nitrobenzyl)guanidine ester (more polar isomer from Example 66) was converted to the target compound. ESI-MS m/z 452 (M+H)⁺.

### Example 66: (4R,5S,6S)-3-((1-((1R,4R)-4-(guanidinomethyl)cyclohexyl)azetidin-3-yl)thio)-6-((R)-1-hydroxyethyl)-4-methyl-7-oxo-1-azabicyclo[3.2.0]hept-2-ene-2-carboxylic acid

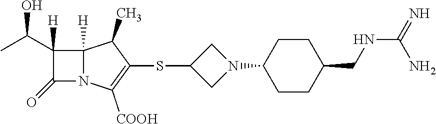 [SMILES: C[C@@H](O)[C@H]1C(=O)N2C(C(=O)O)=C(SC3CN([C@H]4CC[C@H](CNC(=N)N)CC4)C3)[C@H](C)[C@H]12]

### Step 1, Synthesis of 1,3-bis(4-nitrobenzyl)-1-((4-oxocyclohexyl)methyl)guanidine ester

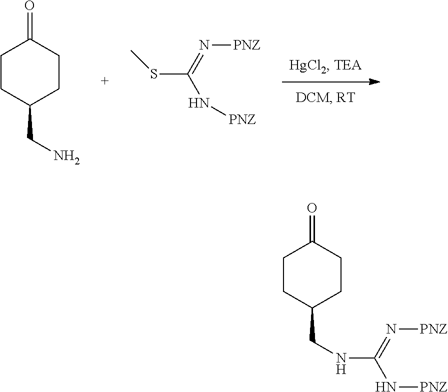

By following the same reaction procedures as described in Step 1 of Example 9, 1,3-bis(4-nitrobenzyl)-1-((4-oxocyclohexyl)methyl)guanidine ester was obtained. ESI-MS m/z 528 (M+H)⁺.

### Step 2. Synthesis of (4R,5S,6S)-3-((1-((1R,4R)-4-(guanidinomethyl)cyclohexyl)azetidin-3-yl)thio)-6-((R)-1-hydroxyethyl)-4-methyl-7-oxo-1-azabicyclo[3.2.0]hept-2-ene-2-carboxylic acid

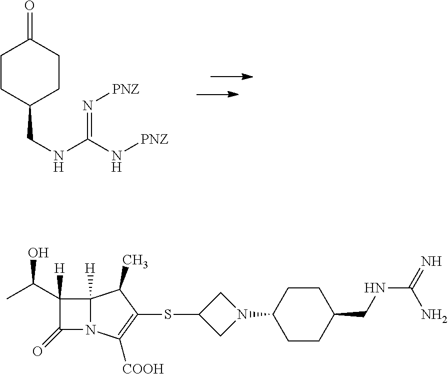

By following the same reaction procedures as described in Steps 9 and 10 of Example 1, 1-(((1R,4R)-4-(3-mercaptoazetidin-1-yl)cyclohexyl)methyl)-1,3-bis(4-nitrobenzyl)guanidine ester (less polar isomer prepared from 1,3-bis(4-nitrobenzyl)-1-((4-oxocyclohexyl)methyl)guanidine ester via Step 8 of Example 1) was converted to the target compound. ESI-MS m/z 452 (M+H)⁺.

### Example 67: (4R,5S,6S)-3-(((1S,3R)-3-(((1S,3S)-3-aminocyclopentyl)amino)cyclobutyl)thio)-6-((R)-1-hydroxyethyl)-4-methyl-7-oxo-1-azabicyclo[3.2.0]hept-2-ene-2-carboxylic acid

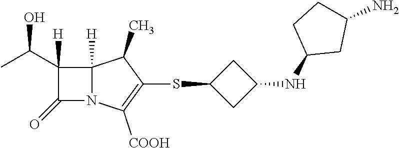 [SMILES: C[C@@H](O)[C@H]1C(=O)N2C(C(=O)O)=C(S[C@H]3C[C@H](N[C@H]4CC[C@H](N)C4)C3)[C@H](C)[C@H]12]

By following the same reaction procedures as described in Steps 9 and 10 of Example 1, 4-nitrobenzyl ((1S,3S)-3-(((1R,3S)-3-mercaptocyclobutyl)amino)cyclopentyl)carbamate (less polar isomer prepared from (1S,3S)-3-aminocyclopentan-1-ol via Steps 6, 7, and 8 of Example 1) was converted to the target compound. ESI-MS m/z 396 (M+H)⁺.

### Example 68: (4R,5S,6S)-3-(((1R,3R)-3-(((1R,4R)-4-guanidinocyclohexyl)amino)cyclobutyl)thio)-6-((R)-1-hydroxyethyl)-4-methyl-7-oxo-1-azabicyclo[3.2.0]hept-2-ene-2-carboxylic acid

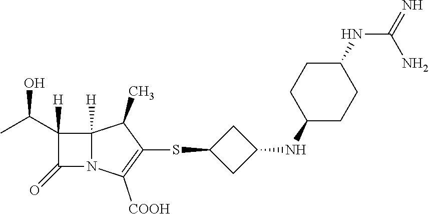 [SMILES: C[C@@H](O)[C@H]1C(=O)N2C(C(=O)O)=C(S[C@H]3C[C@H](N[C@H]4CC[C@H](NC(=N)N)CC4)C3)[C@H](C)[C@H]12]

### Step 1. Synthesis of 1-((1S,4S)-4-hydroxycyclohexyl)-1,3-bis(4-nitrobenzyl)guanidine ester

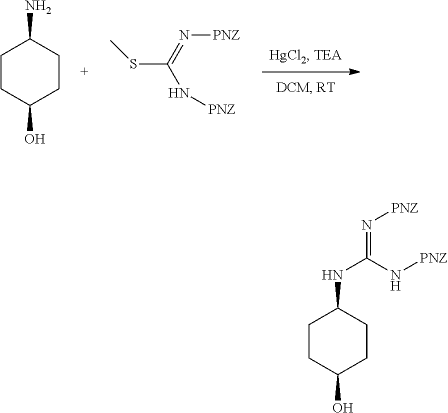

By following the same reaction procedures as described in Step 1 of Example 9, 1-((1S,4S)-4-hydroxycyclohexyl)-1,3-bis(4-nitrobenzyl)guanidine ester was obtained. ESI-MS m/z 516 (M+H)⁺.

### Step 2. Synthesis of (4R,5S,6S)-3-(((1R,3R)-3-(((1R,4R)-4-guanidinocyclohexyl)amino)cyclobutyl)thio)-6-((R)-1-hydroxyethyl)-4-methyl-7-oxo-1-azabicyclo[3.2.0]hept-2-ene-2-carboxylic acid

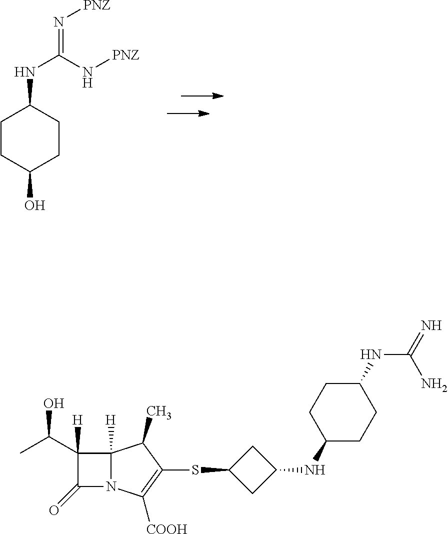

By following the same reaction procedures as described in Steps 9 and 10 of Example 1, 1-((1R,4R)-4-(((1R,3R)-3-mercaptocyclobutyl)amino)cyclohexyl)-1,3-bis(4-nitrobenzyl)guanidine ester (less polar isomer prepared from 1-((1S,4S)-4-hydroxycyclohexyl)-1,3-bis(4-nitrobenzyl)guanidine ester via Step 7 and 8 of Example 1) was converted to the target compound. ESI-MS m/z 452 (M+H)⁺.

### Example 69: (4R,5S,6S)-3-(((1R,3R)-3-(((1S,4S)-4-guanidinocyclohexyl)amino)cyclobutyl)thio)-6-((R)-1-hydroxyethyl)-4-methyl-7-oxo-1-azabicyclo[3.2.0]hept-2-ene-2-carboxylic acid

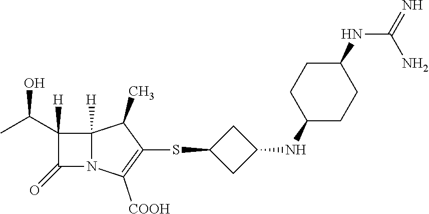 [SMILES: C[C@@H](O)[C@H]1C(=O)N2C(C(=O)O)=C(S[C@H]3C[C@H](N[C@H]4CC[C@@H](NC(=N)N)CC4)C3)[C@H](C)[C@H]12]

By following the same reaction procedures as described in Steps 9 and 10 of Example 1, 1-((1S,4S)-4-(((1R,3R)-3-mercaptocyclobutyl)amino)cyclohexyl)-1,3-bis(4-nitrobenzyl)guanidine ester (more polar isomer from Example 69) was converted to the target compound. ESI-MS m/z 452 (M+H)⁺.

### Example 70: (4R,5S,6S)-3-(((1R,3R)-3-(((1R,3S)-3-aminocyclopentyl)amino)cyclobutyl)thio)-6-((R)-1-hydroxyethyl)-4-methyl-7-oxo-1-azabicyclo[3.2.0]hept-2-ene-2-carboxylic acid

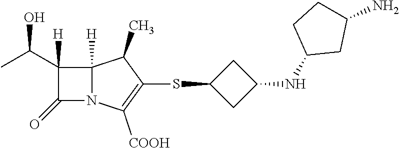 [SMILES: C[C@@H](O)[C@H]1C(=O)N2C(C(=O)O)=C(S[C@H]3C[C@H](N[C@@H]4CC[C@H](N)C4)C3)[C@H](C)[C@H]12]

By following the same reaction procedures as described in Steps 9 and 10 of Example 1, 4-nitrobenzyl ((1S,3R)-3-(((1R,3R)-3-mercaptocyclobutyl)amino)cyclopentyl)carbamate (more polar isomer from Example 67) was converted to the target compound. ESI-MS m/z 396 (M+H)⁺.

### Example 71: (4R,5S,6S)-3-(((1R,3R)-3-(((1R,3R)-3-aminocyclopentyl)amino)cyclobutyl)thio)-6-((R)-1-hydroxyethyl)-4-methyl-7-oxo-1-azabicyclo[3.2.0]hept-2-ene-2-carboxylic acid

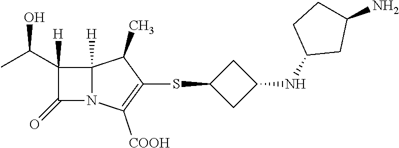 [SMILES: C[C@@H](O)[C@H]1C(=O)N2C(C(=O)O)=C(S[C@H]3C[C@H](N[C@@H]4CC[C@@H](N)C4)C3)[C@H](C)[C@H]12]

By following the same reaction procedures as described in Steps 9 and 10 of Example 1 4-nitrobenzyl ((1R,3R)-3-(((1R,3R)-3-mercaptocyclobutyl)amino)cyclopentyl)carbamate (less polar isomer prepared from (1S,3R)-3-aminocyclopentan-1-ol via Steps 6, 7 and 8 of Example 1) was converted to the target compound. ESI-MS m/z 396 (M+H)⁺.

### Example 72: (4R,5S,6S)-3-(((R)-1-(((1S,4S)-4-aminocyclohexyl)methyl)pyrrolidin-3-yl)thio)-6-((R)-1-hydroxyethyl)-4-methyl-7-oxo-1-azabicyclo[3.2.0]hept-2-ene-2-carboxylic acid

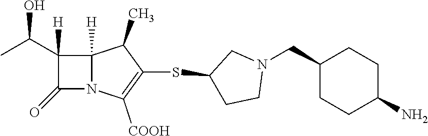 [SMILES: C[C@@H](O)[C@H]1C(=O)N2C(C(=O)O)=C(S[C@@H]3CCN(C[C@H]4CC[C@@H](N)CC4)C3)[C@H](C)[C@H]12]

By following the same reaction procedures as described in Steps 6, 7, 8, 9, and 10 of Example 1, ((1S,4S)-4-aminocyclohexyl)methanol was converted to the target compound. ESI-MS m/z 424 (M+H)⁺.

### Example 73: (4R,5S,6S)-3-(((1S,3R)-3-(((1S,3S)-3-guanidinocyclopentyl)amino)cyclobutyl)thio)-6-((R)-1-hydroxyethyl)-4-methyl-7-oxo-1-azabicyclo[3.2.0]hept-2-ene-2-carboxylic acid

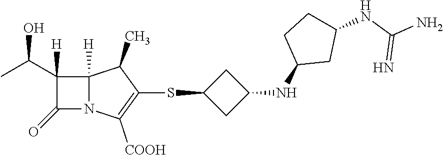 [SMILES: C[C@@H](O)[C@H]1C(=O)N2C(C(=O)O)=C(S[C@H]3C[C@H](N[C@H]4CC[C@H](NC(=N)N)C4)C3)[C@H](C)[C@H]12]

### Step 1. Synthesis of 1-((1S,3S)-3-hydroxycyclopentyl)guanidine, bis(4-nitrophenylmethyl) ester

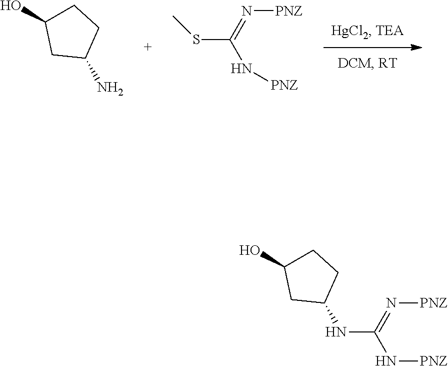

By following the same reaction procedures as described in Step 1 of Example 9, 1-((1S,3S)-3-hydroxycyclopentyl)guanidine, bis(4-nitrophenylmethyl) ester was obtained. ESI-MS m/z 502 (M+H)⁺.

### Step 2. Synthesis of (4R,5S,6S)-3-(((1S,3R)-3-(((1S,3S)-3-guanidinocyclopentyl)amino)cyclobutyl)thio)-6-((R)-1-hydroxyethyl)-4-methyl-7-oxo-1-azabicyclo[3.2.0]hept-2-ene-2-carboxylic acid

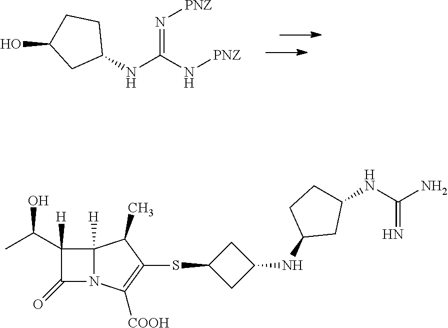

By following the same reaction procedures as described in Steps 9 and 10 of Example 1, 1-((1S,3S)-3-(((1R,3S)-3-mercaptocyclobutyl)amino)cyclopentyl)-1,3-bis(4-nitrobenzyl)guanidine ester (less polar isomer prepared from 1-((1S,3S)-3-hydroxycyclopentyl)guanidine, bis(4-nitrophenylmethyl) ester via Step 7, 8 of Example 1) was converted to the target compound. ESI-MS m/z 438 (M+H)⁺.

### Example 74: (4R,5S,6S)-3-(((1S,3R)-3-(((1S,3R)-3-aminocyclopentyl)amino)cyclobutyl)thio)-6-((R)-1-hydroxyethyl)-4-methyl-7-oxo-1-azabicyclo[3.2.0]hept-2-ene-2-carboxylic acid

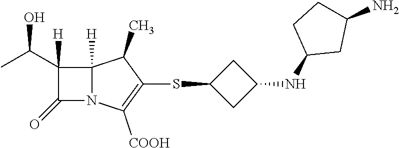 [SMILES: C[C@@H](O)[C@H]1C(=O)N2C(C(=O)O)=C(S[C@H]3C[C@H](N[C@H]4CC[C@@H](N)C4)C3)[C@H](C)[C@H]12]

By following the same reaction procedures as described in Steps 9 and 10 of Example 1, 4-nitrobenzyl ((1R,3S)-3-(((1R,3S)-3-mercaptocyclobutyl)amino)cyclopentyl)carbamate (more polar isomer from Example 71) was converted to the target compound. ESI-MS m/z 396 (M+H)⁺.

### Example 75: (4R,5S,6S)-3-(((1R,3S)-3-(((1S,3S)-3-aminocyclopentyl)amino)cyclobutyl)thio)-6-((R)-1-hydroxyethyl)-4-methyl-7-oxo-1-azabicyclo[3.2.0]hept-2-ene-2-carboxylic acid

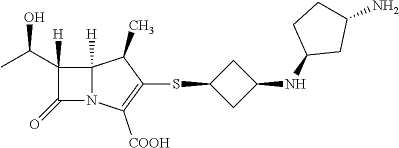 [SMILES: C[C@@H](O)[C@H]1C(=O)N2C(C(=O)O)=C(S[C@H]3C[C@@H](N[C@H]4CC[C@H](N)C4)C3)[C@H](C)[C@H]12]

By following the same reaction procedures as described in Steps 9 and 10 of Example 1, 4-nitrobenzyl ((1S,3S)-3-(((1S,3R)-3-mercaptocyclobutyl)amino)cyclopentyl)carbamate (less polar isomer prepared from (1S,3S)-3-aminocyclopentan-1-ol via Steps 6, 7, and 8 of Example 1) was converted to the target compound. ESI-MS m/z 396 (M+H)⁺.

### Example 76: (4R,5S,6S)-3-(((1R,3S)-3-(((1S,3R)-3-aminocyclopentyl)amino)cyclobutyl)thio)-6-((R)-1-hydroxyethyl)-4-methyl-7-oxo-1-azabicyclo[3.2.0]hept-2-ene-2-carboxylic acid

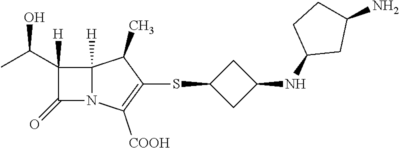 [SMILES: C[C@@H](O)[C@H]1C(=O)N2C(C(=O)O)=C(S[C@H]3C[C@@H](N[C@H]4CC[C@@H](N)C4)C3)[C@H](C)[C@H]12]

By following the same reaction procedures as described in Steps 9 and 10 of Example 1, 4-nitrobenzyl ((1R,3S)-3-(((1S,3R)-3-mercaptocyclobutyl)amino)cyclopentyl)carbamate (more polar isomer from Example 78) was converted to the target compound. ESI-MS m/z 396 (M+H)⁺.

### Example 77: (4R,5S,6S)-3-(((1S,3S)-3-(((1R,3S)-3-aminocyclopentyl)amino)cyclobutyl)thio)-6-((R)-1-hydroxyethyl)-4-methyl-7-oxo-1-azabicyclo[3.2.0]hept-2-ene-2-carboxylic acid

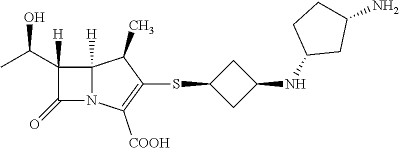 [SMILES: C[C@@H](O)[C@H]1C(=O)N2C(C(=O)O)=C(S[C@H]3C[C@@H](N[C@@H]4CC[C@H](N)C4)C3)[C@H](C)[C@H]12]

By following the same reaction procedures as described in Steps 9 and 10 of Example 1, 4-nitrobenzyl ((1S,3R)-3-(((1S,3S)-3-mercaptocyclobutyl)amino)cyclopentyl)carbamate (more polar isomer from Example 75) was converted to the target compound. ESI-MS m/z 396 (M+H)⁺.

### Example 78: (4R,5S,6S)-3-(((1S,3S)-3-(((1R,3R)-3-aminocyclopentyl)amino)cyclobutyl)thio)-6-((R)-1-hydroxyethyl)-4-methyl-7-oxo-1-azabicyclo[3.2.0]hept-2-ene-2-carboxylic acid

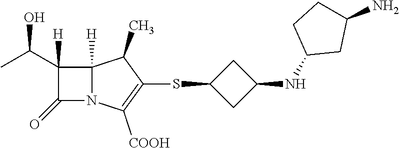 [SMILES: C[C@@H](O)[C@H]1C(=O)N2C(C(=O)O)=C(S[C@H]3C[C@@H](N[C@@H]4CC[C@@H](N)C4)C3)[C@H](C)[C@H]12]

By following the same reaction procedures as described in Steps 9 and 10 of Example 1, 4-nitrobenzyl ((1R,3R)-3-(((1S,3S)-3-mercaptocyclobutyl)amino)cyclopentyl)carbamate (less polar isomer prepared from (1S,3R)-3-aminocyclopentan-1-ol via Steps 6, 7, and 8 of Example 1) was converted to the target compound. ESI-MS m/z 396 (M+H)⁺.

### Example 79: (4R,5S,6S)-3-(((1R,3R)-3-(((1R,3S)-3-guanidinocyclopentyl)amino)cyclobutyl)thio)-6-((R)-1-hydroxyethyl)-4-methyl-7-oxo-1-azabicyclo[3.2.0]hept-2-ene-2-carboxylic acid

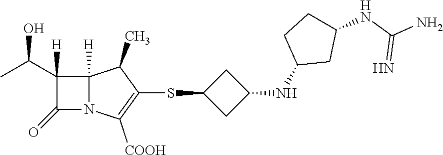 [SMILES: C[C@@H](O)[C@H]1C(=O)N2C(C(=O)O)=C(S[C@H]3C[C@H](N[C@@H]4CC[C@H](NC(=N)N)C4)C3)[C@H](C)[C@H]12]

By following the same reaction procedures as described in Steps 9 and 10 of Example 11-((1S,3R)-3-(((1R,3R)-3-mercaptocyclobutyl)amino)cyclopentyl)-1,3-bis(4-nitrobenzyl)guanidine ester (more polar isomer from Example 73) was converted to the target compound. ESI-MS m/z 438 (M+H)⁺.

### Example A1: Parenteral Composition of a Compound Described Herein, or a Pharmaceutically Acceptable Salt, Solvate, or Stereoisomer Thereof

To prepare a parenteral pharmaceutical composition suitable for administration by injection, 100 mg of a compound disclosed herein, or a pharmaceutically acceptable salt, solvate, or stereoisomer thereof, or a water soluble pharmaceutically acceptable salt thereof, is dissolved in DMSO and then mixed with 10 mL of 0.9% sterile saline solution. The mixture is incorporated into a dosage unit suitable for administration by injection.

### Example A2: Oral Composition of a Compounds Described Herein, or a Pharmaceutically Acceptable Salt, Solvate, or Stereoisomer Thereof

To prepare a pharmaceutical composition for oral delivery, 400 mg of compound described herein, or a pharmaceutically acceptable salt, solvate, or stereoisomer thereof and the following ingredients are mixed intimately and pressed into single scored tablets.

### Tablet Formulation

| | Quantity |
|---|---|
| | per tablet |
| Ingredient | (mg) |
| compound | 400 |
| cornstarch | 50 |
| croscarmellose sodium | 25 |
| lactose | 120 |
| magnesium stearate | 5 |

The following ingredients are mixed intimately and loaded into a hard-shell gelatin capsule.

### Capsule Formulation

| | Quantity |
|---|---|
| | per capsule |
| Ingredient | (mg) |
| compound | 200 |
| lactose spray dried | 148 |
| magnesium stearate | 2 |

### BIOLOGICAL EXAMPLES

### Example B1: In Vitro Antibacterial Assays

To determine the ability of test compounds to inhibit the growth of bacterial strains, classic cell-based broth microdilution MIC assays were employed. MIC assays are performed according to CLSI methods except where otherwise noted (CLSI, 2011 and CLSI, 2009). The reference strains S. aureus ATCC 29213 (methicillin sensitive), S. aureus ATCC 33591 (methicillin-resistant), E. coli ATCC 25922 (wild-type/QC strain), P. aeruginosa ATCC 27853 (wild-type/QC strain), and the clinical isolate P. aeruginosa PA 8 were used to determine the ability of the exemplary carbapenem compounds to inhibit bacterial growth. An expanded panel of methicillin resistance S. aureus (MRSA) and Pseudomonas was also used to determine the ability of exemplary carbapenem compounds to inhibit bacterial growth across a broader representation of those species. To determine activity in MRSA and in addition to S. aureus ATCC 33591, S. aureus USA 300 and USA 600, some of the most common clones found to cause human infection, were also used. To further gauge the impact on the carbapenem compounds due to various resistance mechanisms in Pseudomonas, the following strains were included: P. aeruginosa 35151 (hyper-permeable strain), P. aeruginosa ΔmexAB-OprM (an efflux pump knockout), and P. aeruginosa Paβ-173 (a clinical isolate of reduced carbapenem susceptibility). Briefly, cryo-preserved bacterial cultures of clinical strains are streaked for isolation on appropriate agar medium, in this case Mueller Hinton II agar. Following incubation to allow formation of colonies, these plates are sealed with parafilm and stored refrigerated for up to two weeks. For preparation of assay inocula and to ensure low variability, at least 5 colonies are picked from the agar plates with an inoculating loop and aseptically transferred to a culture tube containing 3 mL of Mueller-Hinton Broth (supplemented with divalent cations to required levels based on Manufacturers' specification). The broth culture is grown for 3-5 hours at 37° C. with shaking at 200 rpm. Meanwhile, 2-fold serial dilutions of test compounds are conducted in a 96 well plate with a final volume of 75 μL per well at 2-fold the final desired concentration. After the dilution plates are set up the growing cultures are then diluted in a cuvette containing MH II broth and the optical density is measured at 600 nm. Inocula are diluted such that 75 μL of this culture in Mueller-Hinton Broth results in a starting bacterial concentration of 5×10⁵ CFU/mL when added to the dilution plates. The plates are incubated 16-20 hours at 37° C. The MIC is read visually as the lowest concentration well with no bacterial growth.

Representative results are shown in Tables 2 and 3, where A represents an MIC>32 μg/mL, B represents an MIC between 8 and 32 μg/mL inclusive, C represents an MIC between 1 and 4 μg/mL inclusive, and D represents an MIC of <1 μg/mL. NT=Not Tested.

TABLE 2
Inhibition of bacterial growth for methicillin-resistant S. aureus
and E. coli strains. Minimum Inhibitory Concentrations of
exemplary compounds (MIC, in μg/mL).

| | S. aureus | S. aureus | S. aureus | E. coli |
|---|---|---|---|---|
| Example | 33591 | USA300 | USA600 | 25922 |
| 1 | B | C | C | D |
| 2 | B | C | C | D |
| 3 | B | C | C | D |
| 4 | B | C | C | D |
| 5 | C | C | C | D |
| 6 | C | C | C | D |
| 7 | B | C | C | D |
| 8 | C | C | C | D |
| 9 | C | D | D | D |
| 10 | C | C | C | D |
| 11 | C | C | D | D |
| 12 | C | C | C | D |
| 13 | B | C | C | D |
| 14 | C | C | C | D |
| 15 | B | C | C | D |
| 16 | B | C | C | D |
| 22 | B | C | C | D |
| 23 | B | C | C | D |
| 24 | B | C | C | D |
| 25 | B | C | C | D |
| 26 | C | C | C | D |
| 27 | C | C | C | D |
| 28 | C | C | D | D |
| 29 | B | C | C | D |
| 30 | B | C | C | D |
| 31 | C | D | C | D |
| 32 | B | C | C | D |
| 33 | C | D | C | D |
| 34 | C | D | C | D |
| 35 | C | C | C | D |
| 36 | C | C | C | D |
| 37 | B | C | C | D |
| 38 | B | C | C | D |
| 39 | B | C | C | D |
| 40 | B | C | C | D |
| 41 | C | C | C | D |
| 42 | B | C | C | D |
| 43 | C | D | D | D |
| 44 | B | C | C | D |
| 45 | C | C | C | D |
| 46 | C | C | C | D |
| 47 | C | C | C | D |
| 48 | B | C | C | D |
| 49 | C | C | C | D |
| 50 | C | C | C | D |
| 51 | C | D | C | D |
| 52 | C | C | D | D |
| 53 | B | C | C | D |
| 54 | B | B | C | D |
| 55 | C | C | C | D |
| 56 | C | C | C | D |
| 57 | C | C | C | D |
| 58 | C | C | C | D |
| 59 | C | C | B | D |
| 60 | B | C | C | D |
| 61 | C | C | C | D |
| 62 | C | C | C | D |
| 63 | C | C | C | D |
| 64 | C | C | C | D |
| 65 | C | C | C | D |
| 66 | C | C | C | D |
| 67 | B | B | C | D |
| 68 | B | C | C | D |
| 69 | B | C | C | D |
| 70 | B | B | C | D |
| 71 | B | C | C | D |
| 72 | B | C | C | D |
| 73 | B | C | C | D |
| 74 | B | B | C | D |
| 75 | B | C | C | D |
| 76 | B | C | C | D |
| 77 | B | C | C | D |
| 78 | B | C | C | D |
| 79 | B | C | C | D |

TABLE 3
Inhibition of bacterial growth for P. aeruginosa strains. Minimum
Inhibitory Concentrations of exemplary compounds (MIC, in μg/mL).

| | P. aeruginosa | P. aeruginosa | P. aeruginosa | P. aeruginosa |
|---|---|---|---|---|
| Example | 27853 | 35151 | ΔmexAB-OprM | Paeβ-17 |
| 1 | B | D | C | A |
| 2 | B | D | C | A |
| 3 | B | D | C | A |
| 4 | B | D | C | A |
| 5 | C | D | C | B |
| 6 | C | D | C | B |
| 7 | B | D | C | B |
| 8 | C | D | C | B |
| 9 | C | D | D | B |
| 10 | B | D | C | B |
| 11 | C | D | C | B |
| 12 | C | D | C | B |
| 13 | B | D | C | B |
| 14 | C | D | C | B |
| 15 | C | D | C | B |
| 16 | B | D | C | B |
| 22 | B | D | C | A |
| 23 | B | D | C | A |
| 24 | B | D | C | A |
| 25 | B | D | C | A |
| 26 | C | D | D | B |
| 27 | B | D | C | B |
| 28 | C | D | D | B |
| 29 | B | D | C | B |
| 30 | B | C | C | A |
| 31 | C | D | D | B |
| 32 | B | D | C | B |
| 33 | C | D | C | B |
| 34 | C | D | C | B |
| 35 | C | D | C | B |
| 36 | C | D | C | B |
| 37 | B | D | C | B |
| 38 | B | D | C | B |
| 39 | B | C | C | A |
| 40 | B | D | C | B |
| 41 | C | D | C | B |
| 42 | B | D | B | B |
| 43 | C | D | C | B |
| 44 | B | D | C | B |
| 45 | C | D | C | B |
| 46 | C | D | C | B |
| 47 | C | D | C | B |
| 48 | B | D | C | B |
| 49 | C | D | C | B |
| 50 | B | D | C | B |
| 51 | C | D | C | B |
| 52 | C | D | C | B |
| 53 | B | D | C | B |
| 54 | B | D | C | B |
| 55 | B | D | C | B |
| 56 | C | D | C | B |
| 57 | C | D | C | B |
| 58 | C | D | C | B |
| 59 | B | D | C | B |
| 60 | B | D | C | B |
| 61 | C | D | C | B |
| 62 | C | D | C | B |
| 63 | C | D | C | B |
| 64 | C | D | C | B |
| 65 | C | D | C | B |
| 66 | C | D | C | B |
| 67 | B | D | C | B |
| 68 | B | D | C | B |
| 69 | B | D | C | B |
| 70 | B | D | C | B |
| 71 | B | D | C | B |
| 72 | C | D | B | C |
| 73 | C | D | B | C |
| 74 | B | D | B | C |
| 75 | C | D | B | C |
| 76 | C | D | B | C |
| 77 | C | D | B | C |
| 78 | C | D | B | C |
| 79 | C | D | B | C |

### Example B2. Expanded MIC Profiling in Clinical Isolates

To evaluate the activity of selected example compounds against clinical isolates of target species, standard cell-based broth microdilution MIC assays were employed. A panel of 20 clinical isolates of P. aeruginosa and 20 clinical isolates of Methicillin Resistant S. aureus (MRSA) from Lankenau Medical Center (Wynnewood, Pa.) were used. In the set of P. aeruginosa isolates, the compounds were also tested in combination with a β-lactamase inhibitor (taniborbactam), to evaluate the potential benefit of inhibiting β-lactamase enzymes. The assay was conducted in Cation Adjusted Mueller Hinton Broth (CAMHB, BD #212322, BD Diagnostic Systems, Sparks, Md.). Bacteria strains were grown for 3-5 hours in CAMHB. Compounds were added to a microtiter plate in 2-fold serial dilutions in CAMHB. For plates receiving β-lactamase inhibitor, taniborbactam was added to obtain a final fixed concentration of 4 μg/mL. Once the test articles were added, the plates were inoculated according to CLSI broth microdilution method. After inoculation, the plates were incubated for 16-20 hours at 37° C. then the Minimal Inhibitory Concentration (MIC) of the test compound is determined visually. Values for concentration required to inhibit growth of 500% (MIC50) and 90% (MIC90) of isolates were determined.

Table 4 shows results for P. aeruginosa MIC testing, and Table 5 shows results for MRSA MIC testing.

TABLE 4
Inhibition of bacterial growth for P. aeruginosa clinical isolates. Minimum
Inhibitory Concentrations of exemplary compounds (MIC, in μg/mL).

| | | Example 28 | | Meropenem |
|---|---|---|---|---|
| | | plus | | plus |
| Strain ID | Example 28 | taniborbactam | Meropenem | taniborbactam |
| Lankenau 11 | 2 | 1 | 1 | 1 |
| Lankenau 12 | 0.5 | 1 | 0.5 | 1 |
| Lankenau 13 | 0.5 | 4 | 1 | 1 |
| Lankenau 14 | 1 | 0.5 | 0.5 | <0.25 |
| Lankenau 15 | 1 | 0.5 | 1 | 2 |
| Lankenau 16 | 1 | 2 | 0.5 | 0.5 |
| Lankenau 17 | 1 | 1 | 1 | 1 |
| Lankenau 18 | 1 | 0.25 | 0.5 | 0.5 |
| Lankenau 19 | 1 | 1 | 1 | 0.5 |
| Lankenau 20 | 1 | 0.25 | 0.5 | 0.5 |
| Lankenau 21 | 1 | 0.5 | 1 | 2 |
| Lankenau 22 | 1 | 0.5 | 0.5 | <0.25 |
| Lankenau 23 | 2 | 0.25 | 0.5 | 0.5 |
| Lankenau 24 | 2 | 2 | 4 | 4 |
| Lankenau 25 | 2 | 2 | 0.5 | 0.5 |
| Lankenau 26 | 2 | 0.5 | <0.25 | <0.25 |
| Lankenau 27 | 2 | 1 | 0.5 | <0.25 |
| Lankenau 28 | 8 | 1 | 0.5 | 0.5 |
| Lankenau 29 | 16 | 0.5 | 0.5 | 0.5 |
| Lankenau 30 | 32 | 1 | 0.5 | 1 |
| MIC50 | 1 | 1 | 0.5 | 0.5 |
| MIC90 | 8 | 2 | 1 | 2 |

TABLE 5
Inhibition of bacterial growth for S. aureus clinical isolates.
Minimum Inhibitory Concentrations of exemplary compounds
(MIC, in μg/mL).

| Strain ID | Example 28 | Meropenem |
|---|---|---|
| LH-SA3 | 0.5 | 4 |
| LH-SA9 | 0.5 | 4 |
| LH-SA23 | 0.5 | 4 |
| LH-SA24 | 0.5 | 4 |
| LH-SA26 | 0.5 | 4 |
| LH-SA29 | 2 | 16 |
| LH-SA33 | 2 | 4 |
| LH-SA34 | 4 | 8 |
| LH-SA37 | 0.25 | 4 |
| LH-SA38 | 4 | 32 |
| LH-SA39 | 2 | 4 |
| LH-SA40 | 2 | 4 |
| LH-SA41 | 2 | 4 |
| LH-SA42 | 0.5 | 8 |
| LH-SA44 | 4 | 8 |
| LH-SA45 | 16 | 16 |
| LH-SA48 | 0.5 | 4 |
| LH-SA49 | 0.5 | 4 |
| LH-SA50 | 0.5 | 4 |
| ATCC-33591 | 2 | 32 |
| MIC50 | 0.5 | 4 |
| MIC90 | 4 | 16 |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A compound of Formula (III), or a pharmaceutically acceptable salt, solvate, or stereoisomer thereof: 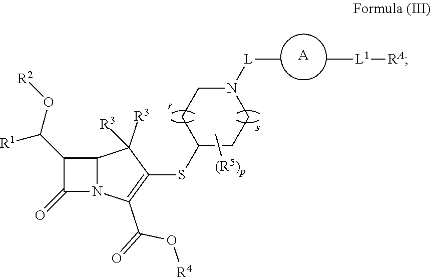
wherein
Ring A is cycloalkyl optionally substituted with one, two, or three oxo, halogen, C₁-C₆ alkyl, C₁-C₆ haloalkyl, —OR^{a}, —CN, —NO₂, —NR^{c}R^{d}, —S(═O)₂R^{b}, —S(═O)₂NR^{c}R^{d}, —C(═O)OR^{a}, —C(═O)R^{b}, —C(═O)NR^{c}R^{d}, —NR^{c}C(═NR^{c})NR^{c}R^{d}, or —NR^{c}C(═NR^{c})R^{b};
L¹ is a bond or C₁-C₆ alkylene optionally substituted with halogen, —OR^{a}, or —NR^{c}R^{d};
R^{A} is halogen, C₁-C₆ alkyl, C₂-C₆ alkenyl, C₂-C₆ alkynyl, C₁-C₆ haloalkyl, C₁-C₆ hydroxyalkyl, —OR^{a}, —CN, —NO₂, —S(═O)₂R¹⁰, —S(═O)₂NR¹⁰R¹¹, —C(═O)OR¹⁰, —C(═O)NR¹⁰R¹¹, —NR¹⁰C(═NR¹¹)NR¹⁰R¹¹, —C(═NR¹⁰)R¹⁰, —NR¹⁰C(═NR¹¹)R¹⁰, —NR¹⁰(CR¹²R¹³)_{w}NR¹⁰R¹¹, —NR¹⁰C(═NR¹¹)NR¹⁰(CR¹²R¹³)_{w}NR¹⁰R¹¹, —C(═NR¹¹)NR¹⁰R¹¹, C₃-C₈ cycloalkyl, C₂-C₈ heterocycloalkyl, aryl, or heteroaryl; wherein each alkyl, alkenyl, alkynyl, cycloalkyl, heterocycloalkyl, aryl, and heteroaryl is independently optionally substituted with one, two, or three R^{A1};
each R^{A1} is independently halogen, C₁-C₆ alkyl, C₁-C₆ haloalkyl, —OR^{a}, —CN, —NO₂, —NR^{W}R^{d}, —S(═O)₂R^{b}, —S(═O)₂NR^{W}R^{d}, —C(═O)OR^{a}, —C(═O)R^{b}, —C(═O)NR^{c}R^{d}, —NR^{c}C(═NR)NR^{W}R^{d}, or —NR^{c}C(═NR^{c})R^{b};
L is a bond or C₁-C₆ alkylene optionally substituted with halogen, —OR^{a}, or —NR^{c}R^{d};
R¹ is C₁-C₆ alkyl, C₁-C₆ haloalkyl, C₂-C₆ alkenyl, or C₂-C₆ alkynyl;
R² is hydrogen, C₁-C₆ alkyl, C₁-C₆ haloalkyl, C₂-C₆ alkenyl, C₂-C₆ alkynyl, or —Si(R^{b})₃;
each R³ is independently hydrogen, C₁-C₆ alkyl, C₁-C₆ haloalkyl, C₂-C₆ alkenyl, C₂-C₆ alkynyl, or —OR^{a};
R⁴ is hydrogen, C₁-C₆ alkyl, C₁-C₆ haloalkyl, C₂-C₆ alkenyl, C₂-C₆ alkynyl, C₃-C₈ cycloalkyl, C₂-C₈ heterocycloalkyl, aryl, heteroaryl, —(C₁-C₆ alkyl)(C₃-C₈ cycloalkyl), —(C₁-C₆ alkyl)(C₂-C₈ heterocycloalkyl), —(C₁-C₆ alkyl)(aryl), or —(C₁-C₆ alkyl)(heteroaryl);
each R⁵ is independently halogen, C₁-C₆ alkyl, C₁-C₆ haloalkyl, C₂-C₆ alkenyl, C₂-C₆ alkynyl, —OR^{a}, —CN, —NO₂, —S(═O)₂R^{b}, —S(═O)₂NR^{c}R^{d}, —C(═O)OR^{a}, —C(═O)R^{b}, —C(═O)NR^{c}R^{d}, —NR^{c}R^{d}, C₃-C₈ cycloalkyl, or C₂-C₈ heterocycloalkyl;
or two R⁵ on the same carbon are taken together to form an oxo;
each R¹⁰ and R¹¹ is independently hydrogen, C₁-C₆ alkyl, C₁-C₆ haloalkyl, C₂-C₆ alkenyl, C₂-C₆ alkynyl, C₃-C₈ cycloalkyl, C₂-C₈ heterocycloalkyl, aryl, heteroaryl, —(C₁-C₆ alkyl)(C₃-C₈ cycloalkyl), —(C₁-C₆ alkyl)(C₂-C₈ heterocycloalkyl), —(C₁-C₆ alkyl)(aryl), or —(C₁-C₆ alkyl)(heteroaryl);
or R¹⁰ and R¹¹ are taken together with the nitrogen to which they are attached to form a C₂-C₈ heterocycloalkyl;
each R¹² and R¹³ is independently hydrogen, halogen, —CN, —OR^{a}, —NR^{c}R^{d}, C₁-C₆ alkyl, C₁-C₆ haloalkyl, C₂-C₆ alkenyl, C₂-C₆ alkynyl, C₃-C₈ cycloalkyl, C₂-C₈ heterocycloalkyl, aryl, or heteroaryl;
each R^{a} is independently hydrogen, C₁-C₆ alkyl, C₁-C₆ haloalkyl, C₂-C₆ alkenyl, C₂-C₆ alkynyl, C₃-C₈ cycloalkyl, or C₂-C₈ heterocycloalkyl;
each R^{b} is independently C₁-C₆ alkyl, C₁-C₆ haloalkyl, C₂-C₆ alkenyl, C₂-C₆ alkynyl, C₃-C₈ cycloalkyl, or C₂-C₈ heterocycloalkyl;
each R^{c} and R^{d} are independently hydrogen, C₁-C₆ alkyl, C₁-C₆ haloalkyl, C₂-C₆ alkenyl, C₂-C₆ alkynyl, C₃-C₈ cycloalkyl, or C₂-C₈ heterocycloalkyl;
or R^{c} and R^{d} are taken together with the nitrogen to which they are attached to form a C₂-C₈ heterocycloalkyl;
w is 2-4;
r is 1-2;
s is 0-2; and
p is 0-5.

2. The compound of claim 1, wherein R¹ is C₁-C₆ alkyl.

3. The compound of claim 1 or 2, wherein R² is hydrogen.

4. The compound of any one of claims 1-3, wherein each R³ is independently hydrogen or C₁-C₆ alkyl.

5. The compound of any one of claims 1-4, wherein R⁴ is hydrogen.

6. The compound of any one of claims 1-5, wherein the compound of Formula (III), or a pharmaceutically acceptable salt, solvate, or stereoisomer thereof, is of Formula (IIIa):

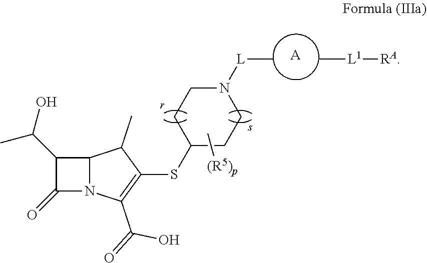

7. The compound of any one of claims 1-6, wherein the compound of Formula (III), or a pharmaceutically acceptable salt, solvate, or stereoisomer thereof, is of Formula (IIIb):

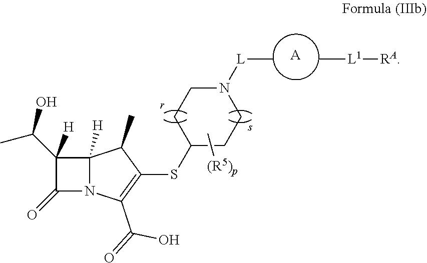

8. The compound of any one of claims 1-7, wherein p is 0.

9. The compound of any one of claims 1-8, wherein r is 1 and s is 0.

10. The compound of any one of claims 1-8, wherein r is 1 and s is 1.

11. The compound of any one of claims 1-10, wherein L is a bond.

12. The compound of any one of claims 1-10, wherein L is C₁ alkylene.

13. The compound of any one of claims 1-12, wherein Ring A is cyclohexyl.

14. The compound of any one of claims 1-13, wherein L¹ is a bond.

15. The compound of any one of claims 1-13, wherein L¹ is C₁ alkylene.

16. The compound of any one of claims 1-15, wherein R^{A} is —S(═O)₂NR¹⁰R¹¹, —C(═O)NR¹⁰R¹¹, —NR¹⁰C(═NR¹¹)NR¹⁰R¹¹, —C(═NR¹⁰)R¹⁰, —NR¹⁰C(═NR¹¹)R¹⁰, —NR¹⁰(CR¹²R¹³)_{w}NR¹⁰R¹¹, —NR¹⁰C(═NR¹¹)NR¹⁰(CR¹²R¹³)_{w}NR¹⁰R¹¹, or —C(═NR¹¹)NR¹⁰R¹¹.

17. The compound of any one of claims 1-16, wherein R^{A} is —NR¹⁰C(═NR¹¹)NR¹⁰R¹¹, —C(═NR¹⁰)R¹⁰, or —NR¹⁰C(═NR¹¹)R¹⁰.

18. The compound of any one of claims 1-17, wherein R^{A} is NR¹⁰C(═NR¹¹)NR¹⁰R¹¹.

19. The compound of any one of claims 1-18, wherein each R¹⁰ and R¹¹ is independently hydrogen or C₁-C₆ alkyl.

20. The compound of any one of claims 1-19, wherein each R¹⁰ and R¹¹ is hydrogen.

21. The compound of any one of claims 1-20, wherein each R¹² and R¹³ is independently hydrogen, halogen, or C₁-C₆ alkyl.

22. The compound of any one of claims 1-21, wherein each R¹² and R¹³ is hydrogen.

23. The compound of claim 1 selected from the group consisting of: 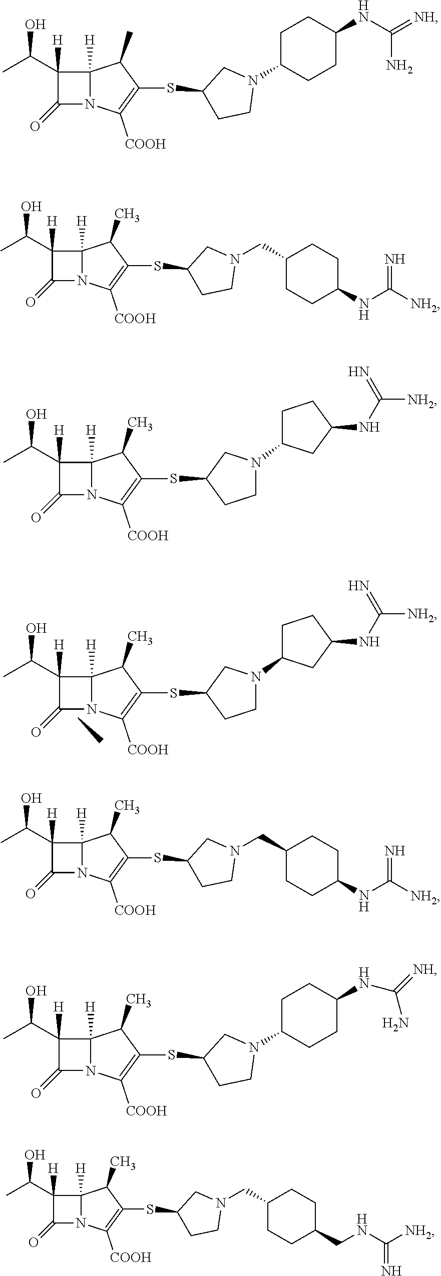 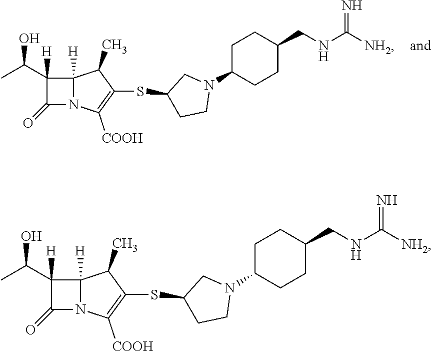 [SMILES: C[C@@H](O)[C@H]1C(=O)N2C(C(=O)O)=C(S[C@@H]3CCN([C@H]4CC[C@@H](CNC(=N)N)CC4)C3)[C@H](C)[C@H]12 ; "and" ; C[C@@H](O)[C@H]1C(=O)N2C(C(=O)O)=C(S[C@@H]3CCN([C@H]4CC[C@H](CNC(=N)N)CC4)C3)[C@H](C)[C@H]12]
or a pharmaceutically acceptable salt, solvate, or stereoisomer thereof.

24. A compound of Formula (I), or a pharmaceutically acceptable salt, solvate, or stereoisomer thereof: 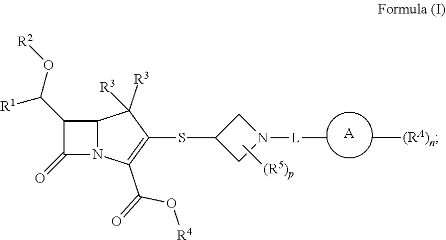
wherein
Ring A is cyclobutyl or cyclopentyl;
each R^{A} is independently halogen, C₁-C₆ alkyl, C₂-C₆ alkenyl, C₂-C₆ alkynyl, C₁-C₆ haloalkyl, C₁-C₆ hydroxyalkyl, —(CR¹²R¹³)ᵥOR¹⁰, —(CR¹²R¹³)ᵥNR¹⁰R¹¹, —CN, —NO₂, —(CR¹²R¹³)ᵥS(═O)₂R¹⁰, —(CR¹²R¹³)ᵥS(═O)₂NR¹⁰R¹¹, —(CR¹²R¹³)ᵥC(═O)OR¹⁰, —(CR¹²R¹³)ᵥC(═O)NR¹⁰R¹¹, —(CR¹²R¹³)ᵥNR¹⁰C(═NR¹¹)NR¹⁰R¹¹, —(CR¹²R¹³)ᵥC(═NR¹⁰)R¹⁰, —(CR¹²R¹³)ᵥNR¹⁰C(═NR¹¹)R¹⁰, —(CR¹²R¹³)ᵥNR¹⁰(CR¹²R¹³)_{w}NR¹⁰R¹¹, —(CR¹²R¹³)ᵥ(NR¹⁰C(═NR¹¹)NR¹⁰(CR¹²R¹³)_{w}NR¹⁰R¹¹, —(CR¹²R¹³)ᵥC(═NR¹¹)NR¹⁰R¹¹, C₃-C₈ cycloalkyl, C₂-C₈ heterocycloalkyl, aryl, or heteroaryl;
wherein each alkyl, alkenyl, alkynyl, cycloalkyl, heterocycloalkyl, aryl, and heteroaryl is independently optionally substituted with one, two, or three R^{A1};
each R^{A1} is independently halogen, C₁-C₆ alkyl, C₁-C₆ haloalkyl, —OR^{a}, —CN, —NO₂, —NR^{c}R^{d}, —S(═O)₂R^{b}, —S(═O)₂NR^{c}R^{d}, —C(═O)OR^{a}, —C(═O)R^{b}, —C(═O)NR^{c}R^{d}, —NR^{c}C(═NR)NR^{c}R^{d}, or —NR^{c}C(═NR^{c})R^{b};
L is a bond or C₁-C₆ alkylene optionally substituted with halogen, —OR^{a}, —CN, —NO₂, or —NR^{c}R^{d};
R¹ is C₁-C₆ alkyl, C₁-C₆ haloalkyl, C₂-C₆ alkenyl, or C₂-C₆ alkynyl;
R² is hydrogen, C₁-C₆ alkyl, C₁-C₆ haloalkyl, C₂-C₆ alkenyl, C₂-C₆ alkynyl, or —Si(R^{b})₃;
each R³ is independently hydrogen, C₁-C₆ alkyl, C₁-C₆ haloalkyl, C₂-C₆ alkenyl, C₂-C₆ alkynyl, or —OR^{a};
R⁴ is hydrogen, C₁-C₆ alkyl, C₁-C₆ haloalkyl, C₂-C₆ alkenyl, C₂-C₆ alkynyl, C₃-C₈ cycloalkyl, C₂-C₈ heterocycloalkyl, aryl, heteroaryl, —(C₁-C₆ alkyl)(C₃-C₈ cycloalkyl), —(C₁-C₆ alkyl)(C₂-C₈ heterocycloalkyl), —(C₁-C₆ alkyl)(aryl), or —(C₁-C₆ alkyl)(heteroaryl);
each R⁵ is independently halogen, C₁-C₆ alkyl, C₁-C₆ haloalkyl, C₂-C₆ alkenyl, C₂-C₆ alkynyl, —OR^{a}, —CN, —NO₂, —S(═O)₂R^{b}, —S(═O)₂NR^{c}R^{d}, —C(═O)OR^{a}, —C(═O)R^{b}, —C(═O)NR^{c}R^{d}, —NR^{c}R^{d}, C₃-C₈ cycloalkyl, or C₂-C₈ heterocycloalkyl;
or two R⁵ on the same carbon are taken together to form an oxo;
each R¹⁰ and R¹¹ is independently hydrogen, C₁-C₆ alkyl, C₁-C₆ haloalkyl, C₂-C₆ alkenyl, C₂-C₆ alkynyl, C₃-C₈ cycloalkyl, C₂-C₈ heterocycloalkyl, aryl, heteroaryl, —(C₁-C₆ alkyl)(C₃-C₈ cycloalkyl), —(C₁-C₆ alkyl)(C₂-C₈ heterocycloalkyl), —(C₁-C₆ alkyl)(aryl), or —(C₁-C₆ alkyl)(heteroaryl);
or R¹⁰ and R¹¹ are taken together with the nitrogen to which they are attached to form a C₂-C₈ heterocycloalkyl;
each R¹² and R¹³ is independently hydrogen, halogen, —CN, —OR^{a}, —NR^{c}R^{d}, C₁-C₆ alkyl, C₁-C₆ haloalkyl, C₂-C₆ alkenyl, C₂-C₆ alkynyl, C₃-C₈ cycloalkyl, C₂-C₈ heterocycloalkyl, aryl, or heteroaryl;
each R^{a} is independently hydrogen, C₁-C₆ alkyl, C₁-C₆ haloalkyl, C₂-C₆ alkenyl, C₂-C₆ alkynyl, C₃-C₈ cycloalkyl, or C₂-C₈ heterocycloalkyl;
each R^{b} is independently C₁-C₆ alkyl, C₁-C₆ haloalkyl, C₂-C₆ alkenyl, C₂-C₆ alkynyl, C₃-C₈ cycloalkyl, or C₂-C₈ heterocycloalkyl;
each R^{c} and R^{d} are independently hydrogen, C₁-C₆ alkyl, C₁-C₆ haloalkyl, C₂-C₆ alkenyl, C₂-C₆ alkynyl, C₃-C₈ cycloalkyl, or C₂-C₈ heterocycloalkyl;
or R and R^{a} are taken together with the nitrogen to which they are attached to form a C₂-C₈ heterocycloalkyl;
v is 0-4;
w is 2-4;
n is 0-4; and
p is 0-5.

25. The compound of claim 24, wherein R¹ is C₁-C₆ alkyl.

26. The compound of claim 24 or 25, wherein R² is hydrogen.

27. The compound of any one of claims 24-26, wherein each R³ is independently hydrogen or C₁-C₆ alkyl.

28. The compound of any one of claims 24-27, wherein R⁴ is hydrogen.

29. The compound of any one of claims 24-28, wherein the compound of Formula (I), or a pharmaceutically acceptable salt, solvate, or stereoisomer thereof, is of Formula (Ia):

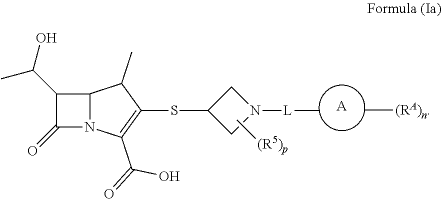

30. The compound of any one of claims 24-29, wherein the compound of Formula (I), or a pharmaceutically acceptable salt, solvate, or stereoisomer thereof, is of Formula (Ib):

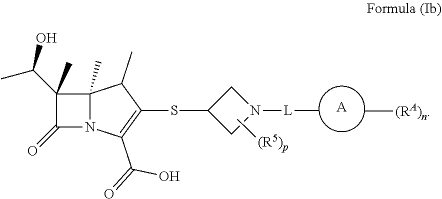

31. The compound of any one of claims 24-30, wherein p is 0.

32. The compound of any one of claims 24-31, wherein L is a bond.

33. The compound of any one of claims 24-32, wherein L is C₁ alkylene.

34. The compound of any one of claims 24-33, wherein Ring A is cyclobutyl.

35. The compound of any one of claims 24-33, wherein Ring A is cyclopentyl.

36. The compound of any one of claims 24-35, wherein n is 1 or 2.

37. The compound of any one of claims 24-36, wherein n is 1.

38. The compound of any one of claims 24-37, wherein each R^{A} is independently —(CR¹²R¹³)ᵥNR¹⁰R¹¹, —(CR¹²R¹³)ᵥS(═O)₂NR¹⁰R¹¹, —(CR¹²R¹³)ᵥC(═O)NR¹⁰R¹¹, —(CR¹²R¹³)ᵥNR¹⁰C(═NR¹¹)NR¹⁰R¹¹, —(CR¹²R¹³)ᵥC(═NR¹⁰)R¹⁰, —(CR¹²R¹³)ᵥNR¹⁰C(═NR¹¹)R¹⁰, —(CR¹²R¹³)ᵥNR¹⁰(CR¹²R¹³)_{w}NR¹⁰R¹¹, —(CR¹²R¹³)ᵥ(NR¹⁰C(═NR¹¹)NR¹⁰(CR¹²R¹³)_{w}NR¹⁰R¹¹, or —(CR¹²R¹³)ᵥC(═NR¹¹)NR¹⁰R¹¹.

39. The compound of any one of claims 24-38, wherein each R^{A} is independently —(CR¹²R¹³)ᵥNR¹⁰R¹¹, —(CR¹²R¹³)ᵥNR¹⁰C(═NR¹¹)NR¹⁰R¹¹, —(CR¹²R¹³)ᵥC(═NR¹⁰)R¹⁰, —(CR¹²R¹³)ᵥNR¹⁰C(═NR¹¹)R¹⁰, —(CR¹²R¹³)ᵥNR¹⁰(CR¹²R¹³)_{w}NR¹⁰R¹¹, —(CR¹²R¹³)ᵥ(NR¹⁰C(═NR¹¹)NR¹⁰(CR¹²R¹³)_{w}NR¹⁰R¹¹, or —(CR¹²R¹³)ᵥC(═NR¹¹)NR¹⁰R¹¹.

40. The compound of any one of claims 24-39, wherein each R^{A} is independently —(CR¹²R¹³)ᵥNR¹⁰R¹¹, —(CR¹²R¹³)ᵥNR¹⁰C(═NR¹¹)NR¹⁰R¹¹, —(CR¹²R¹³)ᵥC(═NR¹⁰)R¹⁰, —(CR¹²R¹³)ᵥNR¹⁰C(═NR¹¹)R¹⁰.

41. The compound of any one of claims 24-40, wherein each R^{A} is independently —(CR¹²R¹³)ᵥNR¹⁰R¹¹ or —(CR¹²R¹³)ᵥNR¹⁰C(═NR¹¹)NR¹⁰R¹¹.

42. The compound of any one of claims 24-41, wherein v is 0 or 1.

43. The compound of any one of claims 24-42, wherein v is 0.

44. The compound of any one of claims 24-43, wherein each R¹⁰ and R¹¹ is independently hydrogen or C₁-C₆ alkyl.

45. The compound of any one of claims 24-44, wherein each R¹⁰ and R¹¹ is hydrogen.

46. The compound of any one of claims 24-45, wherein each R¹² and R¹³ is independently hydrogen, halogen, or C₁-C₆ alkyl.

47. The compound of any one of claims 24-46, wherein each R¹² and R¹³ is hydrogen.

48. The compound of claim 24 selected from the group consisting of: 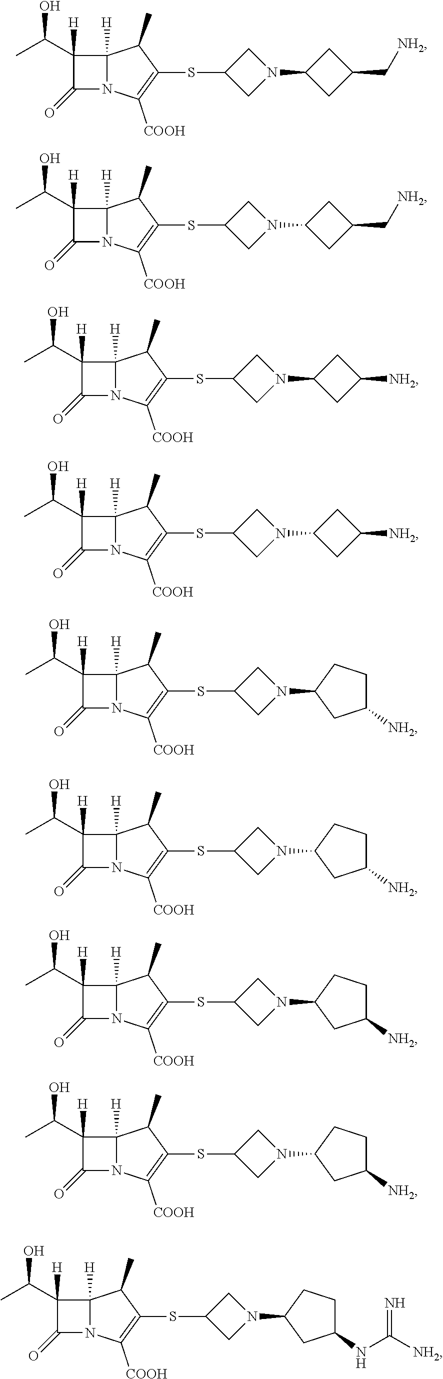 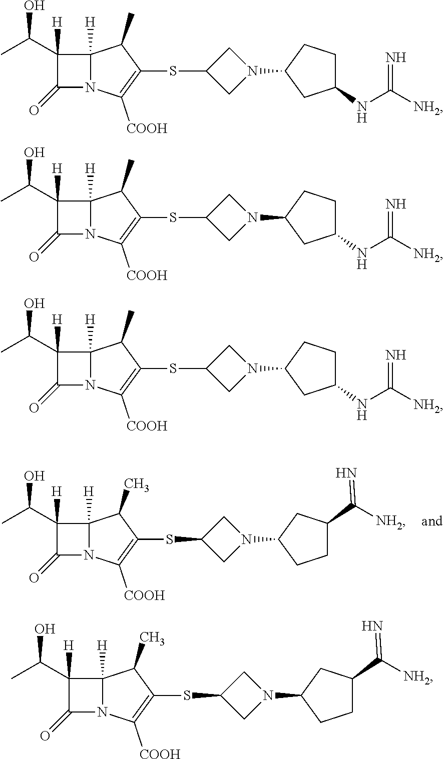
or a pharmaceutically acceptable salt, solvate, or stereoisomer thereof.

49. A compound of Formula (II), or a pharmaceutically acceptable salt, solvate, or stereoisomer thereof: 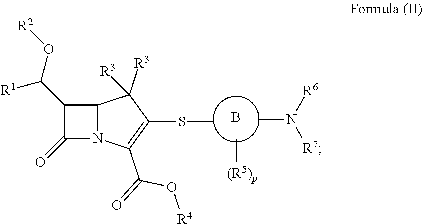
wherein
Ring B is C₃-C₈ cycloalkyl;
R¹ is C₁-C₆ alkyl, C₁-C₆ haloalkyl, C₂-C₆ alkenyl, or C₂-C₆ alkynyl;
R² is hydrogen, C₁-C₆ alkyl, C₁-C₆ haloalkyl, C₂-C₆ alkenyl, C₂-C₆ alkynyl, or —Si(R^{b})₃;
each R³ is independently hydrogen, C₁-C₆ alkyl, C₁-C₆ haloalkyl, C₂-C₆ alkenyl, C₂-C₆ alkynyl, or —OR^{a};
R⁴ is hydrogen, C₁-C₆ alkyl, C₁-C₆ haloalkyl, C₂-C₆ alkenyl, C₂-C₆ alkynyl, C₃-C₈ cycloalkyl, C₂-C₈ heterocycloalkyl, aryl, heteroaryl, —(C₁-C₆ alkyl)(C₃-C₈ cycloalkyl), —(C₁-C₆ alkyl)(C₂-C₈ heterocycloalkyl), —(C₁-C₆ alkyl)(aryl), or —(C₁-C₆ alkyl)(heteroaryl);
each R⁵ is independently halogen, C₁-C₆ alkyl, C₁-C₆ haloalkyl, C₂-C₆ alkenyl, C₂-C₆ alkynyl, —OR^{a}, —CN, —NO₂, —S(═O)₂R^{b}, —S(═O)₂NR^{c}R^{d}, —C(═O)OR^{a}, —C(═O)R^{b}, —C(═O)NR^{c}R^{d}, —NR^{c}R^{d}, C₁-C₆ alkyl, C₂-C₆ alkenyl, C₂-C₆ alkynyl, C₃-C₈ cycloalkyl, or C₂-C₈ heterocycloalkyl;
or two R⁵ on the same carbon are taken together to form an oxo;
R⁶ is hydrogen, C₁-C₆ alkyl, C₁-C₆ haloalkyl, C₂-C₆ alkenyl, C₂-C₆ alkynyl, C₃-C₈ cycloalkyl, or C₂-C₈ heterocycloalkyl;
R⁷ is C₃-C₈ cycloalkyl, C₂-C₈ heterocycloalkyl, aryl, and heteroaryl; each independently optionally substituted with one, two, or three R^{7a};
or R⁶ and R⁷ are taken together with the nitrogen to which they are attached to form a C₂-C₈ heterocycloalkyl optionally substituted with one, two, or three R^{7a};
each R^{7a} is independently halogen, C₁-C₆ alkyl, C₂-C₆ alkenyl, C₂-C₆ alkynyl, C₁-C₆ haloalkyl, C₁-C₆ hydroxyalkyl, —(CR¹²R¹³)ᵥOR¹⁰, —(CR¹²R¹³)ᵥNR¹⁰R¹¹, —CN, —NO₂, —(CR¹²R¹³)ᵥS(═O)₂R¹⁰, —(CR¹²R¹³)ᵥS(═O)₂NR¹⁰R¹¹, —(CR¹²R¹³)ᵥC(═O)OR¹⁰, —(CR¹²R¹³)ᵥC(═O)NR¹⁰R¹¹, —(CR¹²R¹³)ᵥNR¹⁰C(═NR¹¹)NR¹⁰R¹¹, —(CR¹²R¹³)ᵥC(═NR¹⁰)R¹⁰, —(CR¹²R¹³)ᵥNR¹⁰C(═NR¹¹)R¹⁰, —(CR¹²R¹³)ᵥNR¹⁰(CR¹²R¹³)_{w}NR¹⁰R¹¹, —(CR¹²R¹³)ᵥ(NR¹⁰C(═NR¹¹)NR¹⁰(CR¹²R¹³)_{w}NR¹⁰R¹¹, —(CR¹²R¹³)ᵥC(═NR¹¹)NR¹⁰R¹¹, C₃-C₈ cycloalkyl, C₂-C₈ heterocycloalkyl, aryl, or heteroaryl;
wherein each alkyl, alkenyl, alkynyl, cycloalkyl, heterocycloalkyl, aryl, and heteroaryl is independently optionally substituted with one, two, or three R^{7b};
each R^{7b} is independently halogen, C₁-C₆ alkyl, C₁-C₆ haloalkyl, —OR^{a}, —CN, —NO₂, —NR^{c}R^{d}, —S(═O)₂R^{b}, —S(═O)₂NR^{c}R^{d}, —C(═O)OR^{a}, —C(═O)R^{b}, —C(═O)NR^{c}R^{d}, —NR^{c}C(═NR^{c})NR^{c}R^{d}, or —NR^{c}C(═NR^{c})R^{b};
each R¹⁰ and R¹¹ is independently hydrogen, C₁-C₆ alkyl, C₁-C₆ haloalkyl, C₂-C₆ alkenyl, C₂-C₆ alkynyl, C₃-C₈ cycloalkyl, C₂-C₈ heterocycloalkyl, aryl, heteroaryl, —(C₁-C₆ alkyl)(C₃-C₈ cycloalkyl), —(C₁-C₆ alkyl)(C₂-C₈ heterocycloalkyl), —(C₁-C₆ alkyl)(aryl), or —(C₁-C₆ alkyl)(heteroaryl);
or R¹⁰ and R¹¹ are taken together with the nitrogen to which they are attached to form a C₂-C₈ heterocycloalkyl;
each R¹² and R¹³ is independently hydrogen, halogen, —CN, —OR^{a}, —NR^{c}R^{c}, C₁-C₆ alkyl, C₁-C₆ haloalkyl, C₂-C₆ alkenyl, C₂-C₆ alkynyl, C₃-C₈ cycloalkyl, C₂-C₈ heterocycloalkyl, aryl, or heteroaryl;
each R^{a} is independently hydrogen, C₁-C₆ alkyl, C₁-C₆ haloalkyl, C₂-C₆ alkenyl, C₂-C₆ alkynyl, C₃-C₈ cycloalkyl, or C₂-C₈ heterocycloalkyl;
each R^{b} is independently C₁-C₆ alkyl, C₁-C₆ haloalkyl, C₂-C₆ alkenyl, C₂-C₆ alkynyl, C₃-C₈ cycloalkyl, or C₂-C₈ heterocycloalkyl;
each R^{c} and R^{d} are independently hydrogen, C₁-C₆ alkyl, C₁-C₆ haloalkyl, C₂-C₆ alkenyl, C₂-C₆ alkynyl, C₃-C₈ cycloalkyl, or C₂-C₈ heterocycloalkyl;
or R^{c} and R^{d} are taken together with the nitrogen to which they are attached to form a C₂-C₈ heterocycloalkyl;
v is 0-4;
w is 2-4; and
p is 0-5.

50. The compound of claim 49, wherein R¹ is C₁-C₆ alkyl.

51. The compound of claim 49 or 50, wherein R² is hydrogen.

52. The compound of any one of claims 49-51, wherein each R³ is independently hydrogen or C₁-C₆ alkyl.

53. The compound of any one of claims 49-52, wherein R⁴ is hydrogen.

54. The compound of any one of claims 49-53, wherein the compound of Formula (II), or a pharmaceutically acceptable salt, solvate, or stereoisomer thereof, is of Formula (IIa):

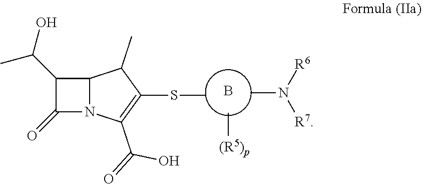

55. The compound of any one of claims 49-54, wherein the compound of Formula (II), or a pharmaceutically acceptable salt, solvate, or stereoisomer thereof, is of Formula (IIb):

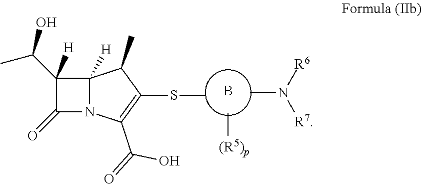

56. The compound of any one of claims 49-55, wherein p is 0.

57. The compound of any one of claims 49-56, wherein R⁷ is C₃-C₈ cycloalkyl or C₂-C₈ heterocycloalkyl; each independently optionally substituted with one, two, or three R^{7a}.

58. The compound of any one of claims 49-57, wherein the compound of Formula (II), or a pharmaceutically acceptable salt, solvate, or stereoisomer thereof, is of Formula (IIc): 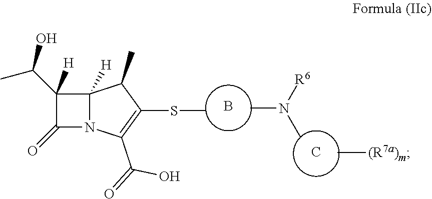
wherein
Ring C is C₃-C₈ cycloalkyl or C₂-C₈ heterocycloalkyl;
each R^{7a} is independently halogen, C₁-C₆ alkyl, C₂-C₆ alkenyl, C₂-C₆ alkynyl, C₁-C₆ haloalkyl, C₁-C₆ hydroxyalkyl, —(CR¹²R¹³)ᵥOR¹⁰, —(CR¹²R¹³)ᵥNR¹⁰R¹¹, —CN, —NO₂, —(CR¹²R¹³)ᵥS(═O)₂R¹⁰, —(CR¹²R¹³)ᵥS(═O)₂NR¹⁰R¹¹, —(CR¹²R¹³)ᵥC(═O)OR¹⁰, —(CR¹²R¹³)ᵥC(═O)NR¹⁰R¹¹, —(CR¹²R¹³)ᵥNR¹⁰C(═NR¹¹)NR¹⁰R¹¹, —(CR¹²R¹³)ᵥC(═NR¹⁰)R¹⁰, —(CR¹²R¹³)ᵥNR¹⁰C(═NR¹¹)R¹⁰, —(CR¹²R¹³)ᵥNR¹⁰(CR¹²R¹³)_{w}NR¹⁰R¹¹, —(CR¹²R¹³)ᵥ(NR¹⁰C(═NR¹¹)NR¹⁰(CR¹²R¹³)_{w}NR¹⁰R¹¹, —(CR¹²R¹³)ᵥC(═NR¹¹)NR¹⁰R¹¹, C₃-C₈ cycloalkyl, C₂-C₈ heterocycloalkyl, aryl, or heteroaryl;
wherein each alkyl, alkenyl, alkynyl, cycloalkyl, heterocycloalkyl, aryl, and heteroaryl is independently optionally substituted with one, two, or three R^{7b};
each R^{7b} is independently halogen, C₁-C₆ alkyl, C₁-C₆ haloalkyl, —OR^{a}, —CN, —NO₂, —NR^{c}R^{d}, —S(═O)₂R^{b}, —S(═O)₂NR^{c}R^{d}, —C(═O)OR^{a}, —C(═O)R^{b}, —C(═O)NR^{c}R^{d}, —NR^{c}C(═NR)NR^{c}R^{d}, or —NR^{c}C(═NR^{c})R^{b};
each R¹⁰ and R¹¹ is independently hydrogen, C₁-C₆ alkyl, C₁-C₆ haloalkyl, C₂-C₆ alkenyl, C₂-C₆ alkynyl, C₃-C₈ cycloalkyl, C₂-C₈ heterocycloalkyl, aryl, heteroaryl, —(C₁-C₆ alkyl)(C₃-C₈ cycloalkyl), —(C₁-C₆ alkyl)(C₂-C₈ heterocycloalkyl), —(C₁-C₆ alkyl)(aryl), or —(C₁-C₆ alkyl)(heteroaryl);
or R¹⁰ and R¹¹ are taken together with the nitrogen to which they are attached to form a C₂-C₈ heterocycloalkyl;
each R¹² and R¹³ is independently hydrogen, halogen, —CN, —OR^{a}, —NR^{c}R^{d}, C₁-C₆ alkyl, C₁-C₆ haloalkyl, C₂-C₆ alkenyl, C₂-C₆ alkynyl, C₃-C₈ cycloalkyl, C₂-C₈ heterocycloalkyl, aryl, or heteroaryl;
v is 0-4;
w is 2-4; and
m is 0-3.

59. The compound of any one of claims 49-58, wherein R⁶ is hydrogen.

60. The compound of any one of claims 49-56, wherein R⁶ and R⁷ are taken together with the nitrogen to which they are attached to form a C₂-C₈ heterocycloalkyl optionally substituted with one, two, or three R^{7a}.

61. The compound of any one of claims 49-56 or 60, wherein the compound of Formula (II), or a pharmaceutically acceptable salt, solvate, or stereoisomer thereof, is of Formula (IId): 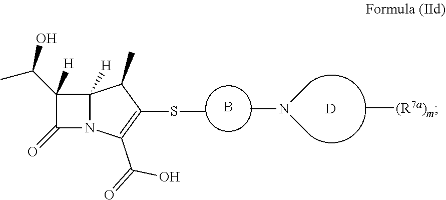
wherein
Ring D is C₂-C₆ heterocycloalkyl;
each R^{7a} is independently halogen, C₁-C₆ alkyl, C₂-C₆ alkenyl, C₂-C₆ alkynyl, C₁-C₆ haloalkyl, C₁-C₆ hydroxyalkyl, —(CR¹²R¹³)ᵥOR¹⁰, —(CR¹²R¹³)ᵥNR¹⁰R¹¹, —CN, —NO₂, —(CR¹²R¹³)ᵥS(═O)₂R¹⁰, —(CR¹²R¹³)ᵥS(═O)₂NR¹⁰R¹¹, —(CR¹²R¹³)ᵥC(═O)OR¹⁰, —(CR¹²R¹³)ᵥC(═O)NR¹⁰R¹¹, —(CR¹²R¹³)ᵥNR¹⁰C(═NR¹¹)NR¹⁰R¹¹, —(CR¹²R¹³)ᵥC(═NR¹⁰)R¹⁰, —(CR¹²R¹³)ᵥNR¹⁰C(═NR¹¹)R¹⁰, —(CR¹²R¹³)ᵥNR¹⁰(CR¹²R¹³)_{w}NR¹⁰R¹¹, —(CR¹²R¹³)ᵥ(NR¹⁰C(═NR¹¹)NR¹⁰(CR¹²R¹³)_{w}NR¹⁰R¹¹, —(CR¹²R¹³)ᵥC(═NR¹¹)NR¹⁰R¹¹, C₃-C₈ cycloalkyl, C₂-C₈ heterocycloalkyl, aryl, or heteroaryl;
wherein each alkyl, alkenyl, alkynyl, cycloalkyl, heterocycloalkyl, aryl, and heteroaryl is independently optionally substituted with one, two, or three R^{7b};
each R^{7b} is independently halogen, C₁-C₆ alkyl, C₁-C₆ haloalkyl, —OR^{a}, —CN, —NO₂, —NR^{c}R^{d}, —S(═O)₂R^{b}, —S(═O)₂NR^{c}R^{d}, —C(═O)OR^{a}, —C(═O)R^{b}, —C(═O)NR^{c}R^{d}, —NR^{c}C(═NR)NR^{c}R^{d}, or —NR^{c}C(═NR^{c})R^{b};
each R¹⁰ and R¹¹ is independently hydrogen, C₁-C₆ alkyl, C₁-C₆ haloalkyl, C₂-C₆ alkenyl, C₂-C₆ alkynyl, C₃-C₈ cycloalkyl, C₂-C₈ heterocycloalkyl, aryl, heteroaryl, —(C₁-C₆ alkyl)(C₃-C₈ cycloalkyl), —(C₁-C₆ alkyl)(C₂-C₈ heterocycloalkyl), —(C₁-C₆ alkyl)(aryl), or —(C₁-C₆ alkyl)(heteroaryl);
or R¹⁰ and R¹¹ are taken together with the nitrogen to which they are attached to form a C₂-C₈ heterocycloalkyl;
each R¹² and R¹³ is independently hydrogen, halogen, —CN, —OR^{a}, —NR^{c}R^{d}, C₁-C₆ alkyl, C₁-C₆ haloalkyl, C₂-C₆ alkenyl, C₂-C₆ alkynyl, C₃-C₈ cycloalkyl, C₂-C₈ heterocycloalkyl, aryl, or heteroaryl;
v is 0-4;
w is 2-4; and
m is 0-3.

62. The compound of claim 61, wherein Ring D is pyrrolidine or piperidine.

63. The compound of claim 61 or 62, wherein Ring D is piperidine.

64. The compound of any one of claims 61-63, wherein m is 0-2.

65. The compound of any one of claims 61-64, wherein m is 0 or 1.

66. The compound of any one of claims 61-65, wherein m is 0.

67. The compound of any one of claims 61-65, wherein m is 1.

68. The compound of any one of claims 49-67, wherein Ring B is cyclobutyl, cyclopentyl, or cyclohexyl.

69. The compound of any one of claims 49-68, wherein Ring B is cyclobutyl.

70. The compound of any one of claims 49-68, wherein Ring B is cyclopentyl.

71. The compound of any one of claims 49-70, wherein each R^{7a} is independently —(CR¹²R¹³)ᵥNR¹⁰R¹¹, —(CR¹²R¹³)'S(═O)₂NR¹⁰R¹¹, —(CR¹²R¹³)ᵥC(═O)NR¹⁰R¹¹, —(CR¹²R¹³)ᵥNR¹⁰C(═NR¹¹)NR¹⁰R¹¹, —(CR¹²R¹³)ᵥC(═NR¹⁰)R¹⁰, —(CR¹²R¹³)ᵥNR¹⁰C(═NR¹¹)R¹⁰, —(CR¹²R¹³)ᵥNR¹⁰(CR¹²R¹³)_{w}NR¹⁰R¹¹, —(CR¹²R¹³)ᵥ(NR¹⁰C(═NR¹¹)NR¹⁰(CR¹²R¹³)_{w}NR¹⁰R¹¹, or —(CR¹²R¹³)ᵥC(═NR¹¹)NR¹⁰R¹¹.

72. The compound of any one of claims 49-71, wherein each R^{7a} is independently —(CR¹²R¹³)ᵥNR¹⁰R¹¹, —(CR¹²R¹³)ᵥNR¹⁰C(═NR¹¹)NR¹⁰R¹¹, —(CR¹²R¹³)ᵥC(═NR¹⁰)R¹⁰, —(CR¹²R¹³)ᵥNR¹⁰C(═NR¹¹)R¹⁰, —(CR¹²R¹³)ᵥNR¹⁰(CR¹²R¹³)_{w}NR¹⁰R¹¹, —(CR¹²R¹³)ᵥ(NR¹⁰C(═NR¹¹)NR¹⁰(CR¹²R¹³)_{w}NR¹⁰R¹¹, or —(CR¹²R¹³)ᵥC(═NR¹¹)NR¹⁰R¹¹.

73. The compound of any one of claims 49-72, wherein each R^{7a} is independently —(CR¹²R¹³)ᵥNR¹⁰R¹¹, —(CR¹²R¹³)ᵥNR¹⁰C(═NR¹¹)NR¹⁰R¹¹, —(CR¹²R¹³)ᵥC(═NR¹⁰)R¹⁰, —(CR¹²R¹³)ᵥNR¹⁰C(═NR¹¹)R¹⁰, or —(CR¹²R¹³)ᵥC(═NR¹¹)NR¹⁰R¹¹.

74. The compound of any one of claims 49-73, wherein each R^{7a} is independently —(CR¹²R¹³)ᵥNR¹⁰R¹¹, —(CR¹²R¹³)ᵥNR¹⁰C(═NR¹¹)NR¹⁰R¹¹, or —(CR¹²R¹³)ᵥC(═NR¹¹)NR¹⁰R¹¹.

75. The compound of any one of claims 49-74, wherein v is 0 or 1.

76. The compound of any one of claims 49-75, wherein v is 0.

77. The compound of any one of claims 49-76, wherein each R¹⁰ and R¹¹ is independently hydrogen or C₁-C₆ alkyl.

78. The compound of any one of claims 49-77, wherein each R¹⁰ and R¹¹ is hydrogen.

79. The compound of any one of claims 49-78, wherein each R¹² and R¹³ is independently hydrogen, halogen, or C₁-C₆ alkyl.

80. The compound of any one of claims 49-79, wherein each R¹² and R¹³ is hydrogen.

81. The compound of claim 49 selected from the group consisting of: 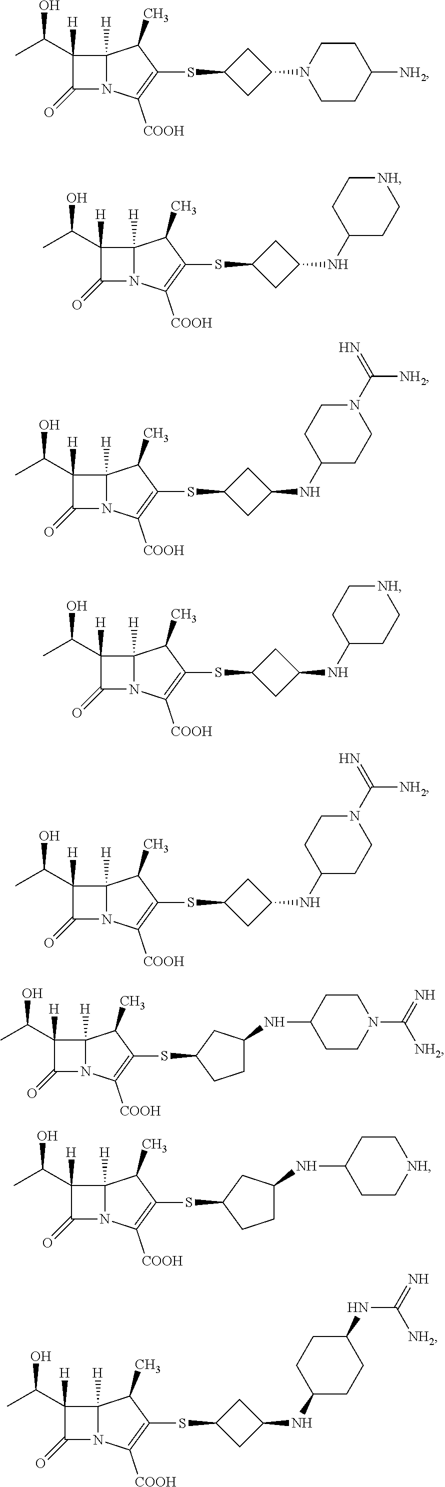 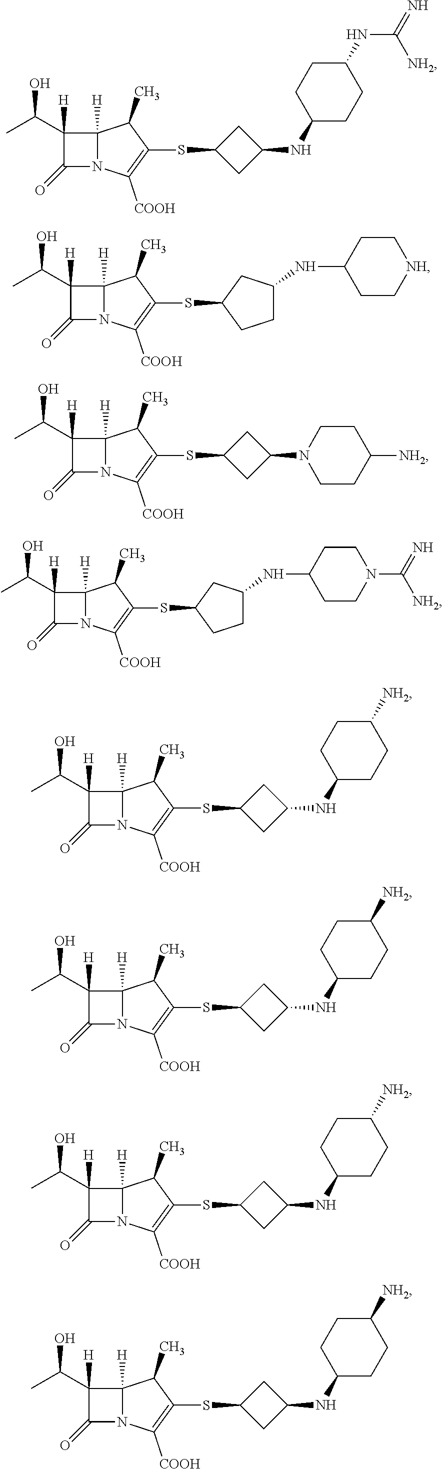 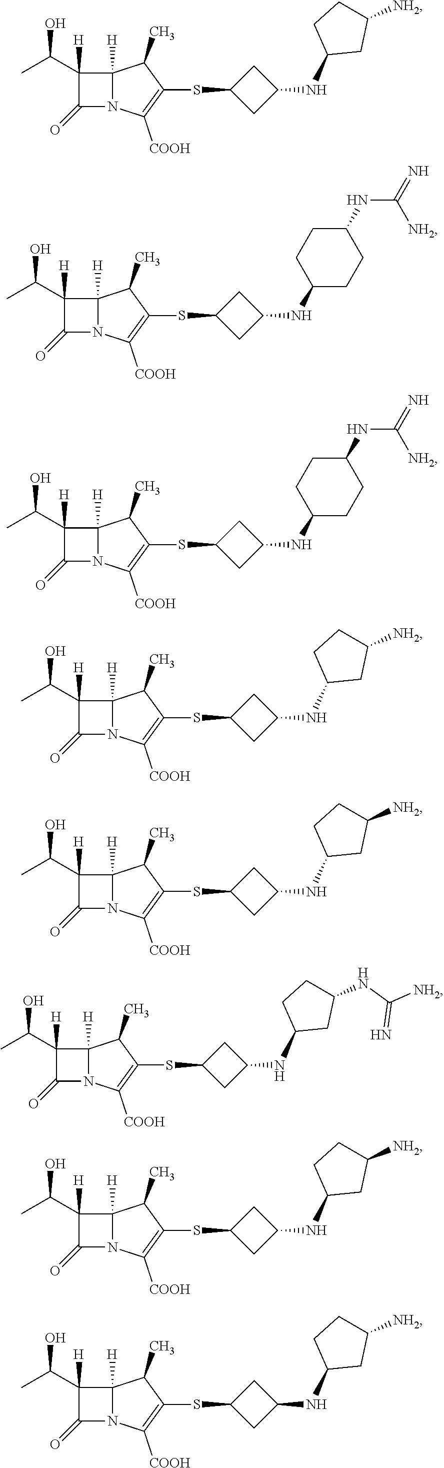 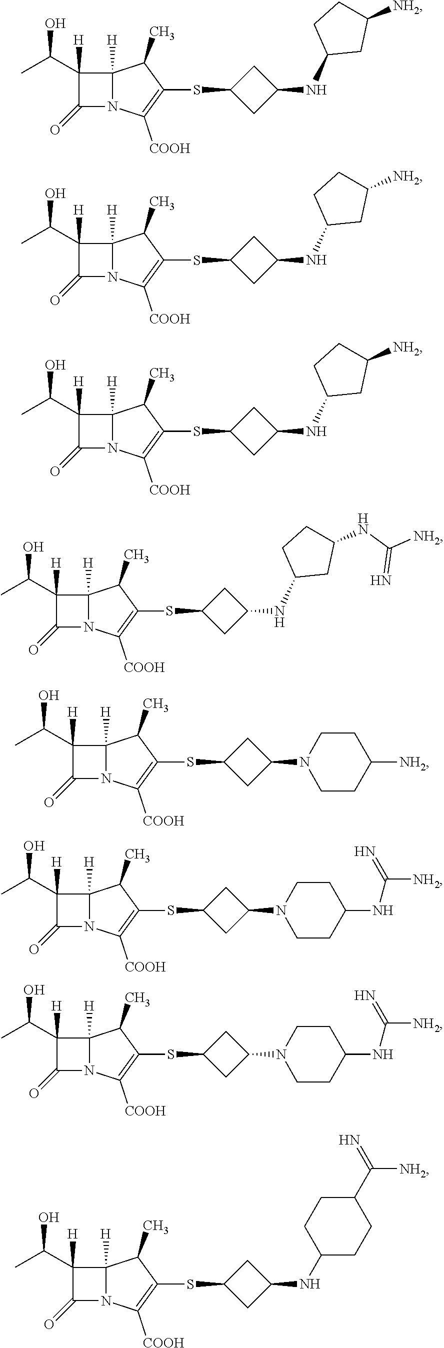 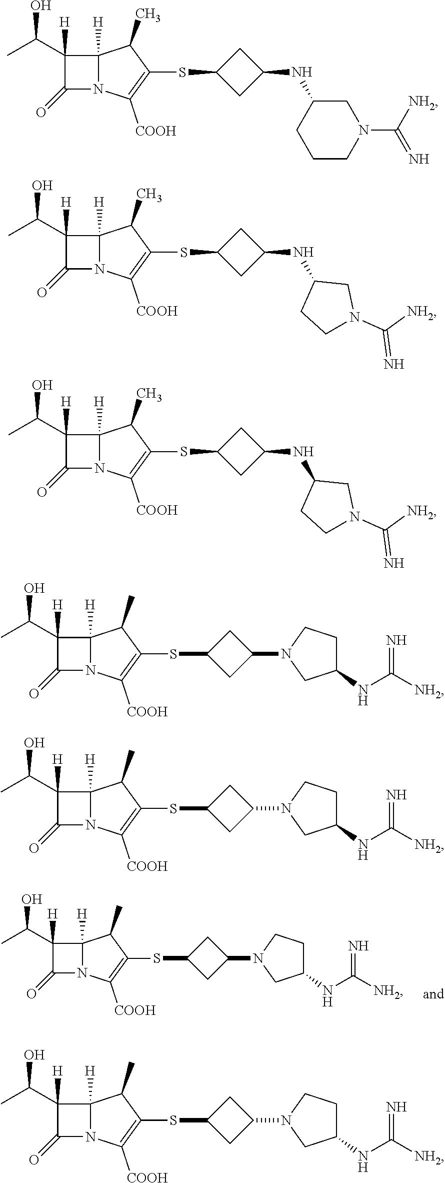
or a pharmaceutically acceptable salt, solvate, or stereoisomer thereof.

82. A compound selected from the group consisting of: 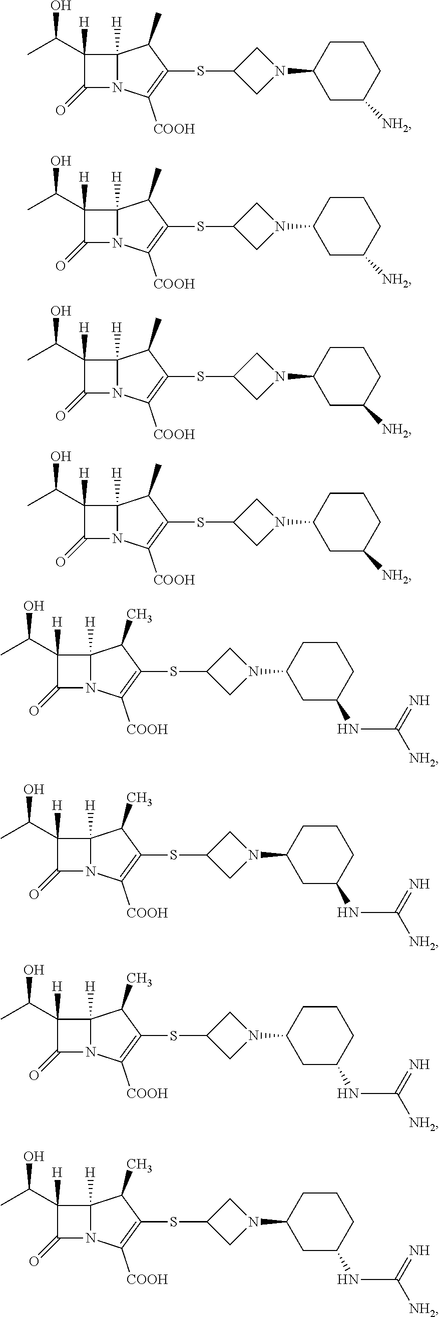 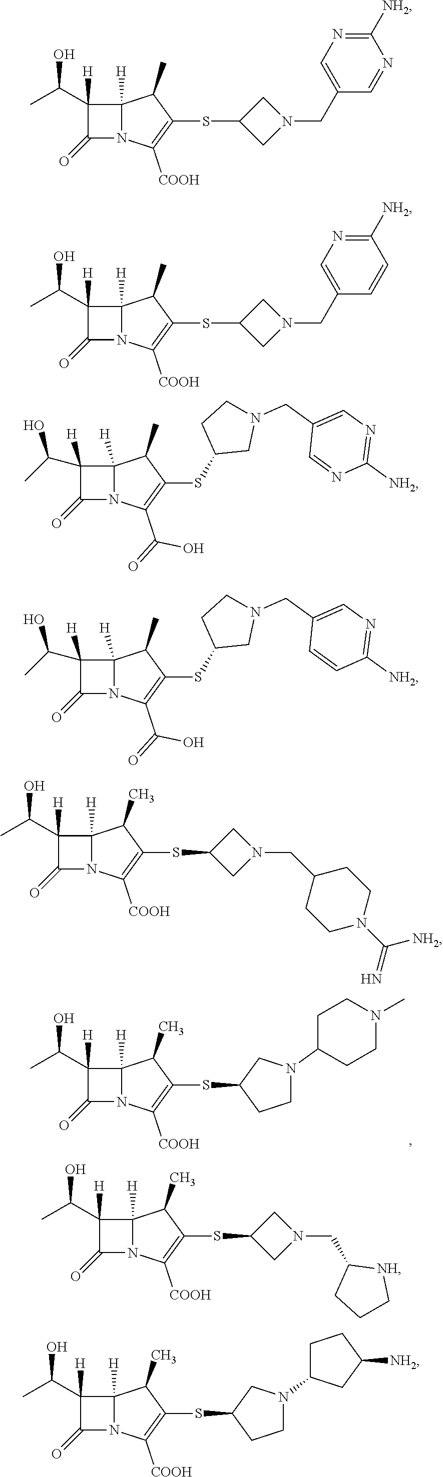 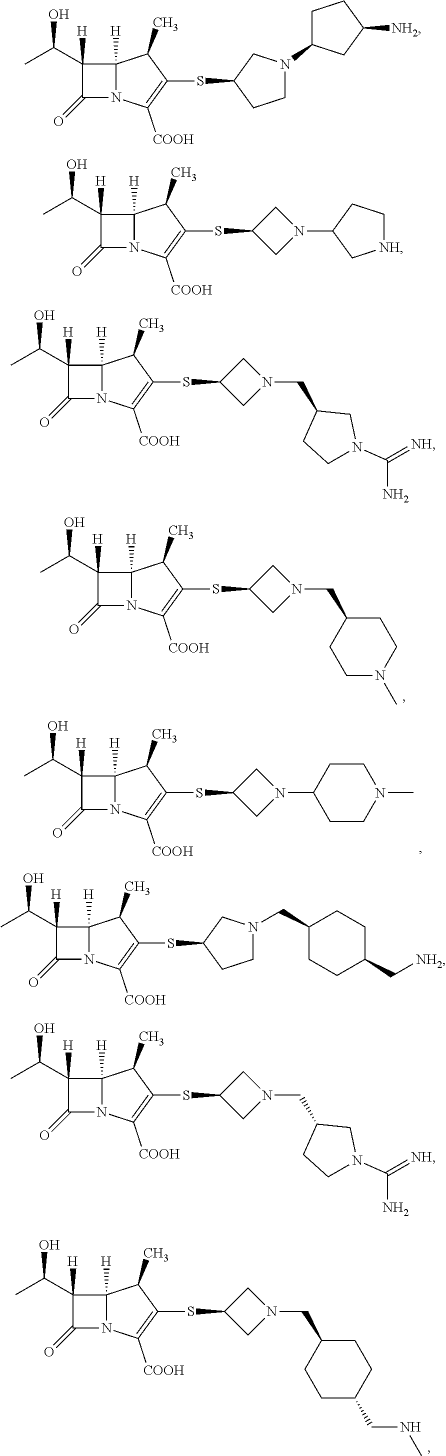 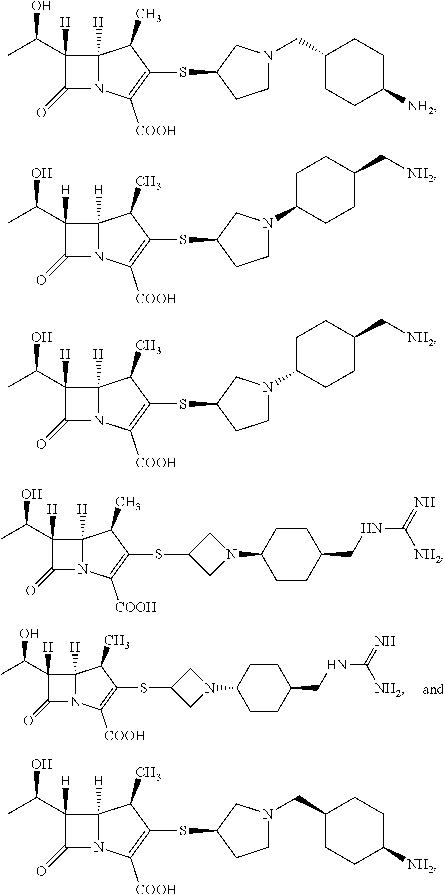
or a pharmaceutically acceptable salt, solvate, or stereoisomer thereof.

83. A pharmaceutical composition comprising a compound of any one of claims 1-82, or a pharmaceutically acceptable salt, solvate, or stereoisomer thereof and a pharmaceutically acceptable excipient.

84. A pharmaceutical composition comprising a compound of any one of claims 1-82, or a pharmaceutically acceptable salt, solvate, or stereoisomer thereof, a β-lactamase inhibitor; and a pharmaceutically acceptable excipient.

85. A method of treating a bacterial infection in a subject, comprising administering to the subject an effective amount of a compound of any one of claims 1-82, or a pharmaceutically acceptable salt, solvate, or stereoisomer thereof.

86. A method of treating a bacterial infection in a subject, comprising administering to the subject an effective amount of a compound of any one of claims 1-82, or a pharmaceutically acceptable salt, solvate, or stereoisomer thereof in combination with a β-lactamase inhibitor.

87. A method of treating a bacterial infection in a subject, comprising administering to the subject a pharmaceutical composition of claim 83.

88. A method of treating a bacterial infection in a subject, comprising administering to the subject a pharmaceutical composition of claim 84.

89. The method of any one of claims 85-88, wherein the bacterial infection is caused by gram-negative bacteria.

90. The method of any one of claims 85-88, wherein the bacterial infection is caused by gram-positive bacteria.

91. The method of any one of claims 85-88, wherein the bacterial infection is caused by multidrug-resistant (MDR) bacteria.

92. The method of any one of claims 85-88, wherein the bacterial infection is cause by carbapenem resistant Enterobacteriaceae (CRE).
